(19) United States
(12) Patent Application Publication
(10) Pub. No.: US 2024/0101646 A1
(43) Pub. Date: Mar. 28, 2024

(54) SARS-COV-2 CORONAVIRUS ANTIBODIES AND USES THEREOF

(71) Applicant: VANDERBILT UNIVERSITY, Nashville, TN (US)

(72) Inventors: Ivelin Stefanov GEORGIEV, Nashville, TN (US); Andrea R. SHIAKOLAS, Nashville, TN (US); Kevin J. KRAMER, Nashville, TN (US)

(21) Appl. No.: 18/273,625

(22) PCT Filed: Jan. 21, 2022
(86) PCT No.: PCT/US2022/013291

§ 371 Date: Jul. 21, 2023

Related U.S. Application Data
(60) Provisional application No. 63/140,379, filed on Jan. 22, 2021.
(60) Provisional application No. 63/165,860, filed on Mar. 25, 2021.
(60) Provisional application No. 63/172,981, filed on Apr. 9, 2021.
(60) Provisional application No. 63/175,243, filed on Apr. 15, 2021.
(60) Provisional application No. 63/195,789, filed on Jun. 2, 2021.
(60) Provisional application No. 63/299,605, filed on Jan. 14, 2022.

(51) Int. Cl. C07K 16/10; A61P 31/14

(52) U.S. Cl. CPC C07K 16/1003; A61P 31/14; C07K 2317/76

(57) ABSTRACT

The present disclosure relates to antibodies and uses thereof for treating, preventing, and detecting coronavirus infection.

### CROSS REFERENCE TO RELATED APPLICATIONS

This application claims the priority benefit of U.S. Provisional Application No. 63/140,379, filed Jan. 22, 2021, U.S. Provisional Application No. 63/165,860, filed Mar. 25, 2021, U.S. Provisional Application No. 63/172,981, filed Apr. 9, 2021, U.S. Provisional Application No. 63/175,243, filed Apr. 15, 2021, U.S. Provisional Application No. 63/195,789, filed Jun. 2, 2021, and U.S. Provisional Application No. 63/299,605, filed Jan. 14, 2022, which are expressly incorporated herein by reference in their entireties.

### STATEMENT REGARDING FEDERALLY SPONSORED RESEARCH

This invention was made with government support under Grant No. 3 RO1 AI131722-04S1 by the National Institutes of Health. The government has certain rights in the invention.

### FIELD

The present disclosure relates to antibodies and uses thereof for treating, preventing, and detecting coronavirus infection.

### BACKGROUND

SARS-CoV-2, or the 2019 novel coronavirus (COVID-19), is a significant pandemic threat that has resulted in over 96,000,000 diagnosed cases including over 2,000,000 deaths as of Jan. 19, 2021. Initially detected in Wuhan, China, human-human transmission has resulted in confirmed cases all over the world. On Jan. 30, 2020, the World Health Organization declared a Public Health of International Concern due to the COVID-19 outbreak and pronounced it a global pandemic on Mar. 12, 2020. The development of preventive and therapeutic measures that can counteract the ongoing, and any future, coronavirus pandemics is therefore of utmost significance for public health worldwide. What is needed are novel compositions and methods for preventing, treating, and diagnosing SARS-CoV-2 infection.

### SUMMARY

Disclosed herein are recombinant antibodies and uses thereof for preventing, treating, and detecting coronavirus infection. Antibody sequences were obtained from an individual previously infected with a SARS-CoV-2 infection.

In some aspects, disclosed herein is a recombinant antibody, wherein the antibody comprises a light chain variable region (VL) that comprises a light chain complementarity determining region (CDRL)1, CDRL2, and CDRL3 and/or a heavy chain variable region (VH) that comprises a heavy chain complementarity determining region (CDRH)1, CDRH2, and CDRH3, wherein: CDRH3 comprises an amino acid sequence at least 60% identical to SEQ ID NOs: 4969-5796, 13255-14083, 21356-22312, 26193-26205, 26263-26275, or 26289-26318; and CDRL3 comprises an amino acid sequence at least 60% identical to SEQ ID NOs: 7453-8280, 25184-26140, 26232-26244, 26276-26288, or 26319-26348.

In some embodiments, CDRH3 comprises at least one amino acid substitution when compared to SEQ ID NOs: 4969-5796, 13255-14083, 21356-22312, 26193-26205, 26263-26275, or 26289-26318. In some embodiments, CDRL3 comprises at least one amino acid substitution when compared to SEQ ID NOs: 7453-8280, 15742-16570, 25184-26140, 26232-26244, 26276-26288, or 26319-26348.

In some embodiments, CDRH1 comprises an amino acid sequence at least 60% identical to SEQ ID NOs: 3313-4140, 11597-12425, 19442-20398, or 26167-26179; and/or CDRL1 comprises an amino acid sequence at least 60% identical to SEQ ID NOs: 5797-6624, 14084-14912, 23270-24226, or 26206-26218.

In some embodiments, CDRH1 comprises at least one amino acid substitution when compared to SEQ ID NOs: 3313-4140, 11597-12425, 19442-20398, or 26167-26179. In some embodiments, CDRL1 comprises at least one amino acid substitution when compared to SEQ ID NOs: 5797-6624, 14084-14912, 23270-24226, or 26206-26218.

In some embodiments, CDRH2 comprises an amino acid sequence at least 60% identical to SEQ ID NOs: 4141-4968, 12426-13254, 20399-21355, or 26180-26192; and/or CDRL2 comprises an amino acid sequence at least 60% identical to SEQ ID NOs: 6625-7452, 14913-15741, 24227-25183, or 26219-26231.

In some embodiments, CDRH2 comprises at least one amino acid substitution when compared to SEQ ID NOs: 4141-4968, 12426-13254, 20399-21355, or 26180-26192. In some embodiments, CDRL2 comprises at least one amino acid substitution when compared to SEQ ID NOs: 6625-7452, 14913-15741, 24227-25183, or 26219-26231.

In some embodiments, VH comprises an amino acid sequence selected from the group consisting of SEQ ID NOs: 1657-2484, 9939-10767, 18485-19441, and 26141-26153. In some embodiments, VL comprises an amino acid sequence selected from the group consisting of SEQ ID NOs: 2485-3312, 10768-11596, 22313-23269, and 26154-26166.

In some embodiments, the recombinant antibody is selected from Table 1. In some embodiments, the recombinant antibody is selected from Table 2. In some embodiments, the recombinant antibody is selected from Table 3.

In one aspect, disclosed herein is a nucleic acid encoding a recombinant antibody as disclosed herein.

In one aspect, disclosed herein is a recombinant expression cassette or plasmid comprising a sequence to express a recombinant antibody as disclosed herein.

In one aspect, disclosed herein is a host cell comprising an expression cassette or a plasmid as disclosed herein.

In one aspect, disclosed herein is a method of producing an antibody, comprising cultivating or maintaining a host cell under conditions to produce the antibody.

In one aspect, disclosed herein is a method of treating a coronavirus infection in a subject, comprising administering to the subject a therapeutically effective amount of a recombinant antibody as disclosed herein. In some embodiments, the coronavirus is SARS-CoV-2.

In some aspects, disclosed herein is a method for detecting a coronavirus infection in a subject, comprising: providing a biological sample from the subject, and detecting a coronavirus antigen in the biological sample with an antibody that specifically binds to the coronavirus antigen, wherein the antibody is from any aspect as disclosed herein, and wherein the presence of the coronavirus antigen in the biological sample indicates the subject is infected with a coronavirus. In some embodiments, the coronavirus is SARS-CoV-2.

### BRIEF DESCRIPTION OF DRAWINGS

The accompanying figures, which are incorporated in and constitute a part of this specification, illustrate aspects described below.

FIGS. 1A-1B show LIBRA-seq antigen titration for identification of potent antibodies. To create affinity-type measurements and identify high potency antibodies using the LIBRA-seq technology, an antigen screening library containing an antigen titration was applied. Six different amounts of oligo-labeled SARS-CoV-2 S protein were included in a screening library. Antibodies with high affinity for SARS-CoV-2 S showed reactivity for S protein added in lower amounts. FIG. 1A shows a schematic depicting the experimental set up—where a titration of oligo-labeled S protein was added to the antigen library and donor PBMCs were used as the cellular input. After incubation, cells with high affinity for the antigen would have many S proteins bound, including those added in low concentrations. FIG. 1B shows, after single cell processing and sequencing, antigen binding can be assessed bioinformatically and which cells have high LIBRA-seq scores for many or all of the Spike antigens included were determined.

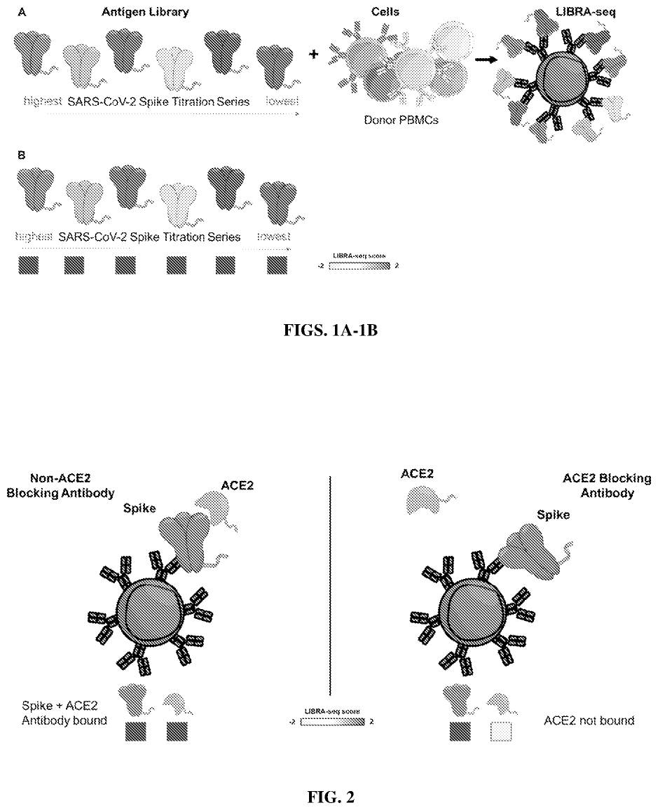

FIG. 2 shows assessment of ligand blocking functionality using LIBRA-seq through identification of ACE2 blocking antibodies. For assessment of ligand blocking functionality using LIBRA-seq, an antigen and its ligand are included in the screening library. If an antibody does not disrupt the interaction between a protein and its receptor, then the LIBRA-seq scores for the protein and the receptor are high (left). If an antibody does block the interaction, then the score for the protein is high and the score for the receptor is low (right). This allows for identification of antibodies that block receptor binding. This can also indicate neutralization potential of the antibodies. This schematic depicts this experimental rationale using SARS-CoV-2 as an example—where oligo labeled spike and oligo-labeled ACE2 (the spike receptor) are included in the antigen screening library.

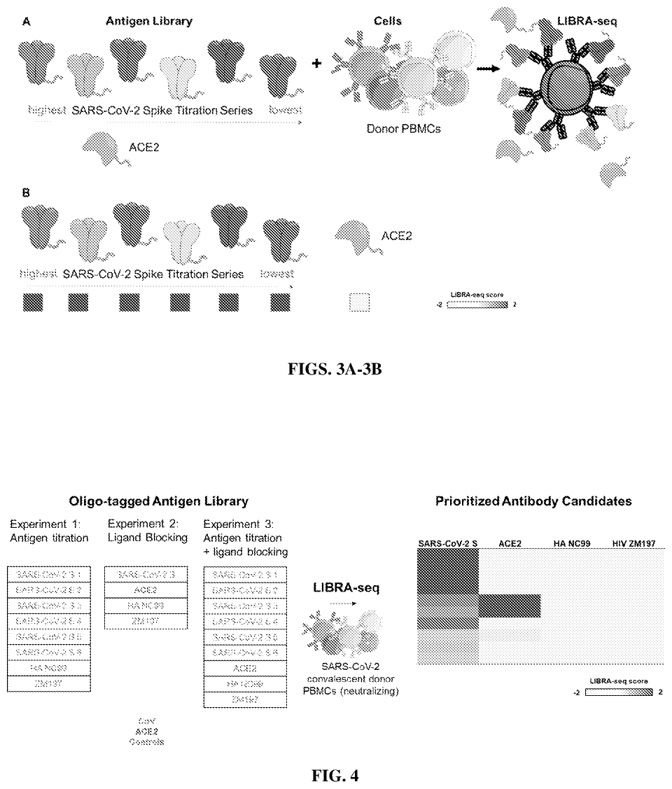

FIGS. 3A-3B show LIBRA-seq antigen titration with ligand blocking for identification of potent antibodies. In this schematic, an antigen titration along with the inclusion of the receptor are included to identify potent antibodies with ligand blocking functionality. FIG. 3A shows schematic depicting the experimental set up—where a titration of oligo-labeled S protein was added to the antigen library along with oligo-labeled ACE2 receptor, and donor PBMCs were used as the cellular input. After incubation, cells with high affinity for the antigen would have many S proteins bound, including those added in low concentrations. Antibodies that can block the receptor-protein interaction would not have ACE2 bound to the spike proteins. Antibodies that do not block the interaction would have ACE2 bound to the spike proteins. FIG. 3B shows, after single cell processing and sequencing, assessment of antigen binding bioinformatically and determination regarding which cells have high LIBRA-seq scores for many or all of the Spike antigens included. Additionally, which cells do or do not have ACE2 bound can be determined. In this example, ACE2 is not bound to spike and therefore has a low LIBRA-seq score, indicating that the antibody is able to block ligand binding.

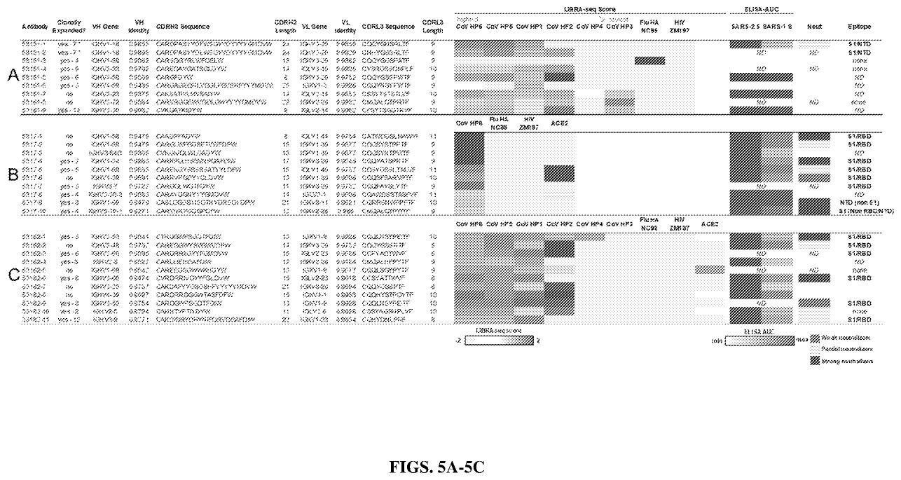

FIG. 4 shows extending LIBRA-seq technology for identification of potent SARS-CoV-2 antibodies. To assess affinity measurements and ligand blocking functionality, three LIBRA-seq experiments were performed. To assess affinity measurements, in experiment 1, the antigen library consisted of an antigen titration of SARS-CoV-2 S protein along with control antigens influenza HA NC99 and HIV ZM197. To assess ligand blocking, in experiment 2, the antigen library consisted of SARS-CoV-2 S protein along with its receptor, ACE2, and control antigens influenza HA NC99 and HIV ZM197. To assess affinity measurements in combination with ligand blocking, in experiment 3, the antigen library consisted of an antigen titration of SARS-CoV-2 S protein, ACE2, and control antigens influenza HA NC99 and HIV ZM197. Each antigen library was incubated with SARS-CoV-2 convalescent donor PBMCs and LIBRA-seq was performed. After single cell processing, next generation sequencing, and bioinformatic analysis, antibody heavy chain and light chain sequence features and antigen LIBRA-seq scores for thousands of cells were assessed. For the antigen titration experiments, antibodies that showed high scores for S protein added in lower amounts were identified. For ligand blocking, antibodies that had high scores for S protein and low scores for ACE2 were identified—showing ligand blocking functionality of these antibodies. Antibodies were prioritized for expression and further testing based on these features (see FIG. 5).

FIGS. 5A-5C show LIBRA-seq enabled prioritization of antibodies with diverse sequence features and functional profiles using antigen titration and ligand blocking features. As described in FIG. 4, three experiments were performed to assess affinity measurements and ligand blocking in the context of SARS-CoV-2. Antibodies were prioritized for expression and characterization utilizing the genetic features of the heavy and light chain sequences (including clonal expansion, VH gene usage, VH identity, CDRH3 sequence and sequence length, VL gene usage, VL identity, CDRL3 sequence and sequence length) and the LIBRA-seq scores for the antigens used in each library. For each experiment, select prioritized antibodies are shown, with their genetic features and LIBRA-seq scores. Each row represents an antibody. LIBRA-seq scores for each antigen in the library are displayed as a heatmap, with LIBRA-seq score of −2 displayed as tan, a score of 0 displayed as white, and a score of 2 displayed as purple These antibodies were expressed, purified, and characterized for binding to SARS-CoV-2 S and SARS-CoV-1 S (shown as ELISA area under the curve (AUC)), and neutralization of SARS-CoV-2. ELISA binding data against the antigens are displayed as a heatmap of the AUC analysis, with AUC of 0 displayed as white, and maximum AUC as purple. Neutralization is shown as weak, partial or strong, as green, yellow and red respectively. Non-neutralizing antibodies are listed as white. Additionally, epitope mapping was performed by testing binding to a variety of S protein subdomains, and determined epitopes are listed. ND stands for not done. HP stands for hexapro and represents the SARS-CoV-2 hexapro S variant that was used in the screening library. FIG. 5A shows that nine antibodies were prioritized and tested from experiment 1 (assessment of affinity measurements using antigen titration). FIG. 5B shows that ten antibodies were prioritized and tested from experiment 2 (assessment of ligand blocking). FIG. 5C shows that eleven antibodies were prioritized and tested from experiment 3 (assessment of affinity measurements combined with ligand blocking). In addition to the select antibodies highlighted here, there are thousands of other antibodies present in the datasets. The sequences in FIG. 5A are CARDPASYYDFWSGYVDYYYYGMDVW (SEQ ID NO: 1), CARDPASYYDLWSGYVDYYYYGMDVW (SEQ ID NO: 2), CARSGGYRLWFGELW (SEQ ID NO: 3), CAREGAVGATSGLDYW (SEQ ID NO: 4), CARGFDYW (SEQ ID NO: 5), CARGAGEQRLVGGLFGVSHFYYYMDVW (SEQ ID NO: 6), CAKSATIVLMVSAIYW (SEQ ID NO: 7), CARVRGGEWVGDLGWYYYYGMDVW (SEQ ID NO: 8), CVKGATKIDYW (SEQ ID NO: 9), CQQYGNSRLTF (SEQ ID NO: 10), CHHYGSSRLTF (SEQ ID NO: 11), CQQYGGSPATF (SEQ ID NO: 12), CYSRDSSGNPLF (SEQ ID NO: 13), CQQYGSSPWTF (SEQ ID NO: 14), CQQYNSYPWTF (SEQ ID NO: 15), CSSYTSTSTLVF (SEQ ID NO: 16), CMQALQTPRTF (SEQ ID NO: 17), CFSYTSGGTRVF (SEQ ID NO: 18). The sequences in FIG. 5B are CAADPFADYW (SEQ ID NO: 19), CARGLWFGDSETVWFDPW (SEQ ID NO: 20), CVKGKIQLWLGADYW (SEQ ID NO: 21), CARKPLLHSSVNPGAFDIW (SEQ ID NO: 22), CAREKGYSSSSSATYYLDFW (SEQ ID NO: 23), CARRVPGDYYCLDVW (SEQ ID NO: 24), CARGGLWGTFDYW (SEQ ID NO: 25), CARAYGGNYYYGMDVW (SEQ ID NO: 26), CASLGGDSYISGTHYDRSGYDPW (SEQ ID NO: 27), CARVNRVGDGPDFW (SEQ ID NO: 28), CATWDDSLNAWVF (SEQ ID NO: 29), CQQSYSTPPTF (SEQ ID NO: 30), CQQSYNTPWTF (SEQ ID NO: 31), CQQYATSPRTF (SEQ ID NO: 32), CQSYDSSLTALVF (SEQ ID NO: 33), CQQSFSARVPTF (SEQ ID NO: 34), CQQFAYSLYTF (SEQ ID NO: 35), CQAWDSSTASFVF (SEQ ID NO: 36), CQRRSNWPPFTF (SEQ ID NO: 37), CMQALQTPWTF (SEQ ID NO: 38). Sequences in FIG. 5C shows CTRGGWPSGDTFDIW (SEQ ID NO: 39), CAREGGWYSVGWVDPW (SEQ ID NO: 40), CARDRRIIGYYFGMDVW (SEQ ID NO 41), CARLLIEHDAFDIW (SEQ ID NO: 42), CAREEGSGWWKHDYW (SEQ ID NO: 43), CVRDRRIVGYYFGLDVW (SEQ ID NO: 44), CAKDAFYYGSGSHFYYYYYMDVW (SEQ ID NO: 45), CARDRRGGGWTASFDFW (SEQ ID NO: 46), CARGGWPSGDTFDIW (SEQ ID NO: 47), CAHHTVPTIYDYW (SEQ ID NO: 48), CAKDIGRYDHYNIFGRVGGAFDIW (SEQ ID NO: 49), CQQYGSSRTF (SEQ ID NO: 50), CCPYADTWVF (SEQ ID NO: 51), CMQALHFPYTF (SEQ ID NO: 52), CQQLSGYPYTF (SEQ ID NO: 53), CCSYATTWVF (SEQ ID NO: 54), CQQYGSSPTF (SEQ ID NO: 55), CQQHYSTPGYTF (SEQ ID NO: 56), CQQLNSYPEITF (SEQ ID NO: 57), CSSYAGSNPLVF (SEQ ID NO: 58), CQHYDNLPRF (SEQ ID NO: 59),

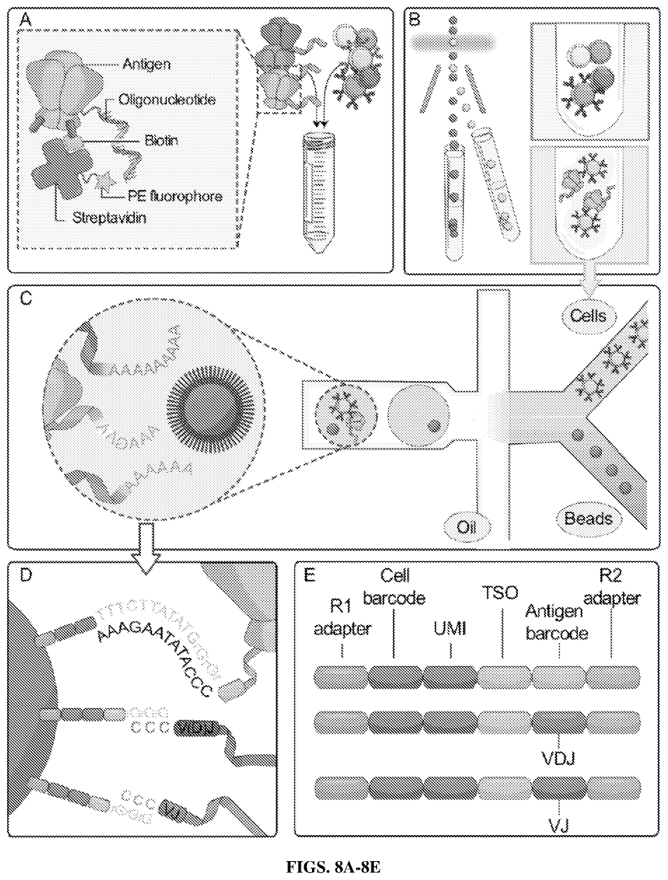

FIGS. 6A-6C show identification of SARS-CoV-2 antibodies using LIBRA-seq antigen titration. Utilizing an antigen titration can lead to affinity-type measurements. By plotting the LIBRA-seq score for the S antigens against the amounts of antigen that were added to the library, a representative “binding curve” is created. FIG. 6A shows, from experiment 1 (assessment of affinity measurements using antigen titration), LIBRA-seq scores for one antibody identified from the SARS-CoV-2 convalescent sample using this method. FIG. 6B shows that these scores are plotted against the antigen amounts utilized in the screening library for the titration. FIG. 6C shows comparison of this example antibody (shown in black) compared a selection of other antibodies (colors) identified from this donor. There are a variety of LIBRA-seq score binding curves that can be used to estimate antigen affinity. Other measurements can be estimated from these curves, like EC50 for example.

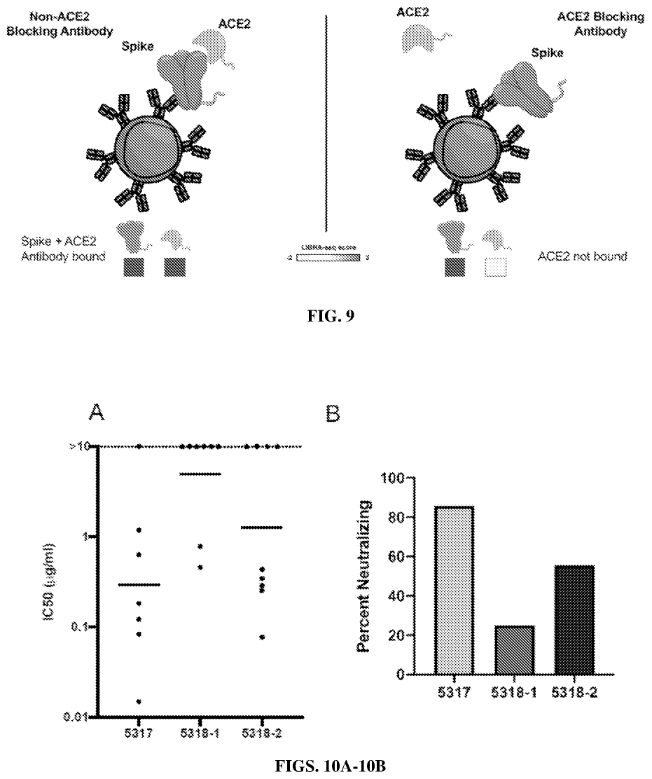

FIG. 7 shows SARS-CoV-2 S titration with ligand blocking for identification of potent antibodies. For experiment 3 (assessment of affinity measurements combined with ligand blocking), all cells identified from the experiment are shown as dots, with LIBRA-seq score for ACE2 on the y-axis and LIBRA-seq Score for SARS-CoV-2 S on the X-axis. Each plot shows the LIBRA-seq scores for one of the SARS-CoV-2 S titration amounts added. These plots are shown from high to low, left to right respectively. With these plots, a SARS-CoV-2 S and ACE2 double positive population (shown with an arrow) can be identified, along with a SARS-CoV-2 S positive/ACE2 negative population (shown with an arrow). This population represents cells that have ligand blocking functionality. Further, since a titration of Spike was included, cells that show high scores for spike added in lower amounts and are also negative for ACE2 can be identified (shown in red circle). This population of cells can be highly potent, ACE2 blocking antibodies.

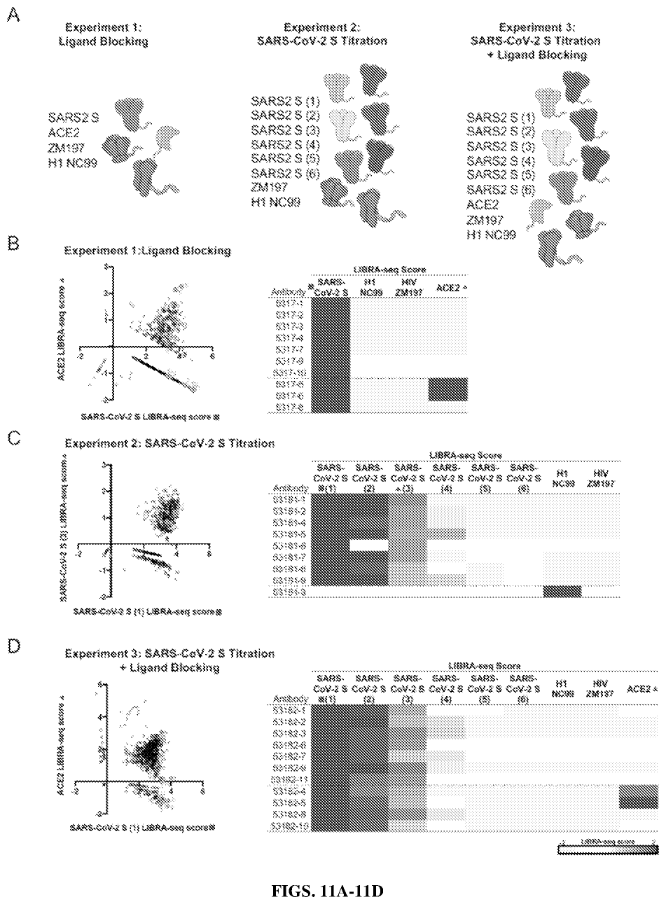

FIGS. 8A-8E show LIBRA-seq assay schematic. The assay consists of the following general steps: FIG. 8A. Antigens are recombinantly produced, biotinylated, and labeled with a DNA “barcode” oligonucleotide. The DNA-barcoded antigens are mixed with cells of interest and labeled with streptavidin fluorophores. FIG. 8B. Antigen positive B cells are bulk sorted and diluted to an appropriate concentration for single cell sequencing. FIG. 8C. Using the 10× Chromium controller, each cell (along with its bound antigens) is isolated in a single cell emulsion droplet along with a bead that has primers for downstream library preparation. FIG. 8D. Bead delivered oligos index both cellular BCR transcripts and antigen barcodes during reverse transcription. FIG. 8E. Library preparation results in amplification of transcripts for each cell that are indexed with the same cell barcode to enable direct mapping of BCR sequence to antigen specificity.

FIG. 9 shows LIBRA-seq with ligand blocking applied to a SARS-CoV-2 convalescent donor sample. An antigen screening library of oligonucleotide-labeled antigens was generated. This consisted of CoV antigens SARS-CoV-2 spike and negative controls. Additionally, oligolabeled ACE2 (the SARS-CoV-2 spike receptor) was also included. This allowed for assessment of ligand blocking functionality from the sequencing experiment. The antigen screening library was mixed with the donor PBMCs, and the LIBRA-seq workflow was executed.

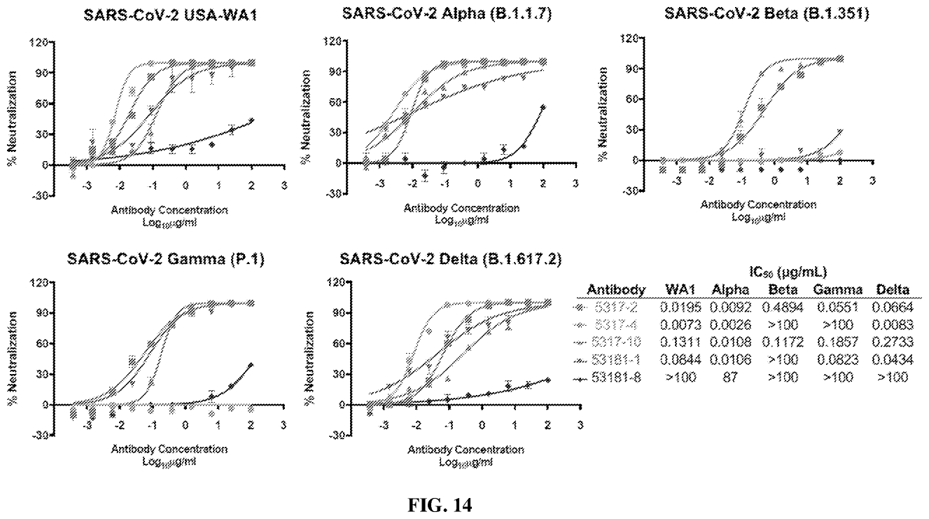

FIGS. 10A-10B show that LIBRA-seq with ligand blocking confirms predicted SARS-CoV-2 neutralization by antibodies at high rates. FIG. 10A. IC50 values (ug/ml) for SARS-CoV-2 neutralization by real time cell analysis (RTCA) with VSV-SARS-CoV-2. Line shown is geometric mean. Non-neutralizing antibodies are shown as >10 ug/ml. FIG. 10B. Percent of confirmed predicted neutralizers (shown in FIG. 12D) are shown. 85.7% of predicted neutralizers were confirmed for 5317 experiment (LIBRA-seq with ligand blocking), whereas 22.2% of antibodies predicted to bind SARS-CoV-2 were neutralizing when no ACE2 was included, for experiment 5318-1. Furthermore, the two neutralizing antibodies were clonally related. 45.4% of predicted neutralizers were confirmed for 5318-2.

FIGS. 11A-11D show antibody discovery using LIBRA-seq with ligand blocking. FIG. 11A shows experimental setup of three LIBRA-seq experiments: experiment 1, LIBRA-seq with ligand blocking; experiment 2, LIBRA-seq with a SARS-CoV-2 S titration; and experiment 3, LIBRA-seq with a SARS-CoV-2 S titration and ligand blocking. For experiment 2 and 3, six different aliquots of S protein were added in a titration series (1-6). (FIGS. 11B-11D) (left) After next-generation sequencing, hundreds of B cells (dots) were recovered that had paired heavy/light chain sequencing information and antigen reactivity information for the three experiments. For experiment 1 (FIG. 11B), 2 (FIG. 11C), and 3 (FIG. 11D), select LIBRA-seq scores for all cells per experiment are shown as open circles (n=828, 829, 957, respectively). Antibodies selected for expression and validation are highlighted and numbered in light blue. (right) LIBRA-seq scores for the selected antibodies for all antigens from each experiment are shown as a heatmap from −2 to 2 (tan to purple); scores outside of this range are shown as the minimum and maximum values. For experiments 1 and 3, antibodies with negative scores for ACE2 are shown above the dotted line while antibodies with positive scores for ACE2 are shown below the dotted line and are controls. For experiment 2, all SARS-CoV-2 reactive antibodies are shown above the dotted line, whereas influenza specific antibody 53181-3 is shown as a control below the dotted line.

FIGS. 12A-12D show validation and characterization of selected antibodies. FIG. 12A. ELISA area under the curve (AUC) values for binding to SARS-CoV-2 recombinant antigen proteins and a negative control influenza hemagglutinin protein are shown for antibodies (rows) in each experiment, calculated from data in FIG. 18B. FIG. 12B. K_{D} (M) of antibodies for SARS-CoV-2 RBD or NTD (based on epitope shown in FIG. 14A) was determined by biolayer interferometry. ND, not done. FIG. 12C. Percent reduction in ACE2 binding by ELISA is shown as a heatmap from 0 to 100% (white to blue) reduction in binding compared to SARS-CoV-2 binding only. FIG. 12D. VSV SARS-CoV-2 neutralization IC₅₀ values are shown as a heatmap from high potency (red) to low potency (green). Non-neutralizing antibodies are shown as white.

FIGS. 13A-13C show assessment of LIBRA-seq with ligand blocking. FIG. 13A. “Predicted Neutralizing Antibodies” were defined as the subset of selected antibodies with negative ACE2 LIBRA-seq scores from experiments 1 (n=7 antibodies) and 3 (n=6 antibodies), and all antibodies with high LIBRA-seq scores (>1) for SARS-CoV-2 S from experiment 2 (n=7 antibodies). The percent of neutralizing antibodies from the set of predicted neutralizers is shown for each experiment. FIG. 13B. The IC₅₀ values (μg/mL) for SARS-CoV-2 neutralization by RTCA with VSV-SARS-CoV-2 (IC₅₀ value for each antibody shown as single dot) are plotted for the set of predicted neutralizers. Horizontal line shown is geometric mean for each experiment. Non-neutralizing antibodies are shown as >10 μg/mL. FIG. 13C. Spearman correlation of ACE2 LIBRA-seq score (x-axis) and % Reduction in ACE2 Binding to SARS-CoV-2 (y-axis) for antibodies from experiments 1 and 3. Spearman r=−0.54, p=0.017 (two-tailed, 95% confidence interval).

FIG. 14 shows antibody neutralization of SARS-CoV-2 variants. Authentic SARS-CoV-2 neutralization for a panel of antibodies is shown against USA-WA1 and variants (Alpha, Beta, Gamma, and Delta). Data represent the % neutralization as mean±SD. The IC₅₀ values calculated in GraphPad prism software by 4-parameter best-fit analysis are shown to the right of the panel.

FIGS. 15A-15B show structural characterization of antibodies 5317-4 and 5317-10. FIG. 15A. 9 Å-resolution cryo-EM structure of Fab-spike complex for 5317-4 Fab (orange) and 5317-10 Fab (pink). Spike protomers are shown in green, blue, and red. FIG. 15B. Fab-spike complex structure modeled with ACE2 (purple).

FIGS. 16A-16H show discovery of cross-reactive ACE2-blocking coronavirus antibodies using LIBRA-seq with ligand blocking. FIG. 16A. Schematic of LIBRA-seq with ligand blocking applied to cross-reactive antibody discovery. FIG. 16B. For identification of cross-reactive coronavirus antibodies with ligand blocking capability, all IgGs recovered from the LIBRA-seq experiment (n=2569) are shown, with LIBRA-seq scores for SARS-CoV (x-axis) and SARS-CoV-2 (y-axis). Each dot represents a cell, and the color of the dots shows the ACE2 LIBRA-seq score, with color heatmap shown on the right. FIG. 16C. Cells selected for expression and validation are shown in blue (ACE2 score <−1) or grey (ACE2 score ≥−1). Of these selected cells, 8 had high LIBRA-seq scores (>1) for SARS-CoV-2 and SARS-CoV and low scores (<−1) for ACE2. Additional candidates with a variety of scores for SARS-CoV-2, SARS-CoV and ACE2 were also selected for expression and validation as controls. FIG. 16D. The 8 IgGs with high LIBRA-seq scores for SARS-CoV-2 and SARS-CoV and low scores for ACE2 are shown above the dotted line. Control antibodies with other LIBRA-seq score patterns are shown below the dotted line. For each antibody, CDR sequences and lengths are shown at the amino acid level and V-gene and J-gene identity are shown at the nucleotide level. LIBRA-seq scores for antigens included in the screening library (SARS-CoV-2 spike, SARS-CoV spike, ACE2, HIV ZM197 Env, influenza hemagglutinin H1 NC99) are shown as a heatmap low (tan)-white-high (purple). Scores outside of this range are shown as the minimum and maximum values. FIG. 16E. ELISA area under the curve (AUC) values from binding to coronavirus spike proteins, influenza hemagglutinin H1 NC99 (negative control), and FIG. 16F. recombinant antigen domains, are shown as a heatmap from minimum (white) to maximum (purple) binding. FIG. 16G. Percent reduction in ACE2 binding by ELISA is shown for SARS-CoV-2 and SARS-CoV spikes, and displayed as a heatmap from 0% (white) to 100% (blue). FIG. 16H. For the 8 IgGs with high LIBRA-seq scores for SARS-CoV-2 and SARS-CoV and low scores for ACE2, the percent reduction in ACE2 binding due to antibody blocking by ELISA is shown for SARS-CoV (x-axis) and SARS-CoV-2 (y-axis). The sequences in FIG. 16D include CARYTSYYDRSGFRRVEYFQHW (SEQ ID NO: 26263), CANMRTNYDIFTGYYPDAFDIW (SEQ ID NO: 26264), CARDVTHAFDLW (SEQ ID NO: 26265), CAKEGARGRGATTSFYYYYMDVW (SEQ ID NO: 26266), CARSTYYYDRSGYSTSDGMDVW (SEQ ID NO: 26267), CAREYSSTVWDNW (SEQ ID NO: 26268), CARPPRGYYDRTGYYNVVHYFQHW (SEQ ID NO: 26269), CARPPRGYYDRSGYYNVLLYFQHW (SEQ ID NO: 26270), CAKSEYSYAYKVHFLDYW (SEQ ID NO: 26271), CAREDTFYFDYW (SEQ ID NO: 26272), CARGGFNYGHGLDYW (SEQ ID NO: 26273), CAKYGWGLLAAAGDAFDIW (SEQ ID NO: 26274), CARSGSYGDRTFDHW (SEQ ID NO: 26275), CQQYGSSPYTF (SEQ ID NO: 26276), CQQYYNWPPWTF (SEQ ID NO: 26277), CQQYNSDLYTF (SEQ ID NO: 26278), CQSYDISLNGWVL (SEQ ID NO: 26279), CQQYGSSPLTF (SEQ ID NO: 26280), CSSYTSSSAYVVF (SEQ ID NO: 26281), CQQYDNLSLTF (SEQ ID NO: 26282), CQQYVNLPLTF (SEQ ID NO: 26283), CQSYDSSNHVLF (SEQ ID NO: 26284), CQQYGTSPSF (SEQ ID NO: 26285), CSSYAGVTNNLIF (SEQ ID NO: 26286), CMQGTHWPRTF (SEQ ID NO: 26287), and CQAWGSSTAVF (SEQ ID NO: 26288).

FIGS. 17A-17C show a schematic representation of LIBRA-seq experiments. FIG. 17A. An antigen screening library of oligonucleotide-labeled antigens was generated. This library consisted of SARS-CoV-2 spike antigens and negative controls. Additionally, oligo-labeled ACE2 (the SARS-CoV-2 spike host cell receptor) was included. The antigen screening library was mixed with donor PBMCs. This approach allowed for assessment of B cell ligand blocking functionality from the sequencing experiment. FIG. 17B. An antigen screening library containing an antigen titration was generated, with a goal of identifying high affinity antibodies from LIBRA-seq. In this experiment, six different amounts of oligo-labeled SARS-CoV-2 S protein, each labeled with a different barcode, were included in a screening library. FIG. 17C. Schematic of LIBRA-seq with S titrations and ACE2 included for ligand blocking.

FIGS. 18A-18D show characterization of LIBRA-seq identified antibodies. FIG. 18A. Genetic characteristics for monoclonal antibodies prioritized for expression and validation. VH, JH, VL, JL inferred gene segment identity is shown at the nucleotide level. CDRH3 and CDRL3 amino acid sequence and length are also shown. FIG. 18B. ELISA binding of antibodies to SARS-CoV-2 spike, SARS-CoV-2 S1, SARS-CoV-2 RBD, SARS-CoV-2 NTD, SARS-CoV-2 S2 and influenza hemagglutinin H1 NC99. Data are represented as mean±SEM of technical duplicates and represent one of at least two independent experiments (n=2). FIG. 18C. ACE2 blocking ELISA. Antibodies were added to spike, and recombinant ACE2 was added and detected. Antibodies that block ACE2 binding show a reduction in absorbance compared to ACE2 binding without competitor (dotted line). ELISAs were performed at one antibody concentration, and data are represented as mean±SEM of technical triplicates and represent one of at least two independent experiments (n=2). FIG. 18D. Antibodies were tested in a VSV SARS-CoV-2 real time cell analysis (RTCA) neutralization assay. Neutralization curves and IC50 values are shown. Data are represented as mean±S.D. of technical triplicates, and represent one of two independent experiments (n=2).

FIGS. 19A-19C show characterization of selected cross-reactive antibodies. FIG. 19A. For the IgGs that showed high LIBRA-seq scores (>1) for both SARS-CoV-2 and SARS-CoV, the percent of cells with low ACE2 scores (<−1) is shown. FIG. 19B. ELISA binding of antibodies to SARS-CoV-2 spike, SARS-CoV spike, influenza hemagglutinin H1 NC99, SARS-CoV-2 S1, SARS-CoV-2 RBD, and SARS-CoV-2 S2. Data are represented as mean±SEM of technical duplicates and represent one of at least two independent experiments (n=2). FIG. 19C. ACE2 blocking ELISA. ACE2 binding without competitor is shown as a dotted line. ELISAs were performed at one antibody concentration, and data are represented as mean±SEM of technical triplicates and represent one of at least two independent experiments (n=2).

FIGS. 20A-20C show LIBRA-seq with antigen titrations for affinity predictions. To create affinity-type measurements and identify high potency antibodies using the LIBRA-seq technology, an antigen screening library containing an antigen titration was applied. FIG. 20A. In this experiment, six different amounts of oligo-labeled SARS-CoV-2 S protein were included in a screening library. Antibodies with high affinity for SARS-CoV-2 S show reactivity (high LIBRA-seq score) for S protein added in lower amounts. FIG. 20B. For 5317 experiment, SARS-CoV-2 spike was added in a single amount. The LIBRA-seq score for S is shown on the y-axis and the affinity is shown on the x axis. FIG. 20C. For 5318-2 experiment, SARS-CoV-2 spike was added in a titration along with ACE2. The area under the curve for the LIBRA-seq score titration curve for SARS-CoV-2 S is shown on the y-axis and the affinity is shown on the x axis. This experimental test highlights the potential to predict affinity from a sequencing experiment.

### DETAILED DESCRIPTION

Therefore, in some aspects, disclosed herein are recombinant antibodies that specifically bind a viral protein of a coronavirus and uses thereof for treating, preventing, inhibiting, reducing, and detecting coronavirus infection, wherein the coronavirus is SARS-CoV-2.

Reference will now be made in detail to the embodiments of the invention, examples of which are illustrated in the drawings and the examples. This invention may, however, be embodied in many different forms and should not be construed as limited to the embodiments set forth herein.

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

The following definitions are provided for the full understanding of terms used in this specification.

### Terminology

The term “about” as used herein when referring to a measurable value such as an amount, a percentage, and the like, is meant to encompass variations of ±20%, ±10%, ±5%, or ±1% from the measurable value.

“Administration” to a subject or “administering” includes any route of introducing or delivering to a subject an agent. Administration can be carried out by any suitable route, including oral, intravenous, intraperitoneal, intranasal, inhalation and the like. Administration includes self-administration and the administration by another.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

As used herein, the term “subject” or “host” can refer to living organisms such as mammals, including, but not limited to humans, livestock, dogs, cats, and other mammals. Administration of the therapeutic agents can be carried out at dosages and for periods of time effective for treatment of a subject. In some embodiments, the subject is a human.

As used herein, the term “antigen” refers to a molecule that is capable of binding to an antibody. In some embodiments, the antigen stimulates an immune response such as by production of antibodies specific for the antigen.

In the present invention, “specific for” and “specificity” means a condition where one of the molecules is involved in selective binding. Accordingly, an antibody that is specific for one antigen selectively binds that antigen and not other antigens.

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof. The antibodies can be tested for their desired activity using the in vitro assays described herein, or by analogous methods, after which their in vivo therapeutic and/or prophylactic activities are tested according to known clinical testing methods. Native antibodies are usually heterotetrameric glycoproteins of about 150,000 daltons, composed of two identical light (L) chains and two identical heavy (H) chains. Each heavy chain has at one end a variable domain (VH) followed by a number of constant domains. Each light chain has a variable domain at one end (VL) and a constant domain at its other end. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

Each antibody molecule is made up of the protein products of two genes: heavy-chain gene and light-chain gene. The heavy-chain gene is constructed through somatic recombination of V, D, and J gene segments. In human, there are 51 VH, 27 DH, 6 JH, 9 CH gene segments on human chromosome 14. The light-chain gene is constructed through somatic recombination of V and J gene segments. There are 40 Vκ, 31 Vλ, 5 Jκ, 4 Jλ gene segments on human chromosome 14 (80 VJ). The heavy-chain constant domains that correspond to the different classes of immunoglobulins are called a, 6, F, 7, and, respectively. The “light chains” of antibodies from any vertebrate species can be assigned to one of two clearly distinct types, called kappa (κ) and lambda (λ), based on the amino acid sequences of their constant domains.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity.

The disclosed monoclonal antibodies can be made using any procedure which produces monoclonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

The monoclonal antibodies may also be made by recombinant DNA methods. DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

As used herein, the term “antibody or antigen binding fragment thereof” or “antibody or fragments thereof” encompasses chimeric antibodies and hybrid antibodies, with dual or multiple antigen or epitope specificities, and fragments, such as F(ab′)2, Fab′, Fab, Fv, sFv, scFv, nanoantibody and the like, including hybrid fragments. Thus, fragments of the antibodies that retain the ability to bind their specific antigens are provided. Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

The terms “antigen binding site”, “binding site” and “binding domain” refer to the specific elements, parts or amino acid residues of a polypeptide, such as an antibody, that bind the antigenic determinant or epitope.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations, κ and λ light chains refer to the two major antibody light chain isotypes.

The term “CDR” as used herein refers to the “complementarity determining regions” of the antibody which consist of the antigen binding loops. (Kabat E. A. et al., (1991) Sequences of proteins of immunological interest. NIH Publication 91-3242). Each of the two variable domains of an antibody Fv fragment contain, for example, three CDRs.

The term “hypervariable region” or “HVR”, as used herein, refers to each of the regions of an antibody variable domain which are hypervariable in sequence and/or form structurally defined loops (“hypervariable loops”). Generally, native four-chain antibodies comprise six HVRs; three in the VH (H1, H2, H3), and three in the VL (L1, L2, L3). HVRs generally comprise amino acid residues from the hypervariable loops and/or from the complementarity determining regions (CDRs), the latter being of highest sequence variability and/or involved in antigen recognition. With the exception of CDR1 in VH, CDRs generally comprise the amino acid residues that form the hypervariable loops. Hypervariable regions (HVRs) are also referred to as “complementarity determining regions” (CDRs), and these terms are used herein interchangeably in reference to portions of the variable region that form the antigen-binding regions. The amino acid sequence boundaries of a CDR can be determined by one of skill in the art using any of a number of known numbering schemes, including those described by Kabat et al., supra (“Kabat” numbering scheme): Al-Lazikani et al., 1997. J. Mol. Biol., 273:927-948 (“Chothia” numbering scheme); MacCallum et al., 1996, J. Mol. Biol, 262:732-745 (“Contact” numbering scheme); Lefranc et al., Dev. Comp. Immunol., 2003, 27:55-77 (“IMGT” numbering scheme); and Honegge and Pluckthun, J. Mol. Biol., 2001, 309:657-70 (“AHo” numbering scheme); each of which is incorporated by reference in its entirety.

“Effective amount” encompasses, without limitation, an amount that can ameliorate, reverse, mitigate, prevent, or diagnose a symptom or sign of a medical condition or disorder. Unless dictated otherwise, explicitly or by context, an “effective amount” is not limited to a minimal amount sufficient to ameliorate a condition. The severity of a disease or disorder, as well as the ability of a treatment to prevent, treat, or mitigate, the disease or disorder can be measured, without implying any limitation, by a biomarker or by a clinical parameter. In some embodiments, the term “effective amount of a recombinant antibody” refers to an amount of a recombinant antibody sufficient to prevent, treat, or mitigate a coronavirus infection (e.g., SARS-CoV-2 infection).

The “fragments” or “functional fragments,” whether attached to other sequences or not, can include insertions, deletions, substitutions, or other selected modifications of particular regions or specific amino acids residues, provided the activity of the fragment is not significantly altered or impaired compared to the nonmodified peptide or protein. These modifications can provide for some additional property, such as to remove or add amino acids capable of disulfide bonding, to increase its bio-longevity, to alter its secretory characteristics, etc. In any case, the functional fragment must possess a bioactive property, such as binding to a coronavirus antigen (e.g., SARS-CoV-2 antigen), and/or ameliorating the viral infection.

The term “identity” or “homology” shall be construed to mean the percentage of nucleotide bases or amino acid residues in the candidate sequence that are identical with the bases or residues of a corresponding sequence to which it is compared, after aligning the sequences and introducing gaps, if necessary to achieve the maximum percent identity for the entire sequence, and not considering any conservative substitutions as part of the sequence identity. A polynucleotide or polynucleotide region (or a polypeptide or polypeptide region) that has a certain percentage (for example, 80%, 85%, 90%, or 95%) of “sequence identity” to another sequence means that, when aligned, that percentage of bases (or amino acids) are the same in comparing the two sequences. This alignment and the percent homology or sequence identity can be determined using software programs known in the art. Such alignment can be provided using, for instance, the method of Needleman et al. (1970) J. Mol. Biol. 48: 443-453, implemented conveniently by computer programs such as the Align program (DNAstar, Inc.).

The term “increased” or “increase” as used herein generally means an increase by a statically significant amount; for example, “increased” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, the terms “nanobody”, “V_{H}H”, “V_{H}H antibody fragment” and “single domain antibody” are used indifferently and designate a variable domain of a single heavy chain of an antibody of the type found in Camelidae, which are without any light chains, such as those derived from Camelids as described in PCT Publication No. WO 94/04678, which is incorporated by reference in its entirety.

The term “reduced”, “reduce”, “reduction”, or “decrease” as used herein generally means a decrease by a statistically significant amount. However, for avoidance of doubt, “reduced” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

“Nucleotide,” “nucleoside,” “nucleotide residue,” and “nucleoside residue,” as used herein, can mean a deoxyribonucleotide, ribonucleotide residue, or another similar nucleoside analogue. A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. A non-limiting example of a nucleotide would be 3′-AMP (3′-adenosine monophosphate) or 5′-GMP (5′-guanosine monophosphate). There are many varieties of these types of molecules available in the art and available herein.

The method and the system disclosed here including the use of primers, which are capable of interacting with the disclosed nucleic acids, such as the antigen barcode as disclosed herein. In certain embodiments the primers are used to support DNA amplification reactions. Typically, the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically, the disclosed primers hybridize with the disclosed nucleic acids or region of the nucleic acids or they hybridize with the complement of the nucleic acids or complement of a region of the nucleic acids.

The term “amplification” refers to the production of one or more copies of a genetic fragment or target sequence, specifically the “amplicon”. As it refers to the product of an amplification reaction, amplicon is used interchangeably with common laboratory terms, such as “PCR product.”

The term “polypeptide” refers to a compound made up of a single chain of D- or L-amino acids or a mixture of D- and L-amino acids joined by peptide bonds.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA.

An “expression cassette” refers to a DNA coding sequence or segment of DNA that code for an expression product that can be inserted into a vector at defined restriction sites. The cassette restriction sites are designed to ensure insertion of the cassette in the proper reading frame. Generally, foreign DNA is inserted at one or more restriction sites of the vector DNA, and then is carried by the vector into a host cell along with the transmissible vector DNA. A segment or sequence of DNA having inserted or added DNA, such as an expression vector, can also be called a “DNA construct”.

Expression vectors comprise the expression cassette and additionally usually comprise an origin for autonomous replication in the host cells or a genome integration site, one or more selectable markers (e.g. an amino acid synthesis gene or a gene conferring resistance to antibiotics such as zeocin, kanamycin, G418 or hygromycin), a number of restriction enzyme cleavage sites, a suitable promoter sequence and a transcription terminator, which components are operably linked together. The term “vector” as used herein includes autonomously replicating nucleotide sequences as well as genome integrating nucleotide sequences. A common type of vector is a “plasmid”, which generally is a self-contained molecule of double-stranded DNA that can readily accept additional (foreign) DNA and which can readily be introduced into a suitable host cell. A plasmid vector often contains coding DNA and promoter DNA and has one or more restriction sites suitable for inserting foreign DNA. Specifically, the term “vector” or “plasmid” refers to a vehicle by which a DNA or RNA sequence (e.g. a foreign gene) can be introduced into a host cell, so as to transform the host and promote expression (e.g. transcription and translation) of the introduced sequence.

The term “host cell” as used herein shall refer to primary subject cells trans-formed to produce a particular recombinant protein, such as an antibody as described herein, and any progeny thereof. It should be understood that not all progeny are exactly identical to the parental cell (due to deliberate or inadvertent mutations or differences in environment), however, such altered progeny are included in these terms, so long as the progeny retain the same functionality as that of the originally transformed cell. The term “host cell line” refers to a cell line of host cells as used for expressing a recombinant gene to produce recombinant polypeptides such as recombinant antibodies. The term “cell line” as used herein refers to an established clone of a particular cell type that has acquired the ability to proliferate over a prolonged period of time. Such host cell or host cell line may be maintained in cell culture and/or cultivated to produce a recombinant polypeptide.

The term “gene” or “gene sequence” refers to the coding sequence or control sequence, or fragments thereof. A gene may include any combination of coding sequence and control sequence, or fragments thereof. Thus, a “gene” as referred to herein may be all or part of a native gene. A polynucleotide sequence as referred to herein may be used interchangeably with the term “gene”, or may include any coding sequence, non-coding sequence or control sequence, fragments thereof, and combinations thereof. The term “gene” or “gene sequence” includes, for example, control sequences upstream of the coding sequence.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents.

As used herein, the term “carrier” encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia, P A, 2005. Examples of physiologically acceptable carriers include saline, glycerol, DMSO, buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ (ICI, Inc.; Bridgewater, New Jersey), polyethylene glycol (PEG), and PLURONICS™ (BASF; Florham Park, NJ). To provide for the administration of such dosages for the desired therapeutic treatment, compositions disclosed herein can advantageously comprise between about 0.1% and 99% by weight of the total of one or more of the subject compounds based on the weight of the total composition including carrier or diluent.

The term “specificity” refers to the number of different types of antigens or antigenic determinants to which a particular antigen-binding molecule (such as the recombinant antibody of the invention) can bind. As used herein, the term “specifically binds,” as used herein with respect to a recombinant antibody refers to the recombinant antibody's preferential binding to one or more epitopes as compared with other epitopes. Specific binding can depend upon binding affinity and the stringency of the conditions under which the binding is conducted. In one example, an antibody specifically binds an epitope when there is high affinity binding under stringent conditions.

It should be understood that the specificity of an antigen-binding molecule (e.g., the recombinant antibodies of the present invention) can be determined based on affinity and/or avidity. The affinity, represented by the equilibrium constant for the dissociation of an antigen with an antigen-binding molecule (K_{D}), is a measure for the binding strength between an antigenic determinant and an antigen-binding site on the antigen-binding molecule: the lesser the value of the K_{D}, the stronger the binding strength between an antigenic determinant and the antigen-binding molecule (alternatively, the affinity can also be expressed as the affinity constant (KA), which is 1/K_{D}). As will be clear to the skilled person (for example on the basis of the further disclosure herein), affinity can be determined in a manner known per se, depending on the specific antigen of interest. Avidity is the measure of the strength of binding between an antigen-binding molecule (such as the recombinant antibodies of the present invention) and the pertinent antigen. Avidity is related to both the affinity between an antigenic determinant and its antigen binding site on the antigen-binding molecule and the number of pertinent binding sites present on the antigen-binding molecule. Typically, antigen-binding proteins (such as the recombinant antibodies of the invention) will bind to their antigen with a dissociation constant (K_{D}) of 10⁵ to 10⁻¹² moles/liter or less, and preferably 10⁻⁷ to 10⁻¹² moles/liter or less, and more preferably 10⁻⁸ to 10⁻¹² moles/liter.

“Therapeutically effective amount” refers to the amount of a composition such as recombinant antibody that will elicit the biological or medical response of a tissue, system, animal, or human that is being sought by the researcher, veterinarian, medical doctor or other clinician over a generalized period of time. In some embodiments, a desired response is reduction of coronaviral titers in a subject. In some embodiments, the desired response is mitigation of coronavirus infection and/or related symptoms. In some instances, a desired biological or medical response is achieved following administration of multiple dosages of the composition to the subject over a period of days, weeks, or years. The therapeutically effective amount will vary depending on the composition, the disorder or conditions and its severity, the route of administration, time of administration, rate of excretion, drug combination, judgment of the treating physician, dosage form, and the age, weight, general health, sex and/or diet of the subject to be treated. The therapeutically effective amount of recombinant antibodies as described herein can be determined by one of ordinary skill in the art.

A therapeutically significant reduction in a symptom is, e.g. at least about 10%, at least about 20%, at least about 30%, at least about 40%, at least about 50%, at least about 60%, at least about 70%, at least about 80%, at least about 90%, at least about 100%, at least about 125%, at least about 150% or more in a measured parameter as compared to a control or non-treated subject. Measured or measurable parameters include clinically detectable markers of disease, for example, elevated or depressed levels of a biological marker, such as decreased viral titers, decreased viral RNA levels, increase in CD4 T lymphocyte counts, and/or prolonged survival of a subject. It will be understood, that the total daily usage of the compositions and formulations as disclosed herein will be decided by the attending physician within the scope of sound medical judgment. The exact amount required will vary depending on factors such as the type of disease being treated.

The terms “treat,” “treating,” “treatment,” and grammatical variations thereof as used herein, include partially or completely delaying, alleviating, mitigating or reducing the intensity of one or more attendant symptoms of infection. Treatments according to the invention may be applied preventively, prophylactically, palliatively or remedially. Prophylactic treatments are administered to a subject prior to onset (e.g., before obvious signs of an infection), during early onset (e.g., upon initial signs and symptoms of an infection), after an established development of an infection, or during chronic infection. Prophylactic administration can occur for several minutes to months prior to the manifestation of an infection.

As used herein, the term “preventing” a disorder or unwanted physiological event in a subject refers specifically to the prevention of the occurrence of symptoms and/or their underlying cause, wherein the subject may or may not exhibit heightened susceptibility to the disorder or event.

### Antibodies and Compositions

In some aspects, disclosed herein is a recombinant antibody, said antibody comprising a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein
CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NOs: 4969-5796, 13255-14083, 21356-22312, 26193-26205, 26263-26275, or 26289-26318; and
CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NOs: 7453-8280, 15742-16570, 25184-26140, 26232-26244, 26276-26288, or 26319-26348.

In some embodiments, the CDRH3 comprises at least one amino acid substitution when compared to SEQ ID NOs: SEQ ID NOs: 4969-5796, 13255-14083, 21356-22312, 26193-26205, 26263-26275, or 26289-26318. In some embodiments, the CDRL3 comprises at least one amino acid substitution when compared to SEQ ID NOs: 7453-8280, 15742-16570, 25184-26140, 26232-26244, 26276-26288, or 26319-26348.

In some embodiments, the CDRH1 comprises an amino acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NOs: 3313-4140, 11597-12425, 19442-20398, or 26167-26179; and CDRL1 comprises an amino acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NOs: 5797-6624, 14084-14912, 23270-24226, or 26206-26218.

In some embodiments, the CDRH1 comprises at least one amino acid substitution when compared to SEQ ID NOs: 3313-4140, 11597-12425, 19442-20398, or 26167-26179. In some embodiments, the CDRH1 comprises at least 1, 2, 3, 4, 5, or 6 substitutions when compared to SEQ ID NOs: 3313-4140, 11597-12425, 19442-20398, or 26167-26179.

In some embodiments, the CDRL1 comprises at least one amino acid substitution when compared to SEQ ID NOs: 5797-6624, 14084-14912, 23270-24226, or 26206-26218. In some embodiments, the CDRL1 comprises at least 1, 2, 3, 4, 5, or 6 substitutions when compared to SEQ ID NOs: 5797-6624, 14084-14912, 23270-24226, or 26206-26218.

In some embodiments, the CDRH2 comprises an amino acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NOs: 4141-4968, 12426-13254, 20399-21355, or 26180-26192; and CDRL2 comprises an amino acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NOs: 6625-7452, 14913-15741, 24227-25183, or 26219-26231.

In some embodiments, the CDRH2 comprises at least one amino acid substitution when compared to SEQ ID NOs: 4141-4968, 12426-13254, 20399-21355, or 26180-26192. In some embodiments, the CDRH2 comprises at least 1, 2, 3, 4, 5, or 6 substitutions when compared to SEQ ID NOs: 4141-4968, 12426-13254, 20399-21355, or 26180-26192.

In some embodiments, the CDRL2 comprises at least one amino acid substitution when compared to SEQ ID NOs: 6625-7452, 14913-15741, 24227-25183, or 26219-26231. In some embodiments, the CDRL2 comprises at least 1, 2, 3, 4, 5, or 6 substitutions when compared to SEQ ID NOs: 6625-7452, 14913-15741, 24227-25183, or 26219-26231.

In some embodiments, VH comprises an amino acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NOs: 1657-2484,9939-10767, 18485-19441, or 26141-26153. In some embodiments, VH comprises an amino acid sequence selected from the group consisting of SEQ ID NOs: 1657-2484, 9939-10767, 18485-19441, and 26141-26153.

In some embodiments, VL comprises an amino acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NOs: 2485-3312, 10768-11596, 22313-23269, or 26154-26166. In some embodiments, VL comprises an amino acid sequence selected from the group consisting of SEQ ID NOs: 2485-3312, 10768-11596, 22313-23269, and 26154-26166.

In some embodiments, a CDR sequence (for example CDRL1, CDRL2, CDRL3, CDRH1, CDRH2, or CDRH3) comprises one amino acid mutation, two amino acid mutations, three amino acid mutations, four amino acid mutations, five amino acid mutations, etc. when compared to a CDR sequence as disclosed herein.

In some embodiments, the recombinant antibody is a monoclonal antibody. In some embodiments, the recombinant antibody is an isolated antibody. In some embodiments, the recombinant antibody is a non-naturally occurring antibody. In some embodiments, the recombinant antibody is an antibody or antigen binding fragment thereof. In some embodiments, combinations of antibodies or antigen binding fragments thereof disclosed herein are used for treating coronavirus infection.

In some embodiments, combinations of antibodies or antigen binding fragments thereof disclosed herein are used for treating SARS-CoV-2 infection.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 4056,
CDRH2 is SEQ ID NO: 4884,
CDRH3 is SEQ ID NO: 5712,
CDRL1 is SEQ ID NO: 6540,
CDRL2 is SEQ ID NO: 7368, and
CDRL3 is SEQ ID NO: 8196.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 3791,
CDRH2 is SEQ ID NO: 4619,
CDRH3 is SEQ ID NO: 5447,
CDRL1 is SEQ ID NO: 6275,
CDRL2 is SEQ ID NO: 7103, and
CDRL3 is SEQ ID NO: 7931.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 3858,
CDRH2 is SEQ ID NO: 4686,
CDRH3 is SEQ ID NO: 5514,
CDRL1 is SEQ ID NO: 6342,
CDRL2 is SEQ ID NO: 7170, and
CDRL3 is SEQ ID NO: 7998.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 3680,
CDRH2 is SEQ ID NO: 4508,
CDRH3 is SEQ ID NO: 5336,
CDRL1 is SEQ ID NO: 6164,
CDRL2 is SEQ ID NO: 6992, and
CDRL3 is SEQ ID NO: 7820.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 3856,
CDRH2 is SEQ ID NO: 4684,
CDRH3 is SEQ ID NO: 5512,
CDRL1 is SEQ ID NO: 6340,
CDRL2 is SEQ ID NO: 7168, and
CDRL3 is SEQ ID NO: 7996.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 3355,
CDRH2 is SEQ ID NO: 4183,
CDRH3 is SEQ ID NO: 5011,
CDRL1 is SEQ ID NO: 5839,
CDRL2 is SEQ ID NO: 6667, and
CDRL3 is SEQ ID NO: 7495.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 3697,
CDRH2 is SEQ ID NO: 4525,
CDRH3 is SEQ ID NO: 5353,
CDRL1 is SEQ ID NO: 6181,
CDRL2 is SEQ ID NO: 7009, and
CDRL3 is SEQ ID NO: 7837.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 3481,
CDRH2 is SEQ ID NO: 4309,
CDRH3 is SEQ ID NO: 5137,
CDRL1 is SEQ ID NO: 5965,
CDRL2 is SEQ ID NO: 6793, and
CDRL3 is SEQ ID NO: 7621.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 3896,
CDRH2 is SEQ ID NO: 4724,
CDRH3 is SEQ ID NO: 5552,
CDRL1 is SEQ ID NO: 6380,
CDRL2 is SEQ ID NO: 7208, and
CDRL3 is SEQ ID NO: 8036.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 3667,
CDRH2 is SEQ ID NO: 4495,
CDRH3 is SEQ ID NO: 5323,
CDRL1 is SEQ ID NO: 6151,
CDRL2 is SEQ ID NO: 6979, and
CDRL3 is SEQ ID NO: 7807.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 12368,
CDRH2 is SEQ ID NO: 13197,
CDRH3 is SEQ ID NO: 14026,
CDRL1 is SEQ ID NO: 14855,
CDRL2 is SEQ ID NO: 15684, and
CDRL3 is SEQ ID NO: 16513.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 11621,
CDRH2 is SEQ ID NO: 12450,
CDRH3 is SEQ ID NO: 13279,
CDRL1 is SEQ ID NO: 14108,
CDRL2 is SEQ ID NO: 14937, and
CDRL3 is SEQ ID NO: 15766.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 11742,
CDRH2 is SEQ ID NO: 12571,
CDRH3 is SEQ ID NO: 13400,
CDRL1 is SEQ ID NO: 14229,
CDRL2 is SEQ ID NO: 15058, and
CDRL3 is SEQ ID NO: 15887.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 11598,
CDRH2 is SEQ ID NO: 12427,
CDRH3 is SEQ ID NO: 13256,
CDRL1 is SEQ ID NO: 14085,
CDRL2 is SEQ ID NO: 14914, and
CDRL3 is SEQ ID NO: 15743.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 12262,
CDRH2 is SEQ ID NO: 13091,
CDRH3 is SEQ ID NO: 13920,
CDRL1 is SEQ ID NO: 14749,
CDRL2 is SEQ ID NO: 15578, and
CDRL3 is SEQ ID NO: 16407.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 11995,
CDRH2 is SEQ ID NO: 12824,
CDRH3 is SEQ ID NO: 13653,
CDRL1 is SEQ ID NO: 14482,
CDRL2 is SEQ ID NO: 15311, and
CDRL3 is SEQ ID NO: 16140.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 12164,
CDRH2 is SEQ ID NO: 12993,
CDRH3 is SEQ ID NO: 13822,
CDRL1 is SEQ ID NO: 14651,
CDRL2 is SEQ ID NO: 15480, and
CDRL3 is SEQ ID NO: 16309.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 11752,
CDRH2 is SEQ ID NO: 12581,
CDRH3 is SEQ ID NO: 13410,
CDRL1 is SEQ ID NO: 14239,
CDRL2 is SEQ ID NO: 15068, and
CDRL3 is SEQ ID NO: 15897.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 11888,
CDRH2 is SEQ ID NO: 12717,
CDRH3 is SEQ ID NO: 13546,
CDRL1 is SEQ ID NO: 14375,
CDRL2 is SEQ ID NO: 15204, and
CDRL3 is SEQ ID NO: 16033.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 20173,
CDRH2 is SEQ ID NO: 21130,
CDRH3 is SEQ ID NO: 22087,
CDRL1 is SEQ ID NO: 24001,
CDRL2 is SEQ ID NO: 24958, and
CDRL3 is SEQ ID NO: 25915.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 20065,
CDRH2 is SEQ ID NO: 21022,
CDRH3 is SEQ ID NO: 21979,
CDRL1 is SEQ ID NO: 23893,
CDRL2 is SEQ ID NO: 24850, and
CDRL3 is SEQ ID NO: 25807.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 20115,
CDRH2 is SEQ ID NO: 21072,
CDRH3 is SEQ ID NO: 22029,
CDRL1 is SEQ ID NO: 23943,
CDRL2 is SEQ ID NO: 24900, and
CDRL3 is SEQ ID NO: 25857.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 19873,
CDRH2 is SEQ ID NO: 20830,
CDRH3 is SEQ ID NO: 21787,
CDRL1 is SEQ ID NO: 23701,
CDRL2 is SEQ ID NO: 24658, and
CDRL3 is SEQ ID NO: 25615.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 19923,
CDRH2 is SEQ ID NO: 20880,
CDRH3 is SEQ ID NO: 21837,
CDRL1 is SEQ ID NO: 23751,
CDRL2 is SEQ ID NO: 24708, and
CDRL3 is SEQ ID NO: 25665.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 19458,
CDRH2 is SEQ ID NO: 20415,
CDRH3 is SEQ ID NO: 21372,
CDRL1 is SEQ ID NO: 23286,
CDRL2 is SEQ ID NO: 24243, and
CDRL3 is SEQ ID NO: 25200.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 20235,
CDRH2 is SEQ ID NO: 21192,
CDRH3 is SEQ ID NO: 22149,
CDRL1 is SEQ ID NO: 24063,
CDRL2 is SEQ ID NO: 25020, and
CDRL3 is SEQ ID NO: 25977.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 19858,
CDRH2 is SEQ ID NO: 20815,
CDRH3 is SEQ ID NO: 21772,
CDRL1 is SEQ ID NO: 23686,
CDRL2 is SEQ ID NO: 24643, and
CDRL3 is SEQ ID NO: 25600.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 19735,
CDRH2 is SEQ ID NO: 20692,
CDRH3 is SEQ ID NO: 21649,
CDRL1 is SEQ ID NO: 23563,
CDRL2 is SEQ ID NO: 24520, and
CDRL3 is SEQ ID NO: 25477.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 19887,
CDRH2 is SEQ ID NO: 20844,
CDRH3 is SEQ ID NO: 21801,
CDRL1 is SEQ ID NO: 23715,
CDRL2 is SEQ ID NO: 24672, and
CDRL3 is SEQ ID NO: 25356.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 19614,
CDRH2 is SEQ ID NO: 20571,
CDRH3 is SEQ ID NO: 21528,
CDRL1 is SEQ ID NO: 23442,
CDRL2 is SEQ ID NO: 24399, and
CDRL3 is SEQ ID NO: 25986.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 26167,
CDRH2 is SEQ ID NO: 26180,
CDRH3 is SEQ ID NO: 26193,
CDRL1 is SEQ ID NO: 26206,
CDRL2 is SEQ ID NO: 26219, and
CDRL3 is SEQ ID NO: 26232.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 26168,
CDRH2 is SEQ ID NO: 26181,
CDRH3 is SEQ ID NO: 26194,
CDRL1 is SEQ ID NO: 26207,
CDRL2 is SEQ ID NO: 26220, and
CDRL3 is SEQ ID NO: 26233.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 26169,
CDRH2 is SEQ ID NO: 26182,
CDRH3 is SEQ ID NO: 26195,
CDRL1 is SEQ ID NO: 26208,
CDRL2 is SEQ ID NO: 26221, and
CDRL3 is SEQ ID NO: 26234.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 26170,
CDRH2 is SEQ ID NO: 26183,
CDRH3 is SEQ ID NO: 26196,
CDRL1 is SEQ ID NO: 26209,
CDRL2 is SEQ ID NO: 26222, and
CDRL3 is SEQ ID NO: 26235.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 26171,
CDRH2 is SEQ ID NO: 26184,
CDRH3 is SEQ ID NO: 26197,
CDRL1 is SEQ ID NO: 26210,
CDRL2 is SEQ ID NO: 26223, and
CDRL3 is SEQ ID NO: 26236.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 26172,
CDRH2 is SEQ ID NO: 26185,
CDRH3 is SEQ ID NO: 26198,
CDRL1 is SEQ ID NO: 26211,
CDRL2 is SEQ ID NO: 26224, and
CDRL3 is SEQ ID NO: 26237.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 26173,
CDRH2 is SEQ ID NO: 26186,
CDRH3 is SEQ ID NO: 26199,
CDRL1 is SEQ ID NO: 26212,
CDRL2 is SEQ ID NO: 26225, and
CDRL3 is SEQ ID NO: 26238.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 26174,
CDRH2 is SEQ ID NO: 26187,
CDRH3 is SEQ ID NO: 26200,
CDRL1 is SEQ ID NO: 26213,
CDRL2 is SEQ ID NO: 26226, and
CDRL3 is SEQ ID NO: 26239.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 26175,
CDRH2 is SEQ ID NO: 26188,
CDRH3 is SEQ ID NO: 26201,
CDRL1 is SEQ ID NO: 26214,
CDRL2 is SEQ ID NO: 26227, and
CDRL3 is SEQ ID NO: 26240.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 26176,
CDRH2 is SEQ ID NO: 26189,
CDRH3 is SEQ ID NO: 26202,
CDRL1 is SEQ ID NO: 26215,
CDRL2 is SEQ ID NO: 26228, and
CDRL3 is SEQ ID NO: 26241.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 26177,
CDRH2 is SEQ ID NO: 26190,
CDRH3 is SEQ ID NO: 26203,
CDRL1 is SEQ ID NO: 26216,
CDRL2 is SEQ ID NO: 26229, and
CDRL3 is SEQ ID NO: 26242.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 26178,
CDRH2 is SEQ ID NO: 26191,
CDRH3 is SEQ ID NO: 26204,
CDRL1 is SEQ ID NO: 26217,
CDRL2 is SEQ ID NO: 26230, and
CDRL3 is SEQ ID NO: 26243.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 26179,
CDRH2 is SEQ ID NO: 26192,
CDRH3 is SEQ ID NO: 26205,
CDRL1 is SEQ ID NO: 26218,
CDRL2 is SEQ ID NO: 26231, and
CDRL3 is SEQ ID NO: 26244.

In some embodiments, the recombinant antibody or antigen binding fragment thereof of any preceding aspect comprises a VH comprising an amino acid sequence selected from SEQ ID NOs: 1657-2484, 9939-10767, 18485-19441, and 26141-26153.

In some embodiments, the recombinant antibody or antigen binding fragment thereof of any preceding aspect comprises a VL comprising an amino acid sequence selected from SEQ ID NOs: 2485-3312, 10768-11596, 22313-23269, and 26154-26166.

### Methods

Disclosed herein are methods for preventing, treating, inhibiting, reducing, or detecting coronavirus infection.

In some aspects, disclosed herein is a method of producing a recombinant antibody comprising cultivating or maintaining the host cell of any preceding aspect under conditions to produce a recombinant antibody as described herein.

In some aspects, disclosed herein is a method of treating, preventing, reducing, and/or inhibiting coronavirus infection, comprising administering to a subject a therapeutically effective amount of a recombinant antibody, wherein the recombinant antibody comprises a light chain variable region (VL) that comprises a light chain complementarity determining region (CDRL)1,
CDRL2, and CDRL3 and a heavy chain variable region (VH) that comprises a heavy chain complementarity determining region (CDRH)1, CDRH2, and CDRH3, wherein CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NOs: 4969-5796, 13255-14083, 21356-22312, 26193-26205, 26263-26275, or 26289-26318; and
CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NOs: 7453-8280, 15742-16570, 25184-26140, 26232-26244, 26276-26288, or 26319-26348.

In some aspects, disclosed herein is a method of treating, preventing, reducing, and/or inhibiting coronavirus infection, comprising administering to a subject a therapeutically effective amount of a recombinant antibody, wherein the recombinant antibody comprises a light chain variable region (VL) that comprises a light chain complementarity determining region (CDRL)1, CDRL2, and CDRL3 or a heavy chain variable region (VH) that comprises a heavy chain complementarity determining region (CDRH)1, CDRH2, and CDRH3, wherein
CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NOs: 4969-5796, 13255-14083, 21356-22312, 26193-26205, 26263-26275, or 26289-26318 or; and
CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NOs: 7453-8280, 15742-16570, 25184-26140, 26232-26244, 26276-26288, or 26319-26348.

In some embodiments, the CDRH3 comprises at least one amino acid substitution when compared to SEQ ID NOs: SEQ ID NOs: 4969-5796, 13255-14083, 21356-22312, 26193-26205, 26263-26275, or 26289-26318 or. In some embodiments, the CDRL3 comprises at least one amino acid substitution when compared to SEQ ID NOs: 7453-8280, 15742-16570, 25184-26140, 26232-26244, 26276-26288, or 26319-26348 or.

In some embodiments, the CDRH1 comprises an amino acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NOs: 3313-4140, 11597-12425, 19442-20398, or 26167-26179; and CDRL1 comprises an amino acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NOs: 5797-6624, 14084-14912, 23270-24226, or 26206-26218.

In some embodiments, the CDRH1 comprises at least one amino acid substitution when compared to SEQ ID NOs: 3313-4140, 11597-12425, 19442-20398, or 26167-26179. In some embodiments, the CDRH1 comprises at least 1, 2, 3, 4, 5, or 6 substitutions when compared to SEQ ID NOs: 3313-4140, 11597-12425, 19442-20398, or 26167-26179.

In some embodiments, the CDRL1 comprises at least one amino acid substitution when compared to SEQ ID NOs: 5797-6624, 14084-14912, 23270-24226, or 26206-26218. In some embodiments, the CDRL1 comprises at least 1, 2, 3, 4, 5, or 6 substitutions when compared to SEQ ID NOs: 5797-6624, 14084-14912, 23270-24226, or 26206-26218.

In some embodiments, the CDRH2 comprises an amino acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NOs: 4141-4968, 12426-13254, 20399-21355, or 26180-26192; and CDRL2 comprises an amino acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NOs: 6625-7452, 14913-15741, 24227-25183, or 26219-26231.

In some embodiments, the CDRH2 comprises at least one amino acid substitution when compared to SEQ ID NOs: 4141-4968, 12426-13254, 20399-21355, or 26180-26192. In some embodiments, the CDRH2 comprises at least 1, 2, 3, 4, 5, or 6 substitutions when compared to SEQ ID NOs: 4141-4968, 12426-13254, 20399-21355, or 26180-26192.

In some embodiments, the CDRL2 comprises at least one amino acid substitution when compared to SEQ ID NOs: 6625-7452, 14913-15741, 24227-25183, or 26219-26231. In some embodiments, the CDRL2 comprises at least 1, 2, 3, 4, 5, or 6 substitutions when compared to SEQ ID NOs: 6625-7452, 14913-15741, 24227-25183, or 26219-26231.

In some embodiments, VH comprises an amino acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NOs: 1657-2484,9939-10767, 18485-19441, or 26141-26153. In some embodiments, VH comprises an amino acid sequence selected from the group consisting of SEQ ID NOs: 1657-2484, 9939-10767, 18485-19441, and 26141-26153.

In some embodiments, VL comprises an amino acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NOs: 2485-3312, 10768-11596, 22313-23269, or 26154-26166. In some embodiments, VL comprises an amino acid sequence selected from the group consisting of SEQ ID NOs: 2485-3312, 10768-11596, 22313-23269, and 26154-26166.

In some embodiments, a CDR sequence (for example CDRL1, CDRL2, CDRL3, CDRH1, CDRH2, or CDRH3) comprises one amino acid mutation, two amino acid mutations, three amino acid mutations, four amino acid mutations, five amino acid mutations, etc. when compared to a CDR sequence as disclosed herein.

In some embodiments, the recombinant antibody is a monoclonal antibody. In some embodiments, the recombinant antibody is an isolated antibody. In some embodiments, the recombinant antibody is an antibody or antigen binding fragment thereof. In some embodiments, combinations of antibodies or antigen binding fragments thereof disclosed herein are used for treating coronavirus infection.

In some embodiments, combinations of antibodies or antigen binding fragments thereof disclosed herein are used for treating SARS-CoV-2 infection.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 4056,
CDRH2 is SEQ ID NO: 4884,
CDRH3 is SEQ ID NO: 5712,
CDRL1 is SEQ ID NO: 6540,
CDRL2 is SEQ ID NO: 7368, and
CDRL3 is SEQ ID NO: 8196.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 3791,
CDRH2 is SEQ ID NO: 4619,
CDRH3 is SEQ ID NO: 5447,
CDRL1 is SEQ ID NO: 6275,
CDRL2 is SEQ ID NO: 7103, and
CDRL3 is SEQ ID NO: 7931.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 3858,
CDRH2 is SEQ ID NO: 4686,
CDRH3 is SEQ ID NO: 5514,
CDRL1 is SEQ ID NO: 6342,
CDRL2 is SEQ ID NO: 7170, and
CDRL3 is SEQ ID NO: 7998.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 3680,
CDRH2 is SEQ ID NO: 4508,
CDRH3 is SEQ ID NO: 5336,
CDRL1 is SEQ ID NO: 6164,
CDRL2 is SEQ ID NO: 6992, and
CDRL3 is SEQ ID NO: 7820.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 3856,
CDRH2 is SEQ ID NO: 4684,
CDRH3 is SEQ ID NO: 5512,
CDRL1 is SEQ ID NO: 6340,
CDRL2 is SEQ ID NO: 7168, and
CDRL3 is SEQ ID NO: 7996.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 3355,
CDRH2 is SEQ ID NO: 4183,
CDRH3 is SEQ ID NO: 5011,
CDRL1 is SEQ ID NO: 5839,
CDRL2 is SEQ ID NO: 6667, and
CDRL3 is SEQ ID NO: 7495.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 3697,
CDRH2 is SEQ ID NO: 4525,
CDRH3 is SEQ ID NO: 5353,
CDRL1 is SEQ ID NO: 6181,
CDRL2 is SEQ ID NO: 7009, and
CDRL3 is SEQ ID NO: 7837.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 3481,
CDRH2 is SEQ ID NO: 4309,
CDRH3 is SEQ ID NO: 5137,
CDRL1 is SEQ ID NO: 5965,
CDRL2 is SEQ ID NO: 6793, and
CDRL3 is SEQ ID NO: 7621.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 3896,
CDRH2 is SEQ ID NO: 4724,
CDRH3 is SEQ ID NO: 5552,
CDRL1 is SEQ ID NO: 6380,
CDRL2 is SEQ ID NO: 7208, and
CDRL3 is SEQ ID NO: 8036.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 3667,
CDRH2 is SEQ ID NO: 4495,
CDRH3 is SEQ ID NO: 5323,
CDRL1 is SEQ ID NO: 6151,
CDRL2 is SEQ ID NO: 6979, and
CDRL3 is SEQ ID NO: 7807.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 12368,
CDRH2 is SEQ ID NO: 13197,
CDRH3 is SEQ ID NO: 14026,
CDRL1 is SEQ ID NO: 14855,
CDRL2 is SEQ ID NO: 15684, and
CDRL3 is SEQ ID NO: 16513.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 11621,
CDRH2 is SEQ ID NO: 12450,
CDRH3 is SEQ ID NO: 13279,
CDRL1 is SEQ ID NO: 14108,
CDRL2 is SEQ ID NO: 14937, and
CDRL3 is SEQ ID NO: 15766.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 11742,
CDRH2 is SEQ ID NO: 12571,
CDRH3 is SEQ ID NO: 13400,
CDRL1 is SEQ ID NO: 14229,
CDRL2 is SEQ ID NO: 15058, and
CDRL3 is SEQ ID NO: 15887.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 11598,
CDRH2 is SEQ ID NO: 12427,
CDRH3 is SEQ ID NO: 13256,
CDRL1 is SEQ ID NO: 14085,
CDRL2 is SEQ ID NO: 14914, and
CDRL3 is SEQ ID NO: 15743.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 12262,
CDRH2 is SEQ ID NO: 13091,
CDRH3 is SEQ ID NO: 13920,
CDRL1 is SEQ ID NO: 14749,
CDRL2 is SEQ ID NO: 15578, and
CDRL3 is SEQ ID NO: 16407.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 11995,
CDRH2 is SEQ ID NO: 12824,
CDRH3 is SEQ ID NO: 13653,
CDRL1 is SEQ ID NO: 14482,
CDRL2 is SEQ ID NO: 15311, and
CDRL3 is SEQ ID NO: 16140.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 12164,
CDRH2 is SEQ ID NO: 12993,
CDRH3 is SEQ ID NO: 13822,
CDRL1 is SEQ ID NO: 14651,
CDRL2 is SEQ ID NO: 15480, and
CDRL3 is SEQ ID NO: 16309.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 11752,
CDRH2 is SEQ ID NO: 12581,
CDRH3 is SEQ ID NO: 13410,
CDRL1 is SEQ ID NO: 14239,
CDRL2 is SEQ ID NO: 15068, and
CDRL3 is SEQ ID NO: 15897.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 11888,
CDRH2 is SEQ ID NO: 12717,
CDRH3 is SEQ ID NO: 13546,
CDRL1 is SEQ ID NO: 14375,
CDRL2 is SEQ ID NO: 15204, and
CDRL3 is SEQ ID NO: 16033.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 20173,
CDRH2 is SEQ ID NO: 21130,
CDRH3 is SEQ ID NO: 22087,
CDRL1 is SEQ ID NO: 24001,
CDRL2 is SEQ ID NO: 24958, and
CDRL3 is SEQ ID NO: 25915.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 20065,
CDRH2 is SEQ ID NO: 21022,
CDRH3 is SEQ ID NO: 21979,
CDRL1 is SEQ ID NO: 23893,
CDRL2 is SEQ ID NO: 24850, and
CDRL3 is SEQ ID NO: 25807.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 20115,
CDRH2 is SEQ ID NO: 21072,
CDRH3 is SEQ ID NO: 22029,
CDRL1 is SEQ ID NO: 23943,
CDRL2 is SEQ ID NO: 24900, and
CDRL3 is SEQ ID NO: 25857.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 19873,
CDRH2 is SEQ ID NO: 20830,
CDRH3 is SEQ ID NO: 21787,
CDRL1 is SEQ ID NO: 23701,
CDRL2 is SEQ ID NO: 24658, and
CDRL3 is SEQ ID NO: 25615.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 19923,
CDRH2 is SEQ ID NO: 20880,
CDRH3 is SEQ ID NO: 21837,
CDRL1 is SEQ ID NO: 23751,
CDRL2 is SEQ ID NO: 24708, and
CDRL3 is SEQ ID NO: 25665.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 19458,
CDRH2 is SEQ ID NO: 20415,
CDRH3 is SEQ ID NO: 21372,
CDRL1 is SEQ ID NO: 23286,
CDRL2 is SEQ ID NO: 24243, and
CDRL3 is SEQ ID NO: 25200.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 20235,
CDRH2 is SEQ ID NO: 21192,
CDRH3 is SEQ ID NO: 22149,
CDRL1 is SEQ ID NO: 24063,
CDRL2 is SEQ ID NO: 25020, and
CDRL3 is SEQ ID NO: 25977.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 19858,
CDRH2 is SEQ ID NO: 20815,
CDRH3 is SEQ ID NO: 21772,
CDRL1 is SEQ ID NO: 23686,
CDRL2 is SEQ ID NO: 24643, and
CDRL3 is SEQ ID NO: 25600.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 19735,
CDRH2 is SEQ ID NO: 20692,
CDRH3 is SEQ ID NO: 21649,
CDRL1 is SEQ ID NO: 23563,
CDRL2 is SEQ ID NO: 24520, and
CDRL3 is SEQ ID NO: 25477.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 19887,
CDRH2 is SEQ ID NO: 20844,
CDRH3 is SEQ ID NO: 21801,
CDRL1 is SEQ ID NO: 23715,
CDRL2 is SEQ ID NO: 24672, and
CDRL3 is SEQ ID NO: 25356.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 19614,
CDRH2 is SEQ ID NO: 20571,
CDRH3 is SEQ ID NO: 21528,
CDRL1 is SEQ ID NO: 23442,
CDRL2 is SEQ ID NO: 24399, and
CDRL3 is SEQ ID NO: 25986.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 26167,
CDRH2 is SEQ ID NO: 26180,
CDRH3 is SEQ ID NO: 26193,
CDRL1 is SEQ ID NO: 26206,
CDRL2 is SEQ ID NO: 26219, and
CDRL3 is SEQ ID NO: 26232.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 26168,
CDRH2 is SEQ ID NO: 26181,
CDRH3 is SEQ ID NO: 26194,
CDRL1 is SEQ ID NO: 26207,
CDRL2 is SEQ ID NO: 26220, and
CDRL3 is SEQ ID NO: 26233.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 26169,
CDRH2 is SEQ ID NO: 26182,
CDRH3 is SEQ ID NO: 26195,
CDRL1 is SEQ ID NO: 26208,
CDRL2 is SEQ ID NO: 26221, and
CDRL3 is SEQ ID NO: 26234.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 26170,
CDRH2 is SEQ ID NO: 26183,
CDRH3 is SEQ ID NO: 26196,
CDRL1 is SEQ ID NO: 26209,
CDRL2 is SEQ ID NO: 26222, and
CDRL3 is SEQ ID NO: 26235.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 26171,
CDRH2 is SEQ ID NO: 26184,
CDRH3 is SEQ ID NO: 26197,
CDRL1 is SEQ ID NO: 26210,
CDRL2 is SEQ ID NO: 26223, and
CDRL3 is SEQ ID NO: 26236.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 26172,
CDRH2 is SEQ ID NO: 26185,
CDRH3 is SEQ ID NO: 26198,
CDRL1 is SEQ ID NO: 26211,
CDRL2 is SEQ ID NO: 26224, and
CDRL3 is SEQ ID NO: 26237.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 26173,
CDRH2 is SEQ ID NO: 26186,
CDRH3 is SEQ ID NO: 26199,
CDRL1 is SEQ ID NO: 26212,
CDRL2 is SEQ ID NO: 26225, and
CDRL3 is SEQ ID NO: 26238.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 26174,
CDRH2 is SEQ ID NO: 26187,
CDRH3 is SEQ ID NO: 26200,
CDRL1 is SEQ ID NO: 26213,
CDRL2 is SEQ ID NO: 26226, and
CDRL3 is SEQ ID NO: 26239.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 26175,
CDRH2 is SEQ ID NO: 26188,
CDRH3 is SEQ ID NO: 26201,
CDRL1 is SEQ ID NO: 26214,
CDRL2 is SEQ ID NO: 26227, and
CDRL3 is SEQ ID NO: 26240.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 26176,
CDRH2 is SEQ ID NO: 26189,
CDRH3 is SEQ ID NO: 26202,
CDRL1 is SEQ ID NO: 26215,
CDRL2 is SEQ ID NO: 26228, and
CDRL3 is SEQ ID NO: 26241.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 26177,
CDRH2 is SEQ ID NO: 26190,
CDRH3 is SEQ ID NO: 26203,
CDRL1 is SEQ ID NO: 26216,
CDRL2 is SEQ ID NO: 26229, and
CDRL3 is SEQ ID NO: 26242.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 26178,
CDRH2 is SEQ ID NO: 26191,
CDRH3 is SEQ ID NO: 26204,
CDRL1 is SEQ ID NO: 26217,
CDRL2 is SEQ ID NO: 26230, and
CDRL3 is SEQ ID NO: 26243.

In some embodiments, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 26179,
CDRH2 is SEQ ID NO: 26192,
CDRH3 is SEQ ID NO: 26205,
CDRL1 is SEQ ID NO: 26218,
CDRL2 is SEQ ID NO: 26231, and
CDRL3 is SEQ ID NO: 26244.

In some embodiments, the recombinant antibody or antigen binding fragment thereof of any preceding aspect comprises a VH comprising an amino acid sequence selected from SEQ ID NOs: 1657-2484, 9939-10767, 18485-19441, and 26141-26153.

In some embodiments, the recombinant antibody or antigen binding fragment thereof of any preceding aspect comprises a VL comprising an amino acid sequence selected from SEQ ID NOs: 2485-3312, 10768-11596, 22313-23269, and 26154-26166.

In some embodiments, the recombinant antibody binds to at least one coronavirus antigen. In some embodiments, the recombinant antibody binds to at least one SARS-CoV-2 antigen.

In some embodiments, the target protein comprises a viral protein. In some embodiments, the viral protein is a coronavirus protein. Coronaviruses constitute the subfamily Orthocoronavirinae, in the family Coronaviridae, order Nidovirales, and realm Riboviria. They are enveloped viruses with a positive-sense single-stranded RNA genome and a nucleocapsid of helical symmetry. The genome size of coronaviruses ranges from approximately 27 to 34 kilobases. The structure of coronavirus generally consists of the following: spike protein, hemagglutinin-esterease dimer (HE), a membrane glycoprotein (M), an envelope protein (E) a nucleoclapid protein (N) and RNA. The coronavirus family comprises genera including, for example, alphacoronavius (e.g., Human coronavirus 229E, Human coronavirus NL63, Miniopterus bat coronavirus 1, Miniopterus bat coronavirus HKU8, Porcine epidemic diarrhea virus, Rhinolophus bat coronavirus HKU2, Scotophilus bat coronavirus 512), betacoronavirus (e.g., SARS-CoV-2, Betacoronavirus 1, Human coronavirus HKU1, Murine coronavirus, Pipistrellus bat coronavirus HKU5, Rousettus bat coronavirus HKU9, Severe acute respiratory syndrome-related coronavirus, Tylonycteris bat coronavirus HKU4, Middle East respiratory syndrome-related coronavirus (MERS), Human coronavirus OC43, Hedgehog coronavirus 1 (EriCoV)), gammacoronavirus (e.g., Beluga whale coronavirus SW1, Infectious bronchitis virus), and deltacoronavirus (e.g., Bulbul coronavirus HKU11, Porcine coronavirus HKU15). In some embodiments, the viral protein is a protein of Severe acute respiratory syndrome-related coronavirus. In some embodiments, the viral protein is a protein of MERS coronavirus.

In some embodiments, the viral protein is a SARS-CoV-2 protein, including, for example, SARS-CoV-2 spike protein, SARS-CoV-2 envelope protein, SARS-CoV-2 membrane protein, or SARS-CoV-2 nucleocapsid protein, or a fragment thereof. In some embodiments, the viral protein is a receptor binding domain of a SARS-CoV-2 spike protein.

In some aspects, disclosed herein is a method of producing a recombinant antibody comprising cultivating or maintaining the host cell of any preceding aspect under conditions to produce said recombinant antibody.

In some aspects, disclosed herein is a method of treating, preventing, reducing, and/or inhibiting coronavirus infection comprising administering to a subject a therapeutically effective amount of the recombinant antibody of any preceding aspect.

In some aspects, disclosed herein is a method of diagnosing a coronavirus infection comprising the use of the recombinant antibody of any preceding aspect. In some aspects, disclosed herein is a kit for diagnosing a coronavirus infection comprising the recombinant antibody of any preceding aspect.

The antibody repertoire characterization done herein is also readily generalizable to other pathogens, and as such, have a broad and lasting impact on the development of countermeasures for established and emerging infectious diseases.

Methods for determining antibody sequences and antigen-antibody specificities are known in the art. See, e.g., International Publication Number: WO 2020/033164, incorporated by reference.

In some aspects, disclosed herein is a method for detecting a coronavirus infection in a subject, comprising: providing a biological sample from the subject, and detecting a coronavirus antigen in the biological sample with an antibody that specifically binds to the coronavirus antigen, wherein the antibody is from any aspect as disclosed herein, and wherein the presence of the coronavirus antigen in the biological sample indicates the subject is infected with a coronavirus.

The biological sample can be from, for example, a throat swab, a nasal swab, a nasopharyngeal swab, an oropharyngeal swab, cells, blood, serum, plasma, saliva, urine, stool, sputum, or nasopharyngeal aspirates.

In some embodiments, the coronavirus infection is caused by SARS-CoV-2. In some embodiments, the method comprises contacting the biological sample with a SARS-CoV-2 antigen. In some embodiments, the SARS-CoV-2 antigen is directly immobilized on a substrate and is detected by an antibody disclosed herein directly or indirectly by a labeled heterologous anti-isotype antibody, wherein the bound antibody can be detected by a detection assay. The SARS-CoV-2 antigen can be selected from the spike (S), envelope (E), membrane (M), and nucleocapsid (N) proteins, or a fragment thereof.

The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a secondary antibody that is labeled a fluorescent probe or with biotin for detection. In vitro techniques for detection of the antibodies of SARS-CoV-2 include enzyme linked immunosorbent assays (ELISAs), Western blots, immunoprecipitations and immunofluorescence, IgM antibody capture enzyme immunoassay (MAC-ELISA), indirect IgG ELISA, indirect fluorescent antibody assay (IFAT), hemagglutination inhibition (HIT), and serum dilution cross-species plaque reduction neutralization tests (PRNTs).

In some embodiments, in vitro techniques for detection of an antigen of SARS-CoV-2 include enzyme linked immunosorbent assays (ELISAs), Western blots, immunoprecipitations and immunofluorescence. Furthermore, in vivo techniques for detection of SARS-CoV-2 include introducing into a subject a labeled antibody directed against the polypeptide. For example, the antibody can be labeled with a radioactive marker whose presence and location can be detected by standard imaging techniques, including autoradiography.

In some embodiments, the levels of the antibodies are determined by immunoassay comprising Enzyme linked immunospot (ELISPOT), Enzyme-linked immunosorbent assay (ELISA), western blot, or a multiplex ELISA assay. In some embodiments, the multiplex ELISA assay is selected from the group consisting of Luminex, Veriplex, LEGENDplex, Bio-Plex, Milliplex MAP, and FirePlex.

The steps of various useful immunodetection methods have been described in the scientific literature, such as, e.g., Maggio et al., Enzyme-Immunoassay, (1987) and Nakamura, et al., Enzyme Immunoassays: Heterogeneous and Homogeneous Systems, Handbook of Experimental Immunology, Vol. 1: Immunochemistry, 27.1-27.20 (1986), each of which is incorporated herein by reference in its entirety and specifically for its teaching regarding immunodetection methods. Immunoassays, in their most simple and direct sense, are binding assays involving binding between antibodies and antigen. Many types and formats of immunoassays are known and all are suitable for detecting the disclosed biomarkers. Examples of immunoassays are enzyme linked immunosorbent assays (ELISAs), radioimmunoassays (RIA), radioimmune precipitation assays (RIPA), immunobead capture assays, Western blotting, dot blotting, gel-shift assays, Flow cytometry, protein arrays, multiplexed bead arrays, magnetic capture, in vivo imaging, fluorescence resonance energy transfer (FRET), and fluorescence recovery/localization after photobleaching (FRAP/FLAP).

The invention also encompasses kits for detecting the presence of SARS-CoV-2 or a polypeptide/antigen thereof in a biological sample. For antibody-based kits, the kit can comprise, for example: (1) a first antibody (e.g., attached to a solid support) which binds to a coronavirus antigen; and, optionally, (2) a second, different antibody which binds to either the coronavirus antigen or the first antibody and is conjugated to a detectable agent.

### EXAMPLES

The following examples are set forth below to illustrate the antibodies, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1. Introduction

The emergence of a novel coronavirus (CoV) SARS-CoV-2, the causative agent of COVID-19, has resulted in a worldwide pandemic, threatening the lives of billions and imposing an immense burden on healthcare systems and the global economy. SARS-CoV-2, the seventh coronavirus known to infect humans, is a member of the Betacoronavirus genus which includes the highly pathogenic SARS-CoV-1 and MERS-CoV, as well as endemic variants OC43-CoV and HKU1-CoV. Recent coronavirus outbreaks and the threat of future emerging zoonotic strains highlight the need for coronavirus therapeutic interventions and vaccine design.

Coronaviruses utilize the homotrimeric Spike (S) protein to engage with cell-surface receptors and gain entry into host cells. S consists of two functional subunits: S1 and S2. S1 facilitates attachment to target cells and is composed of the N-terminal domain (NTD) and the receptor-binding domain (RBD), whereas S2, which encodes the fusion peptide and heptad repeats, promotes viral fusion. To facilitate cell entry, human coronaviruses employ different host factors; however, SARS-CoV-1 and SARS-CoV-2 both utilize the cell-surface receptor, angiotensin converting enzyme 2 (ACE2). Additionally, SARS-CoV-2 S shares 76% amino acid identity with SARS-CoV-1 S. Furthermore, S serves as a dominant antibody target and is a focus of countermeasures for the treatment and prevention of COVID-19 infection. Neutralizing antibodies can be used as preventive or therapeutic treatments. Further, identifying coronavirus antibody epitopes can inform rational design strategies for vaccines and therapies that target highly pathogenic coronaviruses, which can be of value both for the current and potential future outbreaks.

A variety of potent neutralizing antibodies against SARS-CoV-2 have been identified, including multiple antibodies currently in clinical trials for prophylactic and acute treatment of COVID-19. Defining the genetic features, epitope targets, and function of antibodies can provide insights into current therapeutic strategies and can provide alternative approaches for the prevention and treatment of coronavirus infection.

In the examples below, antibody reactivity to SARS-CoV-2 is investigated at monoclonal resolution. To do this, LIBRA-seq (Linking B Cell receptor to antigen specificity through sequencing) is applied, a recently developed high-throughput antibody screening technology that allows for determination of B cell receptor sequence and antigen reactivity simultaneously for many single B cells. From convalescent SARS-CoV-2 donor samples, potent SARS-CoV-2-reactive human antibodies are identified and characterized that target multiple, distinct structural domains of S and demonstrate potent neutralization activity. A better understanding of the epitope specificities and functional characteristics of coronavirus antibodies can translate into strategies for current vaccine design efforts and additional measures to counteract potential future pandemic variants.

### Example 2. LIBRA-Seq Applied to a SARS-CoV-2 Convalescent Donor Sample

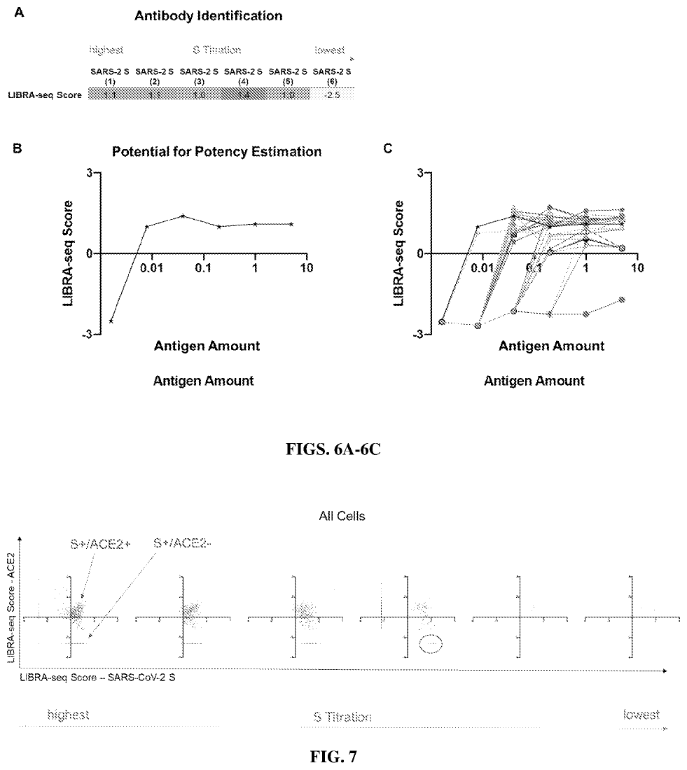

To identify SARS-CoV-2 reactive antibodies, LIBRA-seq was applied to a PBMC sample from a donor previously infected with SARS-CoV-2. Three experiments were performed to identify high-affinity coronavirus antibodies and ultra-potent neutralizing antibodies utilizing multiple features of LIBRA-seq: affinity measurements and ligand blocking functionality. To assess affinity measurements, in experiment 1, the antigen library consisted of an antigen titration of SARS-CoV-2 S protein along with control antigens influenza HA NC99 and HIV ZM197. To assess ligand blocking, in experiment 2, the antigen library consisted of SARS-CoV-2 S protein along with its receptor, ACE2, and control antigens influenza HA NC99 and HIV ZM197. To assess affinity measurements in combination with ligand blocking, in experiment 3, the antigen library consisted of an antigen titration of SARS-CoV-2 S protein, ACE2, and control antigens influenza HA NC99 and HIV ZM197. Each antigen library was incubated with SARS-CoV-2 convalescent donor PBMCs and LIBRA-seq was performed (FIG. 4).

After the antigen screening library was mixed with donor PBMCs, antigen positive B cells were enriched by fluorescence activated cell sorting and processed for single-cell sequencing. After bioinformatic processing, thousands of cells with paired heavy/light chain sequences and antigen reactivity information were recovered. Overall, LIBRA-seq allows rapid screening of PBMCs from a patient sample, with recovery of paired heavy/light chain sequences and antigen reactivity for thousands of single B cells.

### Example 3. Identification of SARS-CoV-2 and SARS-CoV-1 Cross-Reactive Antibodies and Ligand-Blocking Antibodies

In order to identify antibodies that were cross-reactive to multiple coronavirus S proteins, antibodies are prioritized based on their sequence features and LIBRA-seq scores. Antibodies that exhibit diverse sequence features are selected and a number of different variable genes are utilized for expression and characterization. For the antigen titration experiments, antibodies were identified that showed high scores for S protein added in lower amounts. For ligand blocking, antibodies were identified that had high scores for S protein and low scores for ACE2-suggesting ligand blocking functionality of these antibodies. Antibodies were prioritized for expression and further testing based on these features (FIG. 4).

Antibodies are tested for binding to SARS-CoV-1 S and SARS-CoV-2 S by ELISA. Overall, the application of the LIBRA-seq technology identifies a panel of coronavirus antibodies that recognize the coronavirus S antigen.

### Example 4. Coronavirus Antibodies Target Multiple Epitopes on S

To elucidate the epitopes targeted by the cross-reactive antibodies, binding assays to various structural domains of S are performed. Antibody binding to the S1 and S2 subdomains of SARS-CoV-2 is assessed. Additionally, antibody binding to the receptor binding domain (RBD) and N-terminal domain (NTD) is assessed. Many antibodies target the RBD. Some of the cross-reactive antibodies are coronavirus-specific and target multiple, diverse epitopes on the S protein.

### Example 5. Functional Characterization of Coronavirus Antibodies

Next, the antibody panel is characterized. Antibodies are tested for SARS-CoV-2 virus neutralization, and many antibodies exhibit neutralization. Some antibodies are ultra-potent.

In these examples, a set of SARS-CoV-2 antibodies isolated from convalescent SARS-CoV-2 convalescent donors are described.

Given the ongoing SARS-CoV-2 pandemic and for future zoonotic coronavirus pathogens to emerge, coronavirus vaccine and therapeutic development is of paramount importance. Antibodies that can neutralize SARS-CoV-2 can serve as therapies, preventive measures, diagnostic tools, and templates for rational vaccine design strategies.

### Example 6. Methods

Donor Information. Convalescent SARS-CoV-2 PBMC donor samples were purchased from Cellero.

Antigen Purification. A variety of recombinant soluble protein antigens were used in the LIBRA-seq experiment and other experimental assays. Plasmids encoding residues 1-1208 of the SARS-CoV-2 spike with a mutated S1/S2 cleavage site, as well as 6 stabilizing proline mutations at positions (817, 892, 899, 942, 986, 987), and a C-terminal T4-fibritin trimerization motif, an 8× HisTag, and a TwinStrepTag (SARS-CoV-2 HexaPro) and human ACE2 were transiently transfected into FreeStyle293F cells (Thermo Fisher) using polyethylenimine. The coronavirus trimer spike antigen was in a prefusion-stabilized conformation (HexaPro) that better represents neutralization-sensitive epitopes in comparison to their wild-type forms. Transfected supernatants were harvested after 6 days of expression. SARS-CoV-2 HexaPro was purified using StrepTactin resin (IBA). SARS-CoV-2 HexaPro was purified over a Superose6 Increase column (GE Life Sciences). ACE2 was purified in the same manner as SARS-CoV-2 HexaPro Sp using affinity chromatography and size exclusion chromatography.

For recombinant, soluble antigens HIV-1 gp140 SOSIP variant from strain ZM197 (clade C) and influenza hemagglutinin NC99 Y98F trimer, both contained an AviTag and were expressed in Expi293F cells using polyethylenimine (PEI) transfection reagent and cultured. FreeStyle F17 expression medium supplemented with pluronic acid and glutamine was used. The cells were cultured at 37° C. with 8% CO₂ saturation and shaking. After 5-7 days, cultures were centrifuged and supernatant was filtered and run over an affinity column of agarose bound Galanthus nivalis lectin. The column was washed with PBS and antigens were eluted with 30 mL of 1M methyl-a-D-mannopyranoside. Protein elutions were buffer exchanged into PBS, concentrated, and run on a Superdex 200 Increase 10/300 GL Sizing column on the AKTA FPLC system. Fractions corresponding to correctly folded protein were collected, analyzed by SDS-PAGE and antigenicity was characterized by ELISA using known monoclonal antibodies specific to each antigen. Avitagged antigens were biotinylated using BirA biotin ligase (Avidity LLC).

SARS-CoV-2 S1, S2, NTD truncated proteins were purchased from commercial vendor Sino Biological.

DNA-barcoding of Antigens. Oligos that possess 15 bp antigen barcode were used, a sequence capable of annealing to the template switch oligo that is part of the 10× bead-delivered oligos, and contain truncated TruSeq small RNA read 1 sequences in the following structure: 5′-CCTTGGCACCCGAGAATTCCANNNNNNNNNNNNNCCCATATAAGA*A*A-3′ (SEQ ID NO: 26262), where Ns represent the antigen barcode, * represents a phosphorothioate bond. Oligos were ordered from Sigma-Aldrich and IDT with a 5′ amino modification and HPLC purified.

For each antigen, a unique DNA barcode was directly conjugated to the antigen itself. In particular, 5′amino-oligonucleotides were conjugated directly to each antigen using the Solulink Protein-Oligonucleotide Conjugation Kit (TriLink cat no. S-9011) according to manufacturer's instructions. Briefly, the oligo and protein were desalted, and then the amino-oligo was modified with the 4FB crosslinker, and the biotinylated antigen protein was modified with S-HyNic. Then, the 4FB-oligo and the HyNic-antigen were mixed together. This causes a stable bond to form between the protein and the oligonucleotide. The concentration of the antigen-oligo conjugates was determined by a BCA assay, and the HyNic molar substitution ratio of the antigen-oligo conjugates was analyzed using the NanoDrop according to the Solulink protocol guidelines. AKTA FPLC was used to remove excess oligonucleotide from the protein-oligo conjugates, which were also verified using SDS-PAGE with a silver stain. Antigen-oligo conjugates were also used in flow cytometry titration experiments.

Antigen specific B cell sorting. Cells were stained and mixed with DNA-barcoded antigens and other antibodies, and then sorted using fluorescence activated cell sorting (FACS). First, cells were counted and viability was assessed using Trypan Blue. Then, cells were washed 3× with DPBS supplemented with 0.1% Bovine serum albumin (BSA). Cells were resuspended in DPBS-BSA and stained with cell markers including viability dye (Ghost Red 780), CD14-APCCy7, CD3-FITC, CD19-BV711, and IgG-PECy5. Additionally, antigen-oligo conjugates were added to the stain. After staining in the dark for 30 minutes at room temperature, cells were washed 3 times with PBS-BSA at 300 g for 5 minutes. Cells were then incubated for 15 minutes at room temperature with Streptavidin-PE to label cells with bound antigen. Cells were washed with DPBS-BSA, resuspended in DPBS, and sorted by FACS. Antigen positive cells were bulk sorted and delivered to the Vanderbilt Technologies for Advanced Genomics (VANTAGE) sequencing core at an appropriate target concentration for 10× Genomics library preparation and subsequent sequencing. FACS data were analyzed using FlowJo.

Sample preparation, library preparation, and sequencing. Single-cell suspensions were loaded onto the Chromium Controller microfluidics device (10× Genomics) and processed using the B-cell Single Cell V(D)J solution according to manufacturer's suggestions for a target capture of 10,000 B cells per ⅛ 10× cassette, with minor modifications in order to intercept, amplify and purify the antigen barcode libraries as previously described.

Sequence processing and bioinformatic analysis. The previously described pipeline was utilized and modified to use paired-end FASTQ files of oligo libraries as input, processes and annotates reads for cell barcode, UMI, and antigen barcode, and generates a cell barcode—antigen barcode UMI count matrix. BCR contigs were processed using Cell Ranger (10× Genomics) using GRCh38 as reference. Antigen barcode libraries were also processed using Cell Ranger (10× Genomics). The overlapping cell barcodes between the two libraries were used as the basis of the subsequent analysis. Cell barcodes that had only non-functional heavy chain sequences as well as cells with multiple functional heavy chain sequences and/or multiple functional light chain sequences were removed, reasoning that these can be multiplets. Additionally, the BCR contigs (filtered_contigs.fasta file output by Cell Ranger, 10× Genomics) was aligned to IMGT reference genes using HighV-Quest. The output of HighV-Quest was parsed using ChangeO, and merged with an antigen barcode UMI count matrix. Finally, it was determined the LIBRA-seq score for each antigen in the library for every cell.

Antibody Expression and Purification. For each antibody, variable genes were inserted into custom plasmids encoding the constant region for the IgG1 heavy chain as well as respective lambda and kappa light chains (pTwist CMV BetaGlobin WPRE Neo vector, Twist Bioscience). mAbs were expressed in Expi293F mammalian cells (ThermoFisher) by co-transfecting heavy chain and light chain expressing plasmids using PEI transfection reagent and cultured for 5-7 days. Cells were maintained in FreeStyle F17 expression medium supplemented at final concentrations of 0.1% Pluronic Acid F-68 and 20% 4 mM L-Glutamine. These cells were cultured at 37° C. with 8% CO₂ saturation and shaking. After transfection and 5-7 days of culture, cell cultures were centrifuged and supernatant was 0.45 m filtered with Nalgene Rapid Flow Disposable Filter Units with PES membrane. Filtered supernatant was run over a column containing Protein A agarose resin equilibrated with PBS. The column was washed with PBS, and then antibodies were eluted with 100 mM Glycine HCl at 2.7 pH directly into a 1:10 volume of 1M Tris-HCl pH 8.0. Eluted antibodies were buffer exchanged into PBS 3 times using Amicon Ultra centrifugal filter units and concentrated. Antibodies were analyzed by SDS-PAGE.

ELISA. To assess antibody binding, soluble protein was plated at 2 μg/ml overnight at 4° C. The next day, plates were washed three times with PBS supplemented with 0.05% Tween-20 (PBS-T) and coated with 5% milk powder in PBS-T. Plates were incubated for one hour at room temperature and then washed three times with PBS-T. Primary antibodies were diluted in 1% milk in PBS-T, starting at 10 μg/ml with a serial 1:5 dilution and then added to the plate. The plates were incubated at room temperature for one hour and then washed three times in PBS-T. The secondary antibody, goat anti-human IgG conjugated to peroxidase, was added at 1:10,000 dilution in 1% milk in PBS-T to the plates, which were incubated for one hour at room temperature. Plates were washed three times with PBS-T and then developed by adding TMB substrate to each well. The plates were incubated at room temperature for ten minutes, and then 1N sulfuric acid was added to stop the reaction. Plates were read at 450 nm.

### Example 7. Rapid and Efficient Discovery of Potently Neutralizing SARS-CoV-2 Antibodies Using LIBRA-Seq with Ligand Blocking

The emergence of a novel coronavirus (CoV), SARS-CoV-2, has resulted in a worldwide pandemic, threatening the lives of millions and imposing an immense burden on healthcare systems and the global economy. The devastating effects of the COVID-19 pandemic have highlighted the critical need for rapid, high-throughput screening tools for antibody discovery against viral pathogens. Antibodies can be utilized as therapeutic molecules, and studying antibody-antigen interactions can be exploited in vaccine design strategies during both pandemic emergencies and for other health concerns as well. With typical antibody screening tools, hundreds to thousands of antibodies must be screened, expressed, and tested to identify neutralizing antibody candidates for further characterization. In particular, though therapeutic antibody discovery efforts against SARS-CoV-2 have been generally successful, they have been associated with the production of large numbers of antibodies with low hit rates for the identification of lead candidates. Here, antibody-ligand blocking has been incorporated as part of LIBRA-seq, the high throughput sequencing platform for antibody discovery. By using SARS-CoV-2 spike (S) and its receptor ACE2, the LIBRA-seq with ligand blocking technology was applied to convalescent SARS-CoV-2 samples and high rates of neutralizing antibody identification was demonstrated (90% of predictions confirmed), including the discovery of several ultra-potent SARS-CoV-2 antibodies. The antibodies identified targeted diverse epitopes across the S protein and bound to several major circulating S variants. A better understanding of the sequence features, epitopes, and functional characteristics of potent, SARS-CoV-2 neutralizing antibodies can translate into strategies for current vaccine design efforts and additional measures to counteract potential future pandemic variants. Overall, leveraging LIBRA-seq with ligand blocking enables general antibody discovery targeting the disruption of antibody-ligand interactions and can facilitate the creation of better vaccines and therapies in a variety of disease settings.

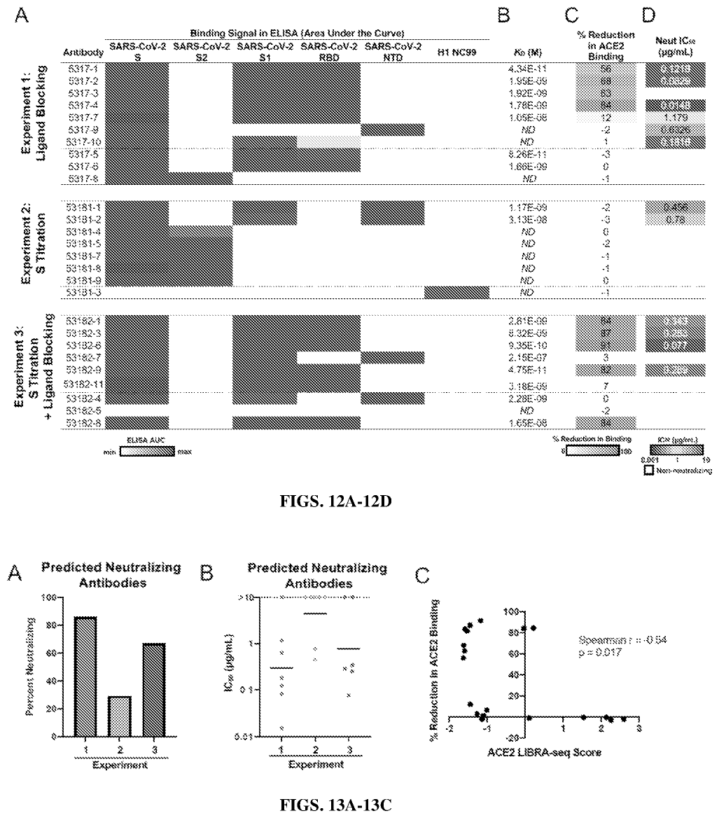

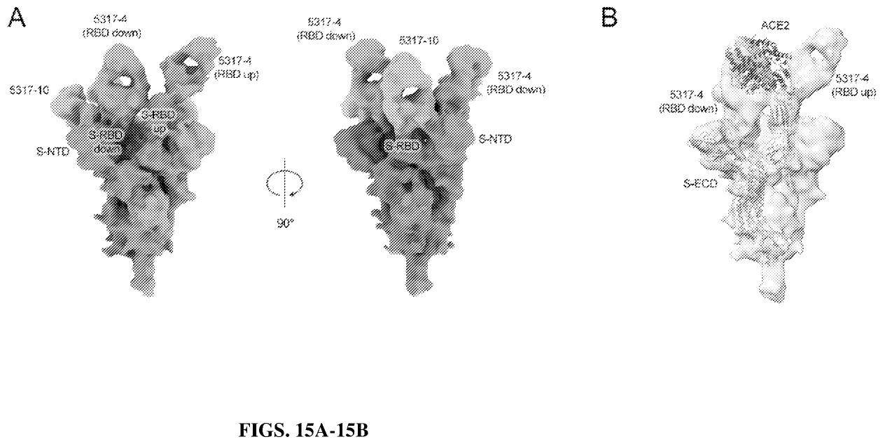

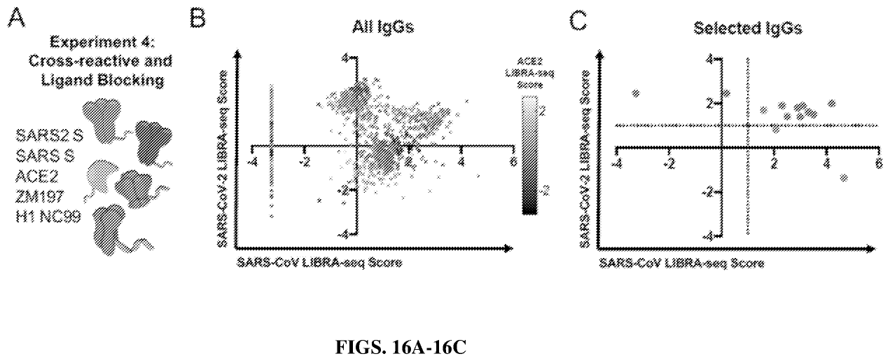

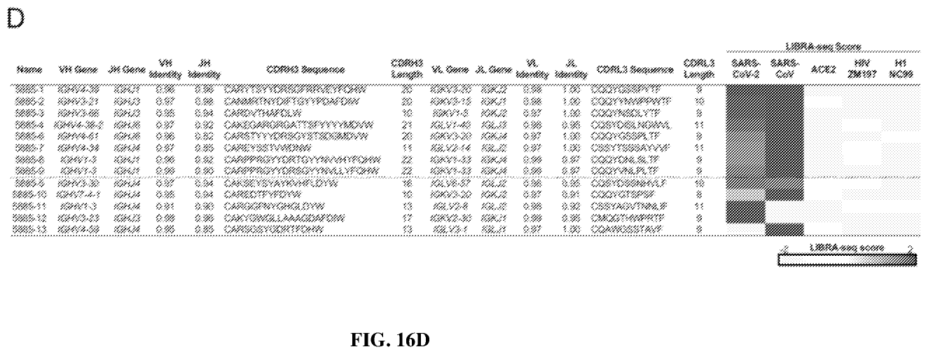

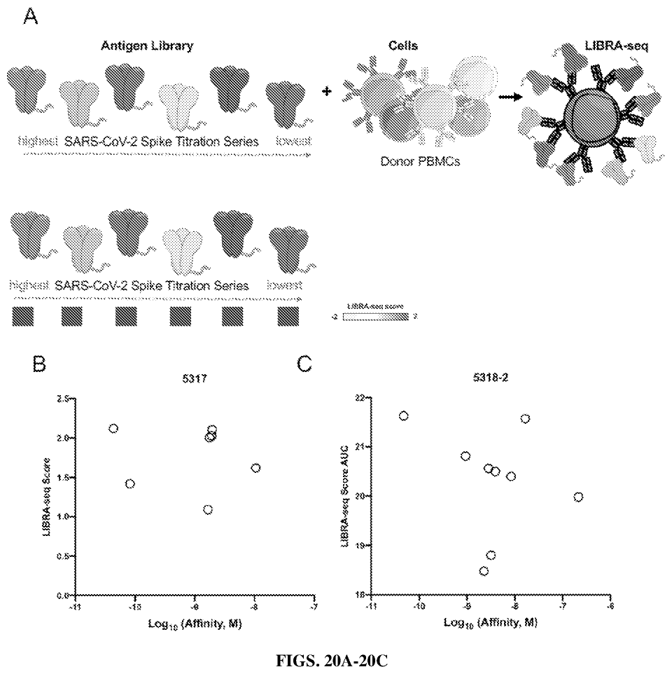

LIBRA-seq turns antibody antigen interactions into “sequenceable events.” This occurs through the use of DNA-barcoded antigens that can be recovered in single cell sequencing data and then bioinformatically mapped to B-cell receptor sequences (FIGS. 8A-8E). LIBRA-seq with ligand blocking allows for rapid and efficient prioritization of lead neutralizing antibody candidates (FIG. 11). Validation and characterization of expressed antibodies is shown in FIG. 12. LIBRA-seq with ligand blocking confirms predicted SARS-CoV-2 neutralization by antibodies at high rates (FIG. 10). Utilizing an antigen titration in LIBRA-seq can lead to affinity predictions from a sequencing experiment (FIG. 20).

This study shows application of the LIBRA-seq with ligand blocking to a SARSCoV-2 convalescent donor PBMC sample led to the rapid identification of potently neutralizing antibodies with high hit rates. The study provides a better understanding of the sequence features, epitopes, and functional characteristics of potent, SARS-CoV-2 neutralizing antibodies may translate into strategies for current vaccine design efforts and additional measures to counteract potential future pandemic variants. Overall, leveraging LIBRA-seq with ligand blocking can enable general antibody discovery targeting the disruption of antibody-ligand interactions and can ultimately facilitate the creation of better vaccines and therapies in a variety of disease settings.

### Example 8. B-Cell Receptor Sequencing with Ligand Blocking Speeds Up Neutralizing Antibody Discovery

Technologies for developing preventive and therapeutic measures that can counteract potential pandemics are of utmost significance for public health. The COVID-19 pandemic has emphasized the importance of rapid countermeasure development. Through pandemic preparedness initiatives, effective SARS-CoV-2 neutralizing antibodies were discovered and validated within months, as were SARS-CoV-2 vaccine candidates. However, even with such unprecedented speed of vaccine and therapeutic development, the pandemic has inflicted devastating worldwide effects. Accelerating actions by weeks or months can make an enormous difference in an exponentially evolving pandemic. Therefore, efficient methods for discovery of effective countermeasures against emerging pathogens can play a critical role in pandemic preparedness for future infectious disease outbreaks.

Antibodies are a major modality for therapy and vaccine design strategies for a wide range of diseases; however, the functional antibody discovery process can be inefficient. Typically, at the screening step, B cells are prioritized based on antigen-recognition, but this often requires time-intensive subsequent monoclonal antibody validation steps for discovery of functional, neutralizing antibodies. This limitation was exemplified by SARS-CoV-2 antibody discovery initiatives, as testing of large numbers of antibodies (frequently hundreds to thousands) was generally required to identify a small fraction of neutralizing antibodies, with a wide range of hit rates when using Spike (S) as an antigen bait (about 2 to 23%) or when using RBD and/or S1 (about 2-55%) in various studies.

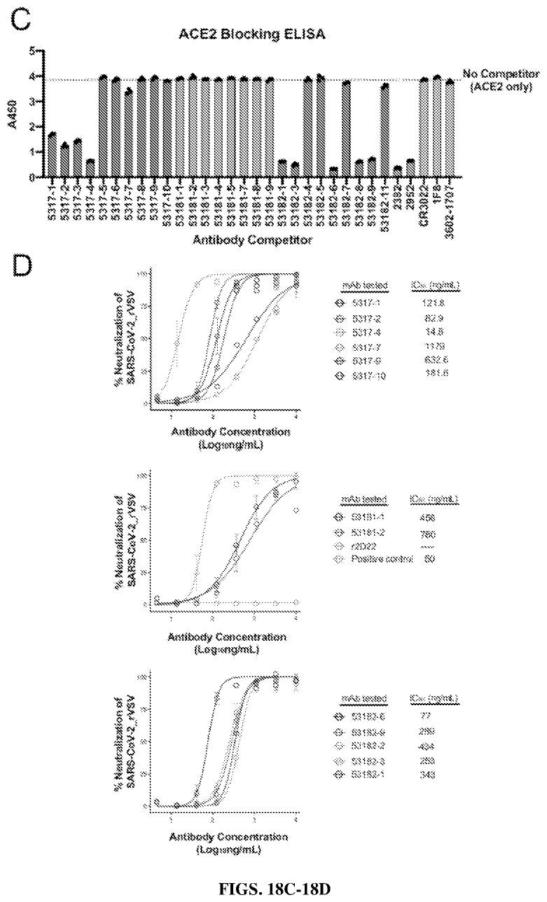

To overcome this limitation, LIBRA-seq with ligand blocking was developed, which is a second-generation LIBRA-seq technology that incorporates a functional readout into the antibody discovery process. LIBRA-seq (linking B cell receptor to antigen specificity through sequencing) uses DNA-barcoded antigens to map antibody sequence to antigen specificity using next-generation sequencing. For LIBRA-seq with ligand blocking, a ligand and its cognate target antigen(s) are each labeled with a unique oligonucleotide barcode (FIG. 17A), enabling the transformation of antigen-ligand interactions into sequence-able events. In these experiments, B cells that can block antigen-ligand interactions have high LIBRA-seq scores for the target antigen(s) and low LIBRA-seq scores for the ligand (FIG. 17A). Therefore, a single high-throughput LIBRA-seq with ligand blocking experiment provides both antigen recognition and ligand blocking information simultaneously for many B cells.

To evaluate this technology, SARS-CoV-2-specific antibodies from B cells from subjects with past SARS-CoV-2 infection were explored, since antibodies that block the interactions of the SARS-CoV-2 S protein with its host receptor angiotensin-converting enzyme 2 (ACE2) are among the most potently neutralizing identified to date. Three LIBRA-seq experiments were performed, with screening libraries that included: experiment 1, ACE2 and SARS-CoV-2 S; experiment 2, a titration series of different aliquots of SARS-CoV-2 S, each labeled with a unique barcode; and experiment 3, ACE2 and a titration series of S (FIG. 11A). The incorporation of a titration series of S antigen in the screening library for experiments 2 and 3 aimed to assess the strength of BCR-antigen interactions (FIGS. 11B and 11C).

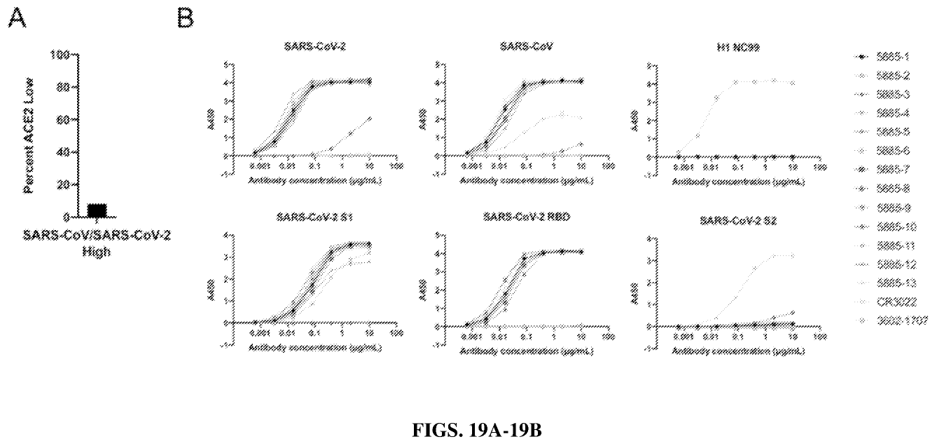

The application of LIBRA-seq resulted in 828, 829, and 957 antigen-specific B cells for the three experiments, respectively. A set of B cells were prioritized for monoclonal antibody production and validation based on the following conditions: for experiments 1 and 3 (with ACE2 in the screening library), B cells with high LIBRA-seq scores for S and low scores for ACE2 were selected; and for experiment 2, B cells that had positive scores for multiple aliquots of S were selected (FIGS. 11B-11D). B cells with high S and high ACE2 scores were also selected as controls from experiments 1 and 3, along with an influenza-specific B cell from experiment 2 (FIGS. 11B-11D). Antibodies with diverse sequence features were prioritized, although some of the selected antibodies appeared to be clonally related (FIG. 18A).

The assay confirmed the predicted antigen specificity for 26/27 (96%) antibodies and mapped the general antibody epitope regions by testing antibodies for binding to recombinant SARS-CoV-2 subdomain proteins (FIG. 12A, FIG. 18B). The majority of antibodies from experiments 1 and 3 (but none from experiment 2) recognized the RBD (FIG. 12A, FIG. 18B). Further, the antibodies had a wide range of affinities for RBD or NTD, including several antibodies with KD<1 nM, although no correlation between LIBRA-seq spike score and affinity was observed (FIG. 12B). Next, the ability of the antibodies to block ACE2 binding to spike was tested. For antibodies predicted to block ACE2 by LIBRA-seq, 57% from experiment 1 and 67% from experiment 3 demonstrated ACE2 blocking via ELISA, whereas no antibodies from experiment 2 blocked ACE2 binding (FIG. 12C, FIG. 18C).

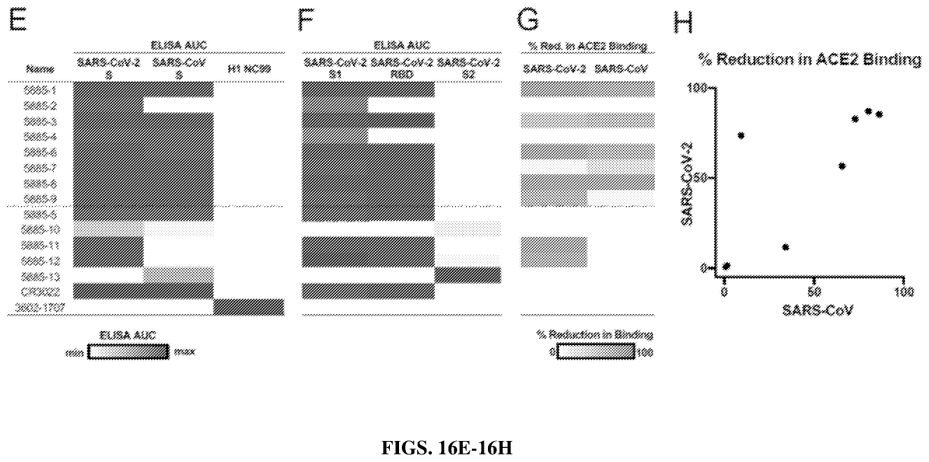

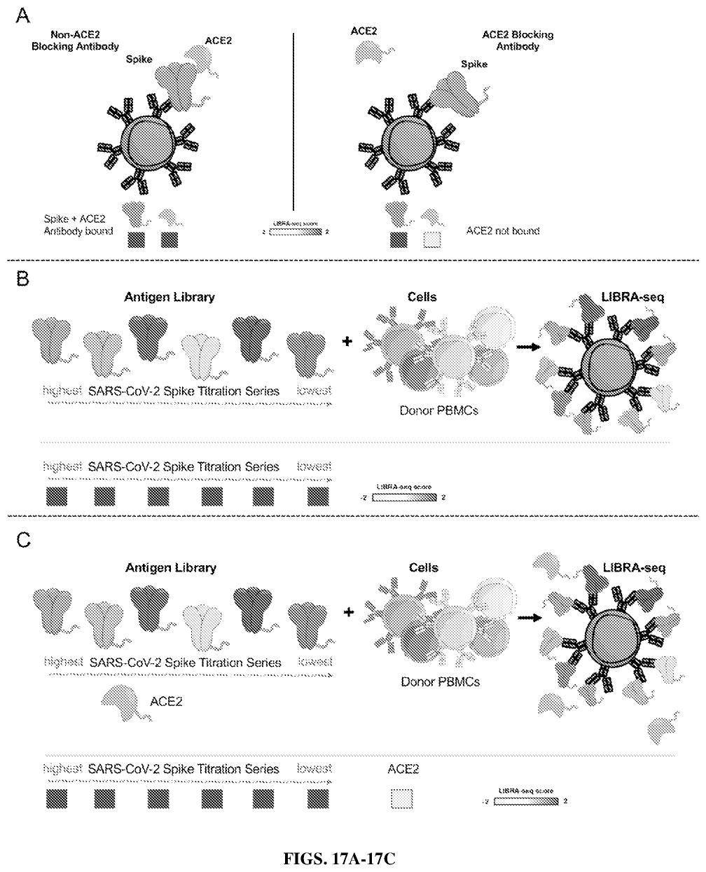

Next, the antibodies were tested in a VSV SARS-CoV-2 chimeric virus neutralization assay (FIG. 12D, FIG. 18D). For antibodies predicted to block ACE2 by LIBRA-seq, 86% from experiment 1 and 67% from experiment 3 were neutralizing, while only two clonally related antibodies (29%) from experiment 2 were neutralizing (FIGS. 13A-13B). For the antibodies from experiments 1 and 3, the ACE2 LIBRA-seq scores were correlated with the percent reduction in ACE2 binding (FIG. 13C, Spearman r=−0.54, p=0.017). Furthermore, several antibodies also showed potent neutralization against authentic SARS-CoV-2 virus in a plaque reduction assay, and in some cases against multiple SARS-CoV-2 variants (FIG. 14). Together, these results highlight the importance of including ligand blocking in LIBRA-seq for selectively identifying potent neutralizing antibodies.

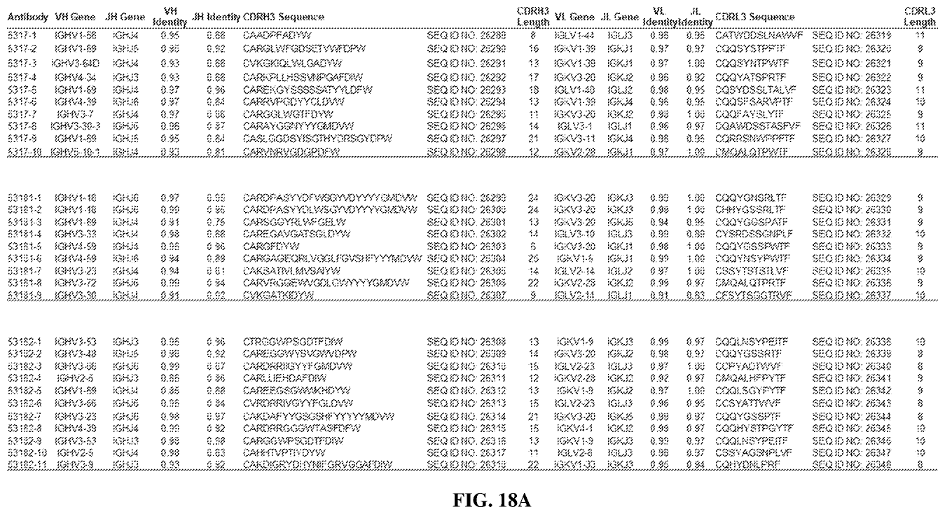

To investigate antibody recognition of SARS-CoV-2 S, a 9 Å-resolution Cryo-EM structure was determined for the antigen-binding fragments of antibodies 5317-4 and 5317-10 bound to the SARS-CoV-2 S extracellular domain (FIG. 15A). 5317-4 was chosen based on its potent neutralization (IC50 value of 7.3 ng/mL against authentic SARS-CoV-2, FIG. 14) and ACE2 competition. The 3D reconstruction revealed that 5317-4 binds to RBD in the “up” and “down” conformations, and its epitope partially overlaps the ACE2 binding footprint (FIGS. 15A-15B). When bound to the RBD in the down conformation, 5317-4 competes with ACE2 binding to the adjacent up RBD (FIG. 15B). 5317-10 was investigated because of its inconclusive epitope, as it bound to S1 but not individual RBD or NTD constructs (FIG. 12A). The map revealed that 5317-10 binds a quaternary epitope that bridges an RBD in the down position and the NTD of an adjacent protomer (FIG. 15A). This mode of recognition can prevent the RBD from transitioning into an ACE2-accessible up position, thereby preventing binding by ACE2.

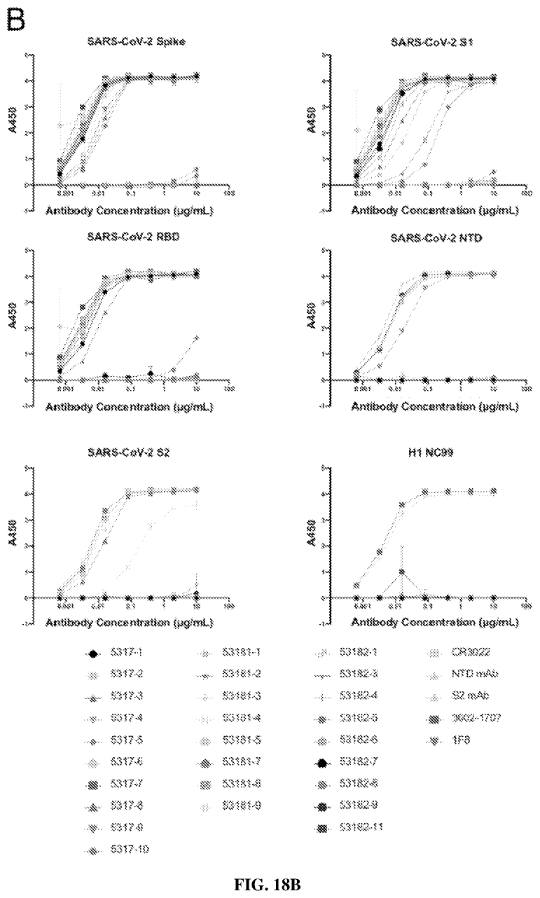

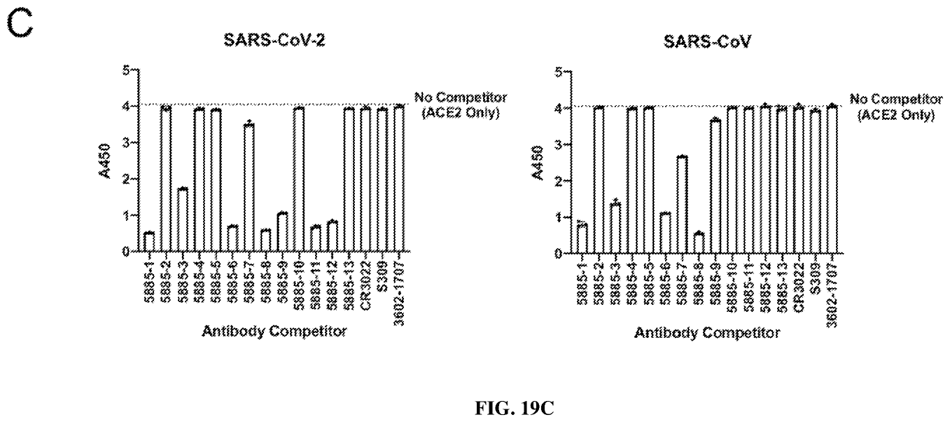

To further demonstrate the utility of LIBRA-seq with ligand blocking, the next experiment was performed to identified antibodies that show cross-reactivity between SARS-CoV-2 and SARS-CoV, and that are capable of blocking spike-ACE2 interactions. To that end, LIBRA-seq was applied to B cells from a subject with past SARS-CoV-2 infection, using an antigen library that included SARS-CoV-2 S, SARS-CoV S, and ACE2 (FIG. 16A). This resulted in 120 IgG+ B cells with high LIBRA-seq scores for both SARS-CoV-2 S and SARS-CoV S (FIG. 16B). Only 8% of these cells were associated with low LIBRA-seq scores for ACE2 (FIG. 19A, highlighting the advantage of including ligand blocking to screen for such rare cells (although also it was noted that information about B cells that show cross-reactivity but are not ACE2 blocking is also retained, enabling characterization of B cells with alternative phenotypes as well). Based on LIBRA-seq antigen and ligand blocking scores, a set of antibodies were produced and validated, including 8 with high scores for both S antigens and low scores for ACE2 (FIGS. 16C-16D). Of these, 100% bound SARS-CoV-2 S, 88% showed the predicted SARS-CoV-2/SARS-CoV cross-reactivity, and 63% demonstrated strong ACE2 blocking ability via ELISA (FIGS. 16E-16H, FIGS. 19B-19C), confirming that LIBRA-seq with ligand blocking efficiently identified ACE2-blocking antibodies with cross-reactivity between multiple coronaviruses.

Together, the results from the four LIBRA-seq experiments reported here showcase the advantages of including ligand blocking as part of the sequencing readout. As with most screening tools, there are limitations to the LIBRA-seq with ligand blocking approach, including the prerequisite for a defined antigen-ligand interaction, as well as the potential for identifying false positives. Nevertheless, through a single high-throughput sequencing experiment, LIBRA-seq with ligand blocking identified potent SARS-CoV-2 antibodies, requiring the subsequent production and validation of less than a dozen antibodies per experiment. The observed hit rates for the discovery of potently neutralizing antibodies are an improvement over what has been reported in the literature, which also typically required the screening of hundreds to thousands of antibody candidates isolated for their reactivity to antigen alone (recombinant S, S1, or RBD). Further, unlike RBD-only discovery efforts, LIBRA-seq with ligand blocking applied to spike antigens has the potential for more comprehensive coverage of antibody epitopes, as evidenced by the discovery of the RBD-NTD antibody in FIG. 15A. Overall, the application of LIBRA-seq with ligand blocking can provide critical advantages for rapid development of therapeutic and preventive countermeasures and presents a general platform with applications to virtually any area where targeting the disruption of antigen-ligand interaction is a prime therapeutic goal.

Methods

Data Availability Statement

All unique reagents generated in this study are available from the corresponding author with a completed Material Transfer Agreement. Sequences for antibodies identified and characterized in this study have been deposited to GenBank (MZ517191-MZ517250, OM001674-OM001699). Raw sequencing data has been deposited to Sequence Read Archive (PRJNA744567, SAMN24369247). Further information and requests for resources and reagents should be directed to the corresponding author, Ivelin Georgiev (Ivelin.Georgiev@Vanderbilt.edu).

Code Availability

Custom scripts used to analyze data in this manuscript are available upon request to the corresponding author.

Donor Information

PBMC samples were purchased from Cellero. The PBMCs were from subjects with past SARS-CoV-2 infection at least 14 days post symptom cessation. For experiment 1, three samples were pooled from donors 523, 527, and 528. For experiments 2 and 3, samples from donor 523 were used for LIBRA-seq. Donor 523 had a plaque reduction neutralization test titer of 1:2,560. For experiment 4 (cross-reactive antibody discovery with ligand blocking), a sample from donor 528 was used for LIBRA-seq.

Antigen Purification

A variety of recombinant soluble protein antigens were used in the LIBRA-seq experiment and other experimental assays.

Plasmids encoding residues 1-1208 of the SARS-CoV-2 spike with a mutated S1/S2 cleavage site, proline substitutions at positions 817, 892, 899, 942, 986 and 987, and a C-terminal T4-fibritin trimerization motif, an 8× HisTag, and a TwinStrepTag (SARS-CoV-2 spike HP); residues 1-1190 of the SARS-CoV spike with proline substitutions at positions 968 and 969, and a C-terminal T4-fibritin trimerization motif, an 8× HisTag, and a TwinStrepTag (SARS-CoV S-2P); and 1-615 of human ACE2 with a C-terminal HRV3C protease cleavage site, a TwinStrepTag and an 8×hisTag (ACE2) were transiently transfected in Expi293F cells using polyethylenimine. Transfected supernatants were harvested 5 days after expression and purified over a StrepTrap column (Cytiva Life Sciences). Both recombinant SARS-CoV-2 S HP and ACE2 were further purified to homogeneity using a Superose6 Increase column (Cytiva Life Sciences).

For the HIV-1 gp140 SOSIP variant from strain ZM197 (clade C) and hemagglutinin from strain A/New Caledonia/20/99 (H1N1) (GenBank ACF41878), recombinant, soluble antigens contained an AviTag and were expressed in Expi293F cells using polyethylenimine transfection reagent and cultured. FreeStyle F17 expression medium supplemented with pluronic acid and glutamine was used. The cells were cultured at 37° C. with 8% C02 saturation and shaking. After 5-7 days, cultures were centrifuged and supernatant was filtered and run over an affinity column of agarose-bound Galanthus nivalis lectin. The column was washed with PBS and antigens were eluted with 30 mL of 1M methyl-a-D-mannopyranoside. Protein elutions were buffer exchanged into PBS, concentrated, and run on a Superdex 200 Increase 10/300 GL Sizing column on the AKTA FPLC system. Fractions corresponding to correctly folded protein were collected, analyzed by SDS-PAGE and antigenicity was characterized by ELISA using known monoclonal antibodies specific to each antigen. AviTagged antigens were biotinylated using BirA biotin ligase (Avidity LLC).

SARS-CoV-2 S1, SARS-CoV-2 S2, SARS-CoV-2 RBD and SARS-CoV-2 NTD proteins were purchased from the commercial vendor, Sino Biological.

DNA-Barcoding of Antigens

This study used oligos that possess 15 bp antigen barcode, a sequence capable of annealing to the template switch oligo that is part of the 10× bead-delivered oligos and contain truncated TruSeq small RNA read 1 sequences in the following structure: 5′-CCTTGGCACCCGAGAATTCCANNNNNNNNNNNNNCCCATATAAGA*A*A-3′ (SEQ ID NO: 26262), where Ns represent the antigen barcode. For each antigen, a unique DNA barcode was directly conjugated to the antigen itself. For Experiment 1, the barcodes included SARS-CoV-2 S (GACAAGTGATCTGCA, SEQ ID NO: 26245), H1 NC99 (TCATTTCCTCCGATT, SEQ ID NO: 26246), ZM197 (TACGCCTATAACTTG; SEQ ID NO: 26247), and ACE2 (CTTCACTCTGTCAGG; SEQ ID NO: 26248). For Experiment 2, the barcodes included SARS-CoV-2 S aliquot 1 (GACAAGTGATCTGCA; SEQ ID NO: 26249), SARS-CoV-2 S aliquot 2 (TGTGTATTCCCTTGT; SEQ ID NO: 26250), SARS-CoV-2 S aliquot 3 (GCAGCGTATAAGTCA; SEQ ID NO: 26251), SARS-CoV-2 S aliquot 4 (GCTCCTTTACACGTA), SARS-CoV-2 S aliquot 5 (AGACTAATAGCTGAC; SEQ ID NO: 26252), SARS-CoV-2 S aliquot 6 (GGTAGCCCTAGAGTA; SEQ ID NO: 26253), H1 NC99 (TCATTTCCTCCGATT; SEQ ID NO: 26254), and ZM197 (TACGCCTATAACTTG; SEQ ID NO: 26255). For Experiment 3, the same barcodes were included as Experiment 2 and also included ACE2 (CTTCACTCTGTCAGG; SEQ ID NO: 26256). For Experiment 4, the barcodes included SARS-CoV-2 S (GCAGCGTATAAGTCA; SEQ ID NO: 26257), SARS-CoV S (GCTCCTTTACACGTA; SEQ ID NO: 26258), ACE2 (TACGCCTATAACTTG; SEQ ID NO: 26259), ZM197 (TCATTTCCTCCGATT; SEQ ID NO: 26260), and H1 NC99 (CTTCACTCTGTCAGG; SEQ ID NO: 26261). In particular, 5′-amino-oligonucleotides were conjugated directly to each antigen using the SoluLINK Protein-Oligonucleotide Conjugation Kit (TriLink cat no. S-9011) according to manufacturer's instructions. Briefly, the oligo and protein were desalted, and then the amino-oligo was modified with the 4FB crosslinker, and the biotinylated antigen protein was modified with S-HyNic. Then, the 4FB-oligo and the HyNic-antigen were mixed. This process causes a stable bond to form between the protein and the oligonucleotide. The concentration of the antigen-oligo conjugates was determined by a BCA assay, and the HyNic molar substitution ratio of the antigen-oligo conjugates was analyzed using the NanoDrop according to the SoluLINK protocol guidelines. AKTA FPLC was used to remove excess oligonucleotide from the protein-oligo conjugates, which were also verified using SDS-PAGE with a silver stain. Antigen-oligo conjugates were also used in flow cytometric titration experiments to determine optimal amounts for antigen-specific B cell sorting.

Antigen-Specific B Cell Sorting

Cells were stained and mixed with DNA-barcoded antigens and other antibodies, and then sorted using fluorescence activated cell sorting (FACS). First, cells were counted, and viability was assessed using trypan blue. Then, cells were washed three times with DPBS supplemented with 0.1% bovine serum albumin (BSA). Cells were resuspended in DPBS-BSA and stained with cell markers including viability dye (Ghost Red 780), CD14-APC-Cy7, CD3-FITC, CD19-BV711, and IgG-PE-Cy5. Additionally, antigen-oligo conjugates were added to the stain. For experiment 1, oligo-labeled SARS-CoV-2 S and three-fold molar excess of oligo-labeled ACE2 was added. For experiment 2, six aliquots of S protein that were each labeled with a unique DNA oligonucleotide were added in a titration series from 5 μg to 0.0016 μg (in 5-fold dilutions). For experiment 3, the same titration series of S was added along with three fold molar excess of ACE2. For experiment 4, SARS-CoV-2 S, SARS-CoV S and three-fold molar excess of oligo-labeled ACE2 was added. The antigen screening library for each of the four experiments also included an influenza virus hemagglutinin and an HIV-1 envelope variant protein as controls.

After staining in the dark for 30 minutes at room temperature, cells were washed three times with DPBS-BSA at 300×g for five minutes. Cells were then incubated for 15 minutes at room temperature with Streptavidin-PE to label cells with bound antigen. Cells were washed three times with DPBS-BSA, resuspended in DPBS, and sorted by FACS. Antigen positive cells were bulk sorted and delivered to the Vanderbilt Technologies for Advanced Genomics (VANTAGE) sequencing core at an appropriate target concentration for 10× Genomics library preparation and subsequent sequencing. Flow cytometry data were analyzed using FlowJo.

Sample Preparation, Library Preparation, and Sequencing

Single-cell suspensions were loaded onto the Chromium Controller microfluidics device (10× Genomics) and processed using the B-cell Single Cell V(D)J solution according to manufacturer's suggestions for a target capture of 10,000-20,000 B cells, with minor modifications to intercept, amplify and purify the antigen barcode libraries1. The 10× Genomics single cell VDJ human B cell assay and target enrichment protocol were completed. cDNA was amplified and additive primers were added to increase the yield of antigen derived transcript products. After cDNA amplification, the antigen derived transcript products were size separated from the mRNA-derived cDNA products using SPRI selection and further purification (per manufacturers protocol). The supernatant fraction contained the antigen-oligo derived cDNA whereas the beads fraction contained the full-length mRNA-derived cDNAs. After purification, the antigen-derived transcripts sequencing library was prepared using a PCR reaction and purified using SPRI purification. The antigen and VDJ libraries were then analyzed, quantified, and sequenced using the Illumina NovaSeq platform.

Sequence Processing and Bioinformatic Analysis

This study used the previously described pipeline to use paired-end FASTQ files of oligo libraries as input, process and annotate reads for cell barcode, UMI, and antigen barcode, and generate a cell barcode—antigen barcode UMI count matrix. BCR contigs were processed using Cell Ranger (10× Genomics) using GRCh38 as reference. Antigen barcode libraries were also processed using Cell Ranger (10× Genomics). The overlapping cell barcodes between the two libraries were used as the basis of the subsequent analysis. Cell barcodes were removed that had only non-functional heavy chain sequences as well as cells with multiple functional heavy chain sequences and/or multiple functional light chain sequences, reasoning that these may be multiplets. Additionally, the BCR contigs were aligned (filtered_contigs.fasta file output by Cell Ranger, 10× Genomics) to IMGT reference genes using HighV-Quest. The output of HighV-Quest was parsed using ChangeO and merged with an antigen barcode UMI count matrix. Finally, for experiments 1-3, the LIBRA-seq score was determined for each antigen in the library by calculating the centered-log ratios (CLR) of each antigen UMI count for each cell. A psedo-count of 1 was added to each UMI count and then the CLR was taken for each antigen for each cell. For experiment 4, the LIBRA-seq scores were calculated as previously described. Briefly, the CLR of each antigen UMI count for each cell was calculated and a Z-score transformation was also performed.

Antibody Expression and Purification

For each antibody, variable genes were inserted into custom plasmids encoding the constant region for the IgG1 heavy chain as well as respective lambda and kappa light chains (pTwist CMV BetaGlobin WPRE Neo vector, Twist Bioscience). Antibodies were expressed in Expi293F mammalian cells (Thermo Fisher Scientific) by co-transfecting heavy chain and light chain expressing plasmids using polyethylenimine transfection reagent and cultured for 5 to 7 days. Cells were maintained in FreeStyle F17 expression medium supplemented at final concentrations of 0.1% Pluronic Acid F-68 and 20% 4 mM L-Glutamine. These cells were cultured at 37° C. with 8% CO₂ saturation and shaking. After transfection and 5-7 days of culture, cell cultures were centrifuged and supernatant was 0.45 m filtered with Nalgene Rapid Flow Disposable Filter Units with PES membrane. Filtered supernatant was run over a column containing Protein A agarose resin equilibrated with PBS. The column was washed with PBS, and then antibodies were eluted with 100 mM Glycine HCl at 2.7 pH directly into a 1:10 volume of 1M Tris-HCl pH 8.0. Eluted antibodies were buffer exchanged into PBS 3 times using Amicon Ultra-centrifugal filter units and concentrated. Antibody plasmids were sequenced. If antibody sequences did not match expected heavy or light chain, antibody was excluded from downstream analysis.

Antibody Expression and Purification

For each antibody, variable genes were inserted into custom plasmids encoding the constant region for the IgG1 heavy chain as well as respective lambda and kappa light chains (pTwist CMV BetaGlobin WPRE Neo vector, Twist Bioscience). Antibodies were expressed in Expi293F mammalian cells (Thermo Fisher Scientific) by co-transfecting heavy chain and light chain expressing plasmids using polyethylenimine transfection reagent and cultured for 5 to 7 days. Cells were maintained in FreeStyle F17 expression medium supplemented at final concentrations of 0.1% Pluronic Acid F-68 and 20% 4 mM L-Glutamine. These cells were cultured at 37° C. with 8% CO₂ saturation and shaking. After transfection and 5-7 days of culture, cell cultures were centrifuged and supernatant was 0.45 m filtered with Nalgene Rapid Flow Disposable Filter Units with PES membrane. Filtered supernatant was run over a column containing Protein A agarose resin equilibrated with PBS. The column was washed with PBS, and then antibodies were eluted with 100 mM Glycine HCl at 2.7 pH directly into a 1:10 volume of 1M Tris-HCl pH 8.0. Eluted antibodies were buffer exchanged into PBS 3 times using Amicon Ultra-centrifugal filter units and concentrated. Antibody plasmids were sequenced. If antibody sequences did not match expected heavy or light chain, antibody was excluded from downstream analysis.

High-Throughput Antibody Expression

For high-throughput production of recombinant antibodies, approaches were used that are designated as microscale. For antibody expression, microscale transfection was performed (˜1 mL per antibody) of CHO cell cultures using the Gibco ExpiCHO Expression System and a protocol for deep 96-well blocks (Thermo Fisher Scientific). In brief, synthesized antibody-encoding DNA (˜2 μg per transfection) was added to OptiPro serum free medium (OptiPro SFM), incubated with ExpiFectamine CHO Reagent and added to 800 μL of ExpiCHO cell cultures into 96-deep-well blocks using a ViaFlo 384 liquid handler (Integra Biosciences). The plates were incubated on an orbital shaker at 1,000 r.p.m. with an orbital diameter of 3 mm at 37° C. in 8% CO₂. The next day after transfection, ExpiFectamine CHO Enhancer and ExpiCHO Feed reagents (Thermo Fisher Scientific) were added to the cells, followed by 4 d incubation for a total of 5 d at 37° C. in 8% CO₂. Culture supernatants were collected after centrifuging the blocks at 450×g for 5 min and were stored at 4° C. until use. For high-throughput microscale antibody purification, fritted deep-well plates were used containing 25 μL of settled protein G resin (GE Healthcare Life Sciences) per well. Clarified culture supernatants were incubated with protein G resin for antibody capturing, washed with PBS using a 96-well plate manifold base (Qiagen) connected to the vacuum and eluted into 96-well PCR plates using 86 μL of 0.1 M glycine-HCL buffer pH 2.7. After neutralization with 14 μL of 1 M Tris-HCl pH 8.0, purified antibodies were buffer-exchanged into PBS using Zeba Spin Desalting Plates (Thermo Fisher Scientific) and stored at 4° C. until use.

ELISA

To assess antibody binding, soluble protein was plated at 2 μg/mL overnight at 4° C. The next day, plates were washed three times with PBS supplemented with 0.05% Tween-20 (PBS-T) and coated with 5% milk powder in PBS-T. Plates were incubated for one hour at room temperature and then washed three times with PBS-T. Primary antibodies were diluted in 1% milk in PBS-T, starting at 10 μg/mL with a serial 1:5 dilution and then added to the plate. The plates were incubated at room temperature for one hour and then washed three times in PBS-T. The secondary antibody, goat anti-human IgG conjugated to peroxidase, was added at 1:10,000 dilution in 1% milk in PBS-T to the plates, which were incubated for one hour at room temperature. Plates were washed three times with PBS-T and then developed by adding TMB substrate to each well. The plates were incubated at room temperature for ten minutes, and then 1N sulfuric acid was added to stop the reaction. Plates were read at 450 nm.

Data are represented as mean±SEM for one ELISA experiment. ELISAs were repeated 2 or more times. If ELISA replicates were inconsistent over more than three experiments, antibody was excluded from in vitro characterization analysis. The area under the curve (AUC) was calculated using Prism software version 8.0.0 (GraphPad).

ACE2 Binding Inhibition Assay

96-well plates were coated with 2 μg/mL purified recombinant SARS-CoV-2 at 4° C. overnight. The next day, plates were washed three times with PBS supplemented with 0.05% Tween-20 (PBS-T) and coated with 5% milk powder in PBS-T. Plates were incubated for one hour at room temperature and then washed three times with PBS-T. Purified anti were diluted in blocking buffer at 10 Ug/mL in triplicate, added to the wells, and incubated at room temperature. Without washing, recombinant human ACE2 protein with a mouse Fc tag was added to wells for a final 0.4 μg/mL concentration of ACE2 and incubated for 40 minutes at room temperature. Plates were washed three times with PBS-T, and bound ACE2 was detected using HRP-conjugated anti-mouse Fe antibody and TMB substrate. The plates were incubated at room temperature for ten minutes, and then 1N sulfuric acid was added to stop the reaction. Plates were read at 450 nm. ACE2 binding without antibody served as a control. Experiment was done in biological replicate and technical triplicates.

BioLayer Interferometry (BLI)

Purified antibodies were immobilized to AHC sensortips (FortdBio) to a response level of approximately 1.4 nm in a buffer composed of 10 mM HEPES pH 7.5, 150 mM NaCl, 3 mM EDTA, 0.05% Tween 20 and 0.1% (w/v) BSA. Immobilized antibodies were then dipped into wells containing two-fold dilutions of either SARS-CoV-2 RBD-SD1 (residues 306-577) or SARS-CoV-2 NTD, ranging in concentration from 10-0.156 nM, to measure association kinetics. Dissociation kinetics were measured by dipping sensortips into wells containing only buffer. Data were reference subtracted and kinetics were calculated in Octet Data Analysis software v10.0 using a 1:1 binding model.

RTCA Method for Initial Screening of Antibody Neutralizing Activity

To screen for neutralizing activity in the panel of recombinantly expressed antibodies, a high-throughput and quantitative RTCA assay and xCeligence RTCA HT Analyzer was used (ACEA Biosciences) that assesses kinetic changes in cell physiology, including virus-induced cytopathic effect (CPE). Twenty PL of cell culture medium (DMEM supplemented with 2% FBS) was added to each well of a 384-well E-plate using a ViaFlo384 liquid handler (Integra Biosciences) to obtain background reading. Six thousand (6,000) Vero-furin cells in 20 μL of cell culture medium were seeded per well, and the plate was placed on the analyzer. Sensograms were visualized using RTCA HT software version 1.0.1 (ACEA Biosciences). For a screening neutralization assay, equal amounts of virus were mixed with micro-scale purified antibodies in a total volume of 40 μL using DMEM supplemented with 2% FBS as a diluent and incubated for 1 h at 37° C. in 5% CO₂. At ˜17-20 h after seeding the cells, the virus-antibody mixtures were added to the cells in 384-well E-plates. Wells containing virus only (in the absence of antibody) and wells containing only Vero cells in medium were included as controls. Plates were measured every 8-12 h for 48-72 h to assess virus neutralization. Micro-scale antibodies were assessed in four 5-fold dilutions (starting from a 1:20 sample dilution), and their concentrations were not normalized. Neutralization was calculated as the percent of maximal cell index in control wells without virus minus cell index in control (virus-only) wells that exhibited maximal CPE at 40-48 h after applying virus—antibody mixture to the cells. An antibody was classified as fully neutralizing if it completely inhibited SARS-CoV-2-induced CPE at the highest tested concentration, while an antibody was classified as partially neutralizing if it delayed but did not fully prevent CPE at the highest tested concentration.

Real-Time Cell Analysis (RTCA) Neutralization Assay

To determine neutralizing activity of IgG, real-time cell analysis (RTCA) assay was used on an xCELLigence RTCA MP Analyzer (ACEA Biosciences Inc.) that measures virus-induced cytopathic effect (CPE). Briefly, 50 μL of cell culture medium (DMEM supplemented with 2% FBS) was added to each well of a 96-well E-plate using a ViaFlo384 liquid handler (Integra Biosciences) to obtain background reading. A suspension of 18,000 Vero-E6 cells in 50 μL of cell culture medium was seeded in each well, and the plate was placed on the analyzer. Measurements were taken automatically every 15 min, and the sensograms were visualized using RTCA software version 2.1.0 (ACEA Biosciences Inc). VSV-SARS-CoV-2 (0.01 MOI, ˜120 PFU per well) was mixed 1:1 with a dilution of antibody in a total volume of 100 μL using DMEM supplemented with 2% FBS as a diluent and incubated for 1 h at 37° C. in 5% CO₂. At 16 h after seeding the cells, the virus-antibody mixtures were added in replicates to the cells in 96-well E-plates. Triplicate wells containing virus only (maximal CPE in the absence of antibody) and wells containing only Vero cells in medium (no-CPE wells) were included as controls. Plates were measured continuously (every 15 min) for 48 h to assess virus neutralization. Normalized cellular index (CI) values at the endpoint (48 h after incubation with the virus) were determined using the RTCA software version 2.1.0 (ACEA Biosciences Inc.). Results are expressed as percent neutralization in a presence of respective antibody relative to control wells with no CPE minus CI values from control wells with maximum CPE. RTCA IC₅₀ values were determined by nonlinear regression analysis using Prism software.

Plaque Reduction Neutralization Test (PRNT)

The virus neutralization with live authentic SARS-CoV-2 virus (USA-WA1) was performed in the BSL-3 facility of the Galveston National Laboratory using Vero E6 cells (ATCC CRL-1586) following the standard procedure. Vero 1E6 cells were cultured in 96-well plates (10 cells/well). Next day, 4-fold serial dilutions of antibodies were made using MEM-2% FBS, as to get an initial concentration of 100 μg/mL. Equal volume of diluted antibodies (60 μL) were mixed gently with original SARS-CoV-2 (USA-WA1) (60 p L containing 200 pfu) and incubated for 1 h at 37° C./5% CO₂ atmosphere. The virus-serum mixture (100 μL) was added to cell monolayer in duplicates and incubated for 1 at h 37° C./5% CO₂ atmosphere. Later, the virus-serum mixture was discarded gently, and cell monolayer was overlaid with 0.6% methylcellulose and incubated for 2 days. The overlay was removed, and the plates were fixed in 4% paraformaldehyde twice following BSL-3 protocol. The plates were stained with 1% crystal violet and virus-induced plaques were counted. The percent neutralization and/or NT₅₀ of antibody was calculated by dividing the plaques counted at each dilution with plaques of virus-only control. For antibodies, the inhibitory concentration at 50% (IC₅₀) values were calculated in Prism software (GraphPad) by plotting the midway point between the upper and lower plateaus of the neutralization curve among dilutions. The Alpha variant virus incorporates the following substitutions: Del 69-70, Del 144, E484K, N501Y, A570D, D614G, P681H, T716I, S982A, D1118H. The Beta variant incorporates the following substitutions: Del 24, Del 242-243, D80A, D215G, K417N, E484K, N501Y, D614G, H665Y, T1027I. The Gamma variant incorporates the following substitutions: L18F, T20N, P26S, D138Y, R190S, K417T, E484K, N501Y, D614G, H655Y, T1027I. The Delta variant incorporates the following substitutions: T19R, G142D, Del 156-157, R158G, L452R, T478K, D614G, P681R, Del 689-691, D950N; the deletion at positions 689-691 has not been observed in nature, and was identified upon one passage of the virus.

Fab Preparation

To generate Fabs, IgGs were incubated with Lys-C at 1:4,000 (weight:weight) overnight at 37° C. EDTA free protease inhibitor (Roche) was dissolved to 25× and then added to the sample at a final 1× concentration. The sample was passed over a Protein A column. The flow-through was collected run on a Superdex 200 Increase 10/300 GL Sizing column on the AKTA FPLC system. Fabs were visualized on SDS-PAGE.

Biolayer Interferometry

Purified mAbs were immobilized to AHC sensortips (FortdBio) to a response level of approximately 1.4 nm in a buffer composed of 10 mM HEPES pH 7.5, 150 mM NaCl, 3 mM EDTA, 0.05% Tween 20 and 0.1% (w/v) BSA. Immobilized mAbs were then dipped into wells containing two-fold dilutions of either SARS-CoV-2 RBD-SD1 or SARS-CoV-2 NTD, ranging in concentration from 10-0.15625 nM, to measure association. Dissociation was measured by dipping sensortips into wells containing only running buffer. Data were reference subtracted and kinetics were calculated in Octet Data Analysis software v10.0 using a 1:1 binding model.

Electron Microscopy Sample Preparation and Data Collection

Purified SARS-CoV-2 S HexaPro ectodomain and Fabs 5317-4 and 5317-10 were combined at a final complex concentration of 0.4 mg/mL. Fab 5317-10 was added to spike and incubated on ice for 30 minutes before the addition of Fab 5317-4 immediately prior to grid deposition and freezing. The complex was deposited on Au-300 1.2/1.3 grids that had been plasma cleaned for 4 minutes in a Solarus 950 plasma cleaner (Gatan) with a 4:1 ratio of O₂/H₂. Excess liquid was blotted for 3 seconds with a force of −4 using a Vitrobot Mark IV (Thermo Fisher) and plunge frozen into liquid ethane. 2,655 micrographs were collected from a single grid with the stage at a 300 tilt using a Titan Krios (Thermo Fisher) equipped with a K3 detector (Gatan). Movies were collected using SerialEM at 29,000× magnification with a corresponding calibrated pixel size of 0.81 Å/pixel.

Cryogenic Electron Microscopy (Cryo-EM)

Motion correction, CTF estimation, particle picking, and 2D classification were performed using cryoSPARC v3.2.0. The final iteration of 2D class averaging distributed 17,710 particles into 50 classes using an uncertainty factor of 3. From that, 13,232 particles were selected and an ab into reconstruction was performed with four classes followed by heterogeneous refinement of those four classes. 6,803 particles from the highest-quality class were used for homogenous refinement of the best volume without imposed symmetry. The resulting volume was used for an additional round of homogenous refinement. To filter out additional junk particles, an ab initio reconstruction was performed with three classes followed by heterogeneous refinement of those three classes. 5,171 particles from the highest-quality class were used for homogenous refinement of the best volume without imposed symmetry, resulting in a final 9 Å map.

Quantification and Statistical Analysis

ELISA error bars (standard error of the mean) were calculated using GraphPad Prism version 8.0.0. Spearman r correlation was performed using GraphPad Prism 8.0.0. ANOVA analysis was performed for neutralization potency comparisons using GraphPad Prism version 8.0.0.

In the examples above, large numbers of antibody sequences were determined (see sequences provided below). The following paired heavy chain and light chain sequences are used herein for methods of treating, preventing, or detecting coronavirus infections.

TABLE 1
Paired heavy and light chains and the CDRs thereof.

| Ab of | Cell Barcode | V-D-J- | | | | Cell Barcode | V-J- | | | |
|---|---|---|---|---|---|---|---|---|---|---|
| Special | and Heavy | REGION | CDRH1 | CDRH2 | CDRH3 | and Light | REGION | CDRL1 | CDRL2 | CDRL3 |
| Interest | Chain (HC) | (HC) SEQ | SEQ | SEQ | SEQ | Chain (LC) | (LC) SEQ | SEQ | SEQ | SEQ |
| Name | designation | ID NO | ID NO | ID NO | ID NO | designation | ID NO | ID NO | ID NO | ID NO |
| 5317-1 | TGAGGGAGTT | 2400 | 4056 | 4884 | 5712 | TGAGGGAGTT | 3228 | 6540 | 7368 | 8196 |
| | GTGGCC.HC | | | | | GTGGCC.LC | | | | |
| 5317-2 | GAGCAGACA | 2135 | 3791 | 4619 | 5447 | GAGCAGACA | 2963 | 6275 | 7103 | 7931 |
| | CGGCGTT.HC | | | | | CGGCGTT.LC | | | | |
| 5317-3 | GCGCCAAAG | 2202 | 3858 | 4686 | 5514 | GCGCCAAAG | 3030 | 6342 | 7170 | 7998 |
| | GGCTTCC.HC | | | | | GGCTTCC.LC | | | | |
| 5317-4 | CTAATGGAGC | 2024 | 3680 | 4508 | 5336 | CTAATGGAGC | 2852 | 6164 | 6992 | 7820 |
| | TAACTC.HC | | | | | TAACTC.LC | | | | |
| 5317-5 | GCGCAGTTCA | 2200 | 3856 | 4684 | 5512 | GCGCAGTTCA | 3028 | 6340 | 7168 | 7996 |
| | GCTGGC.HC | | | | | GCTGGC.LC | | | | |
| 5317-6 | ACAGCCGAG | 1699 | 3355 | 4183 | 5011 | ACAGCCGAG | 2527 | 5839 | 6667 | 7495 |
| | AACAACT.HC | | | | | AACAACT.LC | | | | |
| 5317-7 | CTCACACGTA | 2041 | 3697 | 4525 | 5353 | CTCACACGTA | 2869 | 6181 | 7009 | 7837 |
| | AGGGCT.HC | | | | | AGGGCT.LC | | | | |
| 5317-8 | CAACTAGCAT | 1825 | 3481 | 4309 | 5137 | CAACTAGCAT | 2653 | 5965 | 6793 | 7621 |
| | ACGCTA.HC | | | | | ACGCTA.LC | | | | |
| 5317-9 | GGAGCAAGTT | 2240 | 3896 | 4724 | 5552 | GGAGCAAGTT | 3068 | 6380 | 7208 | 8036 |
| | ATCCGA.HC | | | | | ATCCGA.LC | | | | |
| 5317-10 | CGTCAGGAG | 2011 | 3667 | 4495 | 5323 | CGTCAGGAG | 2839 | 6151 | 6979 | 7807 |
| | ACTAGGC.HC | | | | | ACTAGGC.LC | | | | |
| 53181-1 | TGGACGCCAT | 10710 | 12368 | 13197 | 14026 | TGGACGCCAT | 11539 | 14855 | 15684 | 16513 |
| | GACGGA.HC | | | | | GACGGA.LC | | | | |
| 53181-2 | AAGGAGCAG | 9963 | 11621 | 12450 | 13279 | AAGGAGCAG | 10792 | 14108 | 14937 | 15766 |
| | GGATGGG.HC | | | | | GGATGGG.LC | | | | |
| 53181-3 | AGGTCATTCT | 10084 | 11742 | 12571 | 13400 | AGGTCATTCT | 10913 | 14229 | 15058 | 15887 |
| | GCTTGC.HC | | | | | GCTTGC.LC | | | | |
| 53181-4 | AAACCTGTCG | 9940 | 11598 | 12427 | 13256 | AAACCTGTCG | 10769 | 14085 | 14914 | 15743 |
| | GCGCAT.HC | | | | | GCGCAT.LC | | | | |
| 53181-5 | TACTTACAGT | 10604 | 12262 | 13091 | 13920 | TACTTACAGT | 11433 | 14749 | 15578 | 16407 |
| | GGAGAA.HC | | | | | GGAGAA.LC | | | | |
| 53181-6 | CTCGAGGTCG | 10337 | 11995 | 12824 | 13653 | CTCGAGGTCG | 11166 | 14482 | 15311 | 16140 |
| | CCCTTA.HC | | | | | CCCTTA.LC | | | | |
| 53181-7 | GGGAATGAG | 10506 | 12164 | 12993 | 13822 | GGGAATGAG | 11335 | 14651 | 15480 | 16309 |
| | GGCTTGA.HC | | | | | GGCTTGA.LC | | | | |
| 53181-8 | AGTGGGAAG | 10094 | 11752 | 12581 | 13410 | AGTGGGAAG | 10923 | 14239 | 15068 | 15897 |
| | TGACTCT.HC | | | | | TGACTCT.LC | | | | |
| 53181-9 | CCTACCAGTT | 10230 | 11888 | 12717 | 13546 | CCTACCAGTT | 11059 | 14375 | 15204 | 16033 |
| | CGTGAT.HC | | | | | CGTGAT.LC | | | | |
| 53182-1 | TAAGTGCTCA | 19216 | 20173 | 21130 | 22087 | TAAGTGCTCA | 23044 | 24001 | 24958 | 25915 |
| | CAATGC.HC | | | | | CAATGC.LC | | | | |
| 53182-2 | GGCTCGAAG | 19108 | 20065 | 21022 | 21979 | GGCTCGAAG | 22936 | 23893 | 24850 | 25807 |
| | AGTAATC.HC | | | | | AGTAATC.LC | | | | |
| 53182-3 | GTAGTCACAT | 19158 | 20115 | 21072 | 22029 | GTAGTCACAT | 22986 | 23943 | 24900 | 25857 |
| | GGAATA.HC | | | | | GGAATA.LC | | | | |
| 53182-4 | CTAAGACAGT | 18916 | 19873 | 20830 | 21787 | CTAAGACAGT | 22744 | 23701 | 24658 | 25615 |
| | GCAAGC.HC | | | | | GCAAGC.LC | | | | |
| 53182-5 | CTGCTGTGTC | 18966 | 19923 | 20880 | 21837 | CTGCTGTGTC | 22794 | 23751 | 24708 | 25665 |
| | TTGTCC.HC | | | | | TTGTCC.LC | | | | |
| 53182-6 | AACACGTGTG | 18501 | 19458 | 20415 | 21372 | AACACGTGTG | 22329 | 23286 | 24243 | 25200 |
| | ACCAAG.HC | | | | | ACCAAG.LC | | | | |
| 53182-7 | TCACGAATCA | 19278 | 20235 | 21192 | 22149 | TCACGAATCA | 23106 | 24063 | 25020 | 25977 |
| | GAGGTG.HC | | | | | GAGGTG.LC | | | | |
| 53182-8 | CGTCAGGCAG | 18901 | 19858 | 20815 | 21772 | CGTCAGGCAG | 22729 | 23686 | 24643 | 25600 |
| | GAACGT.HC | | | | | GAACGT.LC | | | | |
| 53182-9 | CATGCCTGTA | 18778 | 19735 | 20692 | 21649 | CATGCCTGTA | 22606 | 23563 | 24520 | 25477 |
| | CGACCC.HC | | | | | CGACCC.LC | | | | |
| 53182-10 | CTAGCCTAGA | 18930 | 19887 | 20844 | 21801 | CTAGCCTAGA | 22758 | 23715 | 24672 | 25629 |
| | AAGTGG.HC | | | | | AAGTGG.LC | | | | |
| 53182-11 | ATCATCTGTIT | 18657 | 19614 | 20571 | 21528 | ATCATCTGTTT | 22485 | 23442 | 24399 | 25356 |
| | GTGTG.HC | | | | | GTGTG.LC | | | | |

TABLE 2
Additional paired heavy and light chains and the CDRs thereof

| SEQ ID NO of | | | | | SEQ ID NO of | | | | |
|---|---|---|---|---|---|---|---|---|---|
| Cell Barcode | | | | | Cell Barcode | | | | |
| and Heavy | VH | CDRH1 | CDRH2 | CDRH3 | and Light | VL | CDRL1 | CDRL2 | CDRL3 |
| Chain (HC) | SEQ ID | SEQ ID | SEQ ID | SEQ ID | Chain (LC) | SEQ ID | SEQ ID | SEQ ID | SEQ ID |
| designation | NO | NO | NO | NO | designation | NO | NO | NO | NO |
| 1 | 1657 | 3313 | 4141 | 4969 | 2 | 2485 | 5797 | 6625 | 7453 |
| 3 | 1658 | 3314 | 4142 | 4970 | 4 | 2486 | 5798 | 6626 | 7454 |
| 5 | 1659 | 3315 | 4143 | 4971 | 6 | 2487 | 5799 | 6627 | 7455 |
| 7 | 1660 | 3316 | 4144 | 4972 | 8 | 2488 | 5800 | 6628 | 7456 |
| 9 | 1661 | 3317 | 4145 | 4973 | 10 | 2489 | 5801 | 6629 | 7457 |
| 11 | 1662 | 3318 | 4146 | 4974 | 12 | 2490 | 5802 | 6630 | 7458 |
| 13 | 1663 | 3319 | 4147 | 4975 | 14 | 2491 | 5803 | 6631 | 7459 |
| 15 | 1664 | 3320 | 4148 | 4976 | 16 | 2492 | 5804 | 6632 | 7460 |
| 17 | 1665 | 3321 | 4149 | 4977 | 18 | 2493 | 5805 | 6633 | 7461 |
| 19 | 1666 | 3322 | 4150 | 4978 | 20 | 2494 | 5806 | 6634 | 7462 |
| 21 | 1667 | 3323 | 4151 | 4979 | 22 | 2495 | 5807 | 6635 | 7463 |
| 23 | 1668 | 3324 | 4152 | 4980 | 24 | 2496 | 5808 | 6636 | 7464 |
| 25 | 1669 | 3325 | 4153 | 4981 | 26 | 2497 | 5809 | 6637 | 7465 |
| 27 | 1670 | 3326 | 4154 | 4982 | 28 | 2498 | 5810 | 6638 | 7466 |
| 29 | 1671 | 3327 | 4155 | 4983 | 30 | 2499 | 5811 | 6639 | 7467 |
| 31 | 1672 | 3328 | 4156 | 4984 | 32 | 2500 | 5812 | 6640 | 7468 |
| 33 | 1673 | 3329 | 4157 | 4985 | 34 | 2501 | 5813 | 6641 | 7469 |
| 35 | 1674 | 3330 | 4158 | 4986 | 36 | 2502 | 5814 | 6642 | 7470 |
| 37 | 1675 | 3331 | 4159 | 4987 | 38 | 2503 | 5815 | 6643 | 7471 |
| 39 | 1676 | 3332 | 4160 | 4988 | 40 | 2504 | 5816 | 6644 | 7472 |
| 41 | 1677 | 3333 | 4161 | 4989 | 42 | 2505 | 5817 | 6645 | 7473 |
| 43 | 1678 | 3334 | 4162 | 4990 | 44 | 2506 | 5818 | 6646 | 7474 |
| 45 | 1679 | 3335 | 4163 | 4991 | 46 | 2507 | 5819 | 6647 | 7475 |
| 47 | 1680 | 3336 | 4164 | 4992 | 48 | 2508 | 5820 | 6648 | 7476 |
| 49 | 1681 | 3337 | 4165 | 4993 | 50 | 2509 | 5821 | 6649 | 7477 |
| 51 | 1682 | 3338 | 4166 | 4994 | 52 | 2510 | 5822 | 6650 | 7478 |
| 53 | 1683 | 3339 | 4167 | 4995 | 54 | 2511 | 5823 | 6651 | 7479 |
| 55 | 1684 | 3340 | 4168 | 4996 | 56 | 2512 | 5824 | 6652 | 7480 |
| 57 | 1685 | 3341 | 4169 | 4997 | 58 | 2513 | 5825 | 6653 | 7481 |
| 59 | 1686 | 3342 | 4170 | 4998 | 60 | 2514 | 5826 | 6654 | 7482 |
| 61 | 1687 | 3343 | 4171 | 4999 | 62 | 2515 | 5827 | 6655 | 7483 |
| 63 | 1688 | 3344 | 4172 | 5000 | 64 | 2516 | 5828 | 6656 | 7484 |
| 65 | 1689 | 3345 | 4173 | 5001 | 66 | 2517 | 5829 | 6657 | 7485 |
| 67 | 1690 | 3346 | 4174 | 5002 | 68 | 2518 | 5830 | 6658 | 7486 |
| 69 | 1691 | 3347 | 4175 | 5003 | 70 | 2519 | 5831 | 6659 | 7487 |
| 71 | 1692 | 3348 | 4176 | 5004 | 72 | 2520 | 5832 | 6660 | 7488 |
| 73 | 1693 | 3349 | 4177 | 5005 | 74 | 2521 | 5833 | 6661 | 7489 |
| 75 | 1694 | 3350 | 4178 | 5006 | 76 | 2522 | 5834 | 6662 | 7490 |
| 77 | 1695 | 3351 | 4179 | 5007 | 78 | 2523 | 5835 | 6663 | 7491 |
| 79 | 1696 | 3352 | 4180 | 5008 | 80 | 2524 | 5836 | 6664 | 7492 |
| 81 | 1697 | 3353 | 4181 | 5009 | 82 | 2525 | 5837 | 6665 | 7493 |
| 83 | 1698 | 3354 | 4182 | 5010 | 84 | 2526 | 5838 | 6666 | 7494 |
| 85 | 1699 | 3355 | 4183 | 5011 | 86 | 2527 | 5839 | 6667 | 7495 |
| 87 | 1700 | 3356 | 4184 | 5012 | 88 | 2528 | 5840 | 6668 | 7496 |
| 89 | 1701 | 3357 | 4185 | 5013 | 90 | 2529 | 5841 | 6669 | 7497 |
| 91 | 1702 | 3358 | 4186 | 5014 | 92 | 2530 | 5842 | 6670 | 7498 |
| 93 | 1703 | 3359 | 4187 | 5015 | 94 | 2531 | 5843 | 6671 | 7499 |
| 95 | 1704 | 3360 | 4188 | 5016 | 96 | 2532 | 5844 | 6672 | 7500 |
| 97 | 1705 | 3361 | 4189 | 5017 | 98 | 2533 | 5845 | 6673 | 7501 |
| 99 | 1706 | 3362 | 4190 | 5018 | 100 | 2534 | 5846 | 6674 | 7502 |
| 101 | 1707 | 3363 | 4191 | 5019 | 102 | 2535 | 5847 | 6675 | 7503 |
| 103 | 1708 | 3364 | 4192 | 5020 | 104 | 2536 | 5848 | 6676 | 7504 |
| 105 | 1709 | 3365 | 4193 | 5021 | 106 | 2537 | 5849 | 6677 | 7505 |
| 107 | 1710 | 3366 | 4194 | 5022 | 108 | 2538 | 5850 | 6678 | 7506 |
| 109 | 1711 | 3367 | 4195 | 5023 | 110 | 2539 | 5851 | 6679 | 7507 |
| 111 | 1712 | 3368 | 4196 | 5024 | 112 | 2540 | 5852 | 6680 | 7508 |
| 113 | 1713 | 3369 | 4197 | 5025 | 114 | 2541 | 5853 | 6681 | 7509 |
| 115 | 1714 | 3370 | 4198 | 5026 | 116 | 2542 | 5854 | 6682 | 7510 |
| 117 | 1715 | 3371 | 4199 | 5027 | 118 | 2543 | 5855 | 6683 | 7511 |
| 119 | 1716 | 3372 | 4200 | 5028 | 120 | 2544 | 5856 | 6684 | 7512 |
| 121 | 1717 | 3373 | 4201 | 5029 | 122 | 2545 | 5857 | 6685 | 7513 |
| 123 | 1718 | 3374 | 4202 | 5030 | 124 | 2546 | 5858 | 6686 | 7514 |
| 125 | 1719 | 3375 | 4203 | 5031 | 126 | 2547 | 5859 | 6687 | 7515 |
| 127 | 1720 | 3376 | 4204 | 5032 | 128 | 2548 | 5860 | 6688 | 7516 |
| 129 | 1721 | 3377 | 4205 | 5033 | 130 | 2549 | 5861 | 6689 | 7517 |
| 131 | 1722 | 3378 | 4206 | 5034 | 132 | 2550 | 5862 | 6690 | 7518 |
| 133 | 1723 | 3379 | 4207 | 5035 | 134 | 2551 | 5863 | 6691 | 7519 |
| 135 | 1724 | 3380 | 4208 | 5036 | 136 | 2552 | 5864 | 6692 | 7520 |
| 137 | 1725 | 3381 | 4209 | 5037 | 138 | 2553 | 5865 | 6693 | 7521 |
| 139 | 1726 | 3382 | 4210 | 5038 | 140 | 2554 | 5866 | 6694 | 7522 |
| 141 | 1727 | 3383 | 4211 | 5039 | 142 | 2555 | 5867 | 6695 | 7523 |
| 143 | 1728 | 3384 | 4212 | 5040 | 144 | 2556 | 5868 | 6696 | 7524 |
| 145 | 1729 | 3385 | 4213 | 5041 | 146 | 2557 | 5869 | 6697 | 7525 |
| 147 | 1730 | 3386 | 4214 | 5042 | 148 | 2558 | 5870 | 6698 | 7526 |
| 149 | 1731 | 3387 | 4215 | 5043 | 150 | 2559 | 5871 | 6699 | 7527 |
| 151 | 1732 | 3388 | 4216 | 5044 | 152 | 2560 | 5872 | 6700 | 7528 |
| 153 | 1733 | 3389 | 4217 | 5045 | 154 | 2561 | 5873 | 6701 | 7529 |
| 155 | 1734 | 3390 | 4218 | 5046 | 156 | 2562 | 5874 | 6702 | 7530 |
| 157 | 1735 | 3391 | 4219 | 5047 | 158 | 2563 | 5875 | 6703 | 7531 |
| 159 | 1736 | 3392 | 4220 | 5048 | 160 | 2564 | 5876 | 6704 | 7532 |
| 161 | 1737 | 3393 | 4221 | 5049 | 162 | 2565 | 5877 | 6705 | 7533 |
| 163 | 1738 | 3394 | 4222 | 5050 | 164 | 2566 | 5878 | 6706 | 7534 |
| 165 | 1739 | 3395 | 4223 | 5051 | 166 | 2567 | 5879 | 6707 | 7535 |
| 167 | 1740 | 3396 | 4224 | 5052 | 168 | 2568 | 5880 | 6708 | 7536 |
| 169 | 1741 | 3397 | 4225 | 5053 | 170 | 2569 | 5881 | 6709 | 7537 |
| 171 | 1742 | 3398 | 4226 | 5054 | 172 | 2570 | 5882 | 6710 | 7538 |
| 173 | 1743 | 3399 | 4227 | 5055 | 174 | 2571 | 5883 | 6711 | 7539 |
| 175 | 1744 | 3400 | 4228 | 5056 | 176 | 2572 | 5884 | 6712 | 7540 |
| 177 | 1745 | 3401 | 4229 | 5057 | 178 | 2573 | 5885 | 6713 | 7541 |
| 179 | 1746 | 3402 | 4230 | 5058 | 180 | 2574 | 5886 | 6714 | 7542 |
| 181 | 1747 | 3403 | 4231 | 5059 | 182 | 2575 | 5887 | 6715 | 7543 |
| 183 | 1748 | 3404 | 4232 | 5060 | 184 | 2576 | 5888 | 6716 | 7544 |
| 185 | 1749 | 3405 | 4233 | 5061 | 186 | 2577 | 5889 | 6717 | 7545 |
| 187 | 1750 | 3406 | 4234 | 5062 | 188 | 2578 | 5890 | 6718 | 7546 |
| 189 | 1751 | 3407 | 4235 | 5063 | 190 | 2579 | 5891 | 6719 | 7547 |
| 191 | 1752 | 3408 | 4236 | 5064 | 192 | 2580 | 5892 | 6720 | 7548 |
| 193 | 1753 | 3409 | 4237 | 5065 | 194 | 2581 | 5893 | 6721 | 7549 |
| 195 | 1754 | 3410 | 4238 | 5066 | 196 | 2582 | 5894 | 6722 | 7550 |
| 197 | 1755 | 3411 | 4239 | 5067 | 198 | 2583 | 5895 | 6723 | 7551 |
| 199 | 1756 | 3412 | 4240 | 5068 | 200 | 2584 | 5896 | 6724 | 7552 |
| 201 | 1757 | 3413 | 4241 | 5069 | 202 | 2585 | 5897 | 6725 | 7553 |
| 203 | 1758 | 3414 | 4242 | 5070 | 204 | 2586 | 5898 | 6726 | 7554 |
| 205 | 1759 | 3415 | 4243 | 5071 | 206 | 2587 | 5899 | 6727 | 7555 |
| 207 | 1760 | 3416 | 4244 | 5072 | 208 | 2588 | 5900 | 6728 | 7556 |
| 209 | 1761 | 3417 | 4245 | 5073 | 210 | 2589 | 5901 | 6729 | 7557 |
| 211 | 1762 | 3418 | 4246 | 5074 | 212 | 2590 | 5902 | 6730 | 7558 |
| 213 | 1763 | 3419 | 4247 | 5075 | 214 | 2591 | 5903 | 6731 | 7559 |
| 215 | 1764 | 3420 | 4248 | 5076 | 216 | 2592 | 5904 | 6732 | 7560 |
| 217 | 1765 | 3421 | 4249 | 5077 | 218 | 2593 | 5905 | 6733 | 7561 |
| 219 | 1766 | 3422 | 4250 | 5078 | 220 | 2594 | 5906 | 6734 | 7562 |
| 221 | 1767 | 3423 | 4251 | 5079 | 222 | 2595 | 5907 | 6735 | 7563 |
| 223 | 1768 | 3424 | 4252 | 5080 | 224 | 2596 | 5908 | 6736 | 7564 |
| 225 | 1769 | 3425 | 4253 | 5081 | 226 | 2597 | 5909 | 6737 | 7565 |
| 227 | 1770 | 3426 | 4254 | 5082 | 228 | 2598 | 5910 | 6738 | 7566 |
| 229 | 1771 | 3427 | 4255 | 5083 | 230 | 2599 | 5911 | 6739 | 7567 |
| 231 | 1772 | 3428 | 4256 | 5084 | 232 | 2600 | 5912 | 6740 | 7568 |
| 233 | 1773 | 3429 | 4257 | 5085 | 234 | 2601 | 5913 | 6741 | 7569 |
| 235 | 1774 | 3430 | 4258 | 5086 | 236 | 2602 | 5914 | 6742 | 7570 |
| 237 | 1775 | 3431 | 4259 | 5087 | 238 | 2603 | 5915 | 6743 | 7571 |
| 239 | 1776 | 3432 | 4260 | 5088 | 240 | 2604 | 5916 | 6744 | 7572 |
| 241 | 1777 | 3433 | 4261 | 5089 | 242 | 2605 | 5917 | 6745 | 7573 |
| 243 | 1778 | 3434 | 4262 | 5090 | 244 | 2606 | 5918 | 6746 | 7574 |
| 245 | 1779 | 3435 | 4263 | 5091 | 246 | 2607 | 5919 | 6747 | 7575 |
| 247 | 1780 | 3436 | 4264 | 5092 | 248 | 2608 | 5920 | 6748 | 7576 |
| 249 | 1781 | 3437 | 4265 | 5093 | 250 | 2609 | 5921 | 6749 | 7577 |
| 251 | 1782 | 3438 | 4266 | 5094 | 252 | 2610 | 5922 | 6750 | 7578 |
| 253 | 1783 | 3439 | 4267 | 5095 | 254 | 2611 | 5923 | 6751 | 7579 |
| 255 | 1784 | 3440 | 4268 | 5096 | 256 | 2612 | 5924 | 6752 | 7580 |
| 257 | 1785 | 3441 | 4269 | 5097 | 258 | 2613 | 5925 | 6753 | 7581 |
| 259 | 1786 | 3442 | 4270 | 5098 | 260 | 2614 | 5926 | 6754 | 7582 |
| 261 | 1787 | 3443 | 4271 | 5099 | 262 | 2615 | 5927 | 6755 | 7583 |
| 263 | 1788 | 3444 | 4272 | 5100 | 264 | 2616 | 5928 | 6756 | 7584 |
| 265 | 1789 | 3445 | 4273 | 5101 | 266 | 2617 | 5929 | 6757 | 7585 |
| 267 | 1790 | 3446 | 4274 | 5102 | 268 | 2618 | 5930 | 6758 | 7586 |
| 269 | 1791 | 3447 | 4275 | 5103 | 270 | 2619 | 5931 | 6759 | 7587 |
| 271 | 1792 | 3448 | 4276 | 5104 | 272 | 2620 | 5932 | 6760 | 7588 |
| 273 | 1793 | 3449 | 4277 | 5105 | 274 | 2621 | 5933 | 6761 | 7589 |
| 275 | 1794 | 3450 | 4278 | 5106 | 276 | 2622 | 5934 | 6762 | 7590 |
| 277 | 1795 | 3451 | 4279 | 5107 | 278 | 2623 | 5935 | 6763 | 7591 |
| 279 | 1796 | 3452 | 4280 | 5108 | 280 | 2624 | 5936 | 6764 | 7592 |
| 281 | 1797 | 3453 | 4281 | 5109 | 282 | 2625 | 5937 | 6765 | 7593 |
| 283 | 1798 | 3454 | 4282 | 5110 | 284 | 2626 | 5938 | 6766 | 7594 |
| 285 | 1799 | 3455 | 4283 | 5111 | 286 | 2627 | 5939 | 6767 | 7595 |
| 287 | 1800 | 3456 | 4284 | 5112 | 288 | 2628 | 5940 | 6768 | 7596 |
| 289 | 1801 | 3457 | 4285 | 5113 | 290 | 2629 | 5941 | 6769 | 7597 |
| 291 | 1802 | 3458 | 4286 | 5114 | 292 | 2630 | 5942 | 6770 | 7598 |
| 293 | 1803 | 3459 | 4287 | 5115 | 294 | 2631 | 5943 | 6771 | 7599 |
| 295 | 1804 | 3460 | 4288 | 5116 | 296 | 2632 | 5944 | 6772 | 7600 |
| 297 | 1805 | 3461 | 4289 | 5117 | 298 | 2633 | 5945 | 6773 | 7601 |
| 299 | 1806 | 3462 | 4290 | 5118 | 300 | 2634 | 5946 | 6774 | 7602 |
| 301 | 1807 | 3463 | 4291 | 5119 | 302 | 2635 | 5947 | 6775 | 7603 |
| 303 | 1808 | 3464 | 4292 | 5120 | 304 | 2636 | 5948 | 6776 | 7604 |
| 305 | 1809 | 3465 | 4293 | 5121 | 306 | 2637 | 5949 | 6777 | 7605 |
| 307 | 1810 | 3466 | 4294 | 5122 | 308 | 2638 | 5950 | 6778 | 7606 |
| 309 | 1811 | 3467 | 4295 | 5123 | 310 | 2639 | 5951 | 6779 | 7607 |
| 311 | 1812 | 3468 | 4296 | 5124 | 312 | 2640 | 5952 | 6780 | 7608 |
| 313 | 1813 | 3469 | 4297 | 5125 | 314 | 2641 | 5953 | 6781 | 7609 |
| 315 | 1814 | 3470 | 4298 | 5126 | 316 | 2642 | 5954 | 6782 | 7610 |
| 317 | 1815 | 3471 | 4299 | 5127 | 318 | 2643 | 5955 | 6783 | 7611 |
| 319 | 1816 | 3472 | 4300 | 5128 | 320 | 2644 | 5956 | 6784 | 7612 |
| 321 | 1817 | 3473 | 4301 | 5129 | 322 | 2645 | 5957 | 6785 | 7613 |
| 323 | 1818 | 3474 | 4302 | 5130 | 324 | 2646 | 5958 | 6786 | 7614 |
| 325 | 1819 | 3475 | 4303 | 5131 | 326 | 2647 | 5959 | 6787 | 7615 |
| 327 | 1820 | 3476 | 4304 | 5132 | 328 | 2648 | 5960 | 6788 | 7616 |
| 329 | 1821 | 3477 | 4305 | 5133 | 330 | 2649 | 5961 | 6789 | 7617 |
| 331 | 1822 | 3478 | 4306 | 5134 | 332 | 2650 | 5962 | 6790 | 7618 |
| 333 | 1823 | 3479 | 4307 | 5135 | 334 | 2651 | 5963 | 6791 | 7619 |
| 335 | 1824 | 3480 | 4308 | 5136 | 336 | 2652 | 5964 | 6792 | 7620 |
| 337 | 1825 | 3481 | 4309 | 5137 | 338 | 2653 | 5965 | 6793 | 7621 |
| 339 | 1826 | 3482 | 4310 | 5138 | 340 | 2654 | 5966 | 6794 | 7622 |
| 341 | 1827 | 3483 | 4311 | 5139 | 342 | 2655 | 5967 | 6795 | 7623 |
| 343 | 1828 | 3484 | 4312 | 5140 | 344 | 2656 | 5968 | 6796 | 7624 |
| 345 | 1829 | 3485 | 4313 | 5141 | 346 | 2657 | 5969 | 6797 | 7625 |
| 347 | 1830 | 3486 | 4314 | 5142 | 348 | 2658 | 5970 | 6798 | 7626 |
| 349 | 1831 | 3487 | 4315 | 5143 | 350 | 2659 | 5971 | 6799 | 7627 |
| 351 | 1832 | 3488 | 4316 | 5144 | 352 | 2660 | 5972 | 6800 | 7628 |
| 353 | 1833 | 3489 | 4317 | 5145 | 354 | 2661 | 5973 | 6801 | 7629 |
| 355 | 1834 | 3490 | 4318 | 5146 | 356 | 2662 | 5974 | 6802 | 7630 |
| 357 | 1835 | 3491 | 4319 | 5147 | 358 | 2663 | 5975 | 6803 | 7631 |
| 359 | 1836 | 3492 | 4320 | 5148 | 360 | 2664 | 5976 | 6804 | 7632 |
| 361 | 1837 | 3493 | 4321 | 5149 | 362 | 2665 | 5977 | 6805 | 7633 |
| 363 | 1838 | 3494 | 4322 | 5150 | 364 | 2666 | 5978 | 6806 | 7634 |
| 365 | 1839 | 3495 | 4323 | 5151 | 366 | 2667 | 5979 | 6807 | 7635 |
| 367 | 1840 | 3496 | 4324 | 5152 | 368 | 2668 | 5980 | 6808 | 7636 |
| 369 | 1841 | 3497 | 4325 | 5153 | 370 | 2669 | 5981 | 6809 | 7637 |
| 371 | 1842 | 3498 | 4326 | 5154 | 372 | 2670 | 5982 | 6810 | 7638 |
| 373 | 1843 | 3499 | 4327 | 5155 | 374 | 2671 | 5983 | 6811 | 7639 |
| 375 | 1844 | 3500 | 4328 | 5156 | 376 | 2672 | 5984 | 6812 | 7640 |
| 377 | 1845 | 3501 | 4329 | 5157 | 378 | 2673 | 5985 | 6813 | 7641 |
| 379 | 1846 | 3502 | 4330 | 5158 | 380 | 2674 | 5986 | 6814 | 7642 |
| 381 | 1847 | 3503 | 4331 | 5159 | 382 | 2675 | 5987 | 6815 | 7643 |
| 383 | 1848 | 3504 | 4332 | 5160 | 384 | 2676 | 5988 | 6816 | 7644 |
| 385 | 1849 | 3505 | 4333 | 5161 | 386 | 2677 | 5989 | 6817 | 7645 |
| 387 | 1850 | 3506 | 4334 | 5162 | 388 | 2678 | 5990 | 6818 | 7646 |
| 389 | 1851 | 3507 | 4335 | 5163 | 390 | 2679 | 5991 | 6819 | 7647 |
| 391 | 1852 | 3508 | 4336 | 5164 | 392 | 2680 | 5992 | 6820 | 7648 |
| 393 | 1853 | 3509 | 4337 | 5165 | 394 | 2681 | 5993 | 6821 | 7649 |
| 395 | 1854 | 3510 | 4338 | 5166 | 396 | 2682 | 5994 | 6822 | 7650 |
| 397 | 1855 | 3511 | 4339 | 5167 | 398 | 2683 | 5995 | 6823 | 7651 |
| 399 | 1856 | 3512 | 4340 | 5168 | 400 | 2684 | 5996 | 6824 | 7652 |
| 401 | 1857 | 3513 | 4341 | 5169 | 402 | 2685 | 5997 | 6825 | 7653 |
| 403 | 1858 | 3514 | 4342 | 5170 | 404 | 2686 | 5998 | 6826 | 7654 |
| 405 | 1859 | 3515 | 4343 | 5171 | 406 | 2687 | 5999 | 6827 | 7655 |
| 407 | 1860 | 3516 | 4344 | 5172 | 408 | 2688 | 6000 | 6828 | 7656 |
| 409 | 1861 | 3517 | 4345 | 5173 | 410 | 2689 | 6001 | 6829 | 7657 |
| 411 | 1862 | 3518 | 4346 | 5174 | 412 | 2690 | 6002 | 6830 | 7658 |
| 413 | 1863 | 3519 | 4347 | 5175 | 414 | 2691 | 6003 | 6831 | 7659 |
| 415 | 1864 | 3520 | 4348 | 5176 | 416 | 2692 | 6004 | 6832 | 7660 |
| 417 | 1865 | 3521 | 4349 | 5177 | 418 | 2693 | 6005 | 6833 | 7661 |
| 419 | 1866 | 3522 | 4350 | 5178 | 420 | 2694 | 6006 | 6834 | 7662 |
| 421 | 1867 | 3523 | 4351 | 5179 | 422 | 2695 | 6007 | 6835 | 7663 |
| 423 | 1868 | 3524 | 4352 | 5180 | 424 | 2696 | 6008 | 6836 | 7664 |
| 425 | 1869 | 3525 | 4353 | 5181 | 426 | 2697 | 6009 | 6837 | 7665 |
| 427 | 1870 | 3526 | 4354 | 5182 | 428 | 2698 | 6010 | 6838 | 7666 |
| 429 | 1871 | 3527 | 4355 | 5183 | 430 | 2699 | 6011 | 6839 | 7667 |
| 431 | 1872 | 3528 | 4356 | 5184 | 432 | 2700 | 6012 | 6840 | 7668 |
| 433 | 1873 | 3529 | 4357 | 5185 | 434 | 2701 | 6013 | 6841 | 7669 |
| 435 | 1874 | 3530 | 4358 | 5186 | 436 | 2702 | 6014 | 6842 | 7670 |
| 437 | 1875 | 3531 | 4359 | 5187 | 438 | 2703 | 6015 | 6843 | 7671 |
| 439 | 1876 | 3532 | 4360 | 5188 | 440 | 2704 | 6016 | 6844 | 7672 |
| 441 | 1877 | 3533 | 4361 | 5189 | 442 | 2705 | 6017 | 6845 | 7673 |
| 443 | 1878 | 3534 | 4362 | 5190 | 444 | 2706 | 6018 | 6846 | 7674 |
| 445 | 1879 | 3535 | 4363 | 5191 | 446 | 2707 | 6019 | 6847 | 7675 |
| 447 | 1880 | 3536 | 4364 | 5192 | 448 | 2708 | 6020 | 6848 | 7676 |
| 449 | 1881 | 3537 | 4365 | 5193 | 450 | 2709 | 6021 | 6849 | 7677 |
| 451 | 1882 | 3538 | 4366 | 5194 | 452 | 2710 | 6022 | 6850 | 7678 |
| 453 | 1883 | 3539 | 4367 | 5195 | 454 | 2711 | 6023 | 6851 | 7679 |
| 455 | 1884 | 3540 | 4368 | 5196 | 456 | 2712 | 6024 | 6852 | 7680 |
| 457 | 1885 | 3541 | 4369 | 5197 | 458 | 2713 | 6025 | 6853 | 7681 |
| 459 | 1886 | 3542 | 4370 | 5198 | 460 | 2714 | 6026 | 6854 | 7682 |
| 461 | 1887 | 3543 | 4371 | 5199 | 462 | 2715 | 6027 | 6855 | 7683 |
| 463 | 1888 | 3544 | 4372 | 5200 | 464 | 2716 | 6028 | 6856 | 7684 |
| 465 | 1889 | 3545 | 4373 | 5201 | 466 | 2717 | 6029 | 6857 | 7685 |
| 467 | 1890 | 3546 | 4374 | 5202 | 468 | 2718 | 6030 | 6858 | 7686 |
| 469 | 1891 | 3547 | 4375 | 5203 | 470 | 2719 | 6031 | 6859 | 7687 |
| 471 | 1892 | 3548 | 4376 | 5204 | 472 | 2720 | 6032 | 6860 | 7688 |
| 473 | 1893 | 3549 | 4377 | 5205 | 474 | 2721 | 6033 | 6861 | 7689 |
| 475 | 1894 | 3550 | 4378 | 5206 | 476 | 2722 | 6034 | 6862 | 7690 |
| 477 | 1895 | 3551 | 4379 | 5207 | 478 | 2723 | 6035 | 6863 | 7691 |
| 479 | 1896 | 3552 | 4380 | 5208 | 480 | 2724 | 6036 | 6864 | 7692 |
| 481 | 1897 | 3553 | 4381 | 5209 | 482 | 2725 | 6037 | 6865 | 7693 |
| 483 | 1898 | 3554 | 4382 | 5210 | 484 | 2726 | 6038 | 6866 | 7694 |
| 485 | 1899 | 3555 | 4383 | 5211 | 486 | 2727 | 6039 | 6867 | 7695 |
| 487 | 1900 | 3556 | 4384 | 5212 | 488 | 2728 | 6040 | 6868 | 7696 |
| 489 | 1901 | 3557 | 4385 | 5213 | 490 | 2729 | 6041 | 6869 | 7697 |
| 491 | 1902 | 3558 | 4386 | 5214 | 492 | 2730 | 6042 | 6870 | 7698 |
| 493 | 1903 | 3559 | 4387 | 5215 | 494 | 2731 | 6043 | 6871 | 7699 |
| 495 | 1904 | 3560 | 4388 | 5216 | 496 | 2732 | 6044 | 6872 | 7700 |
| 497 | 1905 | 3561 | 4389 | 5217 | 498 | 2733 | 6045 | 6873 | 7701 |
| 499 | 1906 | 3562 | 4390 | 5218 | 500 | 2734 | 6046 | 6874 | 7702 |
| 501 | 1907 | 3563 | 4391 | 5219 | 502 | 2735 | 6047 | 6875 | 7703 |
| 503 | 1908 | 3564 | 4392 | 5220 | 504 | 2736 | 6048 | 6876 | 7704 |
| 505 | 1909 | 3565 | 4393 | 5221 | 506 | 2737 | 6049 | 6877 | 7705 |
| 507 | 1910 | 3566 | 4394 | 5222 | 508 | 2738 | 6050 | 6878 | 7706 |
| 509 | 1911 | 3567 | 4395 | 5223 | 510 | 2739 | 6051 | 6879 | 7707 |
| 511 | 1912 | 3568 | 4396 | 5224 | 512 | 2740 | 6052 | 6880 | 7708 |
| 513 | 1913 | 3569 | 4397 | 5225 | 514 | 2741 | 6053 | 6881 | 7709 |
| 515 | 1914 | 3570 | 4398 | 5226 | 516 | 2742 | 6054 | 6882 | 7710 |
| 517 | 1915 | 3571 | 4399 | 5227 | 518 | 2743 | 6055 | 6883 | 7711 |
| 519 | 1916 | 3572 | 4400 | 5228 | 520 | 2744 | 6056 | 6884 | 7712 |
| 521 | 1917 | 3573 | 4401 | 5229 | 522 | 2745 | 6057 | 6885 | 7713 |
| 523 | 1918 | 3574 | 4402 | 5230 | 524 | 2746 | 6058 | 6886 | 7714 |
| 525 | 1919 | 3575 | 4403 | 5231 | 526 | 2747 | 6059 | 6887 | 7715 |
| 527 | 1920 | 3576 | 4404 | 5232 | 528 | 2748 | 6060 | 6888 | 7716 |
| 529 | 1921 | 3577 | 4405 | 5233 | 530 | 2749 | 6061 | 6889 | 7717 |
| 531 | 1922 | 3578 | 4406 | 5234 | 532 | 2750 | 6062 | 6890 | 7718 |
| 533 | 1923 | 3579 | 4407 | 5235 | 534 | 2751 | 6063 | 6891 | 7719 |
| 535 | 1924 | 3580 | 4408 | 5236 | 536 | 2752 | 6064 | 6892 | 7720 |
| 537 | 1925 | 3581 | 4409 | 5237 | 538 | 2753 | 6065 | 6893 | 7721 |
| 539 | 1926 | 3582 | 4410 | 5238 | 540 | 2754 | 6066 | 6894 | 7722 |
| 541 | 1927 | 3583 | 4411 | 5239 | 542 | 2755 | 6067 | 6895 | 7723 |
| 543 | 1928 | 3584 | 4412 | 5240 | 544 | 2756 | 6068 | 6896 | 7724 |
| 545 | 1929 | 3585 | 4413 | 5241 | 546 | 2757 | 6069 | 6897 | 7725 |
| 547 | 1930 | 3586 | 4414 | 5242 | 548 | 2758 | 6070 | 6898 | 7726 |
| 549 | 1931 | 3587 | 4415 | 5243 | 550 | 2759 | 6071 | 6899 | 7727 |
| 551 | 1932 | 3588 | 4416 | 5244 | 552 | 2760 | 6072 | 6900 | 7728 |
| 553 | 1933 | 3589 | 4417 | 5245 | 554 | 2761 | 6073 | 6901 | 7729 |
| 555 | 1934 | 3590 | 4418 | 5246 | 556 | 2762 | 6074 | 6902 | 7730 |
| 557 | 1935 | 3591 | 4419 | 5247 | 558 | 2763 | 6075 | 6903 | 7731 |
| 559 | 1936 | 3592 | 4420 | 5248 | 560 | 2764 | 6076 | 6904 | 7732 |
| 561 | 1937 | 3593 | 4421 | 5249 | 562 | 2765 | 6077 | 6905 | 7733 |
| 563 | 1938 | 3594 | 4422 | 5250 | 564 | 2766 | 6078 | 6906 | 7734 |
| 565 | 1939 | 3595 | 4423 | 5251 | 566 | 2767 | 6079 | 6907 | 7735 |
| 567 | 1940 | 3596 | 4424 | 5252 | 568 | 2768 | 6080 | 6908 | 7736 |
| 569 | 1941 | 3597 | 4425 | 5253 | 570 | 2769 | 6081 | 6909 | 7737 |
| 571 | 1942 | 3598 | 4426 | 5254 | 572 | 2770 | 6082 | 6910 | 7738 |
| 573 | 1943 | 3599 | 4427 | 5255 | 574 | 2771 | 6083 | 6911 | 7739 |
| 575 | 1944 | 3600 | 4428 | 5256 | 576 | 2772 | 6084 | 6912 | 7740 |
| 577 | 1945 | 3601 | 4429 | 5257 | 578 | 2773 | 6085 | 6913 | 7741 |
| 579 | 1946 | 3602 | 4430 | 5258 | 580 | 2774 | 6086 | 6914 | 7742 |
| 581 | 1947 | 3603 | 4431 | 5259 | 582 | 2775 | 6087 | 6915 | 7743 |
| 583 | 1948 | 3604 | 4432 | 5260 | 584 | 2776 | 6088 | 6916 | 7744 |
| 585 | 1949 | 3605 | 4433 | 5261 | 586 | 2777 | 6089 | 6917 | 7745 |
| 587 | 1950 | 3606 | 4434 | 5262 | 588 | 2778 | 6090 | 6918 | 7746 |
| 589 | 1951 | 3607 | 4435 | 5263 | 590 | 2779 | 6091 | 6919 | 7747 |
| 591 | 1952 | 3608 | 4436 | 5264 | 592 | 2780 | 6092 | 6920 | 7748 |
| 593 | 1953 | 3609 | 4437 | 5265 | 594 | 2781 | 6093 | 6921 | 7749 |
| 595 | 1954 | 3610 | 4438 | 5266 | 596 | 2782 | 6094 | 6922 | 7750 |
| 597 | 1955 | 3611 | 4439 | 5267 | 598 | 2783 | 6095 | 6923 | 7751 |
| 599 | 1956 | 3612 | 4440 | 5268 | 600 | 2784 | 6096 | 6924 | 7752 |
| 601 | 1957 | 3613 | 4441 | 5269 | 602 | 2785 | 6097 | 6925 | 7753 |
| 603 | 1958 | 3614 | 4442 | 5270 | 604 | 2786 | 6098 | 6926 | 7754 |
| 605 | 1959 | 3615 | 4443 | 5271 | 606 | 2787 | 6099 | 6927 | 7755 |
| 607 | 1960 | 3616 | 4444 | 5272 | 608 | 2788 | 6100 | 6928 | 7756 |
| 609 | 1961 | 3617 | 4445 | 5273 | 610 | 2789 | 6101 | 6929 | 7757 |
| 611 | 1962 | 3618 | 4446 | 5274 | 612 | 2790 | 6102 | 6930 | 7758 |
| 613 | 1963 | 3619 | 4447 | 5275 | 614 | 2791 | 6103 | 6931 | 7759 |
| 615 | 1964 | 3620 | 4448 | 5276 | 616 | 2792 | 6104 | 6932 | 7760 |
| 617 | 1965 | 3621 | 4449 | 5277 | 618 | 2793 | 6105 | 6933 | 7761 |
| 619 | 1966 | 3622 | 4450 | 5278 | 620 | 2794 | 6106 | 6934 | 7762 |
| 621 | 1967 | 3623 | 4451 | 5279 | 622 | 2795 | 6107 | 6935 | 7763 |
| 623 | 1968 | 3624 | 4452 | 5280 | 624 | 2796 | 6108 | 6936 | 7764 |
| 625 | 1969 | 3625 | 4453 | 5281 | 626 | 2797 | 6109 | 6937 | 7765 |
| 627 | 1970 | 3626 | 4454 | 5282 | 628 | 2798 | 6110 | 6938 | 7766 |
| 629 | 1971 | 3627 | 4455 | 5283 | 630 | 2799 | 6111 | 6939 | 7767 |
| 631 | 1972 | 3628 | 4456 | 5284 | 632 | 2800 | 6112 | 6940 | 7768 |
| 633 | 1973 | 3629 | 4457 | 5285 | 634 | 2801 | 6113 | 6941 | 7769 |
| 635 | 1974 | 3630 | 4458 | 5286 | 636 | 2802 | 6114 | 6942 | 7770 |
| 637 | 1975 | 3631 | 4459 | 5287 | 638 | 2803 | 6115 | 6943 | 7771 |
| 639 | 1976 | 3632 | 4460 | 5288 | 640 | 2804 | 6116 | 6944 | 7772 |
| 641 | 1977 | 3633 | 4461 | 5289 | 642 | 2805 | 6117 | 6945 | 7773 |
| 643 | 1978 | 3634 | 4462 | 5290 | 644 | 2806 | 6118 | 6946 | 7774 |
| 645 | 1979 | 3635 | 4463 | 5291 | 646 | 2807 | 6119 | 6947 | 7775 |
| 647 | 1980 | 3636 | 4464 | 5292 | 648 | 2808 | 6120 | 6948 | 7776 |
| 649 | 1981 | 3637 | 4465 | 5293 | 650 | 2809 | 6121 | 6949 | 7777 |
| 651 | 1982 | 3638 | 4466 | 5294 | 652 | 2810 | 6122 | 6950 | 7778 |
| 653 | 1983 | 3639 | 4467 | 5295 | 654 | 2811 | 6123 | 6951 | 7779 |
| 655 | 1984 | 3640 | 4468 | 5296 | 656 | 2812 | 6124 | 6952 | 7780 |
| 657 | 1985 | 3641 | 4469 | 5297 | 658 | 2813 | 6125 | 6953 | 7781 |
| 659 | 1986 | 3642 | 4470 | 5298 | 660 | 2814 | 6126 | 6954 | 7782 |
| 661 | 1987 | 3643 | 4471 | 5299 | 662 | 2815 | 6127 | 6955 | 7783 |
| 663 | 1988 | 3644 | 4472 | 5300 | 664 | 2816 | 6128 | 6956 | 7784 |
| 665 | 1989 | 3645 | 4473 | 5301 | 666 | 2817 | 6129 | 6957 | 7785 |
| 667 | 1990 | 3646 | 4474 | 5302 | 668 | 2818 | 6130 | 6958 | 7786 |
| 669 | 1991 | 3647 | 4475 | 5303 | 670 | 2819 | 6131 | 6959 | 7787 |
| 671 | 1992 | 3648 | 4476 | 5304 | 672 | 2820 | 6132 | 6960 | 7788 |
| 673 | 1993 | 3649 | 4477 | 5305 | 674 | 2821 | 6133 | 6961 | 7789 |
| 675 | 1994 | 3650 | 4478 | 5306 | 676 | 2822 | 6134 | 6962 | 7790 |
| 677 | 1995 | 3651 | 4479 | 5307 | 678 | 2823 | 6135 | 6963 | 7791 |
| 679 | 1996 | 3652 | 4480 | 5308 | 680 | 2824 | 6136 | 6964 | 7792 |
| 681 | 1997 | 3653 | 4481 | 5309 | 682 | 2825 | 6137 | 6965 | 7793 |
| 683 | 1998 | 3654 | 4482 | 5310 | 684 | 2826 | 6138 | 6966 | 7794 |
| 685 | 1999 | 3655 | 4483 | 5311 | 686 | 2827 | 6139 | 6967 | 7795 |
| 687 | 2000 | 3656 | 4484 | 5312 | 688 | 2828 | 6140 | 6968 | 7796 |
| 689 | 2001 | 3657 | 4485 | 5313 | 690 | 2829 | 6141 | 6969 | 7797 |
| 691 | 2002 | 3658 | 4486 | 5314 | 692 | 2830 | 6142 | 6970 | 7798 |
| 693 | 2003 | 3659 | 4487 | 5315 | 694 | 2831 | 6143 | 6971 | 7799 |
| 695 | 2004 | 3660 | 4488 | 5316 | 696 | 2832 | 6144 | 6972 | 7800 |
| 697 | 2005 | 3661 | 4489 | 5317 | 698 | 2833 | 6145 | 6973 | 7801 |
| 699 | 2006 | 3662 | 4490 | 5318 | 700 | 2834 | 6146 | 6974 | 7802 |
| 701 | 2007 | 3663 | 4491 | 5319 | 702 | 2835 | 6147 | 6975 | 7803 |
| 703 | 2008 | 3664 | 4492 | 5320 | 704 | 2836 | 6148 | 6976 | 7804 |
| 705 | 2009 | 3665 | 4493 | 5321 | 706 | 2837 | 6149 | 6977 | 7805 |
| 707 | 2010 | 3666 | 4494 | 5322 | 708 | 2838 | 6150 | 6978 | 7806 |
| 709 | 2011 | 3667 | 4495 | 5323 | 710 | 2839 | 6151 | 6979 | 7807 |
| 711 | 2012 | 3668 | 4496 | 5324 | 712 | 2840 | 6152 | 6980 | 7808 |
| 713 | 2013 | 3669 | 4497 | 5325 | 714 | 2841 | 6153 | 6981 | 7809 |
| 715 | 2014 | 3670 | 4498 | 5326 | 716 | 2842 | 6154 | 6982 | 7810 |
| 717 | 2015 | 3671 | 4499 | 5327 | 718 | 2843 | 6155 | 6983 | 7811 |
| 719 | 2016 | 3672 | 4500 | 5328 | 720 | 2844 | 6156 | 6984 | 7812 |
| 721 | 2017 | 3673 | 4501 | 5329 | 722 | 2845 | 6157 | 6985 | 7813 |
| 723 | 2018 | 3674 | 4502 | 5330 | 724 | 2846 | 6158 | 6986 | 7814 |
| 725 | 2019 | 3675 | 4503 | 5331 | 726 | 2847 | 6159 | 6987 | 7815 |
| 727 | 2020 | 3676 | 4504 | 5332 | 728 | 2848 | 6160 | 6988 | 7816 |
| 729 | 2021 | 3677 | 4505 | 5333 | 730 | 2849 | 6161 | 6989 | 7817 |
| 731 | 2022 | 3678 | 4506 | 5334 | 732 | 2850 | 6162 | 6990 | 7818 |
| 733 | 2023 | 3679 | 4507 | 5335 | 734 | 2851 | 6163 | 6991 | 7819 |
| 735 | 2024 | 3680 | 4508 | 5336 | 736 | 2852 | 6164 | 6992 | 7820 |
| 737 | 2025 | 3681 | 4509 | 5337 | 738 | 2853 | 6165 | 6993 | 7821 |
| 739 | 2026 | 3682 | 4510 | 5338 | 740 | 2854 | 6166 | 6994 | 7822 |
| 741 | 2027 | 3683 | 4511 | 5339 | 742 | 2855 | 6167 | 6995 | 7823 |
| 743 | 2028 | 3684 | 4512 | 5340 | 744 | 2856 | 6168 | 6996 | 7824 |
| 745 | 2029 | 3685 | 4513 | 5341 | 746 | 2857 | 6169 | 6997 | 7825 |
| 747 | 2030 | 3686 | 4514 | 5342 | 748 | 2858 | 6170 | 6998 | 7826 |
| 749 | 2031 | 3687 | 4515 | 5343 | 750 | 2859 | 6171 | 6999 | 7827 |
| 751 | 2032 | 3688 | 4516 | 5344 | 752 | 2860 | 6172 | 7000 | 7828 |
| 753 | 2033 | 3689 | 4517 | 5345 | 754 | 2861 | 6173 | 7001 | 7829 |
| 755 | 2034 | 3690 | 4518 | 5346 | 756 | 2862 | 6174 | 7002 | 7830 |
| 757 | 2035 | 3691 | 4519 | 5347 | 758 | 2863 | 6175 | 7003 | 7831 |
| 759 | 2036 | 3692 | 4520 | 5348 | 760 | 2864 | 6176 | 7004 | 7832 |
| 761 | 2037 | 3693 | 4521 | 5349 | 762 | 2865 | 6177 | 7005 | 7833 |
| 763 | 2038 | 3694 | 4522 | 5350 | 764 | 2866 | 6178 | 7006 | 7834 |
| 765 | 2039 | 3695 | 4523 | 5351 | 766 | 2867 | 6179 | 7007 | 7835 |
| 767 | 2040 | 3696 | 4524 | 5352 | 768 | 2868 | 6180 | 7008 | 7836 |
| 769 | 2041 | 3697 | 4525 | 5353 | 770 | 2869 | 6181 | 7009 | 7837 |
| 771 | 2042 | 3698 | 4526 | 5354 | 772 | 2870 | 6182 | 7010 | 7838 |
| 773 | 2043 | 3699 | 4527 | 5355 | 774 | 2871 | 6183 | 7011 | 7839 |
| 775 | 2044 | 3700 | 4528 | 5356 | 776 | 2872 | 6184 | 7012 | 7840 |
| 777 | 2045 | 3701 | 4529 | 5357 | 778 | 2873 | 6185 | 7013 | 7841 |
| 779 | 2046 | 3702 | 4530 | 5358 | 780 | 2874 | 6186 | 7014 | 7842 |
| 781 | 2047 | 3703 | 4531 | 5359 | 782 | 2875 | 6187 | 7015 | 7843 |
| 783 | 2048 | 3704 | 4532 | 5360 | 784 | 2876 | 6188 | 7016 | 7844 |
| 785 | 2049 | 3705 | 4533 | 5361 | 786 | 2877 | 6189 | 7017 | 7845 |
| 787 | 2050 | 3706 | 4534 | 5362 | 788 | 2878 | 6190 | 7018 | 7846 |
| 789 | 2051 | 3707 | 4535 | 5363 | 790 | 2879 | 6191 | 7019 | 7847 |
| 791 | 2052 | 3708 | 4536 | 5364 | 792 | 2880 | 6192 | 7020 | 7848 |
| 793 | 2053 | 3709 | 4537 | 5365 | 794 | 2881 | 6193 | 7021 | 7849 |
| 795 | 2054 | 3710 | 4538 | 5366 | 796 | 2882 | 6194 | 7022 | 7850 |
| 797 | 2055 | 3711 | 4539 | 5367 | 798 | 2883 | 6195 | 7023 | 7851 |
| 799 | 2056 | 3712 | 4540 | 5368 | 800 | 2884 | 6196 | 7024 | 7852 |
| 801 | 2057 | 3713 | 4541 | 5369 | 802 | 2885 | 6197 | 7025 | 7853 |
| 803 | 2058 | 3714 | 4542 | 5370 | 804 | 2886 | 6198 | 7026 | 7854 |
| 805 | 2059 | 3715 | 4543 | 5371 | 806 | 2887 | 6199 | 7027 | 7855 |
| 807 | 2060 | 3716 | 4544 | 5372 | 808 | 2888 | 6200 | 7028 | 7856 |
| 809 | 2061 | 3717 | 4545 | 5373 | 810 | 2889 | 6201 | 7029 | 7857 |
| 811 | 2062 | 3718 | 4546 | 5374 | 812 | 2890 | 6202 | 7030 | 7858 |
| 813 | 2063 | 3719 | 4547 | 5375 | 814 | 2891 | 6203 | 7031 | 7859 |
| 815 | 2064 | 3720 | 4548 | 5376 | 816 | 2892 | 6204 | 7032 | 7860 |
| 817 | 2065 | 3721 | 4549 | 5377 | 818 | 2893 | 6205 | 7033 | 7861 |
| 819 | 2066 | 3722 | 4550 | 5378 | 820 | 2894 | 6206 | 7034 | 7862 |
| 821 | 2067 | 3723 | 4551 | 5379 | 822 | 2895 | 6207 | 7035 | 7863 |
| 823 | 2068 | 3724 | 4552 | 5380 | 824 | 2896 | 6208 | 7036 | 7864 |
| 825 | 2069 | 3725 | 4553 | 5381 | 826 | 2897 | 6209 | 7037 | 7865 |
| 827 | 2070 | 3726 | 4554 | 5382 | 828 | 2898 | 6210 | 7038 | 7866 |
| 829 | 2071 | 3727 | 4555 | 5383 | 830 | 2899 | 6211 | 7039 | 7867 |
| 831 | 2072 | 3728 | 4556 | 5384 | 832 | 2900 | 6212 | 7040 | 7868 |
| 833 | 2073 | 3729 | 4557 | 5385 | 834 | 2901 | 6213 | 7041 | 7869 |
| 835 | 2074 | 3730 | 4558 | 5386 | 836 | 2902 | 6214 | 7042 | 7870 |
| 837 | 2075 | 3731 | 4559 | 5387 | 838 | 2903 | 6215 | 7043 | 7871 |
| 839 | 2076 | 3732 | 4560 | 5388 | 840 | 2904 | 6216 | 7044 | 7872 |
| 841 | 2077 | 3733 | 4561 | 5389 | 842 | 2905 | 6217 | 7045 | 7873 |
| 843 | 2078 | 3734 | 4562 | 5390 | 844 | 2906 | 6218 | 7046 | 7874 |
| 845 | 2079 | 3735 | 4563 | 5391 | 846 | 2907 | 6219 | 7047 | 7875 |
| 847 | 2080 | 3736 | 4564 | 5392 | 848 | 2908 | 6220 | 7048 | 7876 |
| 849 | 2081 | 3737 | 4565 | 5393 | 850 | 2909 | 6221 | 7049 | 7877 |
| 851 | 2082 | 3738 | 4566 | 5394 | 852 | 2910 | 6222 | 7050 | 7878 |
| 853 | 2083 | 3739 | 4567 | 5395 | 854 | 2911 | 6223 | 7051 | 7879 |
| 855 | 2084 | 3740 | 4568 | 5396 | 856 | 2912 | 6224 | 7052 | 7880 |
| 857 | 2085 | 3741 | 4569 | 5397 | 858 | 2913 | 6225 | 7053 | 7881 |
| 859 | 2086 | 3742 | 4570 | 5398 | 860 | 2914 | 6226 | 7054 | 7882 |
| 861 | 2087 | 3743 | 4571 | 5399 | 862 | 2915 | 6227 | 7055 | 7883 |
| 863 | 2088 | 3744 | 4572 | 5400 | 864 | 2916 | 6228 | 7056 | 7884 |
| 865 | 2089 | 3745 | 4573 | 5401 | 866 | 2917 | 6229 | 7057 | 7885 |
| 867 | 2090 | 3746 | 4574 | 5402 | 868 | 2918 | 6230 | 7058 | 7886 |
| 869 | 2091 | 3747 | 4575 | 5403 | 870 | 2919 | 6231 | 7059 | 7887 |
| 871 | 2092 | 3748 | 4576 | 5404 | 872 | 2920 | 6232 | 7060 | 7888 |
| 873 | 2093 | 3749 | 4577 | 5405 | 874 | 2921 | 6233 | 7061 | 7889 |
| 875 | 2094 | 3750 | 4578 | 5406 | 876 | 2922 | 6234 | 7062 | 7890 |
| 877 | 2095 | 3751 | 4579 | 5407 | 878 | 2923 | 6235 | 7063 | 7891 |
| 879 | 2096 | 3752 | 4580 | 5408 | 880 | 2924 | 6236 | 7064 | 7892 |
| 881 | 2097 | 3753 | 4581 | 5409 | 882 | 2925 | 6237 | 7065 | 7893 |
| 883 | 2098 | 3754 | 4582 | 5410 | 884 | 2926 | 6238 | 7066 | 7894 |
| 885 | 2099 | 3755 | 4583 | 5411 | 886 | 2927 | 6239 | 7067 | 7895 |
| 887 | 2100 | 3756 | 4584 | 5412 | 888 | 2928 | 6240 | 7068 | 7896 |
| 889 | 2101 | 3757 | 4585 | 5413 | 890 | 2929 | 6241 | 7069 | 7897 |
| 891 | 2102 | 3758 | 4586 | 5414 | 892 | 2930 | 6242 | 7070 | 7898 |
| 893 | 2103 | 3759 | 4587 | 5415 | 894 | 2931 | 6243 | 7071 | 7899 |
| 895 | 2104 | 3760 | 4588 | 5416 | 896 | 2932 | 6244 | 7072 | 7900 |
| 897 | 2105 | 3761 | 4589 | 5417 | 898 | 2933 | 6245 | 7073 | 7901 |
| 899 | 2106 | 3762 | 4590 | 5418 | 900 | 2934 | 6246 | 7074 | 7902 |
| 901 | 2107 | 3763 | 4591 | 5419 | 902 | 2935 | 6247 | 7075 | 7903 |
| 903 | 2108 | 3764 | 4592 | 5420 | 904 | 2936 | 6248 | 7076 | 7904 |
| 905 | 2109 | 3765 | 4593 | 5421 | 906 | 2937 | 6249 | 7077 | 7905 |
| 907 | 2110 | 3766 | 4594 | 5422 | 908 | 2938 | 6250 | 7078 | 7906 |
| 909 | 2111 | 3767 | 4595 | 5423 | 910 | 2939 | 6251 | 7079 | 7907 |
| 911 | 2112 | 3768 | 4596 | 5424 | 912 | 2940 | 6252 | 7080 | 7908 |
| 913 | 2113 | 3769 | 4597 | 5425 | 914 | 2941 | 6253 | 7081 | 7909 |
| 915 | 2114 | 3770 | 4598 | 5426 | 916 | 2942 | 6254 | 7082 | 7910 |
| 917 | 2115 | 3771 | 4599 | 5427 | 918 | 2943 | 6255 | 7083 | 7911 |
| 919 | 2116 | 3772 | 4600 | 5428 | 920 | 2944 | 6256 | 7084 | 7912 |
| 921 | 2117 | 3773 | 4601 | 5429 | 922 | 2945 | 6257 | 7085 | 7913 |
| 923 | 2118 | 3774 | 4602 | 5430 | 924 | 2946 | 6258 | 7086 | 7914 |
| 925 | 2119 | 3775 | 4603 | 5431 | 926 | 2947 | 6259 | 7087 | 7915 |
| 927 | 2120 | 3776 | 4604 | 5432 | 928 | 2948 | 6260 | 7088 | 7916 |
| 929 | 2121 | 3777 | 4605 | 5433 | 930 | 2949 | 6261 | 7089 | 7917 |
| 931 | 2122 | 3778 | 4606 | 5434 | 932 | 2950 | 6262 | 7090 | 7918 |
| 933 | 2123 | 3779 | 4607 | 5435 | 934 | 2951 | 6263 | 7091 | 7919 |
| 935 | 2124 | 3780 | 4608 | 5436 | 936 | 2952 | 6264 | 7092 | 7920 |
| 937 | 2125 | 3781 | 4609 | 5437 | 938 | 2953 | 6265 | 7093 | 7921 |
| 939 | 2126 | 3782 | 4610 | 5438 | 940 | 2954 | 6266 | 7094 | 7922 |
| 941 | 2127 | 3783 | 4611 | 5439 | 942 | 2955 | 6267 | 7095 | 7923 |
| 943 | 2128 | 3784 | 4612 | 5440 | 944 | 2956 | 6268 | 7096 | 7924 |
| 945 | 2129 | 3785 | 4613 | 5441 | 946 | 2957 | 6269 | 7097 | 7925 |
| 947 | 2130 | 3786 | 4614 | 5442 | 948 | 2958 | 6270 | 7098 | 7926 |
| 949 | 2131 | 3787 | 4615 | 5443 | 950 | 2959 | 6271 | 7099 | 7927 |
| 951 | 2132 | 3788 | 4616 | 5444 | 952 | 2960 | 6272 | 7100 | 7928 |
| 953 | 2133 | 3789 | 4617 | 5445 | 954 | 2961 | 6273 | 7101 | 7929 |
| 955 | 2134 | 3790 | 4618 | 5446 | 956 | 2962 | 6274 | 7102 | 7930 |
| 957 | 2135 | 3791 | 4619 | 5447 | 958 | 2963 | 6275 | 7103 | 7931 |
| 959 | 2136 | 3792 | 4620 | 5448 | 960 | 2964 | 6276 | 7104 | 7932 |
| 961 | 2137 | 3793 | 4621 | 5449 | 962 | 2965 | 6277 | 7105 | 7933 |
| 963 | 2138 | 3794 | 4622 | 5450 | 964 | 2966 | 6278 | 7106 | 7934 |
| 965 | 2139 | 3795 | 4623 | 5451 | 966 | 2967 | 6279 | 7107 | 7935 |
| 967 | 2140 | 3796 | 4624 | 5452 | 968 | 2968 | 6280 | 7108 | 7936 |
| 969 | 2141 | 3797 | 4625 | 5453 | 970 | 2969 | 6281 | 7109 | 7937 |
| 971 | 2142 | 3798 | 4626 | 5454 | 972 | 2970 | 6282 | 7110 | 7938 |
| 973 | 2143 | 3799 | 4627 | 5455 | 974 | 2971 | 6283 | 7111 | 7939 |
| 975 | 2144 | 3800 | 4628 | 5456 | 976 | 2972 | 6284 | 7112 | 7940 |
| 977 | 2145 | 3801 | 4629 | 5457 | 978 | 2973 | 6285 | 7113 | 7941 |
| 979 | 2146 | 3802 | 4630 | 5458 | 980 | 2974 | 6286 | 7114 | 7942 |
| 981 | 2147 | 3803 | 4631 | 5459 | 982 | 2975 | 6287 | 7115 | 7943 |
| 983 | 2148 | 3804 | 4632 | 5460 | 984 | 2976 | 6288 | 7116 | 7944 |
| 985 | 2149 | 3805 | 4633 | 5461 | 986 | 2977 | 6289 | 7117 | 7945 |
| 987 | 2150 | 3806 | 4634 | 5462 | 988 | 2978 | 6290 | 7118 | 7946 |
| 989 | 2151 | 3807 | 4635 | 5463 | 990 | 2979 | 6291 | 7119 | 7947 |
| 991 | 2152 | 3808 | 4636 | 5464 | 992 | 2980 | 6292 | 7120 | 7948 |
| 993 | 2153 | 3809 | 4637 | 5465 | 994 | 2981 | 6293 | 7121 | 7949 |
| 995 | 2154 | 3810 | 4638 | 5466 | 996 | 2982 | 6294 | 7122 | 7950 |
| 997 | 2155 | 3811 | 4639 | 5467 | 998 | 2983 | 6295 | 7123 | 7951 |
| 999 | 2156 | 3812 | 4640 | 5468 | 1000 | 2984 | 6296 | 7124 | 7952 |
| 1001 | 2157 | 3813 | 4641 | 5469 | 1002 | 2985 | 6297 | 7125 | 7953 |
| 1003 | 2158 | 3814 | 4642 | 5470 | 1004 | 2986 | 6298 | 7126 | 7954 |
| 1005 | 2159 | 3815 | 4643 | 5471 | 1006 | 2987 | 6299 | 7127 | 7955 |
| 1007 | 2160 | 3816 | 4644 | 5472 | 1008 | 2988 | 6300 | 7128 | 7956 |
| 1009 | 2161 | 3817 | 4645 | 5473 | 1010 | 2989 | 6301 | 7129 | 7957 |
| 1011 | 2162 | 3818 | 4646 | 5474 | 1012 | 2990 | 6302 | 7130 | 7958 |
| 1013 | 2163 | 3819 | 4647 | 5475 | 1014 | 2991 | 6303 | 7131 | 7959 |
| 1015 | 2164 | 3820 | 4648 | 5476 | 1016 | 2992 | 6304 | 7132 | 7960 |
| 1017 | 2165 | 3821 | 4649 | 5477 | 1018 | 2993 | 6305 | 7133 | 7961 |
| 1019 | 2166 | 3822 | 4650 | 5478 | 1020 | 2994 | 6306 | 7134 | 7962 |
| 1021 | 2167 | 3823 | 4651 | 5479 | 1022 | 2995 | 6307 | 7135 | 7963 |
| 1023 | 2168 | 3824 | 4652 | 5480 | 1024 | 2996 | 6308 | 7136 | 7964 |
| 1025 | 2169 | 3825 | 4653 | 5481 | 1026 | 2997 | 6309 | 7137 | 7965 |
| 1027 | 2170 | 3826 | 4654 | 5482 | 1028 | 2998 | 6310 | 7138 | 7966 |
| 1029 | 2171 | 3827 | 4655 | 5483 | 1030 | 2999 | 6311 | 7139 | 7967 |
| 1031 | 2172 | 3828 | 4656 | 5484 | 1032 | 3000 | 6312 | 7140 | 7968 |
| 1033 | 2173 | 3829 | 4657 | 5485 | 1034 | 3001 | 6313 | 7141 | 7969 |
| 1035 | 2174 | 3830 | 4658 | 5486 | 1036 | 3002 | 6314 | 7142 | 7970 |
| 1037 | 2175 | 3831 | 4659 | 5487 | 1038 | 3003 | 6315 | 7143 | 7971 |
| 1039 | 2176 | 3832 | 4660 | 5488 | 1040 | 3004 | 6316 | 7144 | 7972 |
| 1041 | 2177 | 3833 | 4661 | 5489 | 1042 | 3005 | 6317 | 7145 | 7973 |
| 1043 | 2178 | 3834 | 4662 | 5490 | 1044 | 3006 | 6318 | 7146 | 7974 |
| 1045 | 2179 | 3835 | 4663 | 5491 | 1046 | 3007 | 6319 | 7147 | 7975 |
| 1047 | 2180 | 3836 | 4664 | 5492 | 1048 | 3008 | 6320 | 7148 | 7976 |
| 1049 | 2181 | 3837 | 4665 | 5493 | 1050 | 3009 | 6321 | 7149 | 7977 |
| 1051 | 2182 | 3838 | 4666 | 5494 | 1052 | 3010 | 6322 | 7150 | 7978 |
| 1053 | 2183 | 3839 | 4667 | 5495 | 1054 | 3011 | 6323 | 7151 | 7979 |
| 1055 | 2184 | 3840 | 4668 | 5496 | 1056 | 3012 | 6324 | 7152 | 7980 |
| 1057 | 2185 | 3841 | 4669 | 5497 | 1058 | 3013 | 6325 | 7153 | 7981 |
| 1059 | 2186 | 3842 | 4670 | 5498 | 1060 | 3014 | 6326 | 7154 | 7982 |
| 1061 | 2187 | 3843 | 4671 | 5499 | 1062 | 3015 | 6327 | 7155 | 7983 |
| 1063 | 2188 | 3844 | 4672 | 5500 | 1064 | 3016 | 6328 | 7156 | 7984 |
| 1065 | 2189 | 3845 | 4673 | 5501 | 1066 | 3017 | 6329 | 7157 | 7985 |
| 1067 | 2190 | 3846 | 4674 | 5502 | 1068 | 3018 | 6330 | 7158 | 7986 |
| 1069 | 2191 | 3847 | 4675 | 5503 | 1070 | 3019 | 6331 | 7159 | 7987 |
| 1071 | 2192 | 3848 | 4676 | 5504 | 1072 | 3020 | 6332 | 7160 | 7988 |
| 1073 | 2193 | 3849 | 4677 | 5505 | 1074 | 3021 | 6333 | 7161 | 7989 |
| 1075 | 2194 | 3850 | 4678 | 5506 | 1076 | 3022 | 6334 | 7162 | 7990 |
| 1077 | 2195 | 3851 | 4679 | 5507 | 1078 | 3023 | 6335 | 7163 | 7991 |
| 1079 | 2196 | 3852 | 4680 | 5508 | 1080 | 3024 | 6336 | 7164 | 7992 |
| 1081 | 2197 | 3853 | 4681 | 5509 | 1082 | 3025 | 6337 | 7165 | 7993 |
| 1083 | 2198 | 3854 | 4682 | 5510 | 1084 | 3026 | 6338 | 7166 | 7994 |
| 1085 | 2199 | 3855 | 4683 | 5511 | 1086 | 3027 | 6339 | 7167 | 7995 |
| 1087 | 2200 | 3856 | 4684 | 5512 | 1088 | 3028 | 6340 | 7168 | 7996 |
| 1089 | 2201 | 3857 | 4685 | 5513 | 1090 | 3029 | 6341 | 7169 | 7997 |
| 1091 | 2202 | 3858 | 4686 | 5514 | 1092 | 3030 | 6342 | 7170 | 7998 |
| 1093 | 2203 | 3859 | 4687 | 5515 | 1094 | 3031 | 6343 | 7171 | 7999 |
| 1095 | 2204 | 3860 | 4688 | 5516 | 1096 | 3032 | 6344 | 7172 | 8000 |
| 1097 | 2205 | 3861 | 4689 | 5517 | 1098 | 3033 | 6345 | 7173 | 8001 |
| 1099 | 2206 | 3862 | 4690 | 5518 | 1100 | 3034 | 6346 | 7174 | 8002 |
| 1101 | 2207 | 3863 | 4691 | 5519 | 1102 | 3035 | 6347 | 7175 | 8003 |
| 1103 | 2208 | 3864 | 4692 | 5520 | 1104 | 3036 | 6348 | 7176 | 8004 |
| 1105 | 2209 | 3865 | 4693 | 5521 | 1106 | 3037 | 6349 | 7177 | 8005 |
| 1107 | 2210 | 3866 | 4694 | 5522 | 1108 | 3038 | 6350 | 7178 | 8006 |
| 1109 | 2211 | 3867 | 4695 | 5523 | 1110 | 3039 | 6351 | 7179 | 8007 |
| 1111 | 2212 | 3868 | 4696 | 5524 | 1112 | 3040 | 6352 | 7180 | 8008 |
| 1113 | 2213 | 3869 | 4697 | 5525 | 1114 | 3041 | 6353 | 7181 | 8009 |
| 1115 | 2214 | 3870 | 4698 | 5526 | 1116 | 3042 | 6354 | 7182 | 8010 |
| 1117 | 2215 | 3871 | 4699 | 5527 | 1118 | 3043 | 6355 | 7183 | 8011 |
| 1119 | 2216 | 3872 | 4700 | 5528 | 1120 | 3044 | 6356 | 7184 | 8012 |
| 1121 | 2217 | 3873 | 4701 | 5529 | 1122 | 3045 | 6357 | 7185 | 8013 |
| 1123 | 2218 | 3874 | 4702 | 5530 | 1124 | 3046 | 6358 | 7186 | 8014 |
| 1125 | 2219 | 3875 | 4703 | 5531 | 1126 | 3047 | 6359 | 7187 | 8015 |
| 1127 | 2220 | 3876 | 4704 | 5532 | 1128 | 3048 | 6360 | 7188 | 8016 |
| 1129 | 2221 | 3877 | 4705 | 5533 | 1130 | 3049 | 6361 | 7189 | 8017 |
| 1131 | 2222 | 3878 | 4706 | 5534 | 1132 | 3050 | 6362 | 7190 | 8018 |
| 1133 | 2223 | 3879 | 4707 | 5535 | 1134 | 3051 | 6363 | 7191 | 8019 |
| 1135 | 2224 | 3880 | 4708 | 5536 | 1136 | 3052 | 6364 | 7192 | 8020 |
| 1137 | 2225 | 3881 | 4709 | 5537 | 1138 | 3053 | 6365 | 7193 | 8021 |
| 1139 | 2226 | 3882 | 4710 | 5538 | 1140 | 3054 | 6366 | 7194 | 8022 |
| 1141 | 2227 | 3883 | 4711 | 5539 | 1142 | 3055 | 6367 | 7195 | 8023 |
| 1143 | 2228 | 3884 | 4712 | 5540 | 1144 | 3056 | 6368 | 7196 | 8024 |
| 1145 | 2229 | 3885 | 4713 | 5541 | 1146 | 3057 | 6369 | 7197 | 8025 |
| 1147 | 2230 | 3886 | 4714 | 5542 | 1148 | 3058 | 6370 | 7198 | 8026 |
| 1149 | 2231 | 3887 | 4715 | 5543 | 1150 | 3059 | 6371 | 7199 | 8027 |
| 1151 | 2232 | 3888 | 4716 | 5544 | 1152 | 3060 | 6372 | 7200 | 8028 |
| 1153 | 2233 | 3889 | 4717 | 5545 | 1154 | 3061 | 6373 | 7201 | 8029 |
| 1155 | 2234 | 3890 | 4718 | 5546 | 1156 | 3062 | 6374 | 7202 | 8030 |
| 1157 | 2235 | 3891 | 4719 | 5547 | 1158 | 3063 | 6375 | 7203 | 8031 |
| 1159 | 2236 | 3892 | 4720 | 5548 | 1160 | 3064 | 6376 | 7204 | 8032 |
| 1161 | 2237 | 3893 | 4721 | 5549 | 1162 | 3065 | 6377 | 7205 | 8033 |
| 1163 | 2238 | 3894 | 4722 | 5550 | 1164 | 3066 | 6378 | 7206 | 8034 |
| 1165 | 2239 | 3895 | 4723 | 5551 | 1166 | 3067 | 6379 | 7207 | 8035 |
| 1167 | 2240 | 3896 | 4724 | 5552 | 1168 | 3068 | 6380 | 7208 | 8036 |
| 1169 | 2241 | 3897 | 4725 | 5553 | 1170 | 3069 | 6381 | 7209 | 8037 |
| 1171 | 2242 | 3898 | 4726 | 5554 | 1172 | 3070 | 6382 | 7210 | 8038 |
| 1173 | 2243 | 3899 | 4727 | 5555 | 1174 | 3071 | 6383 | 7211 | 8039 |
| 1175 | 2244 | 3900 | 4728 | 5556 | 1176 | 3072 | 6384 | 7212 | 8040 |
| 1177 | 2245 | 3901 | 4729 | 5557 | 1178 | 3073 | 6385 | 7213 | 8041 |
| 1179 | 2246 | 3902 | 4730 | 5558 | 1180 | 3074 | 6386 | 7214 | 8042 |
| 1181 | 2247 | 3903 | 4731 | 5559 | 1182 | 3075 | 6387 | 7215 | 8043 |
| 1183 | 2248 | 3904 | 4732 | 5560 | 1184 | 3076 | 6388 | 7216 | 8044 |
| 1185 | 2249 | 3905 | 4733 | 5561 | 1186 | 3077 | 6389 | 7217 | 8045 |
| 1187 | 2250 | 3906 | 4734 | 5562 | 1188 | 3078 | 6390 | 7218 | 8046 |
| 1189 | 2251 | 3907 | 4735 | 5563 | 1190 | 3079 | 6391 | 7219 | 8047 |
| 1191 | 2252 | 3908 | 4736 | 5564 | 1192 | 3080 | 6392 | 7220 | 8048 |
| 1193 | 2253 | 3909 | 4737 | 5565 | 1194 | 3081 | 6393 | 7221 | 8049 |
| 1195 | 2254 | 3910 | 4738 | 5566 | 1196 | 3082 | 6394 | 7222 | 8050 |
| 1197 | 2255 | 3911 | 4739 | 5567 | 1198 | 3083 | 6395 | 7223 | 8051 |
| 1199 | 2256 | 3912 | 4740 | 5568 | 1200 | 3084 | 6396 | 7224 | 8052 |
| 1201 | 2257 | 3913 | 4741 | 5569 | 1202 | 3085 | 6397 | 7225 | 8053 |
| 1203 | 2258 | 3914 | 4742 | 5570 | 1204 | 3086 | 6398 | 7226 | 8054 |
| 1205 | 2259 | 3915 | 4743 | 5571 | 1206 | 3087 | 6399 | 7227 | 8055 |
| 1207 | 2260 | 3916 | 4744 | 5572 | 1208 | 3088 | 6400 | 7228 | 8056 |
| 1209 | 2261 | 3917 | 4745 | 5573 | 1210 | 3089 | 6401 | 7229 | 8057 |
| 1211 | 2262 | 3918 | 4746 | 5574 | 1212 | 3090 | 6402 | 7230 | 8058 |
| 1213 | 2263 | 3919 | 4747 | 5575 | 1214 | 3091 | 6403 | 7231 | 8059 |
| 1215 | 2264 | 3920 | 4748 | 5576 | 1216 | 3092 | 6404 | 7232 | 8060 |
| 1217 | 2265 | 3921 | 4749 | 5577 | 1218 | 3093 | 6405 | 7233 | 8061 |
| 1219 | 2266 | 3922 | 4750 | 5578 | 1220 | 3094 | 6406 | 7234 | 8062 |
| 1221 | 2267 | 3923 | 4751 | 5579 | 1222 | 3095 | 6407 | 7235 | 8063 |
| 1223 | 2268 | 3924 | 4752 | 5580 | 1224 | 3096 | 6408 | 7236 | 8064 |
| 1225 | 2269 | 3925 | 4753 | 5581 | 1226 | 3097 | 6409 | 7237 | 8065 |
| 1227 | 2270 | 3926 | 4754 | 5582 | 1228 | 3098 | 6410 | 7238 | 8066 |
| 1229 | 2271 | 3927 | 4755 | 5583 | 1230 | 3099 | 6411 | 7239 | 8067 |
| 1231 | 2272 | 3928 | 4756 | 5584 | 1232 | 3100 | 6412 | 7240 | 8068 |
| 1233 | 2273 | 3929 | 4757 | 5585 | 1234 | 3101 | 6413 | 7241 | 8069 |
| 1235 | 2274 | 3930 | 4758 | 5586 | 1236 | 3102 | 6414 | 7242 | 8070 |
| 1237 | 2275 | 3931 | 4759 | 5587 | 1238 | 3103 | 6415 | 7243 | 8071 |
| 1239 | 2276 | 3932 | 4760 | 5588 | 1240 | 3104 | 6416 | 7244 | 8072 |
| 1241 | 2277 | 3933 | 4761 | 5589 | 1242 | 3105 | 6417 | 7245 | 8073 |
| 1243 | 2278 | 3934 | 4762 | 5590 | 1244 | 3106 | 6418 | 7246 | 8074 |
| 1245 | 2279 | 3935 | 4763 | 5591 | 1246 | 3107 | 6419 | 7247 | 8075 |
| 1247 | 2280 | 3936 | 4764 | 5592 | 1248 | 3108 | 6420 | 7248 | 8076 |
| 1249 | 2281 | 3937 | 4765 | 5593 | 1250 | 3109 | 6421 | 7249 | 8077 |
| 1251 | 2282 | 3938 | 4766 | 5594 | 1252 | 3110 | 6422 | 7250 | 8078 |
| 1253 | 2283 | 3939 | 4767 | 5595 | 1254 | 3111 | 6423 | 7251 | 8079 |
| 1255 | 2284 | 3940 | 4768 | 5596 | 1256 | 3112 | 6424 | 7252 | 8080 |
| 1257 | 2285 | 3941 | 4769 | 5597 | 1258 | 3113 | 6425 | 7253 | 8081 |
| 1259 | 2286 | 3942 | 4770 | 5598 | 1260 | 3114 | 6426 | 7254 | 8082 |
| 1261 | 2287 | 3943 | 4771 | 5599 | 1262 | 3115 | 6427 | 7255 | 8083 |
| 1263 | 2288 | 3944 | 4772 | 5600 | 1264 | 3116 | 6428 | 7256 | 8084 |
| 1265 | 2289 | 3945 | 4773 | 5601 | 1266 | 3117 | 6429 | 7257 | 8085 |
| 1267 | 2290 | 3946 | 4774 | 5602 | 1268 | 3118 | 6430 | 7258 | 8086 |
| 1269 | 2291 | 3947 | 4775 | 5603 | 1270 | 3119 | 6431 | 7259 | 8087 |
| 1271 | 2292 | 3948 | 4776 | 5604 | 1272 | 3120 | 6432 | 7260 | 8088 |
| 1273 | 2293 | 3949 | 4777 | 5605 | 1274 | 3121 | 6433 | 7261 | 8089 |
| 1275 | 2294 | 3950 | 4778 | 5606 | 1276 | 3122 | 6434 | 7262 | 8090 |
| 1277 | 2295 | 3951 | 4779 | 5607 | 1278 | 3123 | 6435 | 7263 | 8091 |
| 1279 | 2296 | 3952 | 4780 | 5608 | 1280 | 3124 | 6436 | 7264 | 8092 |
| 1281 | 2297 | 3953 | 4781 | 5609 | 1282 | 3125 | 6437 | 7265 | 8093 |
| 1283 | 2298 | 3954 | 4782 | 5610 | 1284 | 3126 | 6438 | 7266 | 8094 |
| 1285 | 2299 | 3955 | 4783 | 5611 | 1286 | 3127 | 6439 | 7267 | 8095 |
| 1287 | 2300 | 3956 | 4784 | 5612 | 1288 | 3128 | 6440 | 7268 | 8096 |
| 1289 | 2301 | 3957 | 4785 | 5613 | 1290 | 3129 | 6441 | 7269 | 8097 |
| 1291 | 2302 | 3958 | 4786 | 5614 | 1292 | 3130 | 6442 | 7270 | 8098 |
| 1293 | 2303 | 3959 | 4787 | 5615 | 1294 | 3131 | 6443 | 7271 | 8099 |
| 1295 | 2304 | 3960 | 4788 | 5616 | 1296 | 3132 | 6444 | 7272 | 8100 |
| 1297 | 2305 | 3961 | 4789 | 5617 | 1298 | 3133 | 6445 | 7273 | 8101 |
| 1299 | 2306 | 3962 | 4790 | 5618 | 1300 | 3134 | 6446 | 7274 | 8102 |
| 1301 | 2307 | 3963 | 4791 | 5619 | 1302 | 3135 | 6447 | 7275 | 8103 |
| 1303 | 2308 | 3964 | 4792 | 5620 | 1304 | 3136 | 6448 | 7276 | 8104 |
| 1305 | 2309 | 3965 | 4793 | 5621 | 1306 | 3137 | 6449 | 7277 | 8105 |
| 1307 | 2310 | 3966 | 4794 | 5622 | 1308 | 3138 | 6450 | 7278 | 8106 |
| 1309 | 2311 | 3967 | 4795 | 5623 | 1310 | 3139 | 6451 | 7279 | 8107 |
| 1311 | 2312 | 3968 | 4796 | 5624 | 1312 | 3140 | 6452 | 7280 | 8108 |
| 1313 | 2313 | 3969 | 4797 | 5625 | 1314 | 3141 | 6453 | 7281 | 8109 |
| 1315 | 2314 | 3970 | 4798 | 5626 | 1316 | 3142 | 6454 | 7282 | 8110 |
| 1317 | 2315 | 3971 | 4799 | 5627 | 1318 | 3143 | 6455 | 7283 | 8111 |
| 1319 | 2316 | 3972 | 4800 | 5628 | 1320 | 3144 | 6456 | 7284 | 8112 |
| 1321 | 2317 | 3973 | 4801 | 5629 | 1322 | 3145 | 6457 | 7285 | 8113 |
| 1323 | 2318 | 3974 | 4802 | 5630 | 1324 | 3146 | 6458 | 7286 | 8114 |
| 1325 | 2319 | 3975 | 4803 | 5631 | 1326 | 3147 | 6459 | 7287 | 8115 |
| 1327 | 2320 | 3976 | 4804 | 5632 | 1328 | 3148 | 6460 | 7288 | 8116 |
| 1329 | 2321 | 3977 | 4805 | 5633 | 1330 | 3149 | 6461 | 7289 | 8117 |
| 1331 | 2322 | 3978 | 4806 | 5634 | 1332 | 3150 | 6462 | 7290 | 8118 |
| 1333 | 2323 | 3979 | 4807 | 5635 | 1334 | 3151 | 6463 | 7291 | 8119 |
| 1335 | 2324 | 3980 | 4808 | 5636 | 1336 | 3152 | 6464 | 7292 | 8120 |
| 1337 | 2325 | 3981 | 4809 | 5637 | 1338 | 3153 | 6465 | 7293 | 8121 |
| 1339 | 2326 | 3982 | 4810 | 5638 | 1340 | 3154 | 6466 | 7294 | 8122 |
| 1341 | 2327 | 3983 | 4811 | 5639 | 1342 | 3155 | 6467 | 7295 | 8123 |
| 1343 | 2328 | 3984 | 4812 | 5640 | 1344 | 3156 | 6468 | 7296 | 8124 |
| 1345 | 2329 | 3985 | 4813 | 5641 | 1346 | 3157 | 6469 | 7297 | 8125 |
| 1347 | 2330 | 3986 | 4814 | 5642 | 1348 | 3158 | 6470 | 7298 | 8126 |
| 1349 | 2331 | 3987 | 4815 | 5643 | 1350 | 3159 | 6471 | 7299 | 8127 |
| 1351 | 2332 | 3988 | 4816 | 5644 | 1352 | 3160 | 6472 | 7300 | 8128 |
| 1353 | 2333 | 3989 | 4817 | 5645 | 1354 | 3161 | 6473 | 7301 | 8129 |
| 1355 | 2334 | 3990 | 4818 | 5646 | 1356 | 3162 | 6474 | 7302 | 8130 |
| 1357 | 2335 | 3991 | 4819 | 5647 | 1358 | 3163 | 6475 | 7303 | 8131 |
| 1359 | 2336 | 3992 | 4820 | 5648 | 1360 | 3164 | 6476 | 7304 | 8132 |
| 1361 | 2337 | 3993 | 4821 | 5649 | 1362 | 3165 | 6477 | 7305 | 8133 |
| 1363 | 2338 | 3994 | 4822 | 5650 | 1364 | 3166 | 6478 | 7306 | 8134 |
| 1365 | 2339 | 3995 | 4823 | 5651 | 1366 | 3167 | 6479 | 7307 | 8135 |
| 1367 | 2340 | 3996 | 4824 | 5652 | 1368 | 3168 | 6480 | 7308 | 8136 |
| 1369 | 2341 | 3997 | 4825 | 5653 | 1370 | 3169 | 6481 | 7309 | 8137 |
| 1371 | 2342 | 3998 | 4826 | 5654 | 1372 | 3170 | 6482 | 7310 | 8138 |
| 1373 | 2343 | 3999 | 4827 | 5655 | 1374 | 3171 | 6483 | 7311 | 8139 |
| 1375 | 2344 | 4000 | 4828 | 5656 | 1376 | 3172 | 6484 | 7312 | 8140 |
| 1377 | 2345 | 4001 | 4829 | 5657 | 1378 | 3173 | 6485 | 7313 | 8141 |
| 1379 | 2346 | 4002 | 4830 | 5658 | 1380 | 3174 | 6486 | 7314 | 8142 |
| 1381 | 2347 | 4003 | 4831 | 5659 | 1382 | 3175 | 6487 | 7315 | 8143 |
| 1383 | 2348 | 4004 | 4832 | 5660 | 1384 | 3176 | 6488 | 7316 | 8144 |
| 1385 | 2349 | 4005 | 4833 | 5661 | 1386 | 3177 | 6489 | 7317 | 8145 |
| 1387 | 2350 | 4006 | 4834 | 5662 | 1388 | 3178 | 6490 | 7318 | 8146 |
| 1389 | 2351 | 4007 | 4835 | 5663 | 1390 | 3179 | 6491 | 7319 | 8147 |
| 1391 | 2352 | 4008 | 4836 | 5664 | 1392 | 3180 | 6492 | 7320 | 8148 |
| 1393 | 2353 | 4009 | 4837 | 5665 | 1394 | 3181 | 6493 | 7321 | 8149 |
| 1395 | 2354 | 4010 | 4838 | 5666 | 1396 | 3182 | 6494 | 7322 | 8150 |
| 1397 | 2355 | 4011 | 4839 | 5667 | 1398 | 3183 | 6495 | 7323 | 8151 |
| 1399 | 2356 | 4012 | 4840 | 5668 | 1400 | 3184 | 6496 | 7324 | 8152 |
| 1401 | 2357 | 4013 | 4841 | 5669 | 1402 | 3185 | 6497 | 7325 | 8153 |
| 1403 | 2358 | 4014 | 4842 | 5670 | 1404 | 3186 | 6498 | 7326 | 8154 |
| 1405 | 2359 | 4015 | 4843 | 5671 | 1406 | 3187 | 6499 | 7327 | 8155 |
| 1407 | 2360 | 4016 | 4844 | 5672 | 1408 | 3188 | 6500 | 7328 | 8156 |
| 1409 | 2361 | 4017 | 4845 | 5673 | 1410 | 3189 | 6501 | 7329 | 8157 |
| 1411 | 2362 | 4018 | 4846 | 5674 | 1412 | 3190 | 6502 | 7330 | 8158 |
| 1413 | 2363 | 4019 | 4847 | 5675 | 1414 | 3191 | 6503 | 7331 | 8159 |
| 1415 | 2364 | 4020 | 4848 | 5676 | 1416 | 3192 | 6504 | 7332 | 8160 |
| 1417 | 2365 | 4021 | 4849 | 5677 | 1418 | 3193 | 6505 | 7333 | 8161 |
| 1419 | 2366 | 4022 | 4850 | 5678 | 1420 | 3194 | 6506 | 7334 | 8162 |
| 1421 | 2367 | 4023 | 4851 | 5679 | 1422 | 3195 | 6507 | 7335 | 8163 |
| 1423 | 2368 | 4024 | 4852 | 5680 | 1424 | 3196 | 6508 | 7336 | 8164 |
| 1425 | 2369 | 4025 | 4853 | 5681 | 1426 | 3197 | 6509 | 7337 | 8165 |
| 1427 | 2370 | 4026 | 4854 | 5682 | 1428 | 3198 | 6510 | 7338 | 8166 |
| 1429 | 2371 | 4027 | 4855 | 5683 | 1430 | 3199 | 6511 | 7339 | 8167 |
| 1431 | 2372 | 4028 | 4856 | 5684 | 1432 | 3200 | 6512 | 7340 | 8168 |
| 1433 | 2373 | 4029 | 4857 | 5685 | 1434 | 3201 | 6513 | 7341 | 8169 |
| 1435 | 2374 | 4030 | 4858 | 5686 | 1436 | 3202 | 6514 | 7342 | 8170 |
| 1437 | 2375 | 4031 | 4859 | 5687 | 1438 | 3203 | 6515 | 7343 | 8171 |
| 1439 | 2376 | 4032 | 4860 | 5688 | 1440 | 3204 | 6516 | 7344 | 8172 |
| 1441 | 2377 | 4033 | 4861 | 5689 | 1442 | 3205 | 6517 | 7345 | 8173 |
| 1443 | 2378 | 4034 | 4862 | 5690 | 1444 | 3206 | 6518 | 7346 | 8174 |
| 1445 | 2379 | 4035 | 4863 | 5691 | 1446 | 3207 | 6519 | 7347 | 8175 |
| 1447 | 2380 | 4036 | 4864 | 5692 | 1448 | 3208 | 6520 | 7348 | 8176 |
| 1449 | 2381 | 4037 | 4865 | 5693 | 1450 | 3209 | 6521 | 7349 | 8177 |
| 1451 | 2382 | 4038 | 4866 | 5694 | 1452 | 3210 | 6522 | 7350 | 8178 |
| 1453 | 2383 | 4039 | 4867 | 5695 | 1454 | 3211 | 6523 | 7351 | 8179 |
| 1455 | 2384 | 4040 | 4868 | 5696 | 1456 | 3212 | 6524 | 7352 | 8180 |
| 1457 | 2385 | 4041 | 4869 | 5697 | 1458 | 3213 | 6525 | 7353 | 8181 |
| 1459 | 2386 | 4042 | 4870 | 5698 | 1460 | 3214 | 6526 | 7354 | 8182 |
| 1461 | 2387 | 4043 | 4871 | 5699 | 1462 | 3215 | 6527 | 7355 | 8183 |
| 1463 | 2388 | 4044 | 4872 | 5700 | 1464 | 3216 | 6528 | 7356 | 8184 |
| 1465 | 2389 | 4045 | 4873 | 5701 | 1466 | 3217 | 6529 | 7357 | 8185 |
| 1467 | 2390 | 4046 | 4874 | 5702 | 1468 | 3218 | 6530 | 7358 | 8186 |
| 1469 | 2391 | 4047 | 4875 | 5703 | 1470 | 3219 | 6531 | 7359 | 8187 |
| 1471 | 2392 | 4048 | 4876 | 5704 | 1472 | 3220 | 6532 | 7360 | 8188 |
| 1473 | 2393 | 4049 | 4877 | 5705 | 1474 | 3221 | 6533 | 7361 | 8189 |
| 1475 | 2394 | 4050 | 4878 | 5706 | 1476 | 3222 | 6534 | 7362 | 8190 |
| 1477 | 2395 | 4051 | 4879 | 5707 | 1478 | 3223 | 6535 | 7363 | 8191 |
| 1479 | 2396 | 4052 | 4880 | 5708 | 1480 | 3224 | 6536 | 7364 | 8192 |
| 1481 | 2397 | 4053 | 4881 | 5709 | 1482 | 3225 | 6537 | 7365 | 8193 |
| 1483 | 2398 | 4054 | 4882 | 5710 | 1484 | 3226 | 6538 | 7366 | 8194 |
| 1485 | 2399 | 4055 | 4883 | 5711 | 1486 | 3227 | 6539 | 7367 | 8195 |
| 1487 | 2400 | 4056 | 4884 | 5712 | 1488 | 3228 | 6540 | 7368 | 8196 |
| 1489 | 2401 | 4057 | 4885 | 5713 | 1490 | 3229 | 6541 | 7369 | 8197 |
| 1491 | 2402 | 4058 | 4886 | 5714 | 1492 | 3230 | 6542 | 7370 | 8198 |
| 1493 | 2403 | 4059 | 4887 | 5715 | 1494 | 3231 | 6543 | 7371 | 8199 |
| 1495 | 2404 | 4060 | 4888 | 5716 | 1496 | 3232 | 6544 | 7372 | 8200 |
| 1497 | 2405 | 4061 | 4889 | 5717 | 1498 | 3233 | 6545 | 7373 | 8201 |
| 1499 | 2406 | 4062 | 4890 | 5718 | 1500 | 3234 | 6546 | 7374 | 8202 |
| 1501 | 2407 | 4063 | 4891 | 5719 | 1502 | 3235 | 6547 | 7375 | 8203 |
| 1503 | 2408 | 4064 | 4892 | 5720 | 1504 | 3236 | 6548 | 7376 | 8204 |
| 1505 | 2409 | 4065 | 4893 | 5721 | 1506 | 3237 | 6549 | 7377 | 8205 |
| 1507 | 2410 | 4066 | 4894 | 5722 | 1508 | 3238 | 6550 | 7378 | 8206 |
| 1509 | 2411 | 4067 | 4895 | 5723 | 1510 | 3239 | 6551 | 7379 | 8207 |
| 1511 | 2412 | 4068 | 4896 | 5724 | 1512 | 3240 | 6552 | 7380 | 8208 |
| 1513 | 2413 | 4069 | 4897 | 5725 | 1514 | 3241 | 6553 | 7381 | 8209 |
| 1515 | 2414 | 4070 | 4898 | 5726 | 1516 | 3242 | 6554 | 7382 | 8210 |
| 1517 | 2415 | 4071 | 4899 | 5727 | 1518 | 3243 | 6555 | 7383 | 8211 |
| 1519 | 2416 | 4072 | 4900 | 5728 | 1520 | 3244 | 6556 | 7384 | 8212 |
| 1521 | 2417 | 4073 | 4901 | 5729 | 1522 | 3245 | 6557 | 7385 | 8213 |
| 1523 | 2418 | 4074 | 4902 | 5730 | 1524 | 3246 | 6558 | 7386 | 8214 |
| 1525 | 2419 | 4075 | 4903 | 5731 | 1526 | 3247 | 6559 | 7387 | 8215 |
| 1527 | 2420 | 4076 | 4904 | 5732 | 1528 | 3248 | 6560 | 7388 | 8216 |
| 1529 | 2421 | 4077 | 4905 | 5733 | 1530 | 3249 | 6561 | 7389 | 8217 |
| 1531 | 2422 | 4078 | 4906 | 5734 | 1532 | 3250 | 6562 | 7390 | 8218 |
| 1533 | 2423 | 4079 | 4907 | 5735 | 1534 | 3251 | 6563 | 7391 | 8219 |
| 1535 | 2424 | 4080 | 4908 | 5736 | 1536 | 3252 | 6564 | 7392 | 8220 |
| 1537 | 2425 | 4081 | 4909 | 5737 | 1538 | 3253 | 6565 | 7393 | 8221 |
| 1539 | 2426 | 4082 | 4910 | 5738 | 1540 | 3254 | 6566 | 7394 | 8222 |
| 1541 | 2427 | 4083 | 4911 | 5739 | 1542 | 3255 | 6567 | 7395 | 8223 |
| 1543 | 2428 | 4084 | 4912 | 5740 | 1544 | 3256 | 6568 | 7396 | 8224 |
| 1545 | 2429 | 4085 | 4913 | 5741 | 1546 | 3257 | 6569 | 7397 | 8225 |
| 1547 | 2430 | 4086 | 4914 | 5742 | 1548 | 3258 | 6570 | 7398 | 8226 |
| 1549 | 2431 | 4087 | 4915 | 5743 | 1550 | 3259 | 6571 | 7399 | 8227 |
| 1551 | 2432 | 4088 | 4916 | 5744 | 1552 | 3260 | 6572 | 7400 | 8228 |
| 1553 | 2433 | 4089 | 4917 | 5745 | 1554 | 3261 | 6573 | 7401 | 8229 |
| 1555 | 2434 | 4090 | 4918 | 5746 | 1556 | 3262 | 6574 | 7402 | 8230 |
| 1557 | 2435 | 4091 | 4919 | 5747 | 1558 | 3263 | 6575 | 7403 | 8231 |
| 1559 | 2436 | 4092 | 4920 | 5748 | 1560 | 3264 | 6576 | 7404 | 8232 |
| 1561 | 2437 | 4093 | 4921 | 5749 | 1562 | 3265 | 6577 | 7405 | 8233 |
| 1563 | 2438 | 4094 | 4922 | 5750 | 1564 | 3266 | 6578 | 7406 | 8234 |
| 1565 | 2439 | 4095 | 4923 | 5751 | 1566 | 3267 | 6579 | 7407 | 8235 |
| 1567 | 2440 | 4096 | 4924 | 5752 | 1568 | 3268 | 6580 | 7408 | 8236 |
| 1569 | 2441 | 4097 | 4925 | 5753 | 1570 | 3269 | 6581 | 7409 | 8237 |
| 1571 | 2442 | 4098 | 4926 | 5754 | 1572 | 3270 | 6582 | 7410 | 8238 |
| 1573 | 2443 | 4099 | 4927 | 5755 | 1574 | 3271 | 6583 | 7411 | 8239 |
| 1575 | 2444 | 4100 | 4928 | 5756 | 1576 | 3272 | 6584 | 7412 | 8240 |
| 1577 | 2445 | 4101 | 4929 | 5757 | 1578 | 3273 | 6585 | 7413 | 8241 |
| 1579 | 2446 | 4102 | 4930 | 5758 | 1580 | 3274 | 6586 | 7414 | 8242 |
| 1581 | 2447 | 4103 | 4931 | 5759 | 1582 | 3275 | 6587 | 7415 | 8243 |
| 1583 | 2448 | 4104 | 4932 | 5760 | 1584 | 3276 | 6588 | 7416 | 8244 |
| 1585 | 2449 | 4105 | 4933 | 5761 | 1586 | 3277 | 6589 | 7417 | 8245 |
| 1587 | 2450 | 4106 | 4934 | 5762 | 1588 | 3278 | 6590 | 7418 | 8246 |
| 1589 | 2451 | 4107 | 4935 | 5763 | 1590 | 3279 | 6591 | 7419 | 8247 |
| 1591 | 2452 | 4108 | 4936 | 5764 | 1592 | 3280 | 6592 | 7420 | 8248 |
| 1593 | 2453 | 4109 | 4937 | 5765 | 1594 | 3281 | 6593 | 7421 | 8249 |
| 1595 | 2454 | 4110 | 4938 | 5766 | 1596 | 3282 | 6594 | 7422 | 8250 |
| 1597 | 2455 | 4111 | 4939 | 5767 | 1598 | 3283 | 6595 | 7423 | 8251 |
| 1599 | 2456 | 4112 | 4940 | 5768 | 1600 | 3284 | 6596 | 7424 | 8252 |
| 1601 | 2457 | 4113 | 4941 | 5769 | 1602 | 3285 | 6597 | 7425 | 8253 |
| 1603 | 2458 | 4114 | 4942 | 5770 | 1604 | 3286 | 6598 | 7426 | 8254 |
| 1605 | 2459 | 4115 | 4943 | 5771 | 1606 | 3287 | 6599 | 7427 | 8255 |
| 1607 | 2460 | 4116 | 4944 | 5772 | 1608 | 3288 | 6600 | 7428 | 8256 |
| 1609 | 2461 | 4117 | 4945 | 5773 | 1610 | 3289 | 6601 | 7429 | 8257 |
| 1611 | 2462 | 4118 | 4946 | 5774 | 1612 | 3290 | 6602 | 7430 | 8258 |
| 1613 | 2463 | 4119 | 4947 | 5775 | 1614 | 3291 | 6603 | 7431 | 8259 |
| 1615 | 2464 | 4120 | 4948 | 5776 | 1616 | 3292 | 6604 | 7432 | 8260 |
| 1617 | 2465 | 4121 | 4949 | 5777 | 1618 | 3293 | 6605 | 7433 | 8261 |
| 1619 | 2466 | 4122 | 4950 | 5778 | 1620 | 3294 | 6606 | 7434 | 8262 |
| 1621 | 2467 | 4123 | 4951 | 5779 | 1622 | 3295 | 6607 | 7435 | 8263 |
| 1623 | 2468 | 4124 | 4952 | 5780 | 1624 | 3296 | 6608 | 7436 | 8264 |
| 1625 | 2469 | 4125 | 4953 | 5781 | 1626 | 3297 | 6609 | 7437 | 8265 |
| 1627 | 2470 | 4126 | 4954 | 5782 | 1628 | 3298 | 6610 | 7438 | 8266 |
| 1629 | 2471 | 4127 | 4955 | 5783 | 1630 | 3299 | 6611 | 7439 | 8267 |
| 1631 | 2472 | 4128 | 4956 | 5784 | 1632 | 3300 | 6612 | 7440 | 8268 |
| 1633 | 2473 | 4129 | 4957 | 5785 | 1634 | 3301 | 6613 | 7441 | 8269 |
| 1635 | 2474 | 4130 | 4958 | 5786 | 1636 | 3302 | 6614 | 7442 | 8270 |
| 1637 | 2475 | 4131 | 4959 | 5787 | 1638 | 3303 | 6615 | 7443 | 8271 |
| 1639 | 2476 | 4132 | 4960 | 5788 | 1640 | 3304 | 6616 | 7444 | 8272 |
| 1641 | 2477 | 4133 | 4961 | 5789 | 1642 | 3305 | 6617 | 7445 | 8273 |
| 1643 | 2478 | 4134 | 4962 | 5790 | 1644 | 3306 | 6618 | 7446 | 8274 |
| 1645 | 2479 | 4135 | 4963 | 5791 | 1646 | 3307 | 6619 | 7447 | 8275 |
| 1647 | 2480 | 4136 | 4964 | 5792 | 1648 | 3308 | 6620 | 7448 | 8276 |
| 1649 | 2481 | 4137 | 4965 | 5793 | 1650 | 3309 | 6621 | 7449 | 8277 |
| 1651 | 2482 | 4138 | 4966 | 5794 | 1652 | 3310 | 6622 | 7450 | 8278 |
| 1653 | 2483 | 4139 | 4967 | 5795 | 1654 | 3311 | 6623 | 7451 | 8279 |
| 1655 | 2484 | 4140 | 4968 | 5796 | 1656 | 3312 | 6624 | 7452 | 8280 |
| 8281 | 9939 | 11597 | 12426 | 13255 | 8282 | 10768 | 14084 | 14913 | 15742 |
| 8283 | 9940 | 11598 | 12427 | 13256 | 8284 | 10769 | 14085 | 14914 | 15743 |
| 8285 | 9941 | 11599 | 12428 | 13257 | 8286 | 10770 | 14086 | 14915 | 15744 |
| 8287 | 9942 | 11600 | 12429 | 13258 | 8288 | 10771 | 14087 | 14916 | 15745 |
| 8289 | 9943 | 11601 | 12430 | 13259 | 8290 | 10772 | 14088 | 14917 | 15746 |
| 8291 | 9944 | 11602 | 12431 | 13260 | 8292 | 10773 | 14089 | 14918 | 15747 |
| 8293 | 9945 | 11603 | 12432 | 13261 | 8294 | 10774 | 14090 | 14919 | 15748 |
| 8295 | 9946 | 11604 | 12433 | 13262 | 8296 | 10775 | 14091 | 14920 | 15749 |
| 8297 | 9947 | 11605 | 12434 | 13263 | 8298 | 10776 | 14092 | 14921 | 15750 |
| 8299 | 9948 | 11606 | 12435 | 13264 | 8300 | 10777 | 14093 | 14922 | 15751 |
| 8301 | 9949 | 11607 | 12436 | 13265 | 8302 | 10778 | 14094 | 14923 | 15752 |
| 8303 | 9950 | 11608 | 12437 | 13266 | 8304 | 10779 | 14095 | 14924 | 15753 |
| 8305 | 9951 | 11609 | 12438 | 13267 | 8306 | 10780 | 14096 | 14925 | 15754 |
| 8307 | 9952 | 11610 | 12439 | 13268 | 8308 | 10781 | 14097 | 14926 | 15755 |
| 8309 | 9953 | 11611 | 12440 | 13269 | 8310 | 10782 | 14098 | 14927 | 15756 |
| 8311 | 9954 | 11612 | 12441 | 13270 | 8312 | 10783 | 14099 | 14928 | 15757 |
| 8313 | 9955 | 11613 | 12442 | 13271 | 8314 | 10784 | 14100 | 14929 | 15758 |
| 8315 | 9956 | 11614 | 12443 | 13272 | 8316 | 10785 | 14101 | 14930 | 15759 |
| 8317 | 9957 | 11615 | 12444 | 13273 | 8318 | 10786 | 14102 | 14931 | 15760 |
| 8319 | 9958 | 11616 | 12445 | 13274 | 8320 | 10787 | 14103 | 14932 | 15761 |
| 8321 | 9959 | 11617 | 12446 | 13275 | 8322 | 10788 | 14104 | 14933 | 15762 |
| 8323 | 9960 | 11618 | 12447 | 13276 | 8324 | 10789 | 14105 | 14934 | 15763 |
| 8325 | 9961 | 11619 | 12448 | 13277 | 8326 | 10790 | 14106 | 14935 | 15764 |
| 8327 | 9962 | 11620 | 12449 | 13278 | 8328 | 10791 | 14107 | 14936 | 15765 |
| 8329 | 9963 | 11621 | 12450 | 13279 | 8330 | 10792 | 14108 | 14937 | 15766 |
| 8331 | 9964 | 11622 | 12451 | 13280 | 8332 | 10793 | 14109 | 14938 | 15767 |
| 8333 | 9965 | 11623 | 12452 | 13281 | 8334 | 10794 | 14110 | 14939 | 15768 |
| 8335 | 9966 | 11624 | 12453 | 13282 | 8336 | 10795 | 14111 | 14940 | 15769 |
| 8337 | 9967 | 11625 | 12454 | 13283 | 8338 | 10796 | 14112 | 14941 | 15770 |
| 8339 | 9968 | 11626 | 12455 | 13284 | 8340 | 10797 | 14113 | 14942 | 15771 |
| 8341 | 9969 | 11627 | 12456 | 13285 | 8342 | 10798 | 14114 | 14943 | 15772 |
| 8343 | 9970 | 11628 | 12457 | 13286 | 8344 | 10799 | 14115 | 14944 | 15773 |
| 8345 | 9971 | 11629 | 12458 | 13287 | 8346 | 10800 | 14116 | 14945 | 15774 |
| 8347 | 9972 | 11630 | 12459 | 13288 | 8348 | 10801 | 14117 | 14946 | 15775 |
| 8349 | 9973 | 11631 | 12460 | 13289 | 8350 | 10802 | 14118 | 14947 | 15776 |
| 8351 | 9974 | 11632 | 12461 | 13290 | 8352 | 10803 | 14119 | 14948 | 15777 |
| 8353 | 9975 | 11633 | 12462 | 13291 | 8354 | 10804 | 14120 | 14949 | 15778 |
| 8355 | 9976 | 11634 | 12463 | 13292 | 8356 | 10805 | 14121 | 14950 | 15779 |
| 8357 | 9977 | 11635 | 12464 | 13293 | 8358 | 10806 | 14122 | 14951 | 15780 |
| 8359 | 9978 | 11636 | 12465 | 13294 | 8360 | 10807 | 14123 | 14952 | 15781 |
| 8361 | 9979 | 11637 | 12466 | 13295 | 8362 | 10808 | 14124 | 14953 | 15782 |
| 8363 | 9980 | 11638 | 12467 | 13296 | 8364 | 10809 | 14125 | 14954 | 15783 |
| 8365 | 9981 | 11639 | 12468 | 13297 | 8366 | 10810 | 14126 | 14955 | 15784 |
| 8367 | 9982 | 11640 | 12469 | 13298 | 8368 | 10811 | 14127 | 14956 | 15785 |
| 8369 | 9983 | 11641 | 12470 | 13299 | 8370 | 10812 | 14128 | 14957 | 15786 |
| 8371 | 9984 | 11642 | 12471 | 13300 | 8372 | 10813 | 14129 | 14958 | 15787 |
| 8373 | 9985 | 11643 | 12472 | 13301 | 8374 | 10814 | 14130 | 14959 | 15788 |
| 8375 | 9986 | 11644 | 12473 | 13302 | 8376 | 10815 | 14131 | 14960 | 15789 |
| 8377 | 9987 | 11645 | 12474 | 13303 | 8378 | 10816 | 14132 | 14961 | 15790 |
| 8379 | 9988 | 11646 | 12475 | 13304 | 8380 | 10817 | 14133 | 14962 | 15791 |
| 8381 | 9989 | 11647 | 12476 | 13305 | 8382 | 10818 | 14134 | 14963 | 15792 |
| 8383 | 9990 | 11648 | 12477 | 13306 | 8384 | 10819 | 14135 | 14964 | 15793 |
| 8385 | 9991 | 11649 | 12478 | 13307 | 8386 | 10820 | 14136 | 14965 | 15794 |
| 8387 | 9992 | 11650 | 12479 | 13308 | 8388 | 10821 | 14137 | 14966 | 15795 |
| 8389 | 9993 | 11651 | 12480 | 13309 | 8390 | 10822 | 14138 | 14967 | 15796 |
| 8391 | 9994 | 11652 | 12481 | 13310 | 8392 | 10823 | 14139 | 14968 | 15797 |
| 8393 | 9995 | 11653 | 12482 | 13311 | 8394 | 10824 | 14140 | 14969 | 15798 |
| 8395 | 9996 | 11654 | 12483 | 13312 | 8396 | 10825 | 14141 | 14970 | 15799 |
| 8397 | 9997 | 11655 | 12484 | 13313 | 8398 | 10826 | 14142 | 14971 | 15800 |
| 8399 | 9998 | 11656 | 12485 | 13314 | 8400 | 10827 | 14143 | 14972 | 15801 |
| 8401 | 9999 | 11657 | 12486 | 13315 | 8402 | 10828 | 14144 | 14973 | 15802 |
| 8403 | 10000 | 11658 | 12487 | 13316 | 8404 | 10829 | 14145 | 14974 | 15803 |
| 8405 | 10001 | 11659 | 12488 | 13317 | 8406 | 10830 | 14146 | 14975 | 15804 |
| 8407 | 10002 | 11660 | 12489 | 13318 | 8408 | 10831 | 14147 | 14976 | 15805 |
| 8409 | 10003 | 11661 | 12490 | 13319 | 8410 | 10832 | 14148 | 14977 | 15806 |
| 8411 | 10004 | 11662 | 12491 | 13320 | 8412 | 10833 | 14149 | 14978 | 15807 |
| 8413 | 10005 | 11663 | 12492 | 13321 | 8414 | 10834 | 14150 | 14979 | 15808 |
| 8415 | 10006 | 11664 | 12493 | 13322 | 8416 | 10835 | 14151 | 14980 | 15809 |
| 8417 | 10007 | 11665 | 12494 | 13323 | 8418 | 10836 | 14152 | 14981 | 15810 |
| 8419 | 10008 | 11666 | 12495 | 13324 | 8420 | 10837 | 14153 | 14982 | 15811 |
| 8421 | 10009 | 11667 | 12496 | 13325 | 8422 | 10838 | 14154 | 14983 | 15812 |
| 8423 | 10010 | 11668 | 12497 | 13326 | 8424 | 10839 | 14155 | 14984 | 15813 |
| 8425 | 10011 | 11669 | 12498 | 13327 | 8426 | 10840 | 14156 | 14985 | 15814 |
| 8427 | 10012 | 11670 | 12499 | 13328 | 8428 | 10841 | 14157 | 14986 | 15815 |
| 8429 | 10013 | 11671 | 12500 | 13329 | 8430 | 10842 | 14158 | 14987 | 15816 |
| 8431 | 10014 | 11672 | 12501 | 13330 | 8432 | 10843 | 14159 | 14988 | 15817 |
| 8433 | 10015 | 11673 | 12502 | 13331 | 8434 | 10844 | 14160 | 14989 | 15818 |
| 8435 | 10016 | 11674 | 12503 | 13332 | 8436 | 10845 | 14161 | 14990 | 15819 |
| 8437 | 10017 | 11675 | 12504 | 13333 | 8438 | 10846 | 14162 | 14991 | 15820 |
| 8439 | 10018 | 11676 | 12505 | 13334 | 8440 | 10847 | 14163 | 14992 | 15821 |
| 8441 | 10019 | 11677 | 12506 | 13335 | 8442 | 10848 | 14164 | 14993 | 15822 |
| 8443 | 10020 | 11678 | 12507 | 13336 | 8444 | 10849 | 14165 | 14994 | 15823 |
| 8445 | 10021 | 11679 | 12508 | 13337 | 8446 | 10850 | 14166 | 14995 | 15824 |
| 8447 | 10022 | 11680 | 12509 | 13338 | 8448 | 10851 | 14167 | 14996 | 15825 |
| 8449 | 10023 | 11681 | 12510 | 13339 | 8450 | 10852 | 14168 | 14997 | 15826 |
| 8451 | 10024 | 11682 | 12511 | 13340 | 8452 | 10853 | 14169 | 14998 | 15827 |
| 8453 | 10025 | 11683 | 12512 | 13341 | 8454 | 10854 | 14170 | 14999 | 15828 |
| 8455 | 10026 | 11684 | 12513 | 13342 | 8456 | 10855 | 14171 | 15000 | 15829 |
| 8457 | 10027 | 11685 | 12514 | 13343 | 8458 | 10856 | 14172 | 15001 | 15830 |
| 8459 | 10028 | 11686 | 12515 | 13344 | 8460 | 10857 | 14173 | 15002 | 15831 |
| 8461 | 10029 | 11687 | 12516 | 13345 | 8462 | 10858 | 14174 | 15003 | 15832 |
| 8463 | 10030 | 11688 | 12517 | 13346 | 8464 | 10859 | 14175 | 15004 | 15833 |
| 8465 | 10031 | 11689 | 12518 | 13347 | 8466 | 10860 | 14176 | 15005 | 15834 |
| 8467 | 10032 | 11690 | 12519 | 13348 | 8468 | 10861 | 14177 | 15006 | 15835 |
| 8469 | 10033 | 11691 | 12520 | 13349 | 8470 | 10862 | 14178 | 15007 | 15836 |
| 8471 | 10034 | 11692 | 12521 | 13350 | 8472 | 10863 | 14179 | 15008 | 15837 |
| 8473 | 10035 | 11693 | 12522 | 13351 | 8474 | 10864 | 14180 | 15009 | 15838 |
| 8475 | 10036 | 11694 | 12523 | 13352 | 8476 | 10865 | 14181 | 15010 | 15839 |
| 8477 | 10037 | 11695 | 12524 | 13353 | 8478 | 10866 | 14182 | 15011 | 15840 |
| 8479 | 10038 | 11696 | 12525 | 13354 | 8480 | 10867 | 14183 | 15012 | 15841 |
| 8481 | 10039 | 11697 | 12526 | 13355 | 8482 | 10868 | 14184 | 15013 | 15842 |
| 8483 | 10040 | 11698 | 12527 | 13356 | 8484 | 10869 | 14185 | 15014 | 15843 |
| 8485 | 10041 | 11699 | 12528 | 13357 | 8486 | 10870 | 14186 | 15015 | 15844 |
| 8487 | 10042 | 11700 | 12529 | 13358 | 8488 | 10871 | 14187 | 15016 | 15845 |
| 8489 | 10043 | 11701 | 12530 | 13359 | 8490 | 10872 | 14188 | 15017 | 15846 |
| 8491 | 10044 | 11702 | 12531 | 13360 | 8492 | 10873 | 14189 | 15018 | 15847 |
| 8493 | 10045 | 11703 | 12532 | 13361 | 8494 | 10874 | 14190 | 15019 | 15848 |
| 8495 | 10046 | 11704 | 12533 | 13362 | 8496 | 10875 | 14191 | 15020 | 15849 |
| 8497 | 10047 | 11705 | 12534 | 13363 | 8498 | 10876 | 14192 | 1502 | 15850 |
| 8499 | 10048 | 11706 | 12535 | 13364 | 8500 | 10877 | 14193 | 15022 | 15851 |
| 8501 | 10049 | 11707 | 12536 | 13365 | 8502 | 10878 | 14194 | 15023 | 15852 |
| 8503 | 10050 | 11708 | 12537 | 13366 | 8504 | 10879 | 14195 | 15024 | 15853 |
| 8505 | 10051 | 11709 | 12538 | 13367 | 8506 | 10880 | 14196 | 15025 | 15854 |
| 8507 | 10052 | 11710 | 12539 | 13368 | 8508 | 10881 | 14197 | 15026 | 15855 |
| 8509 | 10053 | 11711 | 12540 | 13369 | 8510 | 10882 | 14198 | 15027 | 15856 |
| 8511 | 10054 | 11712 | 12541 | 13370 | 8512 | 10883 | 14199 | 15028 | 15857 |
| 8513 | 10055 | 11713 | 12542 | 13371 | 8514 | 10884 | 14200 | 15029 | 15858 |
| 8515 | 10056 | 11714 | 12543 | 13372 | 8516 | 10885 | 14201 | 15030 | 15859 |
| 8517 | 10057 | 11715 | 12544 | 13373 | 8518 | 10886 | 14202 | 15031 | 15860 |
| 8519 | 10058 | 11716 | 12545 | 13374 | 8520 | 10887 | 14203 | 15032 | 15861 |
| 8521 | 10059 | 11717 | 12546 | 13375 | 8522 | 10888 | 14204 | 15033 | 15862 |
| 8523 | 10060 | 11718 | 12547 | 13376 | 8524 | 10889 | 14205 | 15034 | 15863 |
| 8525 | 10061 | 11719 | 12548 | 13377 | 8526 | 10890 | 14206 | 15035 | 15864 |
| 8527 | 10062 | 11720 | 12549 | 13378 | 8528 | 10891 | 14207 | 15036 | 15865 |
| 8529 | 10063 | 11721 | 12550 | 13379 | 8530 | 10892 | 14208 | 15037 | 15866 |
| 8531 | 10064 | 11722 | 12551 | 13380 | 8532 | 10893 | 14209 | 15038 | 15867 |
| 8533 | 10065 | 11723 | 12552 | 13381 | 8534 | 10894 | 14210 | 15039 | 15868 |
| 8535 | 10066 | 11724 | 12553 | 13382 | 8536 | 10895 | 14211 | 15040 | 15869 |
| 8537 | 10067 | 11725 | 12554 | 13383 | 8538 | 10896 | 14212 | 15041 | 15870 |
| 8539 | 10068 | 11726 | 12555 | 13384 | 8540 | 10897 | 14213 | 15042 | 15871 |
| 8541 | 10069 | 11727 | 12556 | 13385 | 8542 | 10898 | 14214 | 15043 | 15872 |
| 8543 | 10070 | 11728 | 12557 | 13386 | 8544 | 10899 | 14215 | 15044 | 15873 |
| 8545 | 10071 | 11729 | 12558 | 13387 | 8546 | 10900 | 14216 | 15045 | 15874 |
| 8547 | 10072 | 11730 | 12559 | 13388 | 8548 | 10901 | 14217 | 15046 | 15875 |
| 8549 | 10073 | 11731 | 12560 | 13389 | 8550 | 10902 | 14218 | 15047 | 15876 |
| 8551 | 10074 | 11732 | 12561 | 13390 | 8552 | 10903 | 14219 | 15048 | 15877 |
| 8553 | 10075 | 11733 | 12562 | 13391 | 8554 | 10904 | 14220 | 15049 | 15878 |
| 8555 | 10076 | 11734 | 12563 | 13392 | 8556 | 10905 | 14221 | 15050 | 15879 |
| 8557 | 10077 | 11735 | 12564 | 13393 | 8558 | 10906 | 14222 | 15051 | 15880 |
| 8559 | 10078 | 11736 | 12565 | 13394 | 8560 | 10907 | 14223 | 15052 | 15881 |
| 8561 | 10079 | 11737 | 12566 | 13395 | 8562 | 10908 | 14224 | 15053 | 15882 |
| 8563 | 10080 | 11738 | 12567 | 13396 | 8564 | 10909 | 14225 | 15054 | 15883 |
| 8565 | 10081 | 11739 | 12568 | 13397 | 8566 | 10910 | 14226 | 15055 | 15884 |
| 8567 | 10082 | 11740 | 12569 | 13398 | 8568 | 10911 | 14227 | 15056 | 15885 |
| 8569 | 10083 | 11741 | 12570 | 13399 | 8570 | 10912 | 14228 | 15057 | 15886 |
| 8571 | 10084 | 11742 | 12571 | 13400 | 8572 | 10913 | 14229 | 15058 | 15887 |
| 8573 | 10085 | 11743 | 12572 | 13401 | 8574 | 10914 | 14230 | 15059 | 15888 |
| 8575 | 10086 | 11744 | 12573 | 13402 | 8576 | 10915 | 14231 | 15060 | 15889 |
| 8577 | 10087 | 11745 | 12574 | 13403 | 8578 | 10916 | 14232 | 15061 | 15890 |
| 8579 | 10088 | 11746 | 12575 | 13404 | 8580 | 10917 | 14233 | 15062 | 15891 |
| 8581 | 10089 | 11747 | 12576 | 13405 | 8582 | 10918 | 14234 | 15063 | 15892 |
| 8583 | 10090 | 11748 | 12577 | 13406 | 8584 | 10919 | 14235 | 15064 | 15893 |
| 8585 | 10091 | 11749 | 12578 | 13407 | 8586 | 10920 | 14236 | 15065 | 15894 |
| 8587 | 10092 | 11750 | 12579 | 13408 | 8588 | 10921 | 14237 | 15066 | 15895 |
| 8589 | 10093 | 11751 | 12580 | 13409 | 8590 | 10922 | 14238 | 15067 | 15896 |
| 8591 | 10094 | 11752 | 12581 | 13410 | 8592 | 10923 | 14239 | 15068 | 15897 |
| 8593 | 10095 | 11753 | 12582 | 13411 | 8594 | 10924 | 14240 | 15069 | 15898 |
| 8595 | 10096 | 11754 | 12583 | 13412 | 8596 | 10925 | 14241 | 15070 | 15899 |
| 8597 | 10097 | 11755 | 12584 | 13413 | 8598 | 10926 | 14242 | 15071 | 15900 |
| 8599 | 10098 | 11756 | 12585 | 13414 | 8600 | 10927 | 14243 | 15072 | 15901 |
| 8601 | 10099 | 11757 | 12586 | 13415 | 8602 | 10928 | 14244 | 15073 | 15902 |
| 8603 | 10100 | 11758 | 12587 | 13416 | 8604 | 10929 | 14245 | 15074 | 15903 |
| 8605 | 10101 | 11759 | 12588 | 13417 | 8606 | 10930 | 14246 | 15075 | 15904 |
| 8607 | 10102 | 11760 | 12589 | 13418 | 8608 | 10931 | 14247 | 15076 | 15905 |
| 8609 | 10103 | 11761 | 12590 | 13419 | 8610 | 10932 | 14248 | 15077 | 15906 |
| 8611 | 10104 | 11762 | 12591 | 13420 | 8612 | 10933 | 14249 | 15078 | 15907 |
| 8613 | 10105 | 11763 | 12592 | 13421 | 8614 | 10934 | 14250 | 15079 | 15908 |
| 8615 | 10106 | 11764 | 12593 | 13422 | 8616 | 10935 | 14251 | 15080 | 15909 |
| 8617 | 10107 | 11765 | 12594 | 13423 | 8618 | 10936 | 14252 | 15081 | 15910 |
| 8619 | 10108 | 11766 | 12595 | 13424 | 8620 | 10937 | 14253 | 15082 | 15911 |
| 8621 | 10109 | 11767 | 12596 | 13425 | 8622 | 10938 | 14254 | 15083 | 15912 |
| 8623 | 10110 | 11768 | 12597 | 13426 | 8624 | 10939 | 14255 | 15084 | 15913 |
| 8625 | 10111 | 11769 | 12598 | 13427 | 8626 | 10940 | 14256 | 15085 | 15914 |
| 8627 | 10112 | 11770 | 12599 | 13428 | 8628 | 10941 | 14257 | 15086 | 15915 |
| 8629 | 10113 | 11771 | 12600 | 13429 | 8630 | 10942 | 14258 | 15087 | 15916 |
| 8631 | 10114 | 11772 | 12601 | 13430 | 8632 | 10943 | 14259 | 15088 | 15917 |
| 8633 | 10115 | 11773 | 12602 | 13431 | 8634 | 10944 | 14260 | 15089 | 15918 |
| 8635 | 10116 | 11774 | 12603 | 13432 | 8636 | 10945 | 14261 | 15090 | 15919 |
| 8637 | 10117 | 11775 | 12604 | 13433 | 8638 | 10946 | 14262 | 15091 | 15920 |
| 8639 | 10118 | 11776 | 12605 | 13434 | 8640 | 10947 | 14263 | 15092 | 15921 |
| 8641 | 10119 | 11777 | 12606 | 13435 | 8642 | 10948 | 14264 | 15093 | 15922 |
| 8643 | 10120 | 11778 | 12607 | 13436 | 8644 | 10949 | 14265 | 15094 | 15923 |
| 8645 | 10121 | 11779 | 12608 | 13437 | 8646 | 10950 | 14266 | 15095 | 15924 |
| 8647 | 10122 | 11780 | 12609 | 13438 | 8648 | 10951 | 14267 | 15096 | 15925 |
| 8649 | 10123 | 11781 | 12610 | 13439 | 8650 | 10952 | 14268 | 15097 | 15926 |
| 8651 | 10124 | 11782 | 12611 | 13440 | 8652 | 10953 | 14269 | 15098 | 15927 |
| 8653 | 10125 | 11783 | 12612 | 13441 | 8654 | 10954 | 14270 | 15099 | 15928 |
| 8655 | 10126 | 11784 | 12613 | 13442 | 8656 | 10955 | 14271 | 15100 | 15929 |
| 8657 | 10127 | 11785 | 12614 | 13443 | 8658 | 10956 | 14272 | 15101 | 15930 |
| 8659 | 10128 | 11786 | 12615 | 13444 | 8660 | 10957 | 14273 | 15102 | 15931 |
| 8661 | 10129 | 11787 | 12616 | 13445 | 8662 | 10958 | 14274 | 15103 | 15932 |
| 8663 | 10130 | 11788 | 12617 | 13446 | 8664 | 10959 | 14275 | 15104 | 15933 |
| 8665 | 10131 | 11789 | 12618 | 13447 | 8666 | 10960 | 14276 | 15105 | 15934 |
| 8667 | 10132 | 11790 | 12619 | 13448 | 8668 | 10961 | 14277 | 15106 | 15935 |
| 8669 | 10133 | 11791 | 12620 | 13449 | 8670 | 10962 | 14278 | 15107 | 15936 |
| 8671 | 10134 | 11792 | 12621 | 13450 | 8672 | 10963 | 14279 | 15108 | 15937 |
| 8673 | 10135 | 11793 | 12622 | 13451 | 8674 | 10964 | 14280 | 15109 | 15938 |
| 8675 | 10136 | 11794 | 12623 | 13452 | 8676 | 10965 | 14281 | 15110 | 15939 |
| 8677 | 10137 | 11795 | 12624 | 13453 | 8678 | 10966 | 14282 | 15111 | 15940 |
| 8679 | 10138 | 11796 | 12625 | 13454 | 8680 | 10967 | 14283 | 15112 | 15941 |
| 8681 | 10139 | 11797 | 12626 | 13455 | 8682 | 10968 | 14284 | 15113 | 15942 |
| 8683 | 10140 | 11798 | 12627 | 13456 | 8684 | 10969 | 14285 | 15114 | 15943 |
| 8685 | 10141 | 11799 | 12628 | 13457 | 8686 | 10970 | 14286 | 15115 | 15944 |
| 8687 | 10142 | 11800 | 12629 | 13458 | 8688 | 10971 | 14287 | 15116 | 15945 |
| 8689 | 10143 | 11801 | 12630 | 13459 | 8690 | 10972 | 14288 | 15117 | 15946 |
| 8691 | 10144 | 11802 | 12631 | 13460 | 8692 | 10973 | 14289 | 15118 | 15947 |
| 8693 | 10145 | 11803 | 12632 | 13461 | 8694 | 10974 | 14290 | 15119 | 15948 |
| 8695 | 10146 | 11804 | 12633 | 13462 | 8696 | 10975 | 14291 | 15120 | 15949 |
| 8697 | 10147 | 11805 | 12634 | 13463 | 8698 | 10976 | 14292 | 15121 | 15950 |
| 8699 | 10148 | 11806 | 12635 | 13464 | 8700 | 10977 | 14293 | 15122 | 15951 |
| 8701 | 10149 | 11807 | 12636 | 13465 | 8702 | 10978 | 14294 | 15123 | 15952 |
| 8703 | 10150 | 11808 | 12637 | 13466 | 8704 | 10979 | 14295 | 15124 | 15953 |
| 8705 | 10151 | 11809 | 12638 | 13467 | 8706 | 10980 | 14296 | 15125 | 15954 |
| 8707 | 10152 | 11810 | 12639 | 13468 | 8708 | 10981 | 14297 | 15126 | 15955 |
| 8709 | 10153 | 11811 | 12640 | 13469 | 8710 | 10982 | 14298 | 15127 | 15956 |
| 8711 | 10154 | 11812 | 12641 | 13470 | 8712 | 10983 | 14299 | 15128 | 15957 |
| 8713 | 10155 | 11813 | 12642 | 13471 | 8714 | 10984 | 14300 | 15129 | 15958 |
| 8715 | 10156 | 11814 | 12643 | 13472 | 8716 | 10985 | 14301 | 15130 | 15959 |
| 8717 | 10157 | 11815 | 12644 | 13473 | 8718 | 10986 | 14302 | 15131 | 15960 |
| 8719 | 10158 | 11816 | 12645 | 13474 | 8720 | 10987 | 14303 | 15132 | 15961 |
| 8721 | 10159 | 11817 | 12646 | 13475 | 8722 | 10988 | 14304 | 15133 | 15962 |
| 8723 | 10160 | 11818 | 12647 | 13476 | 8724 | 10989 | 14305 | 15134 | 15963 |
| 8725 | 10161 | 11819 | 12648 | 13477 | 8726 | 10990 | 14306 | 15135 | 15964 |
| 8727 | 10162 | 11820 | 12649 | 13478 | 8728 | 10991 | 14307 | 15136 | 15965 |
| 8729 | 10163 | 11821 | 12650 | 13479 | 8730 | 10992 | 14308 | 15137 | 15966 |
| 8731 | 10164 | 11822 | 12651 | 13480 | 8732 | 10993 | 14309 | 15138 | 15967 |
| 8733 | 10165 | 11823 | 12652 | 13481 | 8734 | 10994 | 14310 | 15139 | 15968 |
| 8735 | 10166 | 11824 | 12653 | 13482 | 8736 | 10995 | 14311 | 15140 | 15969 |
| 8737 | 10167 | 11825 | 12654 | 13483 | 8738 | 10996 | 14312 | 15141 | 15970 |
| 8739 | 10168 | 11826 | 12655 | 13484 | 8740 | 10997 | 14313 | 15142 | 15971 |
| 8741 | 10169 | 11827 | 12656 | 13485 | 8742 | 10998 | 14314 | 15143 | 15972 |
| 8743 | 10170 | 11828 | 12657 | 13486 | 8744 | 10999 | 14315 | 15144 | 15973 |
| 8745 | 10171 | 11829 | 12658 | 13487 | 8746 | 11000 | 14316 | 15145 | 15974 |
| 8747 | 10172 | 11830 | 12659 | 13488 | 8748 | 11001 | 14317 | 15146 | 15975 |
| 8749 | 10173 | 11831 | 12660 | 13489 | 8750 | 11002 | 14318 | 15147 | 15976 |
| 8751 | 10174 | 11832 | 12661 | 13490 | 8752 | 11003 | 14319 | 15148 | 15977 |
| 8753 | 10175 | 11833 | 12662 | 13491 | 8754 | 11004 | 14320 | 15149 | 15978 |
| 8755 | 10176 | 11834 | 12663 | 13492 | 8756 | 11005 | 14321 | 15150 | 15979 |
| 8757 | 10177 | 11835 | 12664 | 13493 | 8758 | 11006 | 14322 | 15151 | 15980 |
| 8759 | 10178 | 11836 | 12665 | 13494 | 8760 | 11007 | 14323 | 15152 | 15981 |
| 8761 | 10179 | 11837 | 12666 | 13495 | 8762 | 11008 | 14324 | 15153 | 15982 |
| 8763 | 10180 | 11838 | 12667 | 13496 | 8764 | 11009 | 14325 | 15154 | 15983 |
| 8765 | 10181 | 11839 | 12668 | 13497 | 8766 | 11010 | 14326 | 15155 | 15984 |
| 8767 | 10182 | 11840 | 12669 | 13498 | 8768 | 11011 | 14327 | 15156 | 15985 |
| 8769 | 10183 | 11841 | 12670 | 13499 | 8770 | 11012 | 14328 | 15157 | 15986 |
| 8771 | 10184 | 11842 | 12671 | 13500 | 8772 | 11013 | 14329 | 15158 | 15987 |
| 8773 | 10185 | 11843 | 12672 | 13501 | 8774 | 11014 | 14330 | 15159 | 15988 |
| 8775 | 10186 | 11844 | 12673 | 13502 | 8776 | 11015 | 14331 | 15160 | 15989 |
| 8777 | 10187 | 11845 | 12674 | 13503 | 8778 | 11016 | 14332 | 15161 | 15990 |
| 8779 | 10188 | 11846 | 12675 | 13504 | 8780 | 11017 | 14333 | 15162 | 15991 |
| 8781 | 10189 | 11847 | 12676 | 13505 | 8782 | 11018 | 14334 | 15163 | 15992 |
| 8783 | 10190 | 11848 | 12677 | 13506 | 8784 | 11019 | 14335 | 15164 | 15993 |
| 8785 | 10191 | 11849 | 12678 | 13507 | 8786 | 11020 | 14336 | 15165 | 15994 |
| 8787 | 10192 | 11850 | 12679 | 13508 | 8788 | 11021 | 14337 | 15166 | 15995 |
| 8789 | 10193 | 11851 | 12680 | 13509 | 8790 | 11022 | 14338 | 15167 | 15996 |
| 8791 | 10194 | 11852 | 12681 | 13510 | 8792 | 11023 | 14339 | 15168 | 15997 |
| 8793 | 10195 | 11853 | 12682 | 13511 | 8794 | 11024 | 14340 | 15169 | 15998 |
| 8795 | 10196 | 11854 | 12683 | 13512 | 8796 | 11025 | 14341 | 15170 | 15999 |
| 8797 | 10197 | 11855 | 12684 | 13513 | 8798 | 11026 | 14342 | 15171 | 16000 |
| 8799 | 10198 | 11856 | 12685 | 13514 | 8800 | 11027 | 14343 | 15172 | 16001 |
| 8801 | 10199 | 11857 | 12686 | 13515 | 8802 | 11028 | 14344 | 15173 | 16002 |
| 8803 | 10200 | 11858 | 12687 | 13516 | 8804 | 11029 | 14345 | 15174 | 16003 |
| 8805 | 10201 | 11859 | 12688 | 13517 | 8806 | 11030 | 14346 | 15175 | 16004 |
| 8807 | 10202 | 11860 | 12689 | 13518 | 8808 | 11031 | 14347 | 15176 | 16005 |
| 8809 | 10203 | 11861 | 12690 | 13519 | 8810 | 11032 | 14348 | 15177 | 16006 |
| 8811 | 10204 | 11862 | 12691 | 13520 | 8812 | 11033 | 14349 | 15178 | 16007 |
| 8813 | 10205 | 11863 | 12692 | 13521 | 8814 | 11034 | 14350 | 15179 | 16008 |
| 8815 | 10206 | 11864 | 12693 | 13522 | 8816 | 11035 | 14351 | 15180 | 16009 |
| 8817 | 10207 | 11865 | 12694 | 13523 | 8818 | 11036 | 14352 | 15181 | 16010 |
| 8819 | 10208 | 11866 | 12695 | 13524 | 8820 | 11037 | 14353 | 15182 | 16011 |
| 8821 | 10209 | 11867 | 12696 | 13525 | 8822 | 11038 | 14354 | 15183 | 16012 |
| 8823 | 10210 | 11868 | 12697 | 13526 | 8824 | 11039 | 14355 | 15184 | 16013 |
| 8825 | 10211 | 11869 | 12698 | 13527 | 8826 | 11040 | 14356 | 15185 | 16014 |
| 8827 | 10212 | 11870 | 12699 | 13528 | 8828 | 11041 | 14357 | 15186 | 16015 |
| 8829 | 10213 | 11871 | 12700 | 13529 | 8830 | 11042 | 14358 | 15187 | 16016 |
| 8831 | 10214 | 11872 | 12701 | 13530 | 8832 | 11043 | 14359 | 15188 | 16017 |
| 8833 | 10215 | 11873 | 12702 | 13531 | 8834 | 11044 | 14360 | 15189 | 16018 |
| 8835 | 10216 | 11874 | 12703 | 13532 | 8836 | 11045 | 14361 | 15190 | 16019 |
| 8837 | 10217 | 11875 | 12704 | 13533 | 8838 | 11046 | 14362 | 15191 | 16020 |
| 8839 | 10218 | 11876 | 12705 | 13534 | 8840 | 11047 | 14363 | 15192 | 16021 |
| 8841 | 10219 | 11877 | 12706 | 13535 | 8842 | 11048 | 14364 | 15193 | 16022 |
| 8843 | 10220 | 11878 | 12707 | 13536 | 8844 | 11049 | 14365 | 15194 | 16023 |
| 8845 | 10221 | 11879 | 12708 | 13537 | 8846 | 11050 | 14366 | 15195 | 16024 |
| 8847 | 10222 | 11880 | 12709 | 13538 | 8848 | 11051 | 14367 | 15196 | 16025 |
| 8849 | 10223 | 11881 | 12710 | 13539 | 8850 | 11052 | 14368 | 15197 | 16026 |
| 8851 | 10224 | 11882 | 12711 | 13540 | 8852 | 11053 | 14369 | 15198 | 16027 |
| 8853 | 10225 | 11883 | 12712 | 13541 | 8854 | 11054 | 14370 | 15199 | 16028 |
| 8855 | 10226 | 11884 | 12713 | 13542 | 8856 | 11055 | 14371 | 15200 | 16029 |
| 8857 | 10227 | 11885 | 12714 | 13543 | 8858 | 11056 | 14372 | 15201 | 16030 |
| 8859 | 10228 | 11886 | 12715 | 13544 | 8860 | 11057 | 14373 | 15202 | 16031 |
| 8861 | 10229 | 11887 | 12716 | 13545 | 8862 | 11058 | 14374 | 15203 | 16032 |
| 8863 | 10230 | 11888 | 12717 | 13546 | 8864 | 11059 | 14375 | 15204 | 16033 |
| 8865 | 10231 | 11889 | 12718 | 13547 | 8866 | 11060 | 14376 | 15205 | 16034 |
| 8867 | 10232 | 11890 | 12719 | 13548 | 8868 | 11061 | 14377 | 15206 | 16035 |
| 8869 | 10233 | 11891 | 12720 | 13549 | 8870 | 11062 | 14378 | 15207 | 16036 |
| 8871 | 10234 | 11892 | 12721 | 13550 | 8872 | 11063 | 14379 | 15208 | 16037 |
| 8873 | 10235 | 11893 | 12722 | 13551 | 8874 | 11064 | 14380 | 15209 | 16038 |
| 8875 | 10236 | 11894 | 12723 | 13552 | 8876 | 11065 | 14381 | 15210 | 16039 |
| 8877 | 10237 | 11895 | 12724 | 13553 | 8878 | 11066 | 14382 | 15211 | 16040 |
| 8879 | 10238 | 11896 | 12725 | 13554 | 8880 | 11067 | 14383 | 15212 | 16041 |
| 8881 | 10239 | 11897 | 12726 | 13555 | 8882 | 11068 | 14384 | 15213 | 16042 |
| 8883 | 10240 | 11898 | 12727 | 13556 | 8884 | 11069 | 14385 | 15214 | 16043 |
| 8885 | 10241 | 11899 | 12728 | 13557 | 8886 | 11070 | 14386 | 15215 | 16044 |
| 8887 | 10242 | 11900 | 12729 | 13558 | 8888 | 11071 | 14387 | 15216 | 16045 |
| 8889 | 10243 | 11901 | 12730 | 13559 | 8890 | 11072 | 14388 | 15217 | 16046 |
| 8891 | 10244 | 11902 | 12731 | 13560 | 8892 | 11073 | 14389 | 15218 | 16047 |
| 8893 | 10245 | 11903 | 12732 | 13561 | 8894 | 11074 | 14390 | 15219 | 16048 |
| 8895 | 10246 | 11904 | 12733 | 13562 | 8896 | 11075 | 14391 | 15220 | 16049 |
| 8897 | 10247 | 11905 | 12734 | 13563 | 8898 | 11076 | 14392 | 15221 | 16050 |
| 8899 | 10248 | 11906 | 12735 | 13564 | 8900 | 11077 | 14393 | 15222 | 16051 |
| 8901 | 10249 | 11907 | 12736 | 13565 | 8902 | 11078 | 14394 | 15223 | 16052 |
| 8903 | 10250 | 11908 | 12737 | 13566 | 8904 | 11079 | 14395 | 15224 | 16053 |
| 8905 | 10251 | 11909 | 12738 | 13567 | 8906 | 11080 | 14396 | 15225 | 16054 |
| 8907 | 10252 | 11910 | 12739 | 13568 | 8908 | 11081 | 14397 | 15226 | 16055 |
| 8909 | 10253 | 11911 | 12740 | 13569 | 8910 | 11082 | 14398 | 15227 | 16056 |
| 8911 | 10254 | 11912 | 12741 | 13570 | 8912 | 11083 | 14399 | 15228 | 16057 |
| 8913 | 10255 | 11913 | 12742 | 13571 | 8914 | 11084 | 14400 | 15229 | 16058 |
| 8915 | 10256 | 11914 | 12743 | 13572 | 8916 | 11085 | 14401 | 15230 | 16059 |
| 8917 | 10257 | 11915 | 12744 | 13573 | 8918 | 11086 | 14402 | 15231 | 16060 |
| 8919 | 10258 | 11916 | 12745 | 13574 | 8920 | 11087 | 14403 | 15232 | 16061 |
| 8921 | 10259 | 11917 | 12746 | 13575 | 8922 | 11088 | 14404 | 15233 | 16062 |
| 8923 | 10260 | 11918 | 12747 | 13576 | 8924 | 11089 | 14405 | 15234 | 16063 |
| 8925 | 10261 | 11919 | 12748 | 13577 | 8926 | 11090 | 14406 | 15235 | 16064 |
| 8927 | 10262 | 11920 | 12749 | 13578 | 8928 | 11091 | 14407 | 15236 | 16065 |
| 8929 | 10263 | 11921 | 12750 | 13579 | 8930 | 11092 | 14408 | 15237 | 16066 |
| 8931 | 10264 | 11922 | 12751 | 13580 | 8932 | 11093 | 14409 | 15238 | 16067 |
| 8933 | 10265 | 11923 | 12752 | 13581 | 8934 | 11094 | 14410 | 15239 | 16068 |
| 8935 | 10266 | 11924 | 12753 | 13582 | 8936 | 11095 | 14411 | 15240 | 16069 |
| 8937 | 10267 | 11925 | 12754 | 13583 | 8938 | 11096 | 14412 | 15241 | 16070 |
| 8939 | 10268 | 11926 | 12755 | 13584 | 8940 | 11097 | 14413 | 15242 | 16071 |
| 8941 | 10269 | 11927 | 12756 | 13585 | 8942 | 11098 | 14414 | 15243 | 16072 |
| 8943 | 10270 | 11928 | 12757 | 13586 | 8944 | 11099 | 14415 | 15244 | 16073 |
| 8945 | 10271 | 11929 | 12758 | 13587 | 8946 | 11100 | 14416 | 15245 | 16074 |
| 8947 | 10272 | 11930 | 12759 | 13588 | 8948 | 11101 | 14417 | 15246 | 16075 |
| 8949 | 10273 | 11931 | 12760 | 13589 | 8950 | 11102 | 14418 | 15247 | 16076 |
| 8951 | 10274 | 11932 | 12761 | 13590 | 8952 | 11103 | 14419 | 15248 | 16077 |
| 8953 | 10275 | 11933 | 12762 | 13591 | 8954 | 11104 | 14420 | 15249 | 16078 |
| 8955 | 10276 | 11934 | 12763 | 13592 | 8956 | 11105 | 14421 | 15250 | 16079 |
| 8957 | 10277 | 11935 | 12764 | 13593 | 8958 | 11106 | 14422 | 15251 | 16080 |
| 8959 | 10278 | 11936 | 12765 | 13594 | 8960 | 11107 | 14423 | 15252 | 16081 |
| 8961 | 10279 | 11937 | 12766 | 13595 | 8962 | 11108 | 14424 | 15253 | 16082 |
| 8963 | 10280 | 11938 | 12767 | 13596 | 8964 | 11109 | 14425 | 15254 | 16083 |
| 8965 | 10281 | 11939 | 12768 | 13597 | 8966 | 11110 | 14426 | 15255 | 16084 |
| 8967 | 10282 | 11940 | 12769 | 13598 | 8968 | 11111 | 14427 | 15256 | 16085 |
| 8969 | 10283 | 11941 | 12770 | 13599 | 8970 | 11112 | 14428 | 15257 | 16086 |
| 8971 | 10284 | 11942 | 12771 | 13600 | 8972 | 11113 | 14429 | 15258 | 16087 |
| 8973 | 10285 | 11943 | 12772 | 13601 | 8974 | 11114 | 14430 | 15259 | 16088 |
| 8975 | 10286 | 11944 | 12773 | 13602 | 8976 | 11115 | 14431 | 15260 | 16089 |
| 8977 | 10287 | 11945 | 12774 | 13603 | 8978 | 11116 | 14432 | 15261 | 16090 |
| 8979 | 10288 | 11946 | 12775 | 13604 | 8980 | 11117 | 14433 | 15262 | 16091 |
| 8981 | 10289 | 11947 | 12776 | 13605 | 8982 | 11118 | 14434 | 15263 | 16092 |
| 8983 | 10290 | 11948 | 12777 | 13606 | 8984 | 11119 | 14435 | 15264 | 16093 |
| 8985 | 10291 | 11949 | 12778 | 13607 | 8986 | 11120 | 14436 | 15265 | 16094 |
| 8987 | 10292 | 11950 | 12779 | 13608 | 8988 | 11121 | 14437 | 15266 | 16095 |
| 8989 | 10293 | 11951 | 12780 | 13609 | 8990 | 11122 | 14438 | 15267 | 16096 |
| 8991 | 10294 | 11952 | 12781 | 13610 | 8992 | 11123 | 14439 | 15268 | 16097 |
| 8993 | 10295 | 11953 | 12782 | 13611 | 8994 | 11124 | 14440 | 15269 | 16098 |
| 8995 | 10296 | 11954 | 12783 | 13612 | 8996 | 11125 | 14441 | 15270 | 16099 |
| 8997 | 10297 | 11955 | 12784 | 13613 | 8998 | 11126 | 14442 | 15271 | 16100 |
| 8999 | 10298 | 11956 | 12785 | 13614 | 9000 | 11127 | 14443 | 15272 | 16101 |
| 9001 | 10299 | 11957 | 12786 | 13615 | 9002 | 11128 | 14444 | 15273 | 16102 |
| 9003 | 10300 | 11958 | 12787 | 13616 | 9004 | 11129 | 14445 | 15274 | 16103 |
| 9005 | 10301 | 11959 | 12788 | 13617 | 9006 | 11130 | 14446 | 15275 | 16104 |
| 9007 | 10302 | 11960 | 12789 | 13618 | 9008 | 11131 | 14447 | 15276 | 16105 |
| 9009 | 10303 | 11961 | 12790 | 13619 | 9010 | 11132 | 14448 | 15277 | 16106 |
| 9011 | 10304 | 11962 | 12791 | 13620 | 9012 | 11133 | 14449 | 15278 | 16107 |
| 9013 | 10305 | 11963 | 12792 | 13621 | 9014 | 11134 | 14450 | 15279 | 16108 |
| 9015 | 10306 | 11964 | 12793 | 13622 | 9016 | 11135 | 14451 | 15280 | 16109 |
| 9017 | 10307 | 11965 | 12794 | 13623 | 9018 | 11136 | 14452 | 15281 | 16110 |
| 9019 | 10308 | 11966 | 12795 | 13624 | 9020 | 11137 | 14453 | 15282 | 16111 |
| 9021 | 10309 | 11967 | 12796 | 13625 | 9022 | 11138 | 14454 | 15283 | 16112 |
| 9023 | 10310 | 11968 | 12797 | 13626 | 9024 | 11139 | 14455 | 15284 | 16113 |
| 9025 | 10311 | 11969 | 12798 | 13627 | 9026 | 11140 | 14456 | 15285 | 16114 |
| 9027 | 10312 | 11970 | 12799 | 13628 | 9028 | 11141 | 14457 | 15286 | 16115 |
| 9029 | 10313 | 11971 | 12800 | 13629 | 9030 | 11142 | 14458 | 15287 | 16116 |
| 9031 | 10314 | 11972 | 12801 | 13630 | 9032 | 11143 | 14459 | 15288 | 16117 |
| 9033 | 10315 | 11973 | 12802 | 13631 | 9034 | 11144 | 14460 | 15289 | 16118 |
| 9035 | 10316 | 11974 | 12803 | 13632 | 9036 | 11145 | 14461 | 15290 | 16119 |
| 9037 | 10317 | 11975 | 12804 | 13633 | 9038 | 11146 | 14462 | 15291 | 16120 |
| 9039 | 10318 | 11976 | 12805 | 13634 | 9040 | 11147 | 14463 | 15292 | 16121 |
| 9041 | 10319 | 11977 | 12806 | 13635 | 9042 | 11148 | 14464 | 15293 | 16122 |
| 9043 | 10320 | 11978 | 12807 | 13636 | 9044 | 11149 | 14465 | 15294 | 16123 |
| 9045 | 10321 | 11979 | 12808 | 13637 | 9046 | 11150 | 14466 | 15295 | 16124 |
| 9047 | 10322 | 11980 | 12809 | 13638 | 9048 | 11151 | 14467 | 15296 | 16125 |
| 9049 | 10323 | 11981 | 12810 | 13639 | 9050 | 11152 | 14468 | 15297 | 16126 |
| 9051 | 10324 | 11982 | 12811 | 13640 | 9052 | 11153 | 14469 | 15298 | 16127 |
| 9053 | 10325 | 11983 | 12812 | 13641 | 9054 | 11154 | 14470 | 15299 | 16128 |
| 9055 | 10326 | 11984 | 12813 | 13642 | 9056 | 11155 | 14471 | 15300 | 16129 |
| 9057 | 10327 | 11985 | 12814 | 13643 | 9058 | 11156 | 14472 | 15301 | 16130 |
| 9059 | 10328 | 11986 | 12815 | 13644 | 9060 | 11157 | 14473 | 15302 | 16131 |
| 9061 | 10329 | 11987 | 12816 | 13645 | 9062 | 11158 | 14474 | 15303 | 16132 |
| 9063 | 10330 | 11988 | 12817 | 13646 | 9064 | 11159 | 14475 | 15304 | 16133 |
| 9065 | 10331 | 11989 | 12818 | 13647 | 9066 | 11160 | 14476 | 15305 | 16134 |
| 9067 | 10332 | 11990 | 12819 | 13648 | 9068 | 11161 | 14477 | 15306 | 16135 |
| 9069 | 10333 | 11991 | 12820 | 13649 | 9070 | 11162 | 14478 | 15307 | 16136 |
| 9071 | 10334 | 11992 | 12821 | 13650 | 9072 | 11163 | 14479 | 15308 | 16137 |
| 9073 | 10335 | 11993 | 12822 | 13651 | 9074 | 11164 | 14480 | 15309 | 16138 |
| 9075 | 10336 | 11994 | 12823 | 13652 | 9076 | 11165 | 14481 | 15310 | 16139 |
| 9077 | 10337 | 11995 | 12824 | 13653 | 9078 | 11166 | 14482 | 15311 | 16140 |
| 9079 | 10338 | 11996 | 12825 | 13654 | 9080 | 11167 | 14483 | 15312 | 16141 |
| 9081 | 10339 | 11997 | 12826 | 13655 | 9082 | 11168 | 14484 | 15313 | 16142 |
| 9083 | 10340 | 11998 | 12827 | 13656 | 9084 | 11169 | 14485 | 15314 | 16143 |
| 9085 | 10341 | 11999 | 12828 | 13657 | 9086 | 11170 | 14486 | 15315 | 16144 |
| 9087 | 10342 | 12000 | 12829 | 13658 | 9088 | 11171 | 14487 | 15316 | 16145 |
| 9089 | 10343 | 12001 | 12830 | 13659 | 9090 | 11172 | 14488 | 15317 | 16146 |
| 9091 | 10344 | 12002 | 12831 | 13660 | 9092 | 11173 | 14489 | 15318 | 16147 |
| 9093 | 10345 | 12003 | 12832 | 13661 | 9094 | 11174 | 14490 | 15319 | 16148 |
| 9095 | 10346 | 12004 | 12833 | 13662 | 9096 | 11175 | 14491 | 15320 | 16149 |
| 9097 | 10347 | 12005 | 12834 | 13663 | 9098 | 11176 | 14492 | 15321 | 16150 |
| 9099 | 10348 | 12006 | 12835 | 13664 | 9100 | 11177 | 14493 | 15322 | 16151 |
| 9101 | 10349 | 12007 | 12836 | 13665 | 9102 | 11178 | 14494 | 15323 | 16152 |
| 9103 | 10350 | 12008 | 12837 | 13666 | 9104 | 11179 | 14495 | 15324 | 16153 |
| 9105 | 10351 | 12009 | 12838 | 13667 | 9106 | 11180 | 14496 | 15325 | 16154 |
| 9107 | 10352 | 12010 | 12839 | 13668 | 9108 | 11181 | 14497 | 15326 | 16155 |
| 9109 | 10353 | 12011 | 12840 | 13669 | 9110 | 11182 | 14498 | 15327 | 16156 |
| 9111 | 10354 | 12012 | 12841 | 13670 | 9112 | 11183 | 14499 | 15328 | 16157 |
| 9113 | 10355 | 12013 | 12842 | 13671 | 9114 | 11184 | 14500 | 15329 | 16158 |
| 9115 | 10356 | 12014 | 12843 | 13672 | 9116 | 11185 | 14501 | 15330 | 16159 |
| 9117 | 10357 | 12015 | 12844 | 13673 | 9118 | 11186 | 14502 | 15331 | 16160 |
| 9119 | 10358 | 12016 | 12845 | 13674 | 9120 | 11187 | 14503 | 15332 | 16161 |
| 9121 | 10359 | 12017 | 12846 | 13675 | 9122 | 11188 | 14504 | 15333 | 16162 |
| 9123 | 10360 | 12018 | 12847 | 13676 | 9124 | 11189 | 14505 | 15334 | 16163 |
| 9125 | 10361 | 12019 | 12848 | 13677 | 9126 | 11190 | 14506 | 15335 | 16164 |
| 9127 | 10362 | 12020 | 12849 | 13678 | 9128 | 11191 | 14507 | 15336 | 16165 |
| 9129 | 10363 | 12021 | 12850 | 13679 | 9130 | 11192 | 14508 | 15337 | 16166 |
| 9131 | 10364 | 12022 | 12851 | 13680 | 9132 | 11193 | 14509 | 15338 | 16167 |
| 9133 | 10365 | 12023 | 12852 | 13681 | 9134 | 11194 | 14510 | 15339 | 16168 |
| 9135 | 10366 | 12024 | 12853 | 13682 | 9136 | 11195 | 14511 | 15340 | 16169 |
| 9137 | 10367 | 12025 | 12854 | 13683 | 9138 | 11196 | 14512 | 15341 | 16170 |
| 9139 | 10368 | 12026 | 12855 | 13684 | 9140 | 11197 | 14513 | 15342 | 16171 |
| 9141 | 10369 | 12027 | 12856 | 13685 | 9142 | 11198 | 14514 | 15343 | 16172 |
| 9143 | 10370 | 12028 | 12857 | 13686 | 9144 | 11199 | 14515 | 15344 | 16173 |
| 9145 | 10371 | 12029 | 12858 | 13687 | 9146 | 11200 | 14516 | 15345 | 16174 |
| 9147 | 10372 | 12030 | 12859 | 13688 | 9148 | 11201 | 14517 | 15346 | 16175 |
| 9149 | 10373 | 12031 | 12860 | 13689 | 9150 | 11202 | 14518 | 15347 | 16176 |
| 9151 | 10374 | 12032 | 12861 | 13690 | 9152 | 11203 | 14519 | 15348 | 16177 |
| 9153 | 10375 | 12033 | 12862 | 13691 | 9154 | 11204 | 14520 | 15349 | 16178 |
| 9155 | 10376 | 12034 | 12863 | 13692 | 9156 | 11205 | 14521 | 15350 | 16179 |
| 9157 | 10377 | 12035 | 12864 | 13693 | 9158 | 11206 | 14522 | 15351 | 16180 |
| 9159 | 10378 | 12036 | 12865 | 13694 | 9160 | 11207 | 14523 | 15352 | 16181 |
| 9161 | 10379 | 12037 | 12866 | 13695 | 9162 | 11208 | 14524 | 15353 | 16182 |
| 9163 | 10380 | 12038 | 12867 | 13696 | 9164 | 11209 | 14525 | 15354 | 16183 |
| 9165 | 10381 | 12039 | 12868 | 13697 | 9166 | 11210 | 14526 | 15355 | 16184 |
| 9167 | 10382 | 12040 | 12869 | 13698 | 9168 | 11211 | 14527 | 15356 | 16185 |
| 9169 | 10383 | 12041 | 12870 | 13699 | 9170 | 11212 | 14528 | 15357 | 16186 |
| 9171 | 10384 | 12042 | 12871 | 13700 | 9172 | 11213 | 14529 | 15358 | 16187 |
| 9173 | 10385 | 12043 | 12872 | 13701 | 9174 | 11214 | 14530 | 15359 | 16188 |
| 9175 | 10386 | 12044 | 12873 | 13702 | 9176 | 11215 | 14531 | 15360 | 16189 |
| 9177 | 10387 | 12045 | 12874 | 13703 | 9178 | 11216 | 14532 | 15361 | 16190 |
| 9179 | 10388 | 12046 | 12875 | 13704 | 9180 | 11217 | 14533 | 15362 | 16191 |
| 9181 | 10389 | 12047 | 12876 | 13705 | 9182 | 11218 | 14534 | 15363 | 16192 |
| 9183 | 10390 | 12048 | 12877 | 13706 | 9184 | 11219 | 14535 | 15364 | 16193 |
| 9185 | 10391 | 12049 | 12878 | 13707 | 9186 | 11220 | 14536 | 15365 | 16194 |
| 9187 | 10392 | 12050 | 12879 | 13708 | 9188 | 11221 | 14537 | 15366 | 16195 |
| 9189 | 10393 | 12051 | 12880 | 13709 | 9190 | 11222 | 14538 | 15367 | 16196 |
| 9191 | 10394 | 12052 | 12881 | 13710 | 9192 | 11223 | 14539 | 15368 | 16197 |
| 9193 | 10395 | 12053 | 12882 | 13711 | 9194 | 11224 | 14540 | 15369 | 16198 |
| 9195 | 10396 | 12054 | 12883 | 13712 | 9196 | 11225 | 14541 | 15370 | 16199 |
| 9197 | 10397 | 12055 | 12884 | 13713 | 9198 | 11226 | 14542 | 15371 | 16200 |
| 9199 | 10398 | 12056 | 12885 | 13714 | 9200 | 11227 | 14543 | 15372 | 16201 |
| 9201 | 10399 | 12057 | 12886 | 13715 | 9202 | 11228 | 14544 | 15373 | 16202 |
| 9203 | 10400 | 12058 | 12887 | 13716 | 9204 | 11229 | 14545 | 15374 | 16203 |
| 9205 | 10401 | 12059 | 12888 | 13717 | 9206 | 11230 | 14546 | 15375 | 16204 |
| 9207 | 10402 | 12060 | 12889 | 13718 | 9208 | 11231 | 14547 | 15376 | 16205 |
| 9209 | 10403 | 12061 | 12890 | 13719 | 9210 | 11232 | 14548 | 15377 | 16206 |
| 9211 | 10404 | 12062 | 12891 | 13720 | 9212 | 11233 | 14549 | 15378 | 16207 |
| 9213 | 10405 | 12063 | 12892 | 13721 | 9214 | 11234 | 14550 | 15379 | 16208 |
| 9215 | 10406 | 12064 | 12893 | 13722 | 9216 | 11235 | 14551 | 15380 | 16209 |
| 9217 | 10407 | 12065 | 12894 | 13723 | 9218 | 11236 | 14552 | 15381 | 16210 |
| 9219 | 10408 | 12066 | 12895 | 13724 | 9220 | 11237 | 14553 | 15382 | 16211 |
| 9221 | 10409 | 12067 | 12896 | 13725 | 9222 | 11238 | 14554 | 15383 | 16212 |
| 9223 | 10410 | 12068 | 12897 | 13726 | 9224 | 11239 | 14555 | 15384 | 16213 |
| 9225 | 10411 | 12069 | 12898 | 13727 | 9226 | 11240 | 14556 | 15385 | 16214 |
| 9227 | 10412 | 12070 | 12899 | 13728 | 9228 | 11241 | 14557 | 15386 | 16215 |
| 9229 | 10413 | 12071 | 12900 | 13729 | 9230 | 11242 | 14558 | 15387 | 16216 |
| 9231 | 10414 | 12072 | 12901 | 13730 | 9232 | 11243 | 14559 | 15388 | 16217 |
| 9233 | 10415 | 12073 | 12902 | 13731 | 9234 | 11244 | 14560 | 15389 | 16218 |
| 9235 | 10416 | 12074 | 12903 | 13732 | 9236 | 11245 | 14561 | 15390 | 16219 |
| 9237 | 10417 | 12075 | 12904 | 13733 | 9238 | 11246 | 14562 | 15391 | 16220 |
| 9239 | 10418 | 12076 | 12905 | 13734 | 9240 | 11247 | 14563 | 15392 | 16221 |
| 9241 | 10419 | 12077 | 12906 | 13735 | 9242 | 11248 | 14564 | 15393 | 16222 |
| 9243 | 10420 | 12078 | 12907 | 13736 | 9244 | 11249 | 14565 | 15394 | 16223 |
| 9245 | 10421 | 12079 | 12908 | 13737 | 9246 | 11250 | 14566 | 15395 | 16224 |
| 9247 | 10422 | 12080 | 12909 | 13738 | 9248 | 11251 | 14567 | 15396 | 16225 |
| 9249 | 10423 | 12081 | 12910 | 13739 | 9250 | 11252 | 14568 | 15397 | 16226 |
| 9251 | 10424 | 12082 | 12911 | 13740 | 9252 | 11253 | 14569 | 15398 | 16227 |
| 9253 | 10425 | 12083 | 12912 | 13741 | 9254 | 11254 | 14570 | 15399 | 16228 |
| 9255 | 10426 | 12084 | 12913 | 13742 | 9256 | 11255 | 14571 | 15400 | 16229 |
| 9257 | 10427 | 12085 | 12914 | 13743 | 9258 | 11256 | 14572 | 15401 | 16230 |
| 9259 | 10428 | 12086 | 12915 | 13744 | 9260 | 11257 | 14573 | 15402 | 16231 |
| 9261 | 10429 | 12087 | 12916 | 13745 | 9262 | 11258 | 14574 | 15403 | 16232 |
| 9263 | 10430 | 12088 | 12917 | 13746 | 9264 | 11259 | 14575 | 15404 | 16233 |
| 9265 | 10431 | 12089 | 12918 | 13747 | 9266 | 11260 | 14576 | 15405 | 16234 |
| 9267 | 10432 | 12090 | 12919 | 13748 | 9268 | 11261 | 14577 | 15406 | 16235 |
| 9269 | 10433 | 12091 | 12920 | 13749 | 9270 | 11262 | 14578 | 15407 | 16236 |
| 9271 | 10434 | 12092 | 12921 | 13750 | 9272 | 11263 | 14579 | 15408 | 16237 |
| 9273 | 10435 | 12093 | 12922 | 13751 | 9274 | 11264 | 14580 | 15409 | 16238 |
| 9275 | 10436 | 12094 | 12923 | 13752 | 9276 | 11265 | 14581 | 15410 | 16239 |
| 9277 | 10437 | 12095 | 12924 | 13753 | 9278 | 11266 | 14582 | 15411 | 16240 |
| 9279 | 10438 | 12096 | 12925 | 13754 | 9280 | 11267 | 14583 | 15412 | 16241 |
| 9281 | 10439 | 12097 | 12926 | 13755 | 9282 | 11268 | 14584 | 15413 | 16242 |
| 9283 | 10440 | 12098 | 12927 | 13756 | 9284 | 11269 | 14585 | 15414 | 16243 |
| 9285 | 10441 | 12099 | 12928 | 13757 | 9286 | 11270 | 14586 | 15415 | 16244 |
| 9287 | 10442 | 12100 | 12929 | 13758 | 9288 | 11271 | 14587 | 15416 | 16245 |
| 9289 | 10443 | 12101 | 12930 | 13759 | 9290 | 11272 | 14588 | 15417 | 16246 |
| 9291 | 10444 | 12102 | 12931 | 13760 | 9292 | 11273 | 14589 | 15418 | 16247 |
| 9293 | 10445 | 12103 | 12932 | 13761 | 9294 | 11274 | 14590 | 15419 | 16248 |
| 9295 | 10446 | 12104 | 12933 | 13762 | 9296 | 11275 | 14591 | 15420 | 16249 |
| 9297 | 10447 | 12105 | 12934 | 13763 | 9298 | 11276 | 14592 | 15421 | 16250 |
| 9299 | 10448 | 12106 | 12935 | 13764 | 9300 | 11277 | 14593 | 15422 | 16251 |
| 9301 | 10449 | 12107 | 12936 | 13765 | 9302 | 11278 | 14594 | 15423 | 16252 |
| 9303 | 10450 | 12108 | 12937 | 13766 | 9304 | 11279 | 14595 | 15424 | 16253 |
| 9305 | 10451 | 12109 | 12938 | 13767 | 9306 | 11280 | 14596 | 15425 | 16254 |
| 9307 | 10452 | 12110 | 12939 | 13768 | 9308 | 11281 | 14597 | 15426 | 16255 |
| 9309 | 10453 | 12111 | 12940 | 13769 | 9310 | 11282 | 14598 | 15427 | 16256 |
| 9311 | 10454 | 12112 | 12941 | 13770 | 9312 | 11283 | 14599 | 15428 | 16257 |
| 9313 | 10455 | 12113 | 12942 | 13771 | 9314 | 11284 | 14600 | 15429 | 16258 |
| 9315 | 10456 | 12114 | 12943 | 13772 | 9316 | 11285 | 14601 | 15430 | 16259 |
| 9317 | 10457 | 12115 | 12944 | 13773 | 9318 | 11286 | 14602 | 15431 | 16260 |
| 9319 | 10458 | 12116 | 12945 | 13774 | 9320 | 11287 | 14603 | 15432 | 16261 |
| 9321 | 10459 | 12117 | 12946 | 13775 | 9322 | 11288 | 14604 | 15433 | 16262 |
| 9323 | 10460 | 12118 | 12947 | 13776 | 9324 | 11289 | 14605 | 15434 | 16263 |
| 9325 | 10461 | 12119 | 12948 | 13777 | 9326 | 11290 | 14606 | 15435 | 16264 |
| 9327 | 10462 | 12120 | 12949 | 13778 | 9328 | 11291 | 14607 | 15436 | 16265 |
| 9329 | 10463 | 12121 | 12950 | 13779 | 9330 | 11292 | 14608 | 15437 | 16266 |
| 9331 | 10464 | 12122 | 12951 | 13780 | 9332 | 11293 | 14609 | 15438 | 16267 |
| 9333 | 10465 | 12123 | 12952 | 13781 | 9334 | 11294 | 14610 | 15439 | 16268 |
| 9335 | 10466 | 12124 | 12953 | 13782 | 9336 | 11295 | 14611 | 15440 | 16269 |
| 9337 | 10467 | 12125 | 12954 | 13783 | 9338 | 11296 | 14612 | 15441 | 16270 |
| 9339 | 10468 | 12126 | 12955 | 13784 | 9340 | 11297 | 14613 | 15442 | 16271 |
| 9341 | 10469 | 12127 | 12956 | 13785 | 9342 | 11298 | 14614 | 15443 | 16272 |
| 9343 | 10470 | 12128 | 12957 | 13786 | 9344 | 11299 | 14615 | 15444 | 16273 |
| 9345 | 10471 | 12129 | 12958 | 13787 | 9346 | 11300 | 14616 | 15445 | 16274 |
| 9347 | 10472 | 12130 | 12959 | 13788 | 9348 | 11301 | 14617 | 15446 | 16275 |
| 9349 | 10473 | 12131 | 12960 | 13789 | 9350 | 11302 | 14618 | 15447 | 16276 |
| 9351 | 10474 | 12132 | 12961 | 13790 | 9352 | 11303 | 14619 | 15448 | 16277 |
| 9353 | 10475 | 12133 | 12962 | 13791 | 9354 | 11304 | 14620 | 15449 | 16278 |
| 9355 | 10476 | 12134 | 12963 | 13792 | 9356 | 11305 | 14621 | 15450 | 16279 |
| 9357 | 10477 | 12135 | 12964 | 13793 | 9358 | 11306 | 14622 | 15451 | 16280 |
| 9359 | 10478 | 12136 | 12965 | 13794 | 9360 | 11307 | 14623 | 15452 | 16281 |
| 9361 | 10479 | 12137 | 12966 | 13795 | 9362 | 11308 | 14624 | 15453 | 16282 |
| 9363 | 10480 | 12138 | 12967 | 13796 | 9364 | 11309 | 14625 | 15454 | 16283 |
| 9365 | 10481 | 12139 | 12968 | 13797 | 9366 | 11310 | 14626 | 15455 | 16284 |
| 9367 | 10482 | 12140 | 12969 | 13798 | 9368 | 11311 | 14627 | 15456 | 16285 |
| 9369 | 10483 | 12141 | 12970 | 13799 | 9370 | 11312 | 14628 | 15457 | 16286 |
| 9371 | 10484 | 12142 | 12971 | 13800 | 9372 | 11313 | 14629 | 15458 | 16287 |
| 9373 | 10485 | 12143 | 12972 | 13801 | 9374 | 11314 | 14630 | 15459 | 16288 |
| 9375 | 10486 | 12144 | 12973 | 13802 | 9376 | 11315 | 14631 | 15460 | 16289 |
| 9377 | 10487 | 12145 | 12974 | 13803 | 9378 | 11316 | 14632 | 15461 | 16290 |
| 9379 | 10488 | 12146 | 12975 | 13804 | 9380 | 11317 | 14633 | 15462 | 16291 |
| 9381 | 10489 | 12147 | 12976 | 13805 | 9382 | 11318 | 14634 | 15463 | 16292 |
| 9383 | 10490 | 12148 | 12977 | 13806 | 9384 | 11319 | 14635 | 15464 | 16293 |
| 9385 | 10491 | 12149 | 12978 | 13807 | 9386 | 11320 | 14636 | 15465 | 16294 |
| 9387 | 10492 | 12150 | 12979 | 13808 | 9388 | 11321 | 14637 | 15466 | 16295 |
| 9389 | 10493 | 12151 | 12980 | 13809 | 9390 | 11322 | 14638 | 15467 | 16296 |
| 9391 | 10494 | 12152 | 12981 | 13810 | 9392 | 11323 | 14639 | 15468 | 16297 |
| 9393 | 10495 | 12153 | 12982 | 13811 | 9394 | 11324 | 14640 | 15469 | 16298 |
| 9395 | 10496 | 12154 | 12983 | 13812 | 9396 | 11325 | 14641 | 15470 | 16299 |
| 9397 | 10497 | 12155 | 12984 | 13813 | 9398 | 11326 | 14642 | 15471 | 16300 |
| 9399 | 10498 | 12156 | 12985 | 13814 | 9400 | 11327 | 14643 | 15472 | 16301 |
| 9401 | 10499 | 12157 | 12986 | 13815 | 9402 | 11328 | 14644 | 15473 | 16302 |
| 9403 | 10500 | 12158 | 12987 | 13816 | 9404 | 11329 | 14645 | 15474 | 16303 |
| 9405 | 10501 | 12159 | 12988 | 13817 | 9406 | 11330 | 14646 | 15475 | 16304 |
| 9407 | 10502 | 12160 | 12989 | 13818 | 9408 | 11331 | 14647 | 15476 | 16305 |
| 9409 | 10503 | 12161 | 12990 | 13819 | 9410 | 11332 | 14648 | 15477 | 16306 |
| 9411 | 10504 | 12162 | 12991 | 13820 | 9412 | 11333 | 14649 | 15478 | 16307 |
| 9413 | 10505 | 12163 | 12992 | 13821 | 9414 | 11334 | 14650 | 15479 | 16308 |
| 9415 | 10506 | 12164 | 12993 | 13822 | 9416 | 11335 | 14651 | 15480 | 16309 |
| 9417 | 10507 | 12165 | 12994 | 13823 | 9418 | 11336 | 14652 | 15481 | 16310 |
| 9419 | 10508 | 12166 | 12995 | 13824 | 9420 | 11337 | 14653 | 15482 | 16311 |
| 9421 | 10509 | 12167 | 12996 | 13825 | 9422 | 11338 | 14654 | 15483 | 16312 |
| 9423 | 10510 | 12168 | 12997 | 13826 | 9424 | 11339 | 14655 | 15484 | 16313 |
| 9425 | 10511 | 12169 | 12998 | 13827 | 9426 | 11340 | 14656 | 15485 | 16314 |
| 9427 | 10512 | 12170 | 12999 | 13828 | 9428 | 11341 | 14657 | 15486 | 16315 |
| 9429 | 10513 | 12171 | 13000 | 13829 | 9430 | 11342 | 14658 | 15487 | 16316 |
| 9431 | 10514 | 12172 | 13001 | 13830 | 9432 | 11343 | 14659 | 15488 | 16317 |
| 9433 | 10515 | 12173 | 13002 | 13831 | 9434 | 11344 | 14660 | 15489 | 16318 |
| 9435 | 10516 | 12174 | 13003 | 13832 | 9436 | 11345 | 14661 | 15490 | 16319 |
| 9437 | 10517 | 12175 | 13004 | 13833 | 9438 | 11346 | 14662 | 15491 | 16320 |
| 9439 | 10518 | 12176 | 13005 | 13834 | 9440 | 11347 | 14663 | 15492 | 16321 |
| 9441 | 10519 | 12177 | 13006 | 13835 | 9442 | 11348 | 14664 | 15493 | 16322 |
| 9443 | 10520 | 12178 | 13007 | 13836 | 9444 | 11349 | 14665 | 15494 | 16323 |
| 9445 | 10521 | 12179 | 13008 | 13837 | 9446 | 11350 | 14666 | 15495 | 16324 |
| 9447 | 10522 | 12180 | 13009 | 13838 | 9448 | 11351 | 14667 | 15496 | 16325 |
| 9449 | 10523 | 12181 | 13010 | 13839 | 9450 | 11352 | 14668 | 15497 | 16326 |
| 9451 | 10524 | 12182 | 13011 | 13840 | 9452 | 11353 | 14669 | 15498 | 16327 |
| 9453 | 10525 | 12183 | 13012 | 13841 | 9454 | 11354 | 14670 | 15499 | 16328 |
| 9455 | 10526 | 12184 | 13013 | 13842 | 9456 | 11355 | 14671 | 15500 | 16329 |
| 9457 | 10527 | 12185 | 13014 | 13843 | 9458 | 11356 | 14672 | 15501 | 16330 |
| 9459 | 10528 | 12186 | 13015 | 13844 | 9460 | 11357 | 14673 | 15502 | 16331 |
| 9461 | 10529 | 12187 | 13016 | 13845 | 9462 | 11358 | 14674 | 15503 | 16332 |
| 9463 | 10530 | 12188 | 13017 | 13846 | 9464 | 11359 | 14675 | 15504 | 16333 |
| 9465 | 10531 | 12189 | 13018 | 13847 | 9466 | 11360 | 14676 | 15505 | 16334 |
| 9467 | 10532 | 12190 | 13019 | 13848 | 9468 | 11361 | 14677 | 15506 | 16335 |
| 9469 | 10533 | 12191 | 13020 | 13849 | 9470 | 11362 | 14678 | 15507 | 16336 |
| 9471 | 10534 | 12192 | 13021 | 13850 | 9472 | 11363 | 14679 | 15508 | 16337 |
| 9473 | 10535 | 12193 | 13022 | 13851 | 9474 | 11364 | 14680 | 15509 | 16338 |
| 9475 | 10536 | 12194 | 13023 | 13852 | 9476 | 11365 | 14681 | 15510 | 16339 |
| 9477 | 10537 | 12195 | 13024 | 13853 | 9478 | 11366 | 14682 | 15511 | 16340 |
| 9479 | 10538 | 12196 | 13025 | 13854 | 9480 | 11367 | 14683 | 15512 | 16341 |
| 9481 | 10539 | 12197 | 13026 | 13855 | 9482 | 11368 | 14684 | 15513 | 16342 |
| 9483 | 10540 | 12198 | 13027 | 13856 | 9484 | 11369 | 14685 | 15514 | 16343 |
| 9485 | 10541 | 12199 | 13028 | 13857 | 9486 | 11370 | 14686 | 15515 | 16344 |
| 9487 | 10542 | 12200 | 13029 | 13858 | 9488 | 11371 | 14687 | 15516 | 16345 |
| 9489 | 10543 | 12201 | 13030 | 13859 | 9490 | 11372 | 14688 | 15517 | 16346 |
| 9491 | 10544 | 12202 | 13031 | 13860 | 9492 | 11373 | 14689 | 15518 | 16347 |
| 9493 | 10545 | 12203 | 13032 | 13861 | 9494 | 11374 | 14690 | 15519 | 16348 |
| 9495 | 10546 | 12204 | 13033 | 13862 | 9496 | 11375 | 14691 | 15520 | 16349 |
| 9497 | 10547 | 12205 | 13034 | 13863 | 9498 | 11376 | 14692 | 15521 | 16350 |
| 9499 | 10548 | 12206 | 13035 | 13864 | 9500 | 11377 | 14693 | 15522 | 16351 |
| 9501 | 10549 | 12207 | 13036 | 13865 | 9502 | 11378 | 14694 | 15523 | 16352 |
| 9503 | 10550 | 12208 | 13037 | 13866 | 9504 | 11379 | 14695 | 15524 | 16353 |
| 9505 | 10551 | 12209 | 13038 | 13867 | 9506 | 11380 | 14696 | 15525 | 16354 |
| 9507 | 10552 | 12210 | 13039 | 13868 | 9508 | 11381 | 14697 | 15526 | 16355 |
| 9509 | 10553 | 12211 | 13040 | 13869 | 9510 | 11382 | 14698 | 15527 | 16356 |
| 9511 | 10554 | 12212 | 13041 | 13870 | 9512 | 11383 | 14699 | 15528 | 16357 |
| 9513 | 10555 | 12213 | 13042 | 13871 | 9514 | 11384 | 14700 | 15529 | 16358 |
| 9515 | 10556 | 12214 | 13043 | 13872 | 9516 | 11385 | 14701 | 15530 | 16359 |
| 9517 | 10557 | 12215 | 13044 | 13873 | 9518 | 11386 | 14702 | 15531 | 16360 |
| 9519 | 10558 | 12216 | 13045 | 13874 | 9520 | 11387 | 14703 | 15532 | 16361 |
| 9521 | 10559 | 12217 | 13046 | 13875 | 9522 | 11388 | 14704 | 15533 | 16362 |
| 9523 | 10560 | 12218 | 13047 | 13876 | 9524 | 11389 | 14705 | 15534 | 16363 |
| 9525 | 10561 | 12219 | 13048 | 13877 | 9526 | 11390 | 14706 | 15535 | 16364 |
| 9527 | 10562 | 12220 | 13049 | 13878 | 9528 | 11391 | 14707 | 15536 | 16365 |
| 9529 | 10563 | 12221 | 13050 | 13879 | 9530 | 11392 | 14708 | 15537 | 16366 |
| 9531 | 10564 | 12222 | 13051 | 13880 | 9532 | 11393 | 14709 | 15538 | 16367 |
| 9533 | 10565 | 12223 | 13052 | 13881 | 9534 | 11394 | 14710 | 15539 | 16368 |
| 9535 | 10566 | 12224 | 13053 | 13882 | 9536 | 11395 | 14711 | 15540 | 16369 |
| 9537 | 10567 | 12225 | 13054 | 13883 | 9538 | 11396 | 14712 | 15541 | 16370 |
| 9539 | 10568 | 12226 | 13055 | 13884 | 9540 | 11397 | 14713 | 15542 | 16371 |
| 9541 | 10569 | 12227 | 13056 | 13885 | 9542 | 11398 | 14714 | 15543 | 16372 |
| 9543 | 10570 | 12228 | 13057 | 13886 | 9544 | 11399 | 14715 | 15544 | 16373 |
| 9545 | 10571 | 12229 | 13058 | 13887 | 9546 | 11400 | 14716 | 15545 | 16374 |
| 9547 | 10572 | 12230 | 13059 | 13888 | 9548 | 11401 | 14717 | 15546 | 16375 |
| 9549 | 10573 | 12231 | 13060 | 13889 | 9550 | 11402 | 14718 | 15547 | 16376 |
| 9551 | 10574 | 12232 | 13061 | 13890 | 9552 | 11403 | 14719 | 15548 | 16377 |
| 9553 | 10575 | 12233 | 13062 | 13891 | 9554 | 11404 | 14720 | 15549 | 16378 |
| 9555 | 10576 | 12234 | 13063 | 13892 | 9556 | 11405 | 14721 | 15550 | 16379 |
| 9557 | 10577 | 12235 | 13064 | 13893 | 9558 | 11406 | 14722 | 15551 | 16380 |
| 9559 | 10578 | 12236 | 13065 | 13894 | 9560 | 11407 | 14723 | 15552 | 16381 |
| 9561 | 10579 | 12237 | 13066 | 13895 | 9562 | 11408 | 14724 | 15553 | 16382 |
| 9563 | 10580 | 12238 | 13067 | 13896 | 9564 | 11409 | 14725 | 15554 | 16383 |
| 9565 | 10581 | 12239 | 13068 | 13897 | 9566 | 11410 | 14726 | 15555 | 16384 |
| 9567 | 10582 | 12240 | 13069 | 13898 | 9568 | 11411 | 14727 | 15556 | 16385 |
| 9569 | 10583 | 12241 | 13070 | 13899 | 9570 | 11412 | 14728 | 15557 | 16386 |
| 9571 | 10584 | 12242 | 13071 | 13900 | 9572 | 11413 | 14729 | 15558 | 16387 |
| 9573 | 10585 | 12243 | 13072 | 13901 | 9574 | 11414 | 14730 | 15559 | 16388 |
| 9575 | 10586 | 12244 | 13073 | 13902 | 9576 | 11415 | 14731 | 15560 | 16389 |
| 9577 | 10587 | 12245 | 13074 | 13903 | 9578 | 11416 | 14732 | 15561 | 16390 |
| 9579 | 10588 | 12246 | 13075 | 13904 | 9580 | 11417 | 14733 | 15562 | 16391 |
| 9581 | 10589 | 12247 | 13076 | 13905 | 9582 | 11418 | 14734 | 15563 | 16392 |
| 9583 | 10590 | 12248 | 13077 | 13906 | 9584 | 11419 | 14735 | 15564 | 16393 |
| 9585 | 10591 | 12249 | 13078 | 13907 | 9586 | 11420 | 14736 | 15565 | 16394 |
| 9587 | 10592 | 12250 | 13079 | 13908 | 9588 | 11421 | 14737 | 15566 | 16395 |
| 9589 | 10593 | 12251 | 13080 | 13909 | 9590 | 11422 | 14738 | 15567 | 16396 |
| 9591 | 10594 | 12252 | 13081 | 13910 | 9592 | 11423 | 14739 | 15568 | 16397 |
| 9593 | 10595 | 12253 | 13082 | 13911 | 9594 | 11424 | 14740 | 15569 | 16398 |
| 9595 | 10596 | 12254 | 13083 | 13912 | 9596 | 11425 | 14741 | 15570 | 16399 |
| 9597 | 10597 | 12255 | 13084 | 13913 | 9598 | 11426 | 14742 | 15571 | 16400 |
| 9599 | 10598 | 12256 | 13085 | 13914 | 9600 | 11427 | 14743 | 15572 | 16401 |
| 9601 | 10599 | 12257 | 13086 | 13915 | 9602 | 11428 | 14744 | 15573 | 16402 |
| 9603 | 10600 | 12258 | 13087 | 13916 | 9604 | 11429 | 14745 | 15574 | 16403 |
| 9605 | 10601 | 12259 | 13088 | 13917 | 9606 | 11430 | 14746 | 15575 | 16404 |
| 9607 | 10602 | 12260 | 13089 | 13918 | 9608 | 11431 | 14747 | 15576 | 16405 |
| 9609 | 10603 | 12261 | 13090 | 13919 | 9610 | 11432 | 14748 | 15577 | 16406 |
| 9611 | 10604 | 12262 | 13091 | 13920 | 9612 | 11433 | 14749 | 15578 | 16407 |
| 9613 | 10605 | 12263 | 13092 | 13921 | 9614 | 11434 | 14750 | 15579 | 16408 |
| 9615 | 10606 | 12264 | 13093 | 13922 | 9616 | 11435 | 14751 | 15580 | 16409 |
| 9617 | 10607 | 12265 | 13094 | 13923 | 9618 | 11436 | 14752 | 15581 | 16410 |
| 9619 | 10608 | 12266 | 13095 | 13924 | 9620 | 11437 | 14753 | 15582 | 16411 |
| 9621 | 10609 | 12267 | 13096 | 13925 | 9622 | 11438 | 14754 | 15583 | 16412 |
| 9623 | 10610 | 12268 | 13097 | 13926 | 9624 | 11439 | 14755 | 15584 | 16413 |
| 9625 | 10611 | 12269 | 13098 | 13927 | 9626 | 11440 | 14756 | 15585 | 16414 |
| 9627 | 10612 | 12270 | 13099 | 13928 | 9628 | 11441 | 14757 | 15586 | 16415 |
| 9629 | 10613 | 12271 | 13100 | 13929 | 9630 | 11442 | 14758 | 15587 | 16416 |
| 9631 | 10614 | 12272 | 13101 | 13930 | 9632 | 11443 | 14759 | 15588 | 16417 |
| 9633 | 10615 | 12273 | 13102 | 13931 | 9634 | 11444 | 14760 | 15589 | 16418 |
| 9635 | 10616 | 12274 | 13103 | 13932 | 9636 | 11445 | 14761 | 15590 | 16419 |
| 9637 | 10617 | 12275 | 13104 | 13933 | 9638 | 11446 | 14762 | 15591 | 16420 |
| 9639 | 10618 | 12276 | 13105 | 13934 | 9640 | 11447 | 14763 | 15592 | 16421 |
| 9641 | 10619 | 12277 | 13106 | 13935 | 9642 | 11448 | 14764 | 15593 | 16422 |
| 9643 | 10620 | 12278 | 13107 | 13936 | 9644 | 11449 | 14765 | 15594 | 16423 |
| 9645 | 10621 | 12279 | 13108 | 13937 | 9646 | 11450 | 14766 | 15595 | 16424 |
| 9647 | 10622 | 12280 | 13109 | 13938 | 9648 | 11451 | 14767 | 15596 | 16425 |
| 9649 | 10623 | 12281 | 13110 | 13939 | 9650 | 11452 | 14768 | 15597 | 16426 |
| 9651 | 10624 | 12282 | 13111 | 13940 | 9652 | 11453 | 14769 | 15598 | 16427 |
| 9653 | 10625 | 12283 | 13112 | 13941 | 9654 | 11454 | 14770 | 15599 | 16428 |
| 9655 | 10626 | 12284 | 13113 | 13942 | 9656 | 11455 | 14771 | 15600 | 16429 |
| 9657 | 10627 | 12285 | 13114 | 13943 | 9658 | 11456 | 14772 | 15601 | 16430 |
| 9659 | 10628 | 12286 | 13115 | 13944 | 9660 | 11457 | 14773 | 15602 | 16431 |
| 9661 | 10629 | 12287 | 13116 | 13945 | 9662 | 11458 | 14774 | 15603 | 16432 |
| 9663 | 10630 | 12288 | 13117 | 13946 | 9664 | 11459 | 14775 | 15604 | 16433 |
| 9665 | 10631 | 12289 | 13118 | 13947 | 9666 | 11460 | 14776 | 15605 | 16434 |
| 9667 | 10632 | 12290 | 13119 | 13948 | 9668 | 11461 | 14777 | 15606 | 16435 |
| 9669 | 10633 | 12291 | 13120 | 13949 | 9670 | 11462 | 14778 | 15607 | 16436 |
| 9671 | 10634 | 12292 | 13121 | 13950 | 9672 | 11463 | 14779 | 15608 | 16437 |
| 9673 | 10635 | 12293 | 13122 | 13951 | 9674 | 11464 | 14780 | 15609 | 16438 |
| 9675 | 10636 | 12294 | 13123 | 13952 | 9676 | 11465 | 14781 | 15610 | 16439 |
| 9677 | 10637 | 12295 | 13124 | 13953 | 9678 | 11466 | 14782 | 15611 | 16440 |
| 9679 | 10638 | 12296 | 13125 | 13954 | 9680 | 11467 | 14783 | 15612 | 16441 |
| 9681 | 10639 | 12297 | 13126 | 13955 | 9682 | 11468 | 14784 | 15613 | 16442 |
| 9683 | 10640 | 12298 | 13127 | 13956 | 9684 | 11469 | 14785 | 15614 | 16443 |
| 9685 | 10641 | 12299 | 13128 | 13957 | 9686 | 11470 | 14786 | 15615 | 16444 |
| 9687 | 10642 | 12300 | 13129 | 13958 | 9688 | 11471 | 14787 | 15616 | 16445 |
| 9689 | 10643 | 12301 | 13130 | 13959 | 9690 | 11472 | 14788 | 15617 | 16446 |
| 9691 | 10644 | 12302 | 13131 | 13960 | 9692 | 11473 | 14789 | 15618 | 16447 |
| 9693 | 10645 | 12303 | 13132 | 13961 | 9694 | 11474 | 14790 | 15619 | 16448 |
| 9695 | 10646 | 12304 | 13133 | 13962 | 9696 | 11475 | 14791 | 15620 | 16449 |
| 9697 | 10647 | 12305 | 13134 | 13963 | 9698 | 11476 | 14792 | 15621 | 16450 |
| 9699 | 10648 | 12306 | 13135 | 13964 | 9700 | 11477 | 14793 | 15622 | 16451 |
| 9701 | 10649 | 12307 | 13136 | 13965 | 9702 | 11478 | 14794 | 15623 | 16452 |
| 9703 | 10650 | 12308 | 13137 | 13966 | 9704 | 11479 | 14795 | 15624 | 16453 |
| 9705 | 10651 | 12309 | 13138 | 13967 | 9706 | 11480 | 14796 | 15625 | 16454 |
| 9707 | 10652 | 12310 | 13139 | 13968 | 9708 | 11481 | 14797 | 15626 | 16455 |
| 9709 | 10653 | 12311 | 13140 | 13969 | 9710 | 11482 | 14798 | 15627 | 16456 |
| 9711 | 10654 | 12312 | 13141 | 13970 | 9712 | 11483 | 14799 | 15628 | 16457 |
| 9713 | 10655 | 12313 | 13142 | 13971 | 9714 | 11484 | 14800 | 15629 | 16458 |
| 9715 | 10656 | 12314 | 13143 | 13972 | 9716 | 11485 | 14801 | 15630 | 16459 |
| 9717 | 10657 | 12315 | 13144 | 13973 | 9718 | 11486 | 14802 | 15631 | 16460 |
| 9719 | 10658 | 12316 | 13145 | 13974 | 9720 | 11487 | 14803 | 15632 | 16461 |
| 9721 | 10659 | 12317 | 13146 | 13975 | 9722 | 11488 | 14804 | 15633 | 16462 |
| 9723 | 10660 | 12318 | 13147 | 13976 | 9724 | 11489 | 14805 | 15634 | 16463 |
| 9725 | 10661 | 12319 | 13148 | 13977 | 9726 | 11490 | 14806 | 15635 | 16464 |
| 9727 | 10662 | 12320 | 13149 | 13978 | 9728 | 11491 | 14807 | 15636 | 16465 |
| 9729 | 10663 | 12321 | 13150 | 13979 | 9730 | 11492 | 14808 | 15637 | 16466 |
| 9731 | 10664 | 12322 | 13151 | 13980 | 9732 | 11493 | 14809 | 15638 | 16467 |
| 9733 | 10665 | 12323 | 13152 | 13981 | 9734 | 11494 | 14810 | 15639 | 16468 |
| 9735 | 10666 | 12324 | 13153 | 13982 | 9736 | 11495 | 14811 | 15640 | 16469 |
| 9737 | 10667 | 12325 | 13154 | 13983 | 9738 | 11496 | 14812 | 15641 | 16470 |
| 9739 | 10668 | 12326 | 13155 | 13984 | 9740 | 11497 | 14813 | 15642 | 16471 |
| 9741 | 10669 | 12327 | 13156 | 13985 | 9742 | 11498 | 14814 | 15643 | 16472 |
| 9743 | 10670 | 12328 | 13157 | 13986 | 9744 | 11499 | 14815 | 15644 | 16473 |
| 9745 | 10671 | 12329 | 13158 | 13987 | 9746 | 11500 | 14816 | 15645 | 16474 |
| 9747 | 10672 | 12330 | 13159 | 13988 | 9748 | 11501 | 14817 | 15646 | 16475 |
| 9749 | 10673 | 12331 | 13160 | 13989 | 9750 | 11502 | 14818 | 15647 | 16476 |
| 9751 | 10674 | 12332 | 13161 | 13990 | 9752 | 11503 | 14819 | 15648 | 16477 |
| 9753 | 10675 | 12333 | 13162 | 13991 | 9754 | 11504 | 14820 | 15649 | 16478 |
| 9755 | 10676 | 12334 | 13163 | 13992 | 9756 | 11505 | 14821 | 15650 | 16479 |
| 9757 | 10677 | 12335 | 13164 | 13993 | 9758 | 11506 | 14822 | 15651 | 16480 |
| 9759 | 10678 | 12336 | 13165 | 13994 | 9760 | 11507 | 14823 | 15652 | 16481 |
| 9761 | 10679 | 12337 | 13166 | 13995 | 9762 | 11508 | 14824 | 15653 | 16482 |
| 9763 | 10680 | 12338 | 13167 | 13996 | 9764 | 11509 | 14825 | 15654 | 16483 |
| 9765 | 10681 | 12339 | 13168 | 13997 | 9766 | 11510 | 14826 | 15655 | 16484 |
| 9767 | 10682 | 12340 | 13169 | 13998 | 9768 | 11511 | 14827 | 15656 | 16485 |
| 9769 | 10683 | 12341 | 13170 | 13999 | 9770 | 11512 | 14828 | 15657 | 16486 |
| 9771 | 10684 | 12342 | 13171 | 14000 | 9772 | 11513 | 14829 | 15658 | 16487 |
| 9773 | 10685 | 12343 | 13172 | 14001 | 9774 | 11514 | 14830 | 15659 | 16488 |
| 9775 | 10686 | 12344 | 13173 | 14002 | 9776 | 11515 | 14831 | 15660 | 16489 |
| 9777 | 10687 | 12345 | 13174 | 14003 | 9778 | 11516 | 14832 | 15661 | 16490 |
| 9779 | 10688 | 12346 | 13175 | 14004 | 9780 | 11517 | 14833 | 15662 | 16491 |
| 9781 | 10689 | 12347 | 13176 | 14005 | 9782 | 11518 | 14834 | 15663 | 16492 |
| 9783 | 10690 | 12348 | 13177 | 14006 | 9784 | 11519 | 14835 | 15664 | 16493 |
| 9785 | 10691 | 12349 | 13178 | 14007 | 9786 | 11520 | 14836 | 15665 | 16494 |
| 9787 | 10692 | 12350 | 13179 | 14008 | 9788 | 11521 | 14837 | 15666 | 16495 |
| 9789 | 10693 | 12351 | 13180 | 14009 | 9790 | 11522 | 14838 | 15667 | 16496 |
| 9791 | 10694 | 12352 | 13181 | 14010 | 9792 | 11523 | 14839 | 15668 | 16497 |
| 9793 | 10695 | 12353 | 13182 | 14011 | 9794 | 11524 | 14840 | 15669 | 16498 |
| 9795 | 10696 | 12354 | 13183 | 14012 | 9796 | 11525 | 14841 | 15670 | 16499 |
| 9797 | 10697 | 12355 | 13184 | 14013 | 9798 | 11526 | 14842 | 15671 | 16500 |
| 9799 | 10698 | 12356 | 13185 | 14014 | 9800 | 11527 | 14843 | 15672 | 16501 |
| 9801 | 10699 | 12357 | 13186 | 14015 | 9802 | 11528 | 14844 | 15673 | 16502 |
| 9803 | 10700 | 12358 | 13187 | 14016 | 9804 | 11529 | 14845 | 15674 | 16503 |
| 9805 | 10701 | 12359 | 13188 | 14017 | 9806 | 11530 | 14846 | 15675 | 16504 |
| 9807 | 10702 | 12360 | 13189 | 14018 | 9808 | 11531 | 14847 | 15676 | 16505 |
| 9809 | 10703 | 12361 | 13190 | 14019 | 9810 | 11532 | 14848 | 15677 | 16506 |
| 9811 | 10704 | 12362 | 13191 | 14020 | 9812 | 11533 | 14849 | 15678 | 16507 |
| 9813 | 10705 | 12363 | 13192 | 14021 | 9814 | 11534 | 14850 | 15679 | 16508 |
| 9815 | 10706 | 12364 | 13193 | 14022 | 9816 | 11535 | 14851 | 15680 | 16509 |
| 9817 | 10707 | 12365 | 13194 | 14023 | 9818 | 11536 | 14852 | 15681 | 16510 |
| 9819 | 10708 | 12366 | 13195 | 14024 | 9820 | 11537 | 14853 | 15682 | 16511 |
| 9821 | 10709 | 12367 | 13196 | 14025 | 9822 | 11538 | 14854 | 15683 | 16512 |
| 9823 | 10710 | 12368 | 13197 | 14026 | 9824 | 11539 | 14855 | 15684 | 16513 |
| 9825 | 10711 | 12369 | 13198 | 14027 | 9826 | 11540 | 14856 | 15685 | 16514 |
| 9827 | 10712 | 12370 | 13199 | 14028 | 9828 | 11541 | 14857 | 15686 | 16515 |
| 9829 | 10713 | 12371 | 13200 | 14029 | 9830 | 11542 | 14858 | 15687 | 16516 |
| 9831 | 10714 | 12372 | 13201 | 14030 | 9832 | 11543 | 14859 | 15688 | 16517 |
| 9833 | 10715 | 12373 | 13202 | 14031 | 9834 | 11544 | 14860 | 15689 | 16518 |
| 9835 | 10716 | 12374 | 13203 | 14032 | 9836 | 11545 | 14861 | 15690 | 16519 |
| 9837 | 10717 | 12375 | 13204 | 14033 | 9838 | 11546 | 14862 | 15691 | 16520 |
| 9839 | 10718 | 12376 | 13205 | 14034 | 9840 | 11547 | 14863 | 15692 | 16521 |
| 9841 | 10719 | 12377 | 13206 | 14035 | 9842 | 11548 | 14864 | 15693 | 16522 |
| 9843 | 10720 | 12378 | 13207 | 14036 | 9844 | 11549 | 14865 | 15694 | 16523 |
| 9845 | 10721 | 12379 | 13208 | 14037 | 9846 | 11550 | 14866 | 15695 | 16524 |
| 9847 | 10722 | 12380 | 13209 | 14038 | 9848 | 11551 | 14867 | 15696 | 16525 |
| 9849 | 10723 | 12381 | 13210 | 14039 | 9850 | 11552 | 14868 | 15697 | 16526 |
| 9851 | 10724 | 12382 | 13211 | 14040 | 9852 | 11553 | 14869 | 15698 | 16527 |
| 9853 | 10725 | 12383 | 13212 | 14041 | 9854 | 11554 | 14870 | 15699 | 16528 |
| 9855 | 10726 | 12384 | 13213 | 14042 | 9856 | 11555 | 14871 | 15700 | 16529 |
| 9857 | 10727 | 12385 | 13214 | 14043 | 9858 | 11556 | 14872 | 15701 | 16530 |
| 9859 | 10728 | 12386 | 13215 | 14044 | 9860 | 11557 | 14873 | 15702 | 16531 |
| 9861 | 10729 | 12387 | 13216 | 14045 | 9862 | 11558 | 14874 | 15703 | 16532 |
| 9863 | 10730 | 12388 | 13217 | 14046 | 9864 | 11559 | 14875 | 15704 | 16533 |
| 9865 | 10731 | 12389 | 13218 | 14047 | 9866 | 11560 | 14876 | 15705 | 16534 |
| 9867 | 10732 | 12390 | 13219 | 14048 | 9868 | 11561 | 14877 | 15706 | 16535 |
| 9869 | 10733 | 12391 | 13220 | 14049 | 9870 | 11562 | 14878 | 15707 | 16536 |
| 9871 | 10734 | 12392 | 13221 | 14050 | 9872 | 11563 | 14879 | 15708 | 16537 |
| 9873 | 10735 | 12393 | 13222 | 14051 | 9874 | 11564 | 14880 | 15709 | 16538 |
| 9875 | 10736 | 12394 | 13223 | 14052 | 9876 | 11565 | 14881 | 15710 | 16539 |
| 9877 | 10737 | 12395 | 13224 | 14053 | 9878 | 11566 | 14882 | 15711 | 16540 |
| 9879 | 10738 | 12396 | 13225 | 14054 | 9880 | 11567 | 14883 | 15712 | 16541 |
| 9881 | 10739 | 12397 | 13226 | 14055 | 9882 | 11568 | 14884 | 15713 | 16542 |
| 9883 | 10740 | 12398 | 13227 | 14056 | 9884 | 11569 | 14885 | 15714 | 16543 |
| 9885 | 10741 | 12399 | 13228 | 14057 | 9886 | 11570 | 14886 | 15715 | 16544 |
| 9887 | 10742 | 12400 | 13229 | 14058 | 9888 | 11571 | 14887 | 15716 | 16545 |
| 9889 | 10743 | 12401 | 13230 | 14059 | 9890 | 11572 | 14888 | 15717 | 16546 |
| 9891 | 10744 | 12402 | 13231 | 14060 | 9892 | 11573 | 14889 | 15718 | 16547 |
| 9893 | 10745 | 12403 | 13232 | 14061 | 9894 | 11574 | 14890 | 15719 | 16548 |
| 9895 | 10746 | 12404 | 13233 | 14062 | 9896 | 11575 | 14891 | 15720 | 16549 |
| 9897 | 10747 | 12405 | 13234 | 14063 | 9898 | 11576 | 14892 | 15721 | 16550 |
| 9899 | 10748 | 12406 | 13235 | 14064 | 9900 | 11577 | 14893 | 15722 | 16551 |
| 9901 | 10749 | 12407 | 13236 | 14065 | 9902 | 11578 | 14894 | 15723 | 16552 |
| 9903 | 10750 | 12408 | 13237 | 14066 | 9904 | 11579 | 14895 | 15724 | 16553 |
| 9905 | 10751 | 12409 | 13238 | 14067 | 9906 | 11580 | 14896 | 15725 | 16554 |
| 9907 | 10752 | 12410 | 13239 | 14068 | 9908 | 11581 | 14897 | 15726 | 16555 |
| 9909 | 10753 | 12411 | 13240 | 14069 | 9910 | 11582 | 14898 | 15727 | 16556 |
| 9911 | 10754 | 12412 | 13241 | 14070 | 9912 | 11583 | 14899 | 15728 | 16557 |
| 9913 | 10755 | 12413 | 13242 | 14071 | 9914 | 11584 | 14900 | 15729 | 16558 |
| 9915 | 10756 | 12414 | 13243 | 14072 | 9916 | 11585 | 14901 | 15730 | 16559 |
| 9917 | 10757 | 12415 | 13244 | 14073 | 9918 | 11586 | 14902 | 15731 | 16560 |
| 9919 | 10758 | 12416 | 13245 | 14074 | 9920 | 11587 | 14903 | 15732 | 16561 |
| 9921 | 10759 | 12417 | 13246 | 14075 | 9922 | 11588 | 14904 | 15733 | 16562 |
| 9923 | 10760 | 12418 | 13247 | 14076 | 9924 | 11589 | 14905 | 15734 | 16563 |
| 9925 | 10761 | 12419 | 13248 | 14077 | 9926 | 11590 | 14906 | 15735 | 16564 |
| 9927 | 10762 | 12420 | 13249 | 14078 | 9928 | 11591 | 14907 | 15736 | 16565 |
| 9929 | 10763 | 12421 | 13250 | 14079 | 9930 | 11592 | 14908 | 15737 | 16566 |
| 9931 | 10764 | 12422 | 13251 | 14080 | 9932 | 11593 | 14909 | 15738 | 16567 |
| 9933 | 10765 | 12423 | 13252 | 14081 | 9934 | 11594 | 14910 | 15739 | 16568 |
| 9935 | 10766 | 12424 | 13253 | 14082 | 9936 | 11595 | 14911 | 15740 | 16569 |
| 9937 | 10767 | 12425 | 13254 | 14083 | 9938 | 11596 | 14912 | 15741 | 16570 |
| 16571 | 18485 | 19442 | 20399 | 21356 | 16572 | 22313 | 23270 | 24227 | 25184 |
| 16573 | 18486 | 19443 | 20400 | 21357 | 16574 | 22314 | 23271 | 24228 | 25185 |
| 16575 | 18487 | 19444 | 20401 | 21358 | 16576 | 22315 | 23272 | 24229 | 25186 |
| 16577 | 18488 | 19445 | 20402 | 21359 | 16578 | 22316 | 23273 | 24230 | 25187 |
| 16579 | 18489 | 19446 | 20403 | 21360 | 16580 | 22317 | 23274 | 24231 | 25188 |
| 16581 | 18490 | 19447 | 20404 | 21361 | 16582 | 22318 | 23275 | 24232 | 25189 |
| 16583 | 18491 | 19448 | 20405 | 21362 | 16584 | 22319 | 23276 | 24233 | 25190 |
| 16585 | 18492 | 19449 | 20406 | 21363 | 16586 | 22320 | 23277 | 24234 | 25191 |
| 16587 | 18493 | 19450 | 20407 | 21364 | 16588 | 22321 | 23278 | 24235 | 25192 |
| 16589 | 18494 | 19451 | 20408 | 21365 | 16590 | 22322 | 23279 | 24236 | 25193 |
| 16591 | 18495 | 19452 | 20409 | 21366 | 16592 | 22323 | 23280 | 24237 | 25194 |
| 16593 | 18496 | 19453 | 20410 | 21367 | 16594 | 22324 | 23281 | 24238 | 25195 |
| 16595 | 18497 | 19454 | 20411 | 21368 | 16596 | 22325 | 23282 | 24239 | 25196 |
| 16597 | 18498 | 19455 | 20412 | 21369 | 16598 | 22326 | 23283 | 24240 | 25197 |
| 16599 | 18499 | 19456 | 20413 | 21370 | 16600 | 22327 | 23284 | 24241 | 25198 |
| 16601 | 18500 | 19457 | 20414 | 21371 | 16602 | 22328 | 23285 | 24242 | 25199 |
| 16603 | 18501 | 19458 | 20415 | 21372 | 16604 | 22329 | 23286 | 24243 | 25200 |
| 16605 | 18502 | 19459 | 20416 | 21373 | 16606 | 22330 | 23287 | 24244 | 25201 |
| 16607 | 18503 | 19460 | 20417 | 21374 | 16608 | 22331 | 23288 | 24245 | 25202 |
| 16609 | 18504 | 19461 | 20418 | 21375 | 16610 | 22332 | 23289 | 24246 | 25203 |
| 16611 | 18505 | 19462 | 20419 | 21376 | 16612 | 22333 | 23290 | 24247 | 25204 |
| 16613 | 18506 | 19463 | 20420 | 21377 | 16614 | 22334 | 23291 | 24248 | 25205 |
| 16615 | 18507 | 19464 | 20421 | 21378 | 16616 | 22335 | 23292 | 24249 | 25206 |
| 16617 | 18508 | 19465 | 20422 | 21379 | 16618 | 22336 | 23293 | 24250 | 25207 |
| 16619 | 18509 | 19466 | 20423 | 21380 | 16620 | 22337 | 23294 | 24251 | 25208 |
| 16621 | 18510 | 19467 | 20424 | 21381 | 16622 | 22338 | 23295 | 24252 | 25209 |
| 16623 | 18511 | 19468 | 20425 | 21382 | 16624 | 22339 | 23296 | 24253 | 25210 |
| 16625 | 18512 | 19469 | 20426 | 21383 | 16626 | 22340 | 23297 | 24254 | 25211 |
| 16627 | 18513 | 19470 | 20427 | 21384 | 16628 | 22341 | 23298 | 24255 | 25212 |
| 16629 | 18514 | 19471 | 20428 | 21385 | 16630 | 22342 | 23299 | 24256 | 25213 |
| 16631 | 18515 | 19472 | 20429 | 21386 | 16632 | 22343 | 23300 | 24257 | 25214 |
| 16633 | 18516 | 19473 | 20430 | 21387 | 16634 | 22344 | 23301 | 24258 | 25215 |
| 16635 | 18517 | 19474 | 20431 | 21388 | 16636 | 22345 | 23302 | 24259 | 25216 |
| 16637 | 18518 | 19475 | 20432 | 21389 | 16638 | 22346 | 23303 | 24260 | 25217 |
| 16639 | 18519 | 19476 | 20433 | 21390 | 16640 | 22347 | 23304 | 24261 | 25218 |
| 16641 | 18520 | 19477 | 20434 | 21391 | 16642 | 22348 | 23305 | 24262 | 25219 |
| 16643 | 18521 | 19478 | 20435 | 21392 | 16644 | 22349 | 23306 | 24263 | 25220 |
| 16645 | 18522 | 19479 | 20436 | 21393 | 16646 | 22350 | 23307 | 24264 | 25221 |
| 16647 | 18523 | 19480 | 20437 | 21394 | 16648 | 22351 | 23308 | 24265 | 25222 |
| 16649 | 18524 | 19481 | 20438 | 21395 | 16650 | 22352 | 23309 | 24266 | 25223 |
| 16651 | 18525 | 19482 | 20439 | 21396 | 16652 | 22353 | 23310 | 24267 | 25224 |
| 16653 | 18526 | 19483 | 20440 | 21397 | 16654 | 22354 | 23311 | 24268 | 25225 |
| 16655 | 18527 | 19484 | 20441 | 21398 | 16656 | 22355 | 23312 | 24269 | 25226 |
| 16657 | 18528 | 19485 | 20442 | 21399 | 16658 | 22356 | 23313 | 24270 | 25227 |
| 16659 | 18529 | 19486 | 20443 | 21400 | 16660 | 22357 | 23314 | 24271 | 25228 |
| 16661 | 18530 | 19487 | 20444 | 21401 | 16662 | 22358 | 23315 | 24272 | 25229 |
| 16663 | 18531 | 19488 | 20445 | 21402 | 16664 | 22359 | 23316 | 24273 | 25230 |
| 16665 | 18532 | 19489 | 20446 | 21403 | 16666 | 22360 | 23317 | 24274 | 25231 |
| 16667 | 18533 | 19490 | 20447 | 21404 | 16668 | 22361 | 23318 | 24275 | 25232 |
| 16669 | 18534 | 19491 | 20448 | 21405 | 16670 | 22362 | 23319 | 24276 | 25233 |
| 16671 | 18535 | 19492 | 20449 | 21406 | 16672 | 22363 | 23320 | 24277 | 25234 |
| 16673 | 18536 | 19493 | 20450 | 21407 | 16674 | 22364 | 23321 | 24278 | 25235 |
| 16675 | 18537 | 19494 | 20451 | 21408 | 16676 | 22365 | 23322 | 24279 | 25236 |
| 16677 | 18538 | 19495 | 20452 | 21409 | 16678 | 22366 | 23323 | 24280 | 25237 |
| 16679 | 18539 | 19496 | 20453 | 21410 | 16680 | 22367 | 23324 | 24281 | 25238 |
| 16681 | 18540 | 19497 | 20454 | 21411 | 16682 | 22368 | 23325 | 24282 | 25239 |
| 16683 | 18541 | 19498 | 20455 | 21412 | 16684 | 22369 | 23326 | 24283 | 25240 |
| 16685 | 18542 | 19499 | 20456 | 21413 | 16686 | 22370 | 23327 | 24284 | 25241 |
| 16687 | 18543 | 19500 | 20457 | 21414 | 16688 | 22371 | 23328 | 24285 | 25242 |
| 16689 | 18544 | 19501 | 20458 | 21415 | 16690 | 22372 | 23329 | 24286 | 25243 |
| 16691 | 18545 | 19502 | 20459 | 21416 | 16692 | 22373 | 23330 | 24287 | 25244 |
| 16693 | 18546 | 19503 | 20460 | 21417 | 16694 | 22374 | 23331 | 24288 | 25245 |
| 16695 | 18547 | 19504 | 20461 | 21418 | 16696 | 22375 | 23332 | 24289 | 25246 |
| 16697 | 18548 | 19505 | 20462 | 21419 | 16698 | 22376 | 23333 | 24290 | 25247 |
| 16699 | 18549 | 19506 | 20463 | 21420 | 16700 | 22377 | 23334 | 24291 | 25248 |
| 16701 | 18550 | 19507 | 20464 | 21421 | 16702 | 22378 | 23335 | 24292 | 25249 |
| 16703 | 18551 | 19508 | 20465 | 21422 | 16704 | 22379 | 23336 | 24293 | 25250 |
| 16705 | 18552 | 19509 | 20466 | 21423 | 16706 | 22380 | 23337 | 24294 | 25251 |
| 16707 | 18553 | 19510 | 20467 | 21424 | 16708 | 22381 | 23338 | 24295 | 25252 |
| 16709 | 18554 | 19511 | 20468 | 21425 | 16710 | 22382 | 23339 | 24296 | 25253 |
| 16711 | 18555 | 19512 | 20469 | 21426 | 16712 | 22383 | 23340 | 24297 | 25254 |
| 16713 | 18556 | 19513 | 20470 | 21427 | 16714 | 22384 | 23341 | 24298 | 25255 |
| 16715 | 18557 | 19514 | 20471 | 21428 | 16716 | 22385 | 23342 | 24299 | 25256 |
| 16717 | 18558 | 19515 | 20472 | 21429 | 16718 | 22386 | 23343 | 24300 | 25257 |
| 16719 | 18559 | 19516 | 20473 | 21430 | 16720 | 22387 | 23344 | 24301 | 25258 |
| 16721 | 18560 | 19517 | 20474 | 21431 | 16722 | 22388 | 23345 | 24302 | 25259 |
| 16723 | 18561 | 19518 | 20475 | 21432 | 16724 | 22389 | 23346 | 24303 | 25260 |
| 16725 | 18562 | 19519 | 20476 | 21433 | 16726 | 22390 | 23347 | 24304 | 25261 |
| 16727 | 18563 | 19520 | 20477 | 21434 | 16728 | 22391 | 23348 | 24305 | 25262 |
| 16729 | 18564 | 19521 | 20478 | 21435 | 16730 | 22392 | 23349 | 24306 | 25263 |
| 16731 | 18565 | 19522 | 20479 | 21436 | 16732 | 22393 | 23350 | 24307 | 25264 |
| 16733 | 18566 | 19523 | 20480 | 21437 | 16734 | 22394 | 23351 | 24308 | 25265 |
| 16735 | 18567 | 19524 | 20481 | 21438 | 16736 | 22395 | 23352 | 24309 | 25266 |
| 16737 | 18568 | 19525 | 20482 | 21439 | 16738 | 22396 | 23353 | 24310 | 25267 |
| 16739 | 18569 | 19526 | 20483 | 21440 | 16740 | 22397 | 23354 | 24311 | 25268 |
| 16741 | 18570 | 19527 | 20484 | 21441 | 16742 | 22398 | 23355 | 24312 | 25269 |
| 16743 | 18571 | 19528 | 20485 | 21442 | 16744 | 22399 | 23356 | 24313 | 25270 |
| 16745 | 18572 | 19529 | 20486 | 21443 | 16746 | 22400 | 23357 | 24314 | 25271 |
| 16747 | 18573 | 19530 | 20487 | 21444 | 16748 | 22401 | 23358 | 24315 | 25272 |
| 16749 | 18574 | 19531 | 20488 | 21445 | 16750 | 22402 | 23359 | 24316 | 25273 |
| 16751 | 18575 | 19532 | 20489 | 21446 | 16752 | 22403 | 23360 | 24317 | 25274 |
| 16753 | 18576 | 19533 | 20490 | 21447 | 16754 | 22404 | 23361 | 24318 | 25275 |
| 16755 | 18577 | 19534 | 20491 | 21448 | 16756 | 22405 | 23362 | 24319 | 25276 |
| 16757 | 18578 | 19535 | 20492 | 21449 | 16758 | 22406 | 23363 | 24320 | 25277 |
| 16759 | 18579 | 19536 | 20493 | 21450 | 16760 | 22407 | 23364 | 24321 | 25278 |
| 16761 | 18580 | 19537 | 20494 | 21451 | 16762 | 22408 | 23365 | 24322 | 25279 |
| 16763 | 18581 | 19538 | 20495 | 21452 | 16764 | 22409 | 23366 | 24323 | 25280 |
| 16765 | 18582 | 19539 | 20496 | 21453 | 16766 | 22410 | 23367 | 24324 | 25281 |
| 16767 | 18583 | 19540 | 20497 | 21454 | 16768 | 22411 | 23368 | 24325 | 25282 |
| 16769 | 18584 | 19541 | 20498 | 21455 | 16770 | 22412 | 23369 | 24326 | 25283 |
| 16771 | 18585 | 19542 | 20499 | 21456 | 16772 | 22413 | 23370 | 24327 | 25284 |
| 16773 | 18586 | 19543 | 20500 | 21457 | 16774 | 22414 | 23371 | 24328 | 25285 |
| 16775 | 18587 | 19544 | 20501 | 21458 | 16776 | 22415 | 23372 | 24329 | 25286 |
| 16777 | 18588 | 19545 | 20502 | 21459 | 16778 | 22416 | 23373 | 24330 | 25287 |
| 16779 | 18589 | 19546 | 20503 | 21460 | 16780 | 22417 | 23374 | 24331 | 25288 |
| 16781 | 18590 | 19547 | 20504 | 21461 | 16782 | 22418 | 23375 | 24332 | 25289 |
| 16783 | 18591 | 19548 | 20505 | 21462 | 16784 | 22419 | 23376 | 24333 | 25290 |
| 16785 | 18592 | 19549 | 20506 | 21463 | 16786 | 22420 | 23377 | 24334 | 25291 |
| 16787 | 18593 | 19550 | 20507 | 21464 | 16788 | 22421 | 23378 | 24335 | 25292 |
| 16789 | 18594 | 19551 | 20508 | 21465 | 16790 | 22422 | 23379 | 24336 | 25293 |
| 16791 | 18595 | 19552 | 20509 | 21466 | 16792 | 22423 | 23380 | 24337 | 25294 |
| 16793 | 18596 | 19553 | 20510 | 21467 | 16794 | 22424 | 23381 | 24338 | 25295 |
| 16795 | 18597 | 19554 | 20511 | 21468 | 16796 | 22425 | 23382 | 24339 | 25296 |
| 16797 | 18598 | 19555 | 20512 | 21469 | 16798 | 22426 | 23383 | 24340 | 25297 |
| 16799 | 18599 | 19556 | 20513 | 21470 | 16800 | 22427 | 23384 | 24341 | 25298 |
| 16801 | 18600 | 19557 | 20514 | 21471 | 16802 | 22428 | 23385 | 24342 | 25299 |
| 16803 | 18601 | 19558 | 20515 | 21472 | 16804 | 22429 | 23386 | 24343 | 25300 |
| 16805 | 18602 | 19559 | 20516 | 21473 | 16806 | 22430 | 23387 | 24344 | 25301 |
| 16807 | 18603 | 19560 | 20517 | 21474 | 16808 | 22431 | 23388 | 24345 | 25302 |
| 16809 | 18604 | 19561 | 20518 | 21475 | 16810 | 22432 | 23389 | 24346 | 25303 |
| 16811 | 18605 | 19562 | 20519 | 21476 | 16812 | 22433 | 23390 | 24347 | 25304 |
| 16813 | 18606 | 19563 | 20520 | 21477 | 16814 | 22434 | 23391 | 24348 | 25305 |
| 16815 | 18607 | 19564 | 20521 | 21478 | 16816 | 22435 | 23392 | 24349 | 25306 |
| 16817 | 18608 | 19565 | 20522 | 21479 | 16818 | 22436 | 23393 | 24350 | 25307 |
| 16819 | 18609 | 19566 | 20523 | 21480 | 16820 | 22437 | 23394 | 24351 | 25308 |
| 16821 | 18610 | 19567 | 20524 | 21481 | 16822 | 22438 | 23395 | 24352 | 25309 |
| 16823 | 18611 | 19568 | 20525 | 21482 | 16824 | 22439 | 23396 | 24353 | 25310 |
| 16825 | 18612 | 19569 | 20526 | 21483 | 16826 | 22440 | 23397 | 24354 | 25311 |
| 16827 | 18613 | 19570 | 20527 | 21484 | 16828 | 22441 | 23398 | 24355 | 25312 |
| 16829 | 18614 | 19571 | 20528 | 21485 | 16830 | 22442 | 23399 | 24356 | 25313 |
| 16831 | 18615 | 19572 | 20529 | 21486 | 16832 | 22443 | 23400 | 24357 | 25314 |
| 16833 | 18616 | 19573 | 20530 | 21487 | 16834 | 22444 | 23401 | 24358 | 25315 |
| 16835 | 18617 | 19574 | 20531 | 21488 | 16836 | 22445 | 23402 | 24359 | 25316 |
| 16837 | 18618 | 19575 | 20532 | 21489 | 16838 | 22446 | 23403 | 24360 | 25317 |
| 16839 | 18619 | 19576 | 20533 | 21490 | 16840 | 22447 | 23404 | 24361 | 25318 |
| 16841 | 18620 | 19577 | 20534 | 21491 | 16842 | 22448 | 23405 | 24362 | 25319 |
| 16843 | 18621 | 19578 | 20535 | 21492 | 16844 | 22449 | 23406 | 24363 | 25320 |
| 16845 | 18622 | 19579 | 20536 | 21493 | 16846 | 22450 | 23407 | 24364 | 25321 |
| 16847 | 18623 | 19580 | 20537 | 21494 | 16848 | 22451 | 23408 | 24365 | 25322 |
| 16849 | 18624 | 19581 | 20538 | 21495 | 16850 | 22452 | 23409 | 24366 | 25323 |
| 16851 | 18625 | 19582 | 20539 | 21496 | 16852 | 22453 | 23410 | 24367 | 25324 |
| 16853 | 18626 | 19583 | 20540 | 21497 | 16854 | 22454 | 23411 | 24368 | 25325 |
| 16855 | 18627 | 19584 | 20541 | 21498 | 16856 | 22455 | 23412 | 24369 | 25326 |
| 16857 | 18628 | 19585 | 20542 | 21499 | 16858 | 22456 | 23413 | 24370 | 25327 |
| 16859 | 18629 | 19586 | 20543 | 21500 | 16860 | 22457 | 23414 | 24371 | 25328 |
| 16861 | 18630 | 19587 | 20544 | 21501 | 16862 | 22458 | 23415 | 24372 | 25329 |
| 16863 | 18631 | 19588 | 20545 | 21502 | 16864 | 22459 | 23416 | 24373 | 25330 |
| 16865 | 18632 | 19589 | 20546 | 21503 | 16866 | 22460 | 23417 | 24374 | 25331 |
| 16867 | 18633 | 19590 | 20547 | 21504 | 16868 | 22461 | 23418 | 24375 | 25332 |
| 16869 | 18634 | 19591 | 20548 | 21505 | 16870 | 22462 | 23419 | 24376 | 25333 |
| 16871 | 18635 | 19592 | 20549 | 21506 | 16872 | 22463 | 23420 | 24377 | 25334 |
| 16873 | 18636 | 19593 | 20550 | 21507 | 16874 | 22464 | 23421 | 24378 | 25335 |
| 16875 | 18637 | 19594 | 20551 | 21508 | 16876 | 22465 | 23422 | 24379 | 25336 |
| 16877 | 18638 | 19595 | 20552 | 21509 | 16878 | 22466 | 23423 | 24380 | 25337 |
| 16879 | 18639 | 19596 | 20553 | 21510 | 16880 | 22467 | 23424 | 24381 | 25338 |
| 16881 | 18640 | 19597 | 20554 | 21511 | 16882 | 22468 | 23425 | 24382 | 25339 |
| 16883 | 18641 | 19598 | 20555 | 21512 | 16884 | 22469 | 23426 | 24383 | 25340 |
| 16885 | 18642 | 19599 | 20556 | 21513 | 16886 | 22470 | 23427 | 24384 | 25341 |
| 16887 | 18643 | 19600 | 20557 | 21514 | 16888 | 22471 | 23428 | 24385 | 25342 |
| 16889 | 18644 | 19601 | 20558 | 21515 | 16890 | 22472 | 23429 | 24386 | 25343 |
| 16891 | 18645 | 19602 | 20559 | 21516 | 16892 | 22473 | 23430 | 24387 | 25344 |
| 16893 | 18646 | 19603 | 20560 | 21517 | 16894 | 22474 | 23431 | 24388 | 25345 |
| 16895 | 18647 | 19604 | 20561 | 21518 | 16896 | 22475 | 23432 | 24389 | 25346 |
| 16897 | 18648 | 19605 | 20562 | 21519 | 16898 | 22476 | 23433 | 24390 | 25347 |
| 16899 | 18649 | 19606 | 20563 | 21520 | 16900 | 22477 | 23434 | 24391 | 25348 |
| 16901 | 18650 | 19607 | 20564 | 21521 | 16902 | 22478 | 23435 | 24392 | 25349 |
| 16903 | 18651 | 19608 | 20565 | 21522 | 16904 | 22479 | 23436 | 24393 | 25350 |
| 16905 | 18652 | 19609 | 20566 | 21523 | 16906 | 22480 | 23437 | 24394 | 25351 |
| 16907 | 18653 | 19610 | 20567 | 21524 | 16908 | 22481 | 23438 | 24395 | 25352 |
| 16909 | 18654 | 19611 | 20568 | 21525 | 16910 | 22482 | 23439 | 24396 | 25353 |
| 16911 | 18655 | 19612 | 20569 | 21526 | 16912 | 22483 | 23440 | 24397 | 25354 |
| 16913 | 18656 | 19613 | 20570 | 21527 | 16914 | 22484 | 23441 | 24398 | 25355 |
| 16915 | 18657 | 19614 | 20571 | 21528 | 16916 | 22485 | 23442 | 24399 | 25356 |
| 16917 | 18658 | 19615 | 20572 | 21529 | 16918 | 22486 | 23443 | 24400 | 25357 |
| 16919 | 18659 | 19616 | 20573 | 21530 | 16920 | 22487 | 23444 | 24401 | 25358 |
| 16921 | 18660 | 19617 | 20574 | 21531 | 16922 | 22488 | 23445 | 24402 | 25359 |
| 16923 | 18661 | 19618 | 20575 | 21532 | 16924 | 22489 | 23446 | 24403 | 25360 |
| 16925 | 18662 | 19619 | 20576 | 21533 | 16926 | 22490 | 23447 | 24404 | 25361 |
| 16927 | 18663 | 19620 | 20577 | 21534 | 16928 | 22491 | 23448 | 24405 | 25362 |
| 16929 | 18664 | 19621 | 20578 | 21535 | 16930 | 22492 | 23449 | 24406 | 25363 |
| 16931 | 18665 | 19622 | 20579 | 21536 | 16932 | 22493 | 23450 | 24407 | 25364 |
| 16933 | 18666 | 19623 | 20580 | 21537 | 16934 | 22494 | 23451 | 24408 | 25365 |
| 16935 | 18667 | 19624 | 20581 | 21538 | 16936 | 22495 | 23452 | 24409 | 25366 |
| 16937 | 18668 | 19625 | 20582 | 21539 | 16938 | 22496 | 23453 | 24410 | 25367 |
| 16939 | 18669 | 19626 | 20583 | 21540 | 16940 | 22497 | 23454 | 24411 | 25368 |
| 16941 | 18670 | 19627 | 20584 | 21541 | 16942 | 22498 | 23455 | 24412 | 25369 |
| 16943 | 18671 | 19628 | 20585 | 21542 | 16944 | 22499 | 23456 | 24413 | 25370 |
| 16945 | 18672 | 19629 | 20586 | 21543 | 16946 | 22500 | 23457 | 24414 | 25371 |
| 16947 | 18673 | 19630 | 20587 | 21544 | 16948 | 22501 | 23458 | 24415 | 25372 |
| 16949 | 18674 | 19631 | 20588 | 21545 | 16950 | 22502 | 23459 | 24416 | 25373 |
| 16951 | 18675 | 19632 | 20589 | 21546 | 16952 | 22503 | 23460 | 24417 | 25374 |
| 16953 | 18676 | 19633 | 20590 | 21547 | 16954 | 22504 | 23461 | 24418 | 25375 |
| 16955 | 18677 | 19634 | 20591 | 21548 | 16956 | 22505 | 23462 | 24419 | 25376 |
| 16957 | 18678 | 19635 | 20592 | 21549 | 16958 | 22506 | 23463 | 24420 | 25377 |
| 16959 | 18679 | 19636 | 20593 | 21550 | 16960 | 22507 | 23464 | 24421 | 25378 |
| 16961 | 18680 | 19637 | 20594 | 21551 | 16962 | 22508 | 23465 | 24422 | 25379 |
| 16963 | 18681 | 19638 | 20595 | 21552 | 16964 | 22509 | 23466 | 24423 | 25380 |
| 16965 | 18682 | 19639 | 20596 | 21553 | 16966 | 22510 | 23467 | 24424 | 25381 |
| 16967 | 18683 | 19640 | 20597 | 21554 | 16968 | 22511 | 23468 | 24425 | 25382 |
| 16969 | 18684 | 19641 | 20598 | 21555 | 16970 | 22512 | 23469 | 24426 | 25383 |
| 16971 | 18685 | 19642 | 20599 | 21556 | 16972 | 22513 | 23470 | 24427 | 25384 |
| 16973 | 18686 | 19643 | 20600 | 21557 | 16974 | 22514 | 23471 | 24428 | 25385 |
| 16975 | 18687 | 19644 | 20601 | 21558 | 16976 | 22515 | 23472 | 24429 | 25386 |
| 16977 | 18688 | 19645 | 20602 | 21559 | 16978 | 22516 | 23473 | 24430 | 25387 |
| 16979 | 18689 | 19646 | 20603 | 21560 | 16980 | 22517 | 23474 | 24431 | 25388 |
| 16981 | 18690 | 19647 | 20604 | 21561 | 16982 | 22518 | 23475 | 24432 | 25389 |
| 16983 | 18691 | 19648 | 20605 | 21562 | 16984 | 22519 | 23476 | 24433 | 25390 |
| 16985 | 18692 | 19649 | 20606 | 21563 | 16986 | 22520 | 23477 | 24434 | 25391 |
| 16987 | 18693 | 19650 | 20607 | 21564 | 16988 | 22521 | 23478 | 24435 | 25392 |
| 16989 | 18694 | 19651 | 20608 | 21565 | 16990 | 22522 | 23479 | 24436 | 25393 |
| 16991 | 18695 | 19652 | 20609 | 21566 | 16992 | 22523 | 23480 | 24437 | 25394 |
| 16993 | 18696 | 19653 | 20610 | 21567 | 16994 | 22524 | 23481 | 24438 | 25395 |
| 16995 | 18697 | 19654 | 20611 | 21568 | 16996 | 22525 | 23482 | 24439 | 25396 |
| 16997 | 18698 | 19655 | 20612 | 21569 | 16998 | 22526 | 23483 | 24440 | 25397 |
| 16999 | 18699 | 19656 | 20613 | 21570 | 17000 | 22527 | 23484 | 24441 | 25398 |
| 17001 | 18700 | 19657 | 20614 | 21571 | 17002 | 22528 | 23485 | 24442 | 25399 |
| 17003 | 18701 | 19658 | 20615 | 21572 | 17004 | 22529 | 23486 | 24443 | 25400 |
| 17005 | 18702 | 19659 | 20616 | 21573 | 17006 | 22530 | 23487 | 24444 | 25401 |
| 17007 | 18703 | 19660 | 20617 | 21574 | 17008 | 22531 | 23488 | 24445 | 25402 |
| 17009 | 18704 | 19661 | 20618 | 21575 | 17010 | 22532 | 23489 | 24446 | 25403 |
| 17011 | 18705 | 19662 | 20619 | 21576 | 17012 | 22533 | 23490 | 24447 | 25404 |
| 17013 | 18706 | 19663 | 20620 | 21577 | 17014 | 22534 | 23491 | 24448 | 25405 |
| 17015 | 18707 | 19664 | 20621 | 21578 | 17016 | 22535 | 23492 | 24449 | 25406 |
| 17017 | 18708 | 19665 | 20622 | 21579 | 17018 | 22536 | 23493 | 24450 | 25407 |
| 17019 | 18709 | 19666 | 20623 | 21580 | 17020 | 22537 | 23494 | 24451 | 25408 |
| 17021 | 18710 | 19667 | 20624 | 21581 | 17022 | 22538 | 23495 | 24452 | 25409 |
| 17023 | 18711 | 19668 | 20625 | 21582 | 17024 | 22539 | 23496 | 24453 | 25410 |
| 17025 | 18712 | 19669 | 20626 | 21583 | 17026 | 22540 | 23497 | 24454 | 25411 |
| 17027 | 18713 | 19670 | 20627 | 21584 | 17028 | 22541 | 23498 | 24455 | 25412 |
| 17029 | 18714 | 19671 | 20628 | 21585 | 17030 | 22542 | 23499 | 24456 | 25413 |
| 17031 | 18715 | 19672 | 20629 | 21586 | 17032 | 22543 | 23500 | 24457 | 25414 |
| 17033 | 18716 | 19673 | 20630 | 21587 | 17034 | 22544 | 23501 | 24458 | 25415 |
| 17035 | 18717 | 19674 | 20631 | 21588 | 17036 | 22545 | 23502 | 24459 | 25416 |
| 17037 | 18718 | 19675 | 20632 | 21589 | 17038 | 22546 | 23503 | 24460 | 25417 |
| 17039 | 18719 | 19676 | 20633 | 21590 | 17040 | 22547 | 23504 | 24461 | 25418 |
| 17041 | 18720 | 19677 | 20634 | 21591 | 17042 | 22548 | 23505 | 24462 | 25419 |
| 17043 | 18721 | 19678 | 20635 | 21592 | 17044 | 22549 | 23506 | 24463 | 25420 |
| 17045 | 18722 | 19679 | 20636 | 21593 | 17046 | 22550 | 23507 | 24464 | 25421 |
| 17047 | 18723 | 19680 | 20637 | 21594 | 17048 | 22551 | 23508 | 24465 | 25422 |
| 17049 | 18724 | 19681 | 20638 | 21595 | 17050 | 22552 | 23509 | 24466 | 25423 |
| 17051 | 18725 | 19682 | 20639 | 21596 | 17052 | 22553 | 23510 | 24467 | 25424 |
| 17053 | 18726 | 19683 | 20640 | 21597 | 17054 | 22554 | 23511 | 24468 | 25425 |
| 17055 | 18727 | 19684 | 20641 | 21598 | 17056 | 22555 | 23512 | 24469 | 25426 |
| 17057 | 18728 | 19685 | 20642 | 21599 | 17058 | 22556 | 23513 | 24470 | 25427 |
| 17059 | 18729 | 19686 | 20643 | 21600 | 17060 | 22557 | 23514 | 24471 | 25428 |
| 17061 | 18730 | 19687 | 20644 | 21601 | 17062 | 22558 | 23515 | 24472 | 25429 |
| 17063 | 18731 | 19688 | 20645 | 21602 | 17064 | 22559 | 23516 | 24473 | 25430 |
| 17065 | 18732 | 19689 | 20646 | 21603 | 17066 | 22560 | 23517 | 24474 | 25431 |
| 17067 | 18733 | 19690 | 20647 | 21604 | 17068 | 22561 | 23518 | 24475 | 25432 |
| 17069 | 18734 | 19691 | 20648 | 21605 | 17070 | 22562 | 23519 | 24476 | 25433 |
| 17071 | 18735 | 19692 | 20649 | 21606 | 17072 | 22563 | 23520 | 24477 | 25434 |
| 17073 | 18736 | 19693 | 20650 | 21607 | 17074 | 22564 | 23521 | 24478 | 25435 |
| 17075 | 18737 | 19694 | 20651 | 21608 | 17076 | 22565 | 23522 | 24479 | 25436 |
| 17077 | 18738 | 19695 | 20652 | 21609 | 17078 | 22566 | 23523 | 24480 | 25437 |
| 17079 | 18739 | 19696 | 20653 | 21610 | 17080 | 22567 | 23524 | 24481 | 25438 |
| 17081 | 18740 | 19697 | 20654 | 21611 | 17082 | 22568 | 23525 | 24482 | 25439 |
| 17083 | 18741 | 19698 | 20655 | 21612 | 17084 | 22569 | 23526 | 24483 | 25440 |
| 17085 | 18742 | 19699 | 20656 | 21613 | 17086 | 22570 | 23527 | 24484 | 25441 |
| 17087 | 18743 | 19700 | 20657 | 21614 | 17088 | 22571 | 23528 | 24485 | 25442 |
| 17089 | 18744 | 19701 | 20658 | 21615 | 17090 | 22572 | 23529 | 24486 | 25443 |
| 17091 | 18745 | 19702 | 20659 | 21616 | 17092 | 22573 | 23530 | 24487 | 25444 |
| 17093 | 18746 | 19703 | 20660 | 21617 | 17094 | 22574 | 23531 | 24488 | 25445 |
| 17095 | 18747 | 19704 | 20661 | 21618 | 17096 | 22575 | 23532 | 24489 | 25446 |
| 17097 | 18748 | 19705 | 20662 | 21619 | 17098 | 22576 | 23533 | 24490 | 25447 |
| 17099 | 18749 | 19706 | 20663 | 21620 | 17100 | 22577 | 23534 | 24491 | 25448 |
| 17101 | 18750 | 19707 | 20664 | 21621 | 17102 | 22578 | 23535 | 24492 | 25449 |
| 17103 | 18751 | 19708 | 20665 | 21622 | 17104 | 22579 | 23536 | 24493 | 25450 |
| 17105 | 18752 | 19709 | 20666 | 21623 | 17106 | 22580 | 23537 | 24494 | 25451 |
| 17107 | 18753 | 19710 | 20667 | 21624 | 17108 | 22581 | 23538 | 24495 | 25452 |
| 17109 | 18754 | 19711 | 20668 | 21625 | 17110 | 22582 | 23539 | 24496 | 25453 |
| 17111 | 18755 | 19712 | 20669 | 21626 | 17112 | 22583 | 23540 | 24497 | 25454 |
| 17113 | 18756 | 19713 | 20670 | 21627 | 17114 | 22584 | 23541 | 24498 | 25455 |
| 17115 | 18757 | 19714 | 20671 | 21628 | 17116 | 22585 | 23542 | 24499 | 25456 |
| 17117 | 18758 | 19715 | 20672 | 21629 | 17118 | 22586 | 23543 | 24500 | 25457 |
| 17119 | 18759 | 19716 | 20673 | 21630 | 17120 | 22587 | 23544 | 24501 | 25458 |
| 17121 | 18760 | 19717 | 20674 | 21631 | 17122 | 22588 | 23545 | 24502 | 25459 |
| 17123 | 18761 | 19718 | 20675 | 21632 | 17124 | 22589 | 23546 | 24503 | 25460 |
| 17125 | 18762 | 19719 | 20676 | 21633 | 17126 | 22590 | 23547 | 24504 | 25461 |
| 17127 | 18763 | 19720 | 20677 | 21634 | 17128 | 22591 | 23548 | 24505 | 25462 |
| 17129 | 18764 | 19721 | 20678 | 21635 | 17130 | 22592 | 23549 | 24506 | 25463 |
| 17131 | 18765 | 19722 | 20679 | 21636 | 17132 | 22593 | 23550 | 24507 | 25464 |
| 17133 | 18766 | 19723 | 20680 | 21637 | 17134 | 22594 | 23551 | 24508 | 25465 |
| 17135 | 18767 | 19724 | 20681 | 21638 | 17136 | 22595 | 23552 | 24509 | 25466 |
| 17137 | 18768 | 19725 | 20682 | 21639 | 17138 | 22596 | 23553 | 24510 | 25467 |
| 17139 | 18769 | 19726 | 20683 | 21640 | 17140 | 22597 | 23554 | 24511 | 25468 |
| 17141 | 18770 | 19727 | 20684 | 21641 | 17142 | 22598 | 23555 | 24512 | 25469 |
| 17143 | 18771 | 19728 | 20685 | 21642 | 17144 | 22599 | 23556 | 24513 | 25470 |
| 17145 | 18772 | 19729 | 20686 | 21643 | 17146 | 22600 | 23557 | 24514 | 25471 |
| 17147 | 18773 | 19730 | 20687 | 21644 | 17148 | 22601 | 23558 | 24515 | 25472 |
| 17149 | 18774 | 19731 | 20688 | 21645 | 17150 | 22602 | 23559 | 24516 | 25473 |
| 17151 | 18775 | 19732 | 20689 | 21646 | 17152 | 22603 | 23560 | 24517 | 25474 |
| 17153 | 18776 | 19733 | 20690 | 21647 | 17154 | 22604 | 23561 | 24518 | 25475 |
| 17155 | 18777 | 19734 | 20691 | 21648 | 17156 | 22605 | 23562 | 24519 | 25476 |
| 17157 | 18778 | 19735 | 20692 | 21649 | 17158 | 22606 | 23563 | 24520 | 25477 |
| 17159 | 18779 | 19736 | 20693 | 21650 | 17160 | 22607 | 23564 | 24521 | 25478 |
| 17161 | 18780 | 19737 | 20694 | 21651 | 17162 | 22608 | 23565 | 24522 | 25479 |
| 17163 | 18781 | 19738 | 20695 | 21652 | 17164 | 22609 | 23566 | 24523 | 25480 |
| 17165 | 18782 | 19739 | 20696 | 21653 | 17166 | 22610 | 23567 | 24524 | 25481 |
| 17167 | 18783 | 19740 | 20697 | 21654 | 17168 | 22611 | 23568 | 24525 | 25482 |
| 17169 | 18784 | 19741 | 20698 | 21655 | 17170 | 22612 | 23569 | 24526 | 25483 |
| 17171 | 18785 | 19742 | 20699 | 21656 | 17172 | 22613 | 23570 | 24527 | 25484 |
| 17173 | 18786 | 19743 | 20700 | 21657 | 17174 | 22614 | 23571 | 24528 | 25485 |
| 17175 | 18787 | 19744 | 20701 | 21658 | 17176 | 22615 | 23572 | 24529 | 25486 |
| 17177 | 18788 | 19745 | 20702 | 21659 | 17178 | 22616 | 23573 | 24530 | 25487 |
| 17179 | 18789 | 19746 | 20703 | 21660 | 17180 | 22617 | 23574 | 24531 | 25488 |
| 17181 | 18790 | 19747 | 20704 | 21661 | 17182 | 22618 | 23575 | 24532 | 25489 |
| 17183 | 18791 | 19748 | 20705 | 21662 | 17184 | 22619 | 23576 | 24533 | 25490 |
| 17185 | 18792 | 19749 | 20706 | 21663 | 17186 | 22620 | 23577 | 24534 | 25491 |
| 17187 | 18793 | 19750 | 20707 | 21664 | 17188 | 22621 | 23578 | 24535 | 25492 |
| 17189 | 18794 | 19751 | 20708 | 21665 | 17190 | 22622 | 23579 | 24536 | 25493 |
| 17191 | 18795 | 19752 | 20709 | 21666 | 17192 | 22623 | 23580 | 24537 | 25494 |
| 17193 | 18796 | 19753 | 20710 | 21667 | 17194 | 22624 | 23581 | 24538 | 25495 |
| 17195 | 18797 | 19754 | 20711 | 21668 | 17196 | 22625 | 23582 | 24539 | 25496 |
| 17197 | 18798 | 19755 | 20712 | 21669 | 17198 | 22626 | 23583 | 24540 | 25497 |
| 17199 | 18799 | 19756 | 20713 | 21670 | 17200 | 22627 | 23584 | 24541 | 25498 |
| 17201 | 18800 | 19757 | 20714 | 21671 | 17202 | 22628 | 23585 | 24542 | 25499 |
| 17203 | 18801 | 19758 | 20715 | 21672 | 17204 | 22629 | 23586 | 24543 | 25500 |
| 17205 | 18802 | 19759 | 20716 | 21673 | 17206 | 22630 | 23587 | 24544 | 25501 |
| 17207 | 18803 | 19760 | 20717 | 21674 | 17208 | 22631 | 23588 | 24545 | 25502 |
| 17209 | 18804 | 19761 | 20718 | 21675 | 17210 | 22632 | 23589 | 24546 | 25503 |
| 17211 | 18805 | 19762 | 20719 | 21676 | 17212 | 22633 | 23590 | 24547 | 25504 |
| 17213 | 18806 | 19763 | 20720 | 21677 | 17214 | 22634 | 23591 | 24548 | 25505 |
| 17215 | 18807 | 19764 | 20721 | 21678 | 17216 | 22635 | 23592 | 24549 | 25506 |
| 17217 | 18808 | 19765 | 20722 | 21679 | 17218 | 22636 | 23593 | 24550 | 25507 |
| 17219 | 18809 | 19766 | 20723 | 21680 | 17220 | 22637 | 23594 | 24551 | 25508 |
| 17221 | 18810 | 19767 | 20724 | 21681 | 17222 | 22638 | 23595 | 24552 | 25509 |
| 17223 | 18811 | 19768 | 20725 | 21682 | 17224 | 22639 | 23596 | 24553 | 25510 |
| 17225 | 18812 | 19769 | 20726 | 21683 | 17226 | 22640 | 23597 | 24554 | 25511 |
| 17227 | 18813 | 19770 | 20727 | 21684 | 17228 | 22641 | 23598 | 24555 | 25512 |
| 17229 | 18814 | 19771 | 20728 | 21685 | 17230 | 22642 | 23599 | 24556 | 25513 |
| 17231 | 18815 | 19772 | 20729 | 21686 | 17232 | 22643 | 23600 | 24557 | 25514 |
| 17233 | 18816 | 19773 | 20730 | 21687 | 17234 | 22644 | 23601 | 24558 | 25515 |
| 17235 | 18817 | 19774 | 20731 | 21688 | 17236 | 22645 | 23602 | 24559 | 25516 |
| 17237 | 18818 | 19775 | 20732 | 21689 | 17238 | 22646 | 23603 | 24560 | 25517 |
| 17239 | 18819 | 19776 | 20733 | 21690 | 17240 | 22647 | 23604 | 24561 | 25518 |
| 17241 | 18820 | 19777 | 20734 | 21691 | 17242 | 22648 | 23605 | 24562 | 25519 |
| 17243 | 18821 | 19778 | 20735 | 21692 | 17244 | 22649 | 23606 | 24563 | 25520 |
| 17245 | 18822 | 19779 | 20736 | 21693 | 17246 | 22650 | 23607 | 24564 | 25521 |
| 17247 | 18823 | 19780 | 20737 | 21694 | 17248 | 22651 | 23608 | 24565 | 25522 |
| 17249 | 18824 | 19781 | 20738 | 21695 | 17250 | 22652 | 23609 | 24566 | 25523 |
| 17251 | 18825 | 19782 | 20739 | 21696 | 17252 | 22653 | 23610 | 24567 | 25524 |
| 17253 | 18826 | 19783 | 20740 | 21697 | 17254 | 22654 | 23611 | 24568 | 25525 |
| 17255 | 18827 | 19784 | 20741 | 21698 | 17256 | 22655 | 23612 | 24569 | 25526 |
| 17257 | 18828 | 19785 | 20742 | 21699 | 17258 | 22656 | 23613 | 24570 | 25527 |
| 17259 | 18829 | 19786 | 20743 | 21700 | 17260 | 22657 | 23614 | 24571 | 25528 |
| 17261 | 18830 | 19787 | 20744 | 21701 | 17262 | 22658 | 23615 | 24572 | 25529 |
| 17263 | 18831 | 19788 | 20745 | 21702 | 17264 | 22659 | 23616 | 24573 | 25530 |
| 17265 | 18832 | 19789 | 20746 | 21703 | 17266 | 22660 | 23617 | 24574 | 25531 |
| 17267 | 18833 | 19790 | 20747 | 21704 | 17268 | 22661 | 23618 | 24575 | 25532 |
| 17269 | 18834 | 19791 | 20748 | 21705 | 17270 | 22662 | 23619 | 24576 | 25533 |
| 17271 | 18835 | 19792 | 20749 | 21706 | 17272 | 22663 | 23620 | 24577 | 25534 |
| 17273 | 18836 | 19793 | 20750 | 21707 | 17274 | 22664 | 23621 | 24578 | 25535 |
| 17275 | 18837 | 19794 | 20751 | 21708 | 17276 | 22665 | 23622 | 24579 | 25536 |
| 17277 | 18838 | 19795 | 20752 | 21709 | 17278 | 22666 | 23623 | 24580 | 25537 |
| 17279 | 18839 | 19796 | 20753 | 21710 | 17280 | 22667 | 23624 | 24581 | 25538 |
| 17281 | 18840 | 19797 | 20754 | 21711 | 17282 | 22668 | 23625 | 24582 | 25539 |
| 17283 | 18841 | 19798 | 20755 | 21712 | 17284 | 22669 | 23626 | 24583 | 25540 |
| 17285 | 18842 | 19799 | 20756 | 21713 | 17286 | 22670 | 23627 | 24584 | 25541 |
| 17287 | 18843 | 19800 | 20757 | 21714 | 17288 | 22671 | 23628 | 24585 | 25542 |
| 17289 | 18844 | 19801 | 20758 | 21715 | 17290 | 22672 | 23629 | 24586 | 25543 |
| 17291 | 18845 | 19802 | 20759 | 21716 | 17292 | 22673 | 23630 | 24587 | 25544 |
| 17293 | 18846 | 19803 | 20760 | 21717 | 17294 | 22674 | 23631 | 24588 | 25545 |
| 17295 | 18847 | 19804 | 20761 | 21718 | 17296 | 22675 | 23632 | 24589 | 25546 |
| 17297 | 18848 | 19805 | 20762 | 21719 | 17298 | 22676 | 23633 | 24590 | 25547 |
| 17299 | 18849 | 19806 | 20763 | 21720 | 17300 | 22677 | 23634 | 24591 | 25548 |
| 17301 | 18850 | 19807 | 20764 | 21721 | 17302 | 22678 | 23635 | 24592 | 25549 |
| 17303 | 18851 | 19808 | 20765 | 21722 | 17304 | 22679 | 23636 | 24593 | 25550 |
| 17305 | 18852 | 19809 | 20766 | 21723 | 17306 | 22680 | 23637 | 24594 | 25551 |
| 17307 | 18853 | 19810 | 20767 | 21724 | 17308 | 22681 | 23638 | 24595 | 25552 |
| 17309 | 18854 | 19811 | 20768 | 21725 | 17310 | 22682 | 23639 | 24596 | 25553 |
| 17311 | 18855 | 19812 | 20769 | 21726 | 17312 | 22683 | 23640 | 24597 | 25554 |
| 17313 | 18856 | 19813 | 20770 | 21727 | 17314 | 22684 | 23641 | 24598 | 25555 |
| 17315 | 18857 | 19814 | 20771 | 21728 | 17316 | 22685 | 23642 | 24599 | 25556 |
| 17317 | 18858 | 19815 | 20772 | 21729 | 17318 | 22686 | 23643 | 24600 | 25557 |
| 17319 | 18859 | 19816 | 20773 | 21730 | 17320 | 22687 | 23644 | 24601 | 25558 |
| 17321 | 18860 | 19817 | 20774 | 21731 | 17322 | 22688 | 23645 | 24602 | 25559 |
| 17323 | 18861 | 19818 | 20775 | 21732 | 17324 | 22689 | 23646 | 24603 | 25560 |
| 17325 | 18862 | 19819 | 20776 | 21733 | 17326 | 22690 | 23647 | 24604 | 25561 |
| 17327 | 18863 | 19820 | 20777 | 21734 | 17328 | 22691 | 23648 | 24605 | 25562 |
| 17329 | 18864 | 19821 | 20778 | 21735 | 17330 | 22692 | 23649 | 24606 | 25563 |
| 17331 | 18865 | 19822 | 20779 | 21736 | 17332 | 22693 | 23650 | 24607 | 25564 |
| 17333 | 18866 | 19823 | 20780 | 21737 | 17334 | 22694 | 23651 | 24608 | 25565 |
| 17335 | 18867 | 19824 | 20781 | 21738 | 17336 | 22695 | 23652 | 24609 | 25566 |
| 17337 | 18868 | 19825 | 20782 | 21739 | 17338 | 22696 | 23653 | 24610 | 25567 |
| 17339 | 18869 | 19826 | 20783 | 21740 | 17340 | 22697 | 23654 | 24611 | 25568 |
| 17341 | 18870 | 19827 | 20784 | 21741 | 17342 | 22698 | 23655 | 24612 | 25569 |
| 17343 | 18871 | 19828 | 20785 | 21742 | 17344 | 22699 | 23656 | 24613 | 25570 |
| 17345 | 18872 | 19829 | 20786 | 21743 | 17346 | 22700 | 23657 | 24614 | 25571 |
| 17347 | 18873 | 19830 | 20787 | 21744 | 17348 | 22701 | 23658 | 24615 | 25572 |
| 17349 | 18874 | 19831 | 20788 | 21745 | 17350 | 22702 | 23659 | 24616 | 25573 |
| 17351 | 18875 | 19832 | 20789 | 21746 | 17352 | 22703 | 23660 | 24617 | 25574 |
| 17353 | 18876 | 19833 | 20790 | 21747 | 17354 | 22704 | 23661 | 24618 | 25575 |
| 17355 | 18877 | 19834 | 20791 | 21748 | 17356 | 22705 | 23662 | 24619 | 25576 |
| 17357 | 18878 | 19835 | 20792 | 21749 | 17358 | 22706 | 23663 | 24620 | 25577 |
| 17359 | 18879 | 19836 | 20793 | 21750 | 17360 | 22707 | 23664 | 24621 | 25578 |
| 17361 | 18880 | 19837 | 20794 | 21751 | 17362 | 22708 | 23665 | 24622 | 25579 |
| 17363 | 18881 | 19838 | 20795 | 21752 | 17364 | 22709 | 23666 | 24623 | 25580 |
| 17365 | 18882 | 19839 | 20796 | 21753 | 17366 | 22710 | 23667 | 24624 | 25581 |
| 17367 | 18883 | 19840 | 20797 | 21754 | 17368 | 22711 | 23668 | 24625 | 25582 |
| 17369 | 18884 | 19841 | 20798 | 21755 | 17370 | 22712 | 23669 | 24626 | 25583 |
| 17371 | 18885 | 19842 | 20799 | 21756 | 17372 | 22713 | 23670 | 24627 | 25584 |
| 17373 | 18886 | 19843 | 20800 | 21757 | 17374 | 22714 | 23671 | 24628 | 25585 |
| 17375 | 18887 | 19844 | 20801 | 21758 | 17376 | 22715 | 23672 | 24629 | 25586 |
| 17377 | 18888 | 19845 | 20802 | 21759 | 17378 | 22716 | 23673 | 24630 | 25587 |
| 17379 | 18889 | 19846 | 20803 | 21760 | 17380 | 22717 | 23674 | 24631 | 25588 |
| 17381 | 18890 | 19847 | 20804 | 21761 | 17382 | 22718 | 23675 | 24632 | 25589 |
| 17383 | 18891 | 19848 | 20805 | 21762 | 17384 | 22719 | 23676 | 24633 | 25590 |
| 17385 | 18892 | 19849 | 20806 | 21763 | 17386 | 22720 | 23677 | 24634 | 25591 |
| 17387 | 18893 | 19850 | 20807 | 21764 | 17388 | 22721 | 23678 | 24635 | 25592 |
| 17389 | 18894 | 19851 | 20808 | 21765 | 17390 | 22722 | 23679 | 24636 | 25593 |
| 17391 | 18895 | 19852 | 20809 | 21766 | 17392 | 22723 | 23680 | 24637 | 25594 |
| 17393 | 18896 | 19853 | 20810 | 21767 | 17394 | 22724 | 23681 | 24638 | 25595 |
| 17395 | 18897 | 19854 | 20811 | 21768 | 17396 | 22725 | 23682 | 24639 | 25596 |
| 17397 | 18898 | 19855 | 20812 | 21769 | 17398 | 22726 | 23683 | 24640 | 25597 |
| 17399 | 18899 | 19856 | 20813 | 21770 | 17400 | 22727 | 23684 | 24641 | 25598 |
| 17401 | 18900 | 19857 | 20814 | 21771 | 17402 | 22728 | 23685 | 24642 | 25599 |
| 17403 | 18901 | 19858 | 20815 | 21772 | 17404 | 22729 | 23686 | 24643 | 25600 |
| 17405 | 18902 | 19859 | 20816 | 21773 | 17406 | 22730 | 23687 | 24644 | 25601 |
| 17407 | 18903 | 19860 | 20817 | 21774 | 17408 | 22731 | 23688 | 24645 | 25602 |
| 17409 | 18904 | 19861 | 20818 | 21775 | 17410 | 22732 | 23689 | 24646 | 25603 |
| 17411 | 18905 | 19862 | 20819 | 21776 | 17412 | 22733 | 23690 | 24647 | 25604 |
| 17413 | 18906 | 19863 | 20820 | 21777 | 17414 | 22734 | 23691 | 24648 | 25605 |
| 17415 | 18907 | 19864 | 20821 | 21778 | 17416 | 22735 | 23692 | 24649 | 25606 |
| 17417 | 18908 | 19865 | 20822 | 21779 | 17418 | 22736 | 23693 | 24650 | 25607 |
| 17419 | 18909 | 19866 | 20823 | 21780 | 17420 | 22737 | 23694 | 24651 | 25608 |
| 17421 | 18910 | 19867 | 20824 | 21781 | 17422 | 22738 | 23695 | 24652 | 25609 |
| 17423 | 18911 | 19868 | 20825 | 21782 | 17424 | 22739 | 23696 | 24653 | 25610 |
| 17425 | 18912 | 19869 | 20826 | 21783 | 17426 | 22740 | 23697 | 24654 | 25611 |
| 17427 | 18913 | 19870 | 20827 | 21784 | 17428 | 22741 | 23698 | 24655 | 25612 |
| 17429 | 18914 | 19871 | 20828 | 21785 | 17430 | 22742 | 23699 | 24656 | 25613 |
| 17431 | 18915 | 19872 | 20829 | 21786 | 17432 | 22743 | 23700 | 24657 | 25614 |
| 17433 | 18916 | 19873 | 20830 | 21787 | 17434 | 22744 | 23701 | 24658 | 25615 |
| 17435 | 18917 | 19874 | 20831 | 21788 | 17436 | 22745 | 23702 | 24659 | 25616 |
| 17437 | 18918 | 19875 | 20832 | 21789 | 17438 | 22746 | 23703 | 24660 | 25617 |
| 17439 | 18919 | 19876 | 20833 | 21790 | 17440 | 22747 | 23704 | 24661 | 25618 |
| 17441 | 18920 | 19877 | 20834 | 21791 | 17442 | 22748 | 23705 | 24662 | 25619 |
| 17443 | 18921 | 19878 | 20835 | 21792 | 17444 | 22749 | 23706 | 24663 | 25620 |
| 17445 | 18922 | 19879 | 20836 | 21793 | 17446 | 22750 | 23707 | 24664 | 25621 |
| 17447 | 18923 | 19880 | 20837 | 21794 | 17448 | 22751 | 23708 | 24665 | 25622 |
| 17449 | 18924 | 19881 | 20838 | 21795 | 17450 | 22752 | 23709 | 24666 | 25623 |
| 17451 | 18925 | 19882 | 20839 | 21796 | 17452 | 22753 | 23710 | 24667 | 25624 |
| 17453 | 18926 | 19883 | 20840 | 21797 | 17454 | 22754 | 23711 | 24668 | 25625 |
| 17455 | 18927 | 19884 | 20841 | 21798 | 17456 | 22755 | 23712 | 24669 | 25626 |
| 17457 | 18928 | 19885 | 20842 | 21799 | 17458 | 22756 | 23713 | 24670 | 25627 |
| 17459 | 18929 | 19886 | 20843 | 21800 | 17460 | 22757 | 23714 | 24671 | 25628 |
| 17461 | 18930 | 19887 | 20844 | 21801 | 17462 | 22758 | 23715 | 24672 | 25629 |
| 17463 | 18931 | 19888 | 20845 | 21802 | 17464 | 22759 | 23716 | 24673 | 25630 |
| 17465 | 18932 | 19889 | 20846 | 21803 | 17466 | 22760 | 23717 | 24674 | 25631 |
| 17467 | 18933 | 19890 | 20847 | 21804 | 17468 | 22761 | 23718 | 24675 | 25632 |
| 17469 | 18934 | 19891 | 20848 | 21805 | 17470 | 22762 | 23719 | 24676 | 25633 |
| 17471 | 18935 | 19892 | 20849 | 21806 | 17472 | 22763 | 23720 | 24677 | 25634 |
| 17473 | 18936 | 19893 | 20850 | 21807 | 17474 | 22764 | 23721 | 24678 | 25635 |
| 17475 | 18937 | 19894 | 20851 | 21808 | 17476 | 22765 | 23722 | 24679 | 25636 |
| 17477 | 18938 | 19895 | 20852 | 21809 | 17478 | 22766 | 23723 | 24680 | 25637 |
| 17479 | 18939 | 19896 | 20853 | 21810 | 17480 | 22767 | 23724 | 24681 | 25638 |
| 17481 | 18940 | 19897 | 20854 | 21811 | 17482 | 22768 | 23725 | 24682 | 25639 |
| 17483 | 18941 | 19898 | 20855 | 21812 | 17484 | 22769 | 23726 | 24683 | 25640 |
| 17485 | 18942 | 19899 | 20856 | 21813 | 17486 | 22770 | 23727 | 24684 | 25641 |
| 17487 | 18943 | 19900 | 20857 | 21814 | 17488 | 22771 | 23728 | 24685 | 25642 |
| 17489 | 18944 | 19901 | 20858 | 21815 | 17490 | 22772 | 23729 | 24686 | 25643 |
| 17491 | 18945 | 19902 | 20859 | 21816 | 17492 | 22773 | 23730 | 24687 | 25644 |
| 17493 | 18946 | 19903 | 20860 | 21817 | 17494 | 22774 | 23731 | 24688 | 25645 |
| 17495 | 18947 | 19904 | 20861 | 21818 | 17496 | 22775 | 23732 | 24689 | 25646 |
| 17497 | 18948 | 19905 | 20862 | 21819 | 17498 | 22776 | 23733 | 24690 | 25647 |
| 17499 | 18949 | 19906 | 20863 | 21820 | 17500 | 22777 | 23734 | 24691 | 25648 |
| 17501 | 18950 | 19907 | 20864 | 21821 | 17502 | 22778 | 23735 | 24692 | 25649 |
| 17503 | 18951 | 19908 | 20865 | 21822 | 17504 | 22779 | 23736 | 24693 | 25650 |
| 17505 | 18952 | 19909 | 20866 | 21823 | 17506 | 22780 | 23737 | 24694 | 25651 |
| 17507 | 18953 | 19910 | 20867 | 21824 | 17508 | 22781 | 23738 | 24695 | 25652 |
| 17509 | 18954 | 19911 | 20868 | 21825 | 17510 | 22782 | 23739 | 24696 | 25653 |
| 17511 | 18955 | 19912 | 20869 | 21826 | 17512 | 22783 | 23740 | 24697 | 25654 |
| 17513 | 18956 | 19913 | 20870 | 21827 | 17514 | 22784 | 23741 | 24698 | 25655 |
| 17515 | 18957 | 19914 | 20871 | 21828 | 17516 | 22785 | 23742 | 24699 | 25656 |
| 17517 | 18958 | 19915 | 20872 | 21829 | 17518 | 22786 | 23743 | 24700 | 25657 |
| 17519 | 18959 | 19916 | 20873 | 21830 | 17520 | 22787 | 23744 | 24701 | 25658 |
| 17521 | 18960 | 19917 | 20874 | 21831 | 17522 | 22788 | 23745 | 24702 | 25659 |
| 17523 | 18961 | 19918 | 20875 | 21832 | 17524 | 22789 | 23746 | 24703 | 25660 |
| 17525 | 18962 | 19919 | 20876 | 21833 | 17526 | 22790 | 23747 | 24704 | 25661 |
| 17527 | 18963 | 19920 | 20877 | 21834 | 17528 | 22791 | 23748 | 24705 | 25662 |
| 17529 | 18964 | 19921 | 20878 | 21835 | 17530 | 22792 | 23749 | 24706 | 25663 |
| 17531 | 18965 | 19922 | 20879 | 21836 | 17532 | 22793 | 23750 | 24707 | 25664 |
| 17533 | 18966 | 19923 | 20880 | 21837 | 17534 | 22794 | 23751 | 24708 | 25665 |
| 17535 | 18967 | 19924 | 20881 | 21838 | 17536 | 22795 | 23752 | 24709 | 25666 |
| 17537 | 18968 | 19925 | 20882 | 21839 | 17538 | 22796 | 23753 | 24710 | 25667 |
| 17539 | 18969 | 19926 | 20883 | 21840 | 17540 | 22797 | 23754 | 24711 | 25668 |
| 17541 | 18970 | 19927 | 20884 | 21841 | 17542 | 22798 | 23755 | 24712 | 25669 |
| 17543 | 18971 | 19928 | 20885 | 21842 | 17544 | 22799 | 23756 | 24713 | 25670 |
| 17545 | 18972 | 19929 | 20886 | 21843 | 17546 | 22800 | 23757 | 24714 | 25671 |
| 17547 | 18973 | 19930 | 20887 | 21844 | 17548 | 22801 | 23758 | 24715 | 25672 |
| 17549 | 18974 | 19931 | 20888 | 21845 | 17550 | 22802 | 23759 | 24716 | 25673 |
| 17551 | 18975 | 19932 | 20889 | 21846 | 17552 | 22803 | 23760 | 24717 | 25674 |
| 17553 | 18976 | 19933 | 20890 | 21847 | 17554 | 22804 | 23761 | 24718 | 25675 |
| 17555 | 18977 | 19934 | 20891 | 21848 | 17556 | 22805 | 23762 | 24719 | 25676 |
| 17557 | 18978 | 19935 | 20892 | 21849 | 17558 | 22806 | 23763 | 24720 | 25677 |
| 17559 | 18979 | 19936 | 20893 | 21850 | 17560 | 22807 | 23764 | 24721 | 25678 |
| 17561 | 18980 | 19937 | 20894 | 21851 | 17562 | 22808 | 23765 | 24722 | 25679 |
| 17563 | 18981 | 19938 | 20895 | 21852 | 17564 | 22809 | 23766 | 24723 | 25680 |
| 17565 | 18982 | 19939 | 20896 | 21853 | 17566 | 22810 | 23767 | 24724 | 25681 |
| 17567 | 18983 | 19940 | 20897 | 21854 | 17568 | 22811 | 23768 | 24725 | 25682 |
| 17569 | 18984 | 19941 | 20898 | 21855 | 17570 | 22812 | 23769 | 24726 | 25683 |
| 17571 | 18985 | 19942 | 20899 | 21856 | 17572 | 22813 | 23770 | 24727 | 25684 |
| 17573 | 18986 | 19943 | 20900 | 21857 | 17574 | 22814 | 23771 | 24728 | 25685 |
| 17575 | 18987 | 19944 | 20901 | 21858 | 17576 | 22815 | 23772 | 24729 | 25686 |
| 17577 | 18988 | 19945 | 20902 | 21859 | 17578 | 22816 | 23773 | 24730 | 25687 |
| 17579 | 18989 | 19946 | 20903 | 21860 | 17580 | 22817 | 23774 | 24731 | 25688 |
| 17581 | 18990 | 19947 | 20904 | 21861 | 17582 | 22818 | 23775 | 24732 | 25689 |
| 17583 | 18991 | 19948 | 20905 | 21862 | 17584 | 22819 | 23776 | 24733 | 25690 |
| 17585 | 18992 | 19949 | 20906 | 21863 | 17586 | 22820 | 23777 | 24734 | 25691 |
| 17587 | 18993 | 19950 | 20907 | 21864 | 17588 | 22821 | 23778 | 24735 | 25692 |
| 17589 | 18994 | 19951 | 20908 | 21865 | 17590 | 22822 | 23779 | 24736 | 25693 |
| 17591 | 18995 | 19952 | 20909 | 21866 | 17592 | 22823 | 23780 | 24737 | 25694 |
| 17593 | 18996 | 19953 | 20910 | 21867 | 17594 | 22824 | 23781 | 24738 | 25695 |
| 17595 | 18997 | 19954 | 20911 | 21868 | 17596 | 22825 | 23782 | 24739 | 25696 |
| 17597 | 18998 | 19955 | 20912 | 21869 | 17598 | 22826 | 23783 | 24740 | 25697 |
| 17599 | 18999 | 19956 | 20913 | 21870 | 17600 | 22827 | 23784 | 24741 | 25698 |
| 17601 | 19000 | 19957 | 20914 | 21871 | 17602 | 22828 | 23785 | 24742 | 25699 |
| 17603 | 19001 | 19958 | 20915 | 21872 | 17604 | 22829 | 23786 | 24743 | 25700 |
| 17605 | 19002 | 19959 | 20916 | 21873 | 17606 | 22830 | 23787 | 24744 | 25701 |
| 17607 | 19003 | 19960 | 20917 | 21874 | 17608 | 22831 | 23788 | 24745 | 25702 |
| 17609 | 19004 | 19961 | 20918 | 21875 | 17610 | 22832 | 23789 | 24746 | 25703 |
| 17611 | 19005 | 19962 | 20919 | 21876 | 17612 | 22833 | 23790 | 24747 | 25704 |
| 17613 | 19006 | 19963 | 20920 | 21877 | 17614 | 22834 | 23791 | 24748 | 25705 |
| 17615 | 19007 | 19964 | 20921 | 21878 | 17616 | 22835 | 23792 | 24749 | 25706 |
| 17617 | 19008 | 19965 | 20922 | 21879 | 17618 | 22836 | 23793 | 24750 | 25707 |
| 17619 | 19009 | 19966 | 20923 | 21880 | 17620 | 22837 | 23794 | 24751 | 25708 |
| 17621 | 19010 | 19967 | 20924 | 21881 | 17622 | 22838 | 23795 | 24752 | 25709 |
| 17623 | 19011 | 19968 | 20925 | 21882 | 17624 | 22839 | 23796 | 24753 | 25710 |
| 17625 | 19012 | 19969 | 20926 | 21883 | 17626 | 22840 | 23797 | 24754 | 25711 |
| 17627 | 19013 | 19970 | 20927 | 21884 | 17628 | 22841 | 23798 | 24755 | 25712 |
| 17629 | 19014 | 19971 | 20928 | 21885 | 17630 | 22842 | 23799 | 24756 | 25713 |
| 17631 | 19015 | 19972 | 20929 | 21886 | 17632 | 22843 | 23800 | 24757 | 25714 |
| 17633 | 19016 | 19973 | 20930 | 21887 | 17634 | 22844 | 23801 | 24758 | 25715 |
| 17635 | 19017 | 19974 | 20931 | 21888 | 17636 | 22845 | 23802 | 24759 | 25716 |
| 17637 | 19018 | 19975 | 20932 | 21889 | 17638 | 22846 | 23803 | 24760 | 25717 |
| 17639 | 19019 | 19976 | 20933 | 21890 | 17640 | 22847 | 23804 | 24761 | 25718 |
| 17641 | 19020 | 19977 | 20934 | 21891 | 17642 | 22848 | 23805 | 24762 | 25719 |
| 17643 | 19021 | 19978 | 20935 | 21892 | 17644 | 22849 | 23806 | 24763 | 25720 |
| 17645 | 19022 | 19979 | 20936 | 21893 | 17646 | 22850 | 23807 | 24764 | 25721 |
| 17647 | 19023 | 19980 | 20937 | 21894 | 17648 | 22851 | 23808 | 24765 | 25722 |
| 17649 | 19024 | 19981 | 20938 | 21895 | 17650 | 22852 | 23809 | 24766 | 25723 |
| 17651 | 19025 | 19982 | 20939 | 21896 | 17652 | 22853 | 23810 | 24767 | 25724 |
| 17653 | 19026 | 19983 | 20940 | 21897 | 17654 | 22854 | 23811 | 24768 | 25725 |
| 17655 | 19027 | 19984 | 20941 | 21898 | 17656 | 22855 | 23812 | 24769 | 25726 |
| 17657 | 19028 | 19985 | 20942 | 21899 | 17658 | 22856 | 23813 | 24770 | 25727 |
| 17659 | 19029 | 19986 | 20943 | 21900 | 17660 | 22857 | 23814 | 24771 | 25728 |
| 17661 | 19030 | 19987 | 20944 | 21901 | 17662 | 22858 | 23815 | 24772 | 25729 |
| 17663 | 19031 | 19988 | 20945 | 21902 | 17664 | 22859 | 23816 | 24773 | 25730 |
| 17665 | 19032 | 19989 | 20946 | 21903 | 17666 | 22860 | 23817 | 24774 | 25731 |
| 17667 | 19033 | 19990 | 20947 | 21904 | 17668 | 22861 | 23818 | 24775 | 25732 |
| 17669 | 19034 | 19991 | 20948 | 21905 | 17670 | 22862 | 23819 | 24776 | 25733 |
| 17671 | 19035 | 19992 | 20949 | 21906 | 17672 | 22863 | 23820 | 24777 | 25734 |
| 17673 | 19036 | 19993 | 20950 | 21907 | 17674 | 22864 | 23821 | 24778 | 25735 |
| 17675 | 19037 | 19994 | 20951 | 21908 | 17676 | 22865 | 23822 | 24779 | 25736 |
| 17677 | 19038 | 19995 | 20952 | 21909 | 17678 | 22866 | 23823 | 24780 | 25737 |
| 17679 | 19039 | 19996 | 20953 | 21910 | 17680 | 22867 | 23824 | 24781 | 25738 |
| 17681 | 19040 | 19997 | 20954 | 21911 | 17682 | 22868 | 23825 | 24782 | 25739 |
| 17683 | 19041 | 19998 | 20955 | 21912 | 17684 | 22869 | 23826 | 24783 | 25740 |
| 17685 | 19042 | 19999 | 20956 | 21913 | 17686 | 22870 | 23827 | 24784 | 25741 |
| 17687 | 19043 | 20000 | 20957 | 21914 | 17688 | 22871 | 23828 | 24785 | 25742 |
| 17689 | 19044 | 20001 | 20958 | 21915 | 17690 | 22872 | 23829 | 24786 | 25743 |
| 17691 | 19045 | 20002 | 20959 | 21916 | 17692 | 22873 | 23830 | 24787 | 25744 |
| 17693 | 19046 | 20003 | 20960 | 21917 | 17694 | 22874 | 23831 | 24788 | 25745 |
| 17695 | 19047 | 20004 | 20961 | 21918 | 17696 | 22875 | 23832 | 24789 | 25746 |
| 17697 | 19048 | 20005 | 20962 | 21919 | 17698 | 22876 | 23833 | 24790 | 25747 |
| 17699 | 19049 | 20006 | 20963 | 21920 | 17700 | 22877 | 23834 | 24791 | 25748 |
| 17701 | 19050 | 20007 | 20964 | 21921 | 17702 | 22878 | 23835 | 24792 | 25749 |
| 17703 | 19051 | 20008 | 20965 | 21922 | 17704 | 22879 | 23836 | 24793 | 25750 |
| 17705 | 19052 | 20009 | 20966 | 21923 | 17706 | 22880 | 23837 | 24794 | 25751 |
| 17707 | 19053 | 20010 | 20967 | 21924 | 17708 | 22881 | 23838 | 24795 | 25752 |
| 17709 | 19054 | 20011 | 20968 | 21925 | 17710 | 22882 | 23839 | 24796 | 25753 |
| 17711 | 19055 | 20012 | 20969 | 21926 | 17712 | 22883 | 23840 | 24797 | 25754 |
| 17713 | 19056 | 20013 | 20970 | 21927 | 17714 | 22884 | 23841 | 24798 | 25755 |
| 17715 | 19057 | 20014 | 20971 | 21928 | 17716 | 22885 | 23842 | 24799 | 25756 |
| 17717 | 19058 | 20015 | 20972 | 21929 | 17718 | 22886 | 23843 | 24800 | 25757 |
| 17719 | 19059 | 20016 | 20973 | 21930 | 17720 | 22887 | 23844 | 24801 | 25758 |
| 17721 | 19060 | 20017 | 20974 | 21931 | 17722 | 22888 | 23845 | 24802 | 25759 |
| 17723 | 19061 | 20018 | 20975 | 21932 | 17724 | 22889 | 23846 | 24803 | 25760 |
| 17725 | 19062 | 20019 | 20976 | 21933 | 17726 | 22890 | 23847 | 24804 | 25761 |
| 17727 | 19063 | 20020 | 20977 | 21934 | 17728 | 22891 | 23848 | 24805 | 25762 |
| 17729 | 19064 | 20021 | 20978 | 21935 | 17730 | 22892 | 23849 | 24806 | 25763 |
| 17731 | 19065 | 20022 | 20979 | 21936 | 17732 | 22893 | 23850 | 24807 | 25764 |
| 17733 | 19066 | 20023 | 20980 | 21937 | 17734 | 22894 | 23851 | 24808 | 25765 |
| 17735 | 19067 | 20024 | 20981 | 21938 | 17736 | 22895 | 23852 | 24809 | 25766 |
| 17737 | 19068 | 20025 | 20982 | 21939 | 17738 | 22896 | 23853 | 24810 | 25767 |
| 17739 | 19069 | 20026 | 20983 | 21940 | 17740 | 22897 | 23854 | 24811 | 25768 |
| 17741 | 19070 | 20027 | 20984 | 21941 | 17742 | 22898 | 23855 | 24812 | 25769 |
| 17743 | 19071 | 20028 | 20985 | 21942 | 17744 | 22899 | 23856 | 24813 | 25770 |
| 17745 | 19072 | 20029 | 20986 | 21943 | 17746 | 22900 | 23857 | 24814 | 25771 |
| 17747 | 19073 | 20030 | 20987 | 21944 | 17748 | 22901 | 23858 | 24815 | 25772 |
| 17749 | 19074 | 20031 | 20988 | 21945 | 17750 | 22902 | 23859 | 24816 | 25773 |
| 17751 | 19075 | 20032 | 20989 | 21946 | 17752 | 22903 | 23860 | 24817 | 25774 |
| 17753 | 19076 | 20033 | 20990 | 21947 | 17754 | 22904 | 23861 | 24818 | 25775 |
| 17755 | 19077 | 20034 | 20991 | 21948 | 17756 | 22905 | 23862 | 24819 | 25776 |
| 17757 | 19078 | 20035 | 20992 | 21949 | 17758 | 22906 | 23863 | 24820 | 25777 |
| 17759 | 19079 | 20036 | 20993 | 21950 | 17760 | 22907 | 23864 | 24821 | 25778 |
| 17761 | 19080 | 20037 | 20994 | 21951 | 17762 | 22908 | 23865 | 24822 | 25779 |
| 17763 | 19081 | 20038 | 20995 | 21952 | 17764 | 22909 | 23866 | 24823 | 25780 |
| 17765 | 19082 | 20039 | 20996 | 21953 | 17766 | 22910 | 23867 | 24824 | 25781 |
| 17767 | 19083 | 20040 | 20997 | 21954 | 17768 | 22911 | 23868 | 24825 | 25782 |
| 17769 | 19084 | 20041 | 20998 | 21955 | 17770 | 22912 | 23869 | 24826 | 25783 |
| 17771 | 19085 | 20042 | 20999 | 21956 | 17772 | 22913 | 23870 | 24827 | 25784 |
| 17773 | 19086 | 20043 | 21000 | 21957 | 17774 | 22914 | 23871 | 24828 | 25785 |
| 17775 | 19087 | 20044 | 21001 | 21958 | 17776 | 22915 | 23872 | 24829 | 25786 |
| 17777 | 19088 | 20045 | 21002 | 21959 | 17778 | 22916 | 23873 | 24830 | 25787 |
| 17779 | 19089 | 20046 | 21003 | 21960 | 17780 | 22917 | 23874 | 24831 | 25788 |
| 17781 | 19090 | 20047 | 21004 | 21961 | 17782 | 22918 | 23875 | 24832 | 25789 |
| 17783 | 19091 | 20048 | 21005 | 21962 | 17784 | 22919 | 23876 | 24833 | 25790 |
| 17785 | 19092 | 20049 | 21006 | 21963 | 17786 | 22920 | 23877 | 24834 | 25791 |
| 17787 | 19093 | 20050 | 21007 | 21964 | 17788 | 22921 | 23878 | 24835 | 25792 |
| 17789 | 19094 | 20051 | 21008 | 21965 | 17790 | 22922 | 23879 | 24836 | 25793 |
| 17791 | 19095 | 20052 | 21009 | 21966 | 17792 | 22923 | 23880 | 24837 | 25794 |
| 17793 | 19096 | 20053 | 21010 | 21967 | 17794 | 22924 | 23881 | 24838 | 25795 |
| 17795 | 19097 | 20054 | 21011 | 21968 | 17796 | 22925 | 23882 | 24839 | 25796 |
| 17797 | 19098 | 20055 | 21012 | 21969 | 17798 | 22926 | 23883 | 24840 | 25797 |
| 17799 | 19099 | 20056 | 21013 | 21970 | 17800 | 22927 | 23884 | 24841 | 25798 |
| 17801 | 19100 | 20057 | 21014 | 21971 | 17802 | 22928 | 23885 | 24842 | 25799 |
| 17803 | 19101 | 20058 | 21015 | 21972 | 17804 | 22929 | 23886 | 24843 | 25800 |
| 17805 | 19102 | 20059 | 21016 | 21973 | 17806 | 22930 | 23887 | 24844 | 25801 |
| 17807 | 19103 | 20060 | 21017 | 21974 | 17808 | 22931 | 23888 | 24845 | 25802 |
| 17809 | 19104 | 20061 | 21018 | 21975 | 17810 | 22932 | 23889 | 24846 | 25803 |
| 17811 | 19105 | 20062 | 21019 | 21976 | 17812 | 22933 | 23890 | 24847 | 25804 |
| 17813 | 19106 | 20063 | 21020 | 21977 | 17814 | 22934 | 23891 | 24848 | 25805 |
| 17815 | 19107 | 20064 | 21021 | 21978 | 17816 | 22935 | 23892 | 24849 | 25806 |
| 17817 | 19108 | 20065 | 21022 | 21979 | 17818 | 22936 | 23893 | 24850 | 25807 |
| 17819 | 19109 | 20066 | 21023 | 21980 | 17820 | 22937 | 23894 | 24851 | 25808 |
| 17821 | 19110 | 20067 | 21024 | 21981 | 17822 | 22938 | 23895 | 24852 | 25809 |
| 17823 | 19111 | 20068 | 21025 | 21982 | 17824 | 22939 | 23896 | 24853 | 25810 |
| 17825 | 19112 | 20069 | 21026 | 21983 | 17826 | 22940 | 23897 | 24854 | 25811 |
| 17827 | 19113 | 20070 | 21027 | 21984 | 17828 | 22941 | 23898 | 24855 | 25812 |
| 17829 | 19114 | 20071 | 21028 | 21985 | 17830 | 22942 | 23899 | 24856 | 25813 |
| 17831 | 19115 | 20072 | 21029 | 21986 | 17832 | 22943 | 23900 | 24857 | 25814 |
| 17833 | 19116 | 20073 | 21030 | 21987 | 17834 | 22944 | 23901 | 24858 | 25815 |
| 17835 | 19117 | 20074 | 21031 | 21988 | 17836 | 22945 | 23902 | 24859 | 25816 |
| 17837 | 19118 | 20075 | 21032 | 21989 | 17838 | 22946 | 23903 | 24860 | 25817 |
| 17839 | 19119 | 20076 | 21033 | 21990 | 17840 | 22947 | 23904 | 24861 | 25818 |
| 17841 | 19120 | 20077 | 21034 | 21991 | 17842 | 22948 | 23905 | 24862 | 25819 |
| 17843 | 19121 | 20078 | 21035 | 21992 | 17844 | 22949 | 23906 | 24863 | 25820 |
| 17845 | 19122 | 20079 | 21036 | 21993 | 17846 | 22950 | 23907 | 24864 | 25821 |
| 17847 | 19123 | 20080 | 21037 | 21994 | 17848 | 22951 | 23908 | 24865 | 25822 |
| 17849 | 19124 | 20081 | 21038 | 21995 | 17850 | 22952 | 23909 | 24866 | 25823 |
| 17851 | 19125 | 20082 | 21039 | 21996 | 17852 | 22953 | 23910 | 24867 | 25824 |
| 17853 | 19126 | 20083 | 21040 | 21997 | 17854 | 22954 | 23911 | 24868 | 25825 |
| 17855 | 19127 | 20084 | 21041 | 21998 | 17856 | 22955 | 23912 | 24869 | 25826 |
| 17857 | 19128 | 20085 | 21042 | 21999 | 17858 | 22956 | 23913 | 24870 | 25827 |
| 17859 | 19129 | 20086 | 21043 | 22000 | 17860 | 22957 | 23914 | 24871 | 25828 |
| 17861 | 19130 | 20087 | 21044 | 22001 | 17862 | 22958 | 23915 | 24872 | 25829 |
| 17863 | 19131 | 20088 | 21045 | 22002 | 17864 | 22959 | 23916 | 24873 | 25830 |
| 17865 | 19132 | 20089 | 21046 | 22003 | 17866 | 22960 | 23917 | 24874 | 25831 |
| 17867 | 19133 | 20090 | 21047 | 22004 | 17868 | 22961 | 23918 | 24875 | 25832 |
| 17869 | 19134 | 20091 | 21048 | 22005 | 17870 | 22962 | 23919 | 24876 | 25833 |
| 17871 | 19135 | 20092 | 21049 | 22006 | 17872 | 22963 | 23920 | 24877 | 25834 |
| 17873 | 19136 | 20093 | 21050 | 22007 | 17874 | 22964 | 23921 | 24878 | 25835 |
| 17875 | 19137 | 20094 | 21051 | 22008 | 17876 | 22965 | 23922 | 24879 | 25836 |
| 17877 | 19138 | 20095 | 21052 | 22009 | 17878 | 22966 | 23923 | 24880 | 25837 |
| 17879 | 19139 | 20096 | 21053 | 22010 | 17880 | 22967 | 23924 | 24881 | 25838 |
| 17881 | 19140 | 20097 | 21054 | 22011 | 17882 | 22968 | 23925 | 24882 | 25839 |
| 17883 | 19141 | 20098 | 21055 | 22012 | 17884 | 22969 | 23926 | 24883 | 25840 |
| 17885 | 19142 | 20099 | 21056 | 22013 | 17886 | 22970 | 23927 | 24884 | 25841 |
| 17887 | 19143 | 20100 | 21057 | 22014 | 17888 | 22971 | 23928 | 24885 | 25842 |
| 17889 | 19144 | 20101 | 21058 | 22015 | 17890 | 22972 | 23929 | 24886 | 25843 |
| 17891 | 19145 | 20102 | 21059 | 22016 | 17892 | 22973 | 23930 | 24887 | 25844 |
| 17893 | 19146 | 20103 | 21060 | 22017 | 17894 | 22974 | 23931 | 24888 | 25845 |
| 17895 | 19147 | 20104 | 21061 | 22018 | 17896 | 22975 | 23932 | 24889 | 25846 |
| 17897 | 19148 | 20105 | 21062 | 22019 | 17898 | 22976 | 23933 | 24890 | 25847 |
| 17899 | 19149 | 20106 | 21063 | 22020 | 17900 | 22977 | 23934 | 24891 | 25848 |
| 17901 | 19150 | 20107 | 21064 | 22021 | 17902 | 22978 | 23935 | 24892 | 25849 |
| 17903 | 19151 | 20108 | 21065 | 22022 | 17904 | 22979 | 23936 | 24893 | 25850 |
| 17905 | 19152 | 20109 | 21066 | 22023 | 17906 | 22980 | 23937 | 24894 | 25851 |
| 17907 | 19153 | 20110 | 21067 | 22024 | 17908 | 22981 | 23938 | 24895 | 25852 |
| 17909 | 19154 | 20111 | 21068 | 22025 | 17910 | 22982 | 23939 | 24896 | 25853 |
| 17911 | 19155 | 20112 | 21069 | 22026 | 17912 | 22983 | 23940 | 24897 | 25854 |
| 17913 | 19156 | 20113 | 21070 | 22027 | 17914 | 22984 | 23941 | 24898 | 25855 |
| 17915 | 19157 | 20114 | 21071 | 22028 | 17916 | 22985 | 23942 | 24899 | 25856 |
| 17917 | 19158 | 20115 | 21072 | 22029 | 17918 | 22986 | 23943 | 24900 | 25857 |
| 17919 | 19159 | 20116 | 21073 | 22030 | 17920 | 22987 | 23944 | 24901 | 25858 |
| 17921 | 19160 | 20117 | 21074 | 22031 | 17922 | 22988 | 23945 | 24902 | 25859 |
| 17923 | 19161 | 20118 | 21075 | 22032 | 17924 | 22989 | 23946 | 24903 | 25860 |
| 17925 | 19162 | 20119 | 21076 | 22033 | 17926 | 22990 | 23947 | 24904 | 25861 |
| 17927 | 19163 | 20120 | 21077 | 22034 | 17928 | 22991 | 23948 | 24905 | 25862 |
| 17929 | 19164 | 20121 | 21078 | 22035 | 17930 | 22992 | 23949 | 24906 | 25863 |
| 17931 | 19165 | 20122 | 21079 | 22036 | 17932 | 22993 | 23950 | 24907 | 25864 |
| 17933 | 19166 | 20123 | 21080 | 22037 | 17934 | 22994 | 23951 | 24908 | 25865 |
| 17935 | 19167 | 20124 | 21081 | 22038 | 17936 | 22995 | 23952 | 24909 | 25866 |
| 17937 | 19168 | 20125 | 21082 | 22039 | 17938 | 22996 | 23953 | 24910 | 25867 |
| 17939 | 19169 | 20126 | 21083 | 22040 | 17940 | 22997 | 23954 | 24911 | 25868 |
| 17941 | 19170 | 20127 | 21084 | 22041 | 17942 | 22998 | 23955 | 24912 | 25869 |
| 17943 | 19171 | 20128 | 21085 | 22042 | 17944 | 22999 | 23956 | 24913 | 25870 |
| 17945 | 19172 | 20129 | 21086 | 22043 | 17946 | 23000 | 23957 | 24914 | 25871 |
| 17947 | 19173 | 20130 | 21087 | 22044 | 17948 | 23001 | 23958 | 24915 | 25872 |
| 17949 | 19174 | 20131 | 21088 | 22045 | 17950 | 23002 | 23959 | 24916 | 25873 |
| 17951 | 19175 | 20132 | 21089 | 22046 | 17952 | 23003 | 23960 | 24917 | 25874 |
| 17953 | 19176 | 20133 | 21090 | 22047 | 17954 | 23004 | 23961 | 24918 | 25875 |
| 17955 | 19177 | 20134 | 21091 | 22048 | 17956 | 23005 | 23962 | 24919 | 25876 |
| 17957 | 19178 | 20135 | 21092 | 22049 | 17958 | 23006 | 23963 | 24920 | 25877 |
| 17959 | 19179 | 20136 | 21093 | 22050 | 17960 | 23007 | 23964 | 24921 | 25878 |
| 17961 | 19180 | 20137 | 21094 | 22051 | 17962 | 23008 | 23965 | 24922 | 25879 |
| 17963 | 19181 | 20138 | 21095 | 22052 | 17964 | 23009 | 23966 | 24923 | 25880 |
| 17965 | 19182 | 20139 | 21096 | 22053 | 17966 | 23010 | 23967 | 24924 | 25881 |
| 17967 | 19183 | 20140 | 21097 | 22054 | 17968 | 23011 | 23968 | 24925 | 25882 |
| 17969 | 19184 | 20141 | 21098 | 22055 | 17970 | 23012 | 23969 | 24926 | 25883 |
| 17971 | 19185 | 20142 | 21099 | 22056 | 17972 | 23013 | 23970 | 24927 | 25884 |
| 17973 | 19186 | 20143 | 21100 | 22057 | 17974 | 23014 | 23971 | 24928 | 25885 |
| 17975 | 19187 | 20144 | 21101 | 22058 | 17976 | 23015 | 23972 | 24929 | 25886 |
| 17977 | 19188 | 20145 | 21102 | 22059 | 17978 | 23016 | 23973 | 24930 | 25887 |
| 17979 | 19189 | 20146 | 21103 | 22060 | 17980 | 23017 | 23974 | 24931 | 25888 |
| 17981 | 19190 | 20147 | 21104 | 22061 | 17982 | 23018 | 23975 | 24932 | 25889 |
| 17983 | 19191 | 20148 | 21105 | 22062 | 17984 | 23019 | 23976 | 24933 | 25890 |
| 17985 | 19192 | 20149 | 21106 | 22063 | 17986 | 23020 | 23977 | 24934 | 25891 |
| 17987 | 19193 | 20150 | 21107 | 22064 | 17988 | 23021 | 23978 | 24935 | 25892 |
| 17989 | 19194 | 20151 | 21108 | 22065 | 17990 | 23022 | 23979 | 24936 | 25893 |
| 17991 | 19195 | 20152 | 21109 | 22066 | 17992 | 23023 | 23980 | 24937 | 25894 |
| 17993 | 19196 | 20153 | 21110 | 22067 | 17994 | 23024 | 23981 | 24938 | 25895 |
| 17995 | 19197 | 20154 | 21111 | 22068 | 17996 | 23025 | 23982 | 24939 | 25896 |
| 17997 | 19198 | 20155 | 21112 | 22069 | 17998 | 23026 | 23983 | 24940 | 25897 |
| 17999 | 19199 | 20156 | 21113 | 22070 | 18000 | 23027 | 23984 | 24941 | 25898 |
| 18001 | 19200 | 20157 | 21114 | 22071 | 18002 | 23028 | 23985 | 24942 | 25899 |
| 18003 | 19201 | 20158 | 21115 | 22072 | 18004 | 23029 | 23986 | 24943 | 25900 |
| 18005 | 19202 | 20159 | 21116 | 22073 | 18006 | 23030 | 23987 | 24944 | 25901 |
| 18007 | 19203 | 20160 | 21117 | 22074 | 18008 | 23031 | 23988 | 24945 | 25902 |
| 18009 | 19204 | 20161 | 21118 | 22075 | 18010 | 23032 | 23989 | 24946 | 25903 |
| 18011 | 19205 | 20162 | 21119 | 22076 | 18012 | 23033 | 23990 | 24947 | 25904 |
| 18013 | 19206 | 20163 | 21120 | 22077 | 18014 | 23034 | 23991 | 24948 | 25905 |
| 18015 | 19207 | 20164 | 21121 | 22078 | 18016 | 23035 | 23992 | 24949 | 25906 |
| 18017 | 19208 | 20165 | 21122 | 22079 | 18018 | 23036 | 23993 | 24950 | 25907 |
| 18019 | 19209 | 20166 | 21123 | 22080 | 18020 | 23037 | 23994 | 24951 | 25908 |
| 18021 | 19210 | 20167 | 21124 | 22081 | 18022 | 23038 | 23995 | 24952 | 25909 |
| 18023 | 19211 | 20168 | 21125 | 22082 | 18024 | 23039 | 23996 | 24953 | 25910 |
| 18025 | 19212 | 20169 | 21126 | 22083 | 18026 | 23040 | 23997 | 24954 | 25911 |
| 18027 | 19213 | 20170 | 21127 | 22084 | 18028 | 23041 | 23998 | 24955 | 25912 |
| 18029 | 19214 | 20171 | 21128 | 22085 | 18030 | 23042 | 23999 | 24956 | 25913 |
| 18031 | 19215 | 20172 | 21129 | 22086 | 18032 | 23043 | 24000 | 24957 | 25914 |
| 18033 | 19216 | 20173 | 21130 | 22087 | 18034 | 23044 | 24001 | 24958 | 25915 |
| 18035 | 19217 | 20174 | 21131 | 22088 | 18036 | 23045 | 24002 | 24959 | 25916 |
| 18037 | 19218 | 20175 | 21132 | 22089 | 18038 | 23046 | 24003 | 24960 | 25917 |
| 18039 | 19219 | 20176 | 21133 | 22090 | 18040 | 23047 | 24004 | 24961 | 25918 |
| 18041 | 19220 | 20177 | 21134 | 22091 | 18042 | 23048 | 24005 | 24962 | 25919 |
| 18043 | 19221 | 20178 | 21135 | 22092 | 18044 | 23049 | 24006 | 24963 | 25920 |
| 18045 | 19222 | 20179 | 21136 | 22093 | 18046 | 23050 | 24007 | 24964 | 25921 |
| 18047 | 19223 | 20180 | 21137 | 22094 | 18048 | 23051 | 24008 | 24965 | 25922 |
| 18049 | 19224 | 20181 | 21138 | 22095 | 18050 | 23052 | 24009 | 24966 | 25923 |
| 18051 | 19225 | 20182 | 21139 | 22096 | 18052 | 23053 | 24010 | 24967 | 25924 |
| 18053 | 19226 | 20183 | 21140 | 22097 | 18054 | 23054 | 24011 | 24968 | 25925 |
| 18055 | 19227 | 20184 | 21141 | 22098 | 18056 | 23055 | 24012 | 24969 | 25926 |
| 18057 | 19228 | 20185 | 21142 | 22099 | 18058 | 23056 | 24013 | 24970 | 25927 |
| 18059 | 19229 | 20186 | 21143 | 22100 | 18060 | 23057 | 24014 | 24971 | 25928 |
| 18061 | 19230 | 20187 | 21144 | 22101 | 18062 | 23058 | 24015 | 24972 | 25929 |
| 18063 | 19231 | 20188 | 21145 | 22102 | 18064 | 23059 | 24016 | 24973 | 25930 |
| 18065 | 19232 | 20189 | 21146 | 22103 | 18066 | 23060 | 24017 | 24974 | 25931 |
| 18067 | 19233 | 20190 | 21147 | 22104 | 18068 | 23061 | 24018 | 24975 | 25932 |
| 18069 | 19234 | 20191 | 21148 | 22105 | 18070 | 23062 | 24019 | 24976 | 25933 |
| 18071 | 19235 | 20192 | 21149 | 22106 | 18072 | 23063 | 24020 | 24977 | 25934 |
| 18073 | 19236 | 20193 | 21150 | 22107 | 18074 | 23064 | 24021 | 24978 | 25935 |
| 18075 | 19237 | 20194 | 21151 | 22108 | 18076 | 23065 | 24022 | 24979 | 25936 |
| 18077 | 19238 | 20195 | 21152 | 22109 | 18078 | 23066 | 24023 | 24980 | 25937 |
| 18079 | 19239 | 20196 | 21153 | 22110 | 18080 | 23067 | 24024 | 24981 | 25938 |
| 18081 | 19240 | 20197 | 21154 | 22111 | 18082 | 23068 | 24025 | 24982 | 25939 |
| 18083 | 19241 | 20198 | 21155 | 22112 | 18084 | 23069 | 24026 | 24983 | 25940 |
| 18085 | 19242 | 20199 | 21156 | 22113 | 18086 | 23070 | 24027 | 24984 | 25941 |
| 18087 | 19243 | 20200 | 21157 | 22114 | 18088 | 23071 | 24028 | 24985 | 25942 |
| 18089 | 19244 | 20201 | 21158 | 22115 | 18090 | 23072 | 24029 | 24986 | 25943 |
| 18091 | 19245 | 20202 | 21159 | 22116 | 18092 | 23073 | 24030 | 24987 | 25944 |
| 18093 | 19246 | 20203 | 21160 | 22117 | 18094 | 23074 | 24031 | 24988 | 25945 |
| 18095 | 19247 | 20204 | 21161 | 22118 | 18096 | 23075 | 24032 | 24989 | 25946 |
| 18097 | 19248 | 20205 | 21162 | 22119 | 18098 | 23076 | 24033 | 24990 | 25947 |
| 18099 | 19249 | 20206 | 21163 | 22120 | 18100 | 23077 | 24034 | 24991 | 25948 |
| 18101 | 19250 | 20207 | 21164 | 22121 | 18102 | 23078 | 24035 | 24992 | 25949 |
| 18103 | 19251 | 20208 | 21165 | 22122 | 18104 | 23079 | 24036 | 24993 | 25950 |
| 18105 | 19252 | 20209 | 21166 | 22123 | 18106 | 23080 | 24037 | 24994 | 25951 |
| 18107 | 19253 | 20210 | 21167 | 22124 | 18108 | 23081 | 24038 | 24995 | 25952 |
| 18109 | 19254 | 20211 | 21168 | 22125 | 18110 | 23082 | 24039 | 24996 | 25953 |
| 18111 | 19255 | 20212 | 21169 | 22126 | 18112 | 23083 | 24040 | 24997 | 25954 |
| 18113 | 19256 | 20213 | 21170 | 22127 | 18114 | 23084 | 24041 | 24998 | 25955 |
| 18115 | 19257 | 20214 | 21171 | 22128 | 18116 | 23085 | 24042 | 24999 | 25956 |
| 18117 | 19258 | 20215 | 21172 | 22129 | 18118 | 23086 | 24043 | 25000 | 25957 |
| 18119 | 19259 | 20216 | 21173 | 22130 | 18120 | 23087 | 24044 | 25001 | 25958 |
| 18121 | 19260 | 20217 | 21174 | 22131 | 18122 | 23088 | 24045 | 25002 | 25959 |
| 18123 | 19261 | 20218 | 21175 | 22132 | 18124 | 23089 | 24046 | 25003 | 25960 |
| 18125 | 19262 | 20219 | 21176 | 22133 | 18126 | 23090 | 24047 | 25004 | 25961 |
| 18127 | 19263 | 20220 | 21177 | 22134 | 18128 | 23091 | 24048 | 25005 | 25962 |
| 18129 | 19264 | 20221 | 21178 | 22135 | 18130 | 23092 | 24049 | 25006 | 25963 |
| 18131 | 19265 | 20222 | 21179 | 22136 | 18132 | 23093 | 24050 | 25007 | 25964 |
| 18133 | 19266 | 20223 | 21180 | 22137 | 18134 | 23094 | 24051 | 25008 | 25965 |
| 18135 | 19267 | 20224 | 21181 | 22138 | 18136 | 23095 | 24052 | 25009 | 25966 |
| 18137 | 19268 | 20225 | 21182 | 22139 | 18138 | 23096 | 24053 | 25010 | 25967 |
| 18139 | 19269 | 20226 | 21183 | 22140 | 18140 | 23097 | 24054 | 25011 | 25968 |
| 18141 | 19270 | 20227 | 21184 | 22141 | 18142 | 23098 | 24055 | 25012 | 25969 |
| 18143 | 19271 | 20228 | 21185 | 22142 | 18144 | 23099 | 24056 | 25013 | 25970 |
| 18145 | 19272 | 20229 | 21186 | 22143 | 18146 | 23100 | 24057 | 25014 | 25971 |
| 18147 | 19273 | 20230 | 21187 | 22144 | 18148 | 23101 | 24058 | 25015 | 25972 |
| 18149 | 19274 | 20231 | 21188 | 22145 | 18150 | 23102 | 24059 | 25016 | 25973 |
| 18151 | 19275 | 20232 | 21189 | 22146 | 18152 | 23103 | 24060 | 25017 | 25974 |
| 18153 | 19276 | 20233 | 21190 | 22147 | 18154 | 23104 | 24061 | 25018 | 25975 |
| 18155 | 19277 | 20234 | 21191 | 22148 | 18156 | 23105 | 24062 | 25019 | 25976 |
| 18157 | 19278 | 20235 | 21192 | 22149 | 18158 | 23106 | 24063 | 25020 | 25977 |
| 18159 | 19279 | 20236 | 21193 | 22150 | 18160 | 23107 | 24064 | 25021 | 25978 |
| 18161 | 19280 | 20237 | 21194 | 22151 | 18162 | 23108 | 24065 | 25022 | 25979 |
| 18163 | 19281 | 20238 | 21195 | 22152 | 18164 | 23109 | 24066 | 25023 | 25980 |
| 18165 | 19282 | 20239 | 21196 | 22153 | 18166 | 23110 | 24067 | 25024 | 25981 |
| 18167 | 19283 | 20240 | 21197 | 22154 | 18168 | 23111 | 24068 | 25025 | 25982 |
| 18169 | 19284 | 20241 | 21198 | 22155 | 18170 | 23112 | 24069 | 25026 | 25983 |
| 18171 | 19285 | 20242 | 21199 | 22156 | 18172 | 23113 | 24070 | 25027 | 25984 |
| 18173 | 19286 | 20243 | 21200 | 22157 | 18174 | 23114 | 24071 | 25028 | 25985 |
| 18175 | 19287 | 20244 | 21201 | 22158 | 18176 | 23115 | 24072 | 25029 | 25986 |
| 18177 | 19288 | 20245 | 21202 | 22159 | 18178 | 23116 | 24073 | 25030 | 25987 |
| 18179 | 19289 | 20246 | 21203 | 22160 | 18180 | 23117 | 24074 | 25031 | 25988 |
| 18181 | 19290 | 20247 | 21204 | 22161 | 18182 | 23118 | 24075 | 25032 | 25989 |
| 18183 | 19291 | 20248 | 21205 | 22162 | 18184 | 23119 | 24076 | 25033 | 25990 |
| 18185 | 19292 | 20249 | 21206 | 22163 | 18186 | 23120 | 24077 | 25034 | 25991 |
| 18187 | 19293 | 20250 | 21207 | 22164 | 18188 | 23121 | 24078 | 25035 | 25992 |
| 18189 | 19294 | 20251 | 21208 | 22165 | 18190 | 23122 | 24079 | 25036 | 25993 |
| 18191 | 19295 | 20252 | 21209 | 22166 | 18192 | 23123 | 24080 | 25037 | 25994 |
| 18193 | 19296 | 20253 | 21210 | 22167 | 18194 | 23124 | 24081 | 25038 | 25995 |
| 18195 | 19297 | 20254 | 21211 | 22168 | 18196 | 23125 | 24082 | 25039 | 25996 |
| 18197 | 19298 | 20255 | 21212 | 22169 | 18198 | 23126 | 24083 | 25040 | 25997 |
| 18199 | 19299 | 20256 | 21213 | 22170 | 18200 | 23127 | 24084 | 25041 | 25998 |
| 18201 | 19300 | 20257 | 21214 | 22171 | 18202 | 23128 | 24085 | 25042 | 25999 |
| 18203 | 19301 | 20258 | 21215 | 22172 | 18204 | 23129 | 24086 | 25043 | 26000 |
| 18205 | 19302 | 20259 | 21216 | 22173 | 18206 | 23130 | 24087 | 25044 | 26001 |
| 18207 | 19303 | 20260 | 21217 | 22174 | 18208 | 23131 | 24088 | 25045 | 26002 |
| 18209 | 19304 | 20261 | 21218 | 22175 | 18210 | 23132 | 24089 | 25046 | 26003 |
| 18211 | 19305 | 20262 | 21219 | 22176 | 18212 | 23133 | 24090 | 25047 | 26004 |
| 18213 | 19306 | 20263 | 21220 | 22177 | 18214 | 23134 | 24091 | 25048 | 26005 |
| 18215 | 19307 | 20264 | 21221 | 22178 | 18216 | 23135 | 24092 | 25049 | 26006 |
| 18217 | 19308 | 20265 | 21222 | 22179 | 18218 | 23136 | 24093 | 25050 | 26007 |
| 18219 | 19309 | 20266 | 21223 | 22180 | 18220 | 23137 | 24094 | 25051 | 26008 |
| 18221 | 19310 | 20267 | 21224 | 22181 | 18222 | 23138 | 24095 | 25052 | 26009 |
| 18223 | 19311 | 20268 | 21225 | 22182 | 18224 | 23139 | 24096 | 25053 | 26010 |
| 18225 | 19312 | 20269 | 21226 | 22183 | 18226 | 23140 | 24097 | 25054 | 26011 |
| 18227 | 19313 | 20270 | 21227 | 22184 | 18228 | 23141 | 24098 | 25055 | 26012 |
| 18229 | 19314 | 20271 | 21228 | 22185 | 18230 | 23142 | 24099 | 25056 | 26013 |
| 18231 | 19315 | 20272 | 21229 | 22186 | 18232 | 23143 | 24100 | 25057 | 26014 |
| 18233 | 19316 | 20273 | 21230 | 22187 | 18234 | 23144 | 24101 | 25058 | 26015 |
| 18235 | 19317 | 20274 | 21231 | 22188 | 18236 | 23145 | 24102 | 25059 | 26016 |
| 18237 | 19318 | 20275 | 21232 | 22189 | 18238 | 23146 | 24103 | 25060 | 26017 |
| 18239 | 19319 | 20276 | 21233 | 22190 | 18240 | 23147 | 24104 | 25061 | 26018 |
| 18241 | 19320 | 20277 | 21234 | 22191 | 18242 | 23148 | 24105 | 25062 | 26019 |
| 18243 | 19321 | 20278 | 21235 | 22192 | 18244 | 23149 | 24106 | 25063 | 26020 |
| 18245 | 19322 | 20279 | 21236 | 22193 | 18246 | 23150 | 24107 | 25064 | 26021 |
| 18247 | 19323 | 20280 | 21237 | 22194 | 18248 | 23151 | 24108 | 25065 | 26022 |
| 18249 | 19324 | 20281 | 21238 | 22195 | 18250 | 23152 | 24109 | 25066 | 26023 |
| 18251 | 19325 | 20282 | 21239 | 22196 | 18252 | 23153 | 24110 | 25067 | 26024 |
| 18253 | 19326 | 20283 | 21240 | 22197 | 18254 | 23154 | 24111 | 25068 | 26025 |
| 18255 | 19327 | 20284 | 21241 | 22198 | 18256 | 23155 | 24112 | 25069 | 26026 |
| 18257 | 19328 | 20285 | 21242 | 22199 | 18258 | 23156 | 24113 | 25070 | 26027 |
| 18259 | 19329 | 20286 | 21243 | 22200 | 18260 | 23157 | 24114 | 25071 | 26028 |
| 18261 | 19330 | 20287 | 21244 | 22201 | 18262 | 23158 | 24115 | 25072 | 26029 |
| 18263 | 19331 | 20288 | 21245 | 22202 | 18264 | 23159 | 24116 | 25073 | 26030 |
| 18265 | 19332 | 20289 | 21246 | 22203 | 18266 | 23160 | 24117 | 25074 | 26031 |
| 18267 | 19333 | 20290 | 21247 | 22204 | 18268 | 23161 | 24118 | 25075 | 26032 |
| 18269 | 19334 | 20291 | 21248 | 22205 | 18270 | 23162 | 24119 | 25076 | 26033 |
| 18271 | 19335 | 20292 | 21249 | 22206 | 18272 | 23163 | 24120 | 25077 | 26034 |
| 18273 | 19336 | 20293 | 21250 | 22207 | 18274 | 23164 | 24121 | 25078 | 26035 |
| 18275 | 19337 | 20294 | 21251 | 22208 | 18276 | 23165 | 24122 | 25079 | 26036 |
| 18277 | 19338 | 20295 | 21252 | 22209 | 18278 | 23166 | 24123 | 25080 | 26037 |
| 18279 | 19339 | 20296 | 21253 | 22210 | 18280 | 23167 | 24124 | 25081 | 26038 |
| 18281 | 19340 | 20297 | 21254 | 22211 | 18282 | 23168 | 24125 | 25082 | 26039 |
| 18283 | 19341 | 20298 | 21255 | 22212 | 18284 | 23169 | 24126 | 25083 | 26040 |
| 18285 | 19342 | 20299 | 21256 | 22213 | 18286 | 23170 | 24127 | 25084 | 26041 |
| 18287 | 19343 | 20300 | 21257 | 22214 | 18288 | 23171 | 24128 | 25085 | 26042 |
| 18289 | 19344 | 20301 | 21258 | 22215 | 18290 | 23172 | 24129 | 25086 | 26043 |
| 18291 | 19345 | 20302 | 21259 | 22216 | 18292 | 23173 | 24130 | 25087 | 26044 |
| 18293 | 19346 | 20303 | 21260 | 22217 | 18294 | 23174 | 24131 | 25088 | 26045 |
| 18295 | 19347 | 20304 | 21261 | 22218 | 18296 | 23175 | 24132 | 25089 | 26046 |
| 18297 | 19348 | 20305 | 21262 | 22219 | 18298 | 23176 | 24133 | 25090 | 26047 |
| 18299 | 19349 | 20306 | 21263 | 22220 | 18300 | 23177 | 24134 | 25091 | 26048 |
| 18301 | 19350 | 20307 | 21264 | 22221 | 18302 | 23178 | 24135 | 25092 | 26049 |
| 18303 | 19351 | 20308 | 21265 | 22222 | 18304 | 23179 | 24136 | 25093 | 26050 |
| 18305 | 19352 | 20309 | 21266 | 22223 | 18306 | 23180 | 24137 | 25094 | 26051 |
| 18307 | 19353 | 20310 | 21267 | 22224 | 18308 | 23181 | 24138 | 25095 | 26052 |
| 18309 | 19354 | 20311 | 21268 | 22225 | 18310 | 23182 | 24139 | 25096 | 26053 |
| 18311 | 19355 | 20312 | 21269 | 22226 | 18312 | 23183 | 24140 | 25097 | 26054 |
| 18313 | 19356 | 20313 | 21270 | 22227 | 18314 | 23184 | 24141 | 25098 | 26055 |
| 18315 | 19357 | 20314 | 21271 | 22228 | 18316 | 23185 | 24142 | 25099 | 26056 |
| 18317 | 19358 | 20315 | 21272 | 22229 | 18318 | 23186 | 24143 | 25100 | 26057 |
| 18319 | 19359 | 20316 | 21273 | 22230 | 18320 | 23187 | 24144 | 25101 | 26058 |
| 18321 | 19360 | 20317 | 21274 | 22231 | 18322 | 23188 | 24145 | 25102 | 26059 |
| 18323 | 19361 | 20318 | 21275 | 22232 | 18324 | 23189 | 24146 | 25103 | 26060 |
| 18325 | 19362 | 20319 | 21276 | 22233 | 18326 | 23190 | 24147 | 25104 | 26061 |
| 18327 | 19363 | 20320 | 21277 | 22234 | 18328 | 23191 | 24148 | 25105 | 26062 |
| 18329 | 19364 | 20321 | 21278 | 22235 | 18330 | 23192 | 24149 | 25106 | 26063 |
| 18331 | 19365 | 20322 | 21279 | 22236 | 18332 | 23193 | 24150 | 25107 | 26064 |
| 18333 | 19366 | 20323 | 21280 | 22237 | 18334 | 23194 | 24151 | 25108 | 26065 |
| 18335 | 19367 | 20324 | 21281 | 22238 | 18336 | 23195 | 24152 | 25109 | 26066 |
| 18337 | 19368 | 20325 | 21282 | 22239 | 18338 | 23196 | 24153 | 25110 | 26067 |
| 18339 | 19369 | 20326 | 21283 | 22240 | 18340 | 23197 | 24154 | 25111 | 26068 |
| 18341 | 19370 | 20327 | 21284 | 22241 | 18342 | 23198 | 24155 | 25112 | 26069 |
| 18343 | 19371 | 20328 | 21285 | 22242 | 18344 | 23199 | 24156 | 25113 | 26070 |
| 18345 | 19372 | 20329 | 21286 | 22243 | 18346 | 23200 | 24157 | 25114 | 26071 |
| 18347 | 19373 | 20330 | 21287 | 22244 | 18348 | 23201 | 24158 | 25115 | 26072 |
| 18349 | 19374 | 20331 | 21288 | 22245 | 18350 | 23202 | 24159 | 25116 | 26073 |
| 18351 | 19375 | 20332 | 21289 | 22246 | 18352 | 23203 | 24160 | 25117 | 26074 |
| 18353 | 19376 | 20333 | 21290 | 22247 | 18354 | 23204 | 24161 | 25118 | 26075 |
| 18355 | 19377 | 20334 | 21291 | 22248 | 18356 | 23205 | 24162 | 25119 | 26076 |
| 18357 | 19378 | 20335 | 21292 | 22249 | 18358 | 23206 | 24163 | 25120 | 26077 |
| 18359 | 19379 | 20336 | 21293 | 22250 | 18360 | 23207 | 24164 | 25121 | 26078 |
| 18361 | 19380 | 20337 | 21294 | 22251 | 18362 | 23208 | 24165 | 25122 | 26079 |
| 18363 | 19381 | 20338 | 21295 | 22252 | 18364 | 23209 | 24166 | 25123 | 26080 |
| 18365 | 19382 | 20339 | 21296 | 22253 | 18366 | 23210 | 24167 | 25124 | 26081 |
| 18367 | 19383 | 20340 | 21297 | 22254 | 18368 | 23211 | 24168 | 25125 | 26082 |
| 18369 | 19384 | 20341 | 21298 | 22255 | 18370 | 23212 | 24169 | 25126 | 26083 |
| 18371 | 19385 | 20342 | 21299 | 22256 | 18372 | 23213 | 24170 | 25127 | 26084 |
| 18373 | 19386 | 20343 | 21300 | 22257 | 18374 | 23214 | 24171 | 25128 | 26085 |
| 18375 | 19387 | 20344 | 21301 | 22258 | 18376 | 23215 | 24172 | 25129 | 26086 |
| 18377 | 19388 | 20345 | 21302 | 22259 | 18378 | 23216 | 24173 | 25130 | 26087 |
| 18379 | 19389 | 20346 | 21303 | 22260 | 18380 | 23217 | 24174 | 25131 | 26088 |
| 18381 | 19390 | 20347 | 21304 | 22261 | 18382 | 23218 | 24175 | 25132 | 26089 |
| 18383 | 19391 | 20348 | 21305 | 22262 | 18384 | 23219 | 24176 | 25133 | 26090 |
| 18385 | 19392 | 20349 | 21306 | 22263 | 18386 | 23220 | 24177 | 25134 | 26091 |
| 18387 | 19393 | 20350 | 21307 | 22264 | 18388 | 2322 | 24178 | 25135 | 26092 |
| 18389 | 19394 | 20351 | 21308 | 22265 | 18390 | 23222 | 24179 | 25136 | 26093 |
| 18391 | 19395 | 20352 | 21309 | 22266 | 18392 | 23223 | 24180 | 25137 | 26094 |
| 18393 | 19396 | 20353 | 21310 | 22267 | 18394 | 23224 | 24181 | 25138 | 26095 |
| 18395 | 19397 | 20354 | 21311 | 22268 | 18396 | 23225 | 24182 | 25139 | 26096 |
| 18397 | 19398 | 20355 | 21312 | 22269 | 18398 | 23226 | 24183 | 25140 | 26097 |
| 18399 | 19399 | 20356 | 21313 | 22270 | 18400 | 23227 | 24184 | 25141 | 26098 |
| 18401 | 19400 | 20357 | 21314 | 22271 | 18402 | 23228 | 24185 | 25142 | 26099 |
| 18403 | 19401 | 20358 | 21315 | 22272 | 18404 | 23229 | 24186 | 25143 | 26100 |
| 18405 | 19402 | 20359 | 21316 | 22273 | 18406 | 23230 | 24187 | 25144 | 26101 |
| 18407 | 19403 | 20360 | 21317 | 22274 | 18408 | 23231 | 24188 | 25145 | 26102 |
| 18409 | 19404 | 20361 | 21318 | 22275 | 18410 | 23232 | 24189 | 25146 | 26103 |
| 18411 | 19405 | 20362 | 21319 | 22276 | 18412 | 23233 | 24190 | 25147 | 26104 |
| 18413 | 19406 | 20363 | 21320 | 22277 | 18414 | 23234 | 24191 | 25148 | 26105 |
| 18415 | 19407 | 20364 | 21321 | 22278 | 18416 | 23235 | 24192 | 25149 | 26106 |
| 18417 | 19408 | 20365 | 21322 | 22279 | 18418 | 23236 | 24193 | 25150 | 26107 |
| 18419 | 19409 | 20366 | 21323 | 22280 | 18420 | 23237 | 24194 | 25151 | 26108 |
| 18421 | 19410 | 20367 | 21324 | 22281 | 18422 | 23238 | 24195 | 25152 | 26109 |
| 18423 | 19411 | 20368 | 21325 | 22282 | 18424 | 23239 | 24196 | 25153 | 26110 |
| 18425 | 19412 | 20369 | 21326 | 22283 | 18426 | 23240 | 24197 | 25154 | 26111 |
| 18427 | 19413 | 20370 | 21327 | 22284 | 18428 | 23241 | 24198 | 25155 | 26112 |
| 18429 | 19414 | 20371 | 21328 | 22285 | 18430 | 23242 | 24199 | 25156 | 26113 |
| 18431 | 19415 | 20372 | 21329 | 22286 | 18432 | 23243 | 24200 | 25157 | 26114 |
| 18433 | 19416 | 20373 | 21330 | 22287 | 18434 | 23244 | 24201 | 25158 | 26115 |
| 18435 | 19417 | 20374 | 21331 | 22288 | 18436 | 23245 | 24202 | 25159 | 26116 |
| 18437 | 19418 | 20375 | 21332 | 22289 | 18438 | 23246 | 24203 | 25160 | 26117 |
| 18439 | 19419 | 20376 | 21333 | 22290 | 18440 | 23247 | 24204 | 25161 | 26118 |
| 18441 | 19420 | 20377 | 21334 | 22291 | 18442 | 23248 | 24205 | 25162 | 26119 |
| 18443 | 19421 | 20378 | 21335 | 22292 | 18444 | 23249 | 24206 | 25163 | 26120 |
| 18445 | 19422 | 20379 | 21336 | 22293 | 18446 | 23250 | 24207 | 25164 | 26121 |
| 18447 | 19423 | 20380 | 21337 | 22294 | 18448 | 23251 | 24208 | 25165 | 26122 |
| 18449 | 19424 | 20381 | 21338 | 22295 | 18450 | 23252 | 24209 | 25166 | 26123 |
| 18451 | 19425 | 20382 | 21339 | 22296 | 18452 | 23253 | 24210 | 25167 | 26124 |
| 18453 | 19426 | 20383 | 21340 | 22297 | 18454 | 23254 | 24211 | 25168 | 26125 |
| 18455 | 19427 | 20384 | 21341 | 22298 | 18456 | 23255 | 24212 | 25169 | 26126 |
| 18457 | 19428 | 20385 | 21342 | 22299 | 18458 | 23256 | 24213 | 25170 | 26127 |
| 18459 | 19429 | 20386 | 21343 | 22300 | 18460 | 23257 | 24214 | 25171 | 26128 |
| 18461 | 19430 | 20387 | 21344 | 22301 | 18462 | 23258 | 24215 | 25172 | 26129 |
| 18463 | 19431 | 20388 | 21345 | 22302 | 18464 | 23259 | 24216 | 25173 | 26130 |
| 18465 | 19432 | 20389 | 21346 | 22303 | 18466 | 23260 | 24217 | 25174 | 26131 |
| 18467 | 19433 | 20390 | 21347 | 22304 | 18468 | 23261 | 24218 | 25175 | 26132 |
| 18469 | 19434 | 20391 | 21348 | 22305 | 18470 | 23262 | 24219 | 25176 | 26133 |
| 18471 | 19435 | 20392 | 21349 | 22306 | 18472 | 23263 | 24220 | 25177 | 26134 |
| 18473 | 19436 | 20393 | 21350 | 22307 | 18474 | 23264 | 24221 | 25178 | 26135 |
| 18475 | 19437 | 20394 | 21351 | 22308 | 18476 | 23265 | 24222 | 25179 | 26136 |
| 18477 | 19438 | 20395 | 21352 | 22309 | 18478 | 23266 | 24223 | 25180 | 26137 |
| 18479 | 19439 | 20396 | 21353 | 22310 | 18480 | 23267 | 24224 | 25181 | 26138 |
| 18481 | 19440 | 20397 | 21354 | 22311 | 18482 | 23268 | 24225 | 25182 | 26139 |
| 18483 | 19441 | 20398 | 21355 | 22312 | 18484 | 23269 | 24226 | 25183 | 26140 |

TABLE 3
5885 Antibodies

| Antibody | SEQ ID | | | | Antibody | SEQ ID | | | |
|---|---|---|---|---|---|---|---|---|---|
| Name and | NO of | | | | Name and | NO of | | | |
| Heavy Chain | V-D-J | SEQ ID | SEQ ID | SEQ ID | Light Chain | V-J | SEQ ID | SEQ ID | SEQ ID |
| (HC) | Region | NO of | NO of | NO of | (LC) | Region | NO of | NO of | NO of |
| designation | (HC) | CDRH1 | CDRH2 | CDRH3 | designation | (LC) | CDRL1 | CDRL2 | CDRL3 |
| 5885_1_HC | 26141 | 26167 | 26180 | 26193 | 5885_1_LC | 26154 | 26206 | 26219 | 26232 |
| 5885_2_HC | 26142 | 26168 | 26181 | 26194 | 5885_2_LC | 26155 | 26207 | 26220 | 26233 |
| 5885_3_HC | 26143 | 26169 | 26182 | 26195 | 5885_3_LC | 26156 | 26208 | 26221 | 26234 |
| 5885_4_HC | 26144 | 26170 | 26183 | 26196 | 5885_4_LC | 26157 | 26209 | 26222 | 26235 |
| 5885_5_HC | 26145 | 26171 | 26184 | 26197 | 5885_5_LC | 26158 | 26210 | 26223 | 26236 |
| 5885_6_HC | 26146 | 26172 | 26185 | 26198 | 5885_6_LC | 26159 | 26211 | 26224 | 26237 |
| 5885_7_HC | 26147 | 26173 | 26186 | 26199 | 5885_7_LC | 26160 | 26212 | 26225 | 26238 |
| 5885_8_HC | 26148 | 26174 | 26187 | 26200 | 5885_8_LC | 26161 | 26213 | 26226 | 26239 |
| 5885_9_HC | 26149 | 26175 | 26188 | 26201 | 5885_9_LC | 26162 | 26214 | 26227 | 26240 |
| 5885_10_HC | 26150 | 26176 | 26189 | 26202 | 5885_10_LC | 26163 | 26215 | 26228 | 26241 |
| 5885_11_HC | 26151 | 26177 | 26190 | 26203 | 5885_11_LC | 26164 | 26216 | 26229 | 26242 |
| 5885_12_HC | 26152 | 26178 | 26191 | 26204 | 5885_12_LC | 26165 | 26217 | 26230 | 26243 |
| 5885_13_HC | 26153 | 26179 | 26192 | 26205 | 5885_13_LC | 26166 | 26218 | 26231 | 26244 |

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

1. A recombinant antibody, wherein the antibody comprises a light chain variable region (VL) that comprises a light chain complementarity determining region (CDRL)1, CDRL2, and CDRL3 and/or a heavy chain variable region (VH) that comprises a heavy chain complementarity determining region (CDRH)1, CDRH2, and CDRH3, wherein:
CDRH3 comprises an amino acid sequence at least 60% identical to SEQ ID NOs: 4969-5796, 13255-14083, 21356-22312, 26193-26205, 26263-26275, or 26289-26318; and
CDRL3 comprises an amino acid sequence at least 60% identical to SEQ ID NOs: 7453-8280, 15742-16570, 25184-26140, 26232-26244, 26276-26288, or 26319-26348.

2. The recombinant antibody of claim 1, wherein CDRH3 comprises at least one amino acid substitution when compared to SEQ ID NOs: 4969-5796, 13255-14083, 21356-22312, 26193-26205, 26263-26275, or 26289-26318.

3. The recombinant antibody of claim 1, wherein CDRL3 comprises at least one amino acid substitution when compared to SEQ ID NOs: 7453-8280, 15742-16570, 25184-26140, 26232-26244, 26276-26288, or 26319-26348.

4. The recombinant antibody of claim 1, wherein:
CDRH1 comprises an amino acid sequence at least 60% identical to SEQ ID NOs: 3313-4140, 11597-12425, 19442-20398, or 26167-26179; and/or
CDRL1 comprises an amino acid sequence at least 60% identical to SEQ ID NOs: 5797-6624, 14084-14912, 23270-24226, or 26206-26218.

5. The recombinant antibody of claim 1, wherein CDRH1 comprises at least one amino acid substitution when compared to SEQ ID NOs: 3313-4140, 11597-12425, or 19442-20398, or 26167-26179.

6. The recombinant antibody of claim 1, wherein CDRL1 comprises at least one amino acid substitution when compared to SEQ ID NOs: 5797-6624, 14084-14912, or 23270-24226, or 26206-26218.

7. The recombinant antibody of claim 1, wherein:
CDRH2 comprises an amino acid sequence at least 60% identical to SEQ ID NOs: 4141-4968, 12426-13254, 20399-21355, or 26180-26192; and/or
CDRL2 comprises an amino acid sequence at least 60% identical to SEQ ID NOs: 6625-7452, 14913-15741, 24227-25183, or 26219-26231.

8. The recombinant antibody of claim 1, wherein CDRH2 comprises at least one amino acid substitution when compared to SEQ ID NOs: 4141-4968, 12426-13254, 20399-21355, or 26180-26192.

9. The recombinant antibody of claim 1, wherein CDRL2 comprises at least one amino acid substitution when compared to SEQ ID NOs: 6625-7452, 14913-15741, 24227-25183, or 26219-26231.

10. The recombinant antibody of claim 1, wherein VH comprises an amino acid sequence selected from the group consisting of SEQ ID NOs: 1657-2484, 9939-10767, 18485-19441, and 26141-26153.

11. The recombinant antibody of claim 1, wherein VL comprises an amino acid sequence selected from the group consisting of SEQ ID NOs: 2485-3312, 10768-11596, 22313-23269, and 26154-26166.

12. The recombinant antibody of claim 1, wherein the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 4056,
CDRH2 is SEQ ID NO: 4884,
CDRH3 is SEQ ID NO: 5712,
CDRL1 is SEQ ID NO: 6540,
CDRL2 is SEQ ID NO: 7368, and
CDRL3 is SEQ ID NO: 8196;
or
CDRH1 is SEQ ID NO: 3791,
CDRH2 is SEQ ID NO: 4619,
CDRH3 is SEQ ID NO: 5447,
CDRL1 is SEQ ID NO: 6275,
CDRL2 is SEQ ID NO: 7103, and
CDRL3 is SEQ ID NO: 7931;
or
CDRH1 is SEQ ID NO: 3858,
CDRH2 is SEQ ID NO: 4686,
CDRH3 is SEQ ID NO: 5514,
CDRL1 is SEQ ID NO: 6342,
CDRL2 is SEQ ID NO: 7170, and
CDRL3 is SEQ ID NO: 7998;
or
CDRH1 is SEQ ID NO: 3680,
CDRH2 is SEQ ID NO: 4508,
CDRH3 is SEQ ID NO: 5336,
CDRL1 is SEQ ID NO: 6164,
CDRL2 is SEQ ID NO: 6992, and
CDRL3 is SEQ ID NO: 7820;
or
CDRH1 is SEQ ID NO: 3856,
CDRH2 is SEQ ID NO: 4684,
CDRH3 is SEQ ID NO: 5512,
CDRL1 is SEQ ID NO: 6340,
CDRL2 is SEQ ID NO: 7168, and
CDRL3 is SEQ ID NO: 7996;
or
CDRH1 is SEQ ID NO: 3355,
CDRH2 is SEQ ID NO: 4183,
CDRH3 is SEQ ID NO: 5011,
CDRL1 is SEQ ID NO: 5839,
CDRL2 is SEQ ID NO: 6667, and
CDRL3 is SEQ ID NO: 7495;
or
CDRH1 is SEQ ID NO: 3697,
CDRH2 is SEQ ID NO: 4525,
CDRH3 is SEQ ID NO: 5353,
CDRL1 is SEQ ID NO: 6181,
CDRL2 is SEQ ID NO: 7009, and
CDRL3 is SEQ ID NO: 7837;
or
CDRH1 is SEQ ID NO: 3481,
CDRH2 is SEQ ID NO: 4309,
CDRH3 is SEQ ID NO: 5137,
CDRL1 is SEQ ID NO: 5965,
CDRL2 is SEQ ID NO: 6793, and
CDRL3 is SEQ ID NO: 7621;
or
CDRH1 is SEQ ID NO: 3896,
CDRH2 is SEQ ID NO: 4724,
CDRH3 is SEQ ID NO: 5552,
CDRL1 is SEQ ID NO: 6380,
CDRL2 is SEQ ID NO: 7208, and
CDRL3 is SEQ ID NO: 8036;
or
CDRH1 is SEQ ID NO: 3667,
CDRH2 is SEQ ID NO: 4495,
CDRH3 is SEQ ID NO: 5323,
CDRL1 is SEQ ID NO: 6151,
CDRL2 is SEQ ID NO: 6979, and
CDRL3 is SEQ ID NO: 7807;
or
CDRH1 is SEQ ID NO: 12368,
CDRH2 is SEQ ID NO: 13197,
CDRH3 is SEQ ID NO: 14026,
CDRL1 is SEQ ID NO: 14855,
CDRL2 is SEQ ID NO: 15684, and
CDRL3 is SEQ ID NO: 16513;
or
CDRH1 is SEQ ID NO: 11621,
CDRH2 is SEQ ID NO: 12450,
CDRH3 is SEQ ID NO: 13279,
CDRL1 is SEQ ID NO: 14108,
CDRL2 is SEQ ID NO: 14937, and
CDRL3 is SEQ ID NO: 15766;
or
CDRH1 is SEQ ID NO: 11742,
CDRH2 is SEQ ID NO: 12571,
CDRH3 is SEQ ID NO: 13400,
CDRL1 is SEQ ID NO: 14229,
CDRL2 is SEQ ID NO: 15058, and
CDRL3 is SEQ ID NO: 15887;
or
CDRH1 is SEQ ID NO: 11598,
CDRH2 is SEQ ID NO: 12427,
CDRH3 is SEQ ID NO: 13256,
CDRL1 is SEQ ID NO: 14085,
CDRL2 is SEQ ID NO: 14914, and
CDRL3 is SEQ ID NO: 15743;
or
CDRH1 is SEQ ID NO: 12262,
CDRH2 is SEQ ID NO: 13091,
CDRH3 is SEQ ID NO: 13920,
CDRL1 is SEQ ID NO: 14749,
CDRL2 is SEQ ID NO: 15578, and
CDRL3 is SEQ ID NO: 16407;
or
CDRH1 is SEQ ID NO: 11995,
CDRH2 is SEQ ID NO: 12824,
CDRH3 is SEQ ID NO: 13653,
CDRL1 is SEQ ID NO: 14482,
CDRL2 is SEQ ID NO: 15311, and
CDRL3 is SEQ ID NO: 16140;
or
CDRH1 is SEQ ID NO: 12164,
CDRH2 is SEQ ID NO: 12993,
CDRH3 is SEQ ID NO: 13822,
CDRL1 is SEQ ID NO: 14651,
CDRL2 is SEQ ID NO: 15480, and
CDRL3 is SEQ ID NO: 16309;
or
CDRH1 is SEQ ID NO: 11752,
CDRH2 is SEQ ID NO: 12581,
CDRH3 is SEQ ID NO: 13410,
CDRL1 is SEQ ID NO: 14239,
CDRL2 is SEQ ID NO: 15068, and
CDRL3 is SEQ ID NO: 15897;
or
CDRH1 is SEQ ID NO: 11888,
CDRH2 is SEQ ID NO: 12717,
CDRH3 is SEQ ID NO: 13546,
CDRL1 is SEQ ID NO: 14375,
CDRL2 is SEQ ID NO: 15204, and
CDRL3 is SEQ ID NO: 16033;
or
CDRH1 is SEQ ID NO: 20173,
CDRH2 is SEQ ID NO: 21130,
CDRH3 is SEQ ID NO: 22087,
CDRL1 is SEQ ID NO: 24001,
CDRL2 is SEQ ID NO: 24958, and
CDRL3 is SEQ ID NO: 25915;
or
CDRH1 is SEQ ID NO: 20065,
CDRH2 is SEQ ID NO: 21022,
CDRH3 is SEQ ID NO: 21979,
CDRL1 is SEQ ID NO: 23893,
CDRL2 is SEQ ID NO: 24850, and
CDRL3 is SEQ ID NO: 25807;
or
CDRH1 is SEQ ID NO: 20115,
CDRH2 is SEQ ID NO: 21072,
CDRH3 is SEQ ID NO: 22029,
CDRL1 is SEQ ID NO: 23943,
CDRL2 is SEQ ID NO: 24900, and
CDRL3 is SEQ ID NO: 25857;
or
CDRH1 is SEQ ID NO: 19873,
CDRH2 is SEQ ID NO: 20830,
CDRH3 is SEQ ID NO: 21787,
CDRL1 is SEQ ID NO: 23701,
CDRL2 is SEQ ID NO: 24658, and
CDRL3 is SEQ ID NO: 25615;
or
CDRH1 is SEQ ID NO: 19923,
CDRH2 is SEQ ID NO: 20880,
CDRH3 is SEQ ID NO: 21837,
CDRL1 is SEQ ID NO: 23751,
CDRL2 is SEQ ID NO: 24708, and
CDRL3 is SEQ ID NO: 25665;
or
CDRH1 is SEQ ID NO: 19458,
CDRH2 is SEQ ID NO: 20415,
CDRH3 is SEQ ID NO: 21372,
CDRL1 is SEQ ID NO: 23286,
CDRL2 is SEQ ID NO: 24243, and
CDRL3 is SEQ ID NO: 25200;
or
CDRH1 is SEQ ID NO: 20235,
CDRH2 is SEQ ID NO: 21192,
CDRH3 is SEQ ID NO: 22149,
CDRL1 is SEQ ID NO: 24063,
CDRL2 is SEQ ID NO: 25020, and
CDRL3 is SEQ ID NO: 25977;
or
CDRH1 is SEQ ID NO: 19858,
CDRH2 is SEQ ID NO: 20815,
CDRH3 is SEQ ID NO: 21772,
CDRL1 is SEQ ID NO: 23686,
CDRL2 is SEQ ID NO: 24643, and
CDRL3 is SEQ ID NO: 25600;
or
CDRH1 is SEQ ID NO: 19735,
CDRH2 is SEQ ID NO: 20692,
CDRH3 is SEQ ID NO: 21649,
CDRL1 is SEQ ID NO: 23563,
CDRL2 is SEQ ID NO: 24520, and
CDRL3 is SEQ ID NO: 25477;
or
CDRH1 is SEQ ID NO: 19887,
CDRH2 is SEQ ID NO: 20844,
CDRH3 is SEQ ID NO: 21801,
CDRL1 is SEQ ID NO: 23715,
CDRL2 is SEQ ID NO: 24672, and
CDRL3 is SEQ ID NO: 25356;
or
CDRH1 is SEQ ID NO: 19614,
CDRH2 is SEQ ID NO: 20571,
CDRH3 is SEQ ID NO: 21528,
CDRL1 is SEQ ID NO: 23442,
CDRL2 is SEQ ID NO: 24399, and
CDRL3 is SEQ ID NO: 25986.

13.-41. (canceled)

42. The recombinant antibody of claim 1, wherein the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRH1 is SEQ ID NO: 26167,
CDRH2 is SEQ ID NO: 26180,
CDRH3 is SEQ ID NO: 26193,
CDRL1 is SEQ ID NO: 26206,
CDRL2 is SEQ ID NO: 26219, and
CDRL3 is SEQ ID NO: 26232;
or
CDRH1 is SEQ ID NO: 26168,
CDRH2 is SEQ ID NO: 26181,
CDRH3 is SEQ ID NO: 26194,
CDRL1 is SEQ ID NO: 26207,
CDRL2 is SEQ TD NO: 26220, and
CDRL3 is SEQ ID NO: 26233;
or
CDRH1 is SEQ ID NO: 26169,
CDRH2 is SEQ ID NO: 26182,
CDRH3 is SEQ ID NO: 26195,
CDRL1 is SEQ ID NO: 26208,
CDRL2 is SEQ ID NO: 26221, and
CDRL3 is SEQ ID NO: 26234;
or
CDRH1 is SEQ ID NO: 26170,
CDRH2 is SEQ ID NO: 26183,
CDRH3 is SEQ ID NO: 26196,
CDRL1 is SEQ ID NO: 26209,
CDRL2 is SEQ ID NO: 26222; and
CDRL3 is SEQ ID NO: 26235,
or
CDRH1 is SEQ ID NO: 26171,
CDRH2 is SEQ ID NO: 26184,
CDRH3 is SEQ ID NO: 26197,
CDRL1 is SEQ ID NO: 26210;
CDRL2 is SEQ ID NO: 26223, and
CDRL3 is SEQ ID NO: 26236,
or
CDRH1 is SEQ ID NO: 26172,
CDRH2 is SEQ ID NO: 26185,
CDRH3 is SEQ ID NO: 26198,
CDRL1 is SEQ ID NO: 26211,
CDRL2 is SEQ ID NO: 26224; and
CDRL3 is SEQ ID NO: 26237,
or
CDRH1 is SEQ ID NO: 26173,
CDRH2 is SEQ ID NO: 26186,
CDRH3 is SEQ ID NO: 26199,
CDRL1 is SEQ ID NO: 26212,
CDRL2 is SEQ ID NO: 26225, and
CDRL3 is SEQ ID NO: 26238;
or
CDRH1 is SEQ ID NO: 26174,
CDRH2 is SEQ ID NO: 26187,
CDRH3 is SEQ ID NO: 26200,
CDRL1 is SEQ ID NO: 26213,
CDRL2 is SEQ ID NO: 26226, and
CDRL3 is SEQ ID NO: 26239;
or
CDRH1 is SEQ ID NO: 26175,
CDRH2 is SEQ ID NO: 26188,
CDRH3 is SEQ ID NO: 26201,
CDRL1 is SEQ ID NO: 26214,
CDRL2 is SEQ ID NO: 26227, and
CDRL3 is SEQ ID NO: 26240;
or
CDRH1 is SEQ ID NO: 26176,
CDRH2 is SEQ ID NO: 26189,
CDRH3 is SEQ ID NO: 26202,
CDRL1 is SEQ ID NO: 26215,
CDRL2 is SEQ ID NO: 26228; and
CDRL3 is SEQ TD NO: 26241,
or
CDRH1 is SEQ ID NO: 26177,
CDRH2 is SEQ ID NO: 26190,
CDRH3 is SEQ ID NO: 26203,
CDRL1 is SEQ ID NO: 26216,
CDRL2 is SEQ ID NO: 26229, and
CDRL3 is SEQ ID NO: 26242;
or
CDRH1 is SEQ ID NO: 26178,
CDRH2 is SEQ ID NO: 26191,
CDRH3 is SEQ ID NO: 26204,
CDRL1 is SEQ ID NO: 26217,
CDRL2 is SEQ ID NO: 26230; and
CDRL3 is SEQ ID NO: 26243;
or
CDRH1 is SEQ ID NO: 26179,
CDRH2 is SEQ ID NO: 26192,
CDRH3 is SEQ ID NO: 26205,
CDRL1 is SEQ ID NO: 26218,
CDRL2 is SEQ ID NO: 26231, and
CDRL3 is SEQ ID NO: 26244.

43.-54. (canceled)

55. A nucleic acid encoding the recombinant antibody of claim 1.

56. A recombinant expression cassette or plasmid comprising a sequence to express the recombinant antibody of claim 1.

57. A host cell comprising the expression cassette or the plasmid of claim 56.

58. A method of producing an antibody, comprising cultivating or maintaining the host cell of claim 57 under conditions to produce the antibody.

59. A method of preventing or treating a coronavirus infection in a subject, comprising administering to the subject a therapeutically effective amount of the recombinant antibody of claim 1.

60. The method of claim 59, where the coronavirus is SARS-CoV-2.
